(19) United States
(12) Patent Application Publication
(10) Pub. No.: US 2011/0300128 A1
(43) Pub. Date: Dec. 8, 2011

(54) USE OF FOXP2 AS A MARKER FOR ABNORMAL LYMPHOCYTES AND AS A TARGET FOR THERAPY OF DISORDERS ASSOCIATED WITH ABNORMAL LYMPHOCYTES

(75) Inventors: Alison Banham, Oxfordshire (GB); Andrew Campbell, Oxford (GB); Duncan Gascoyne, Nr Preston (GB); Linden Lyne, Oxon (GB)

(73) Assignee: ISIS INNOVATION LIMITED, Oxford (GB)

(21) Appl. No.: 13/139,028

(22) PCT Filed: Dec. 16, 2009
(86) PCT No.: PCT/GB2009/002887

§ 371 Date: Aug. 10, 2011

(30) Foreign Application Priority Data
Dec. 17, 2008 (GB) 0823020.3

(51) Int. Cl. A61K 39/395; C12N 5/16; G01N 33/574; A61P 7/00; A61K 31/713; A61K 38/02; C07K 16/18; A61P 35/00; C12Q 1/68; G01N 33/566

(52) U.S. Cl. 424/130.1; 435/6.1; 435/6.14; 435/346; 435/6.12; 435/7.1; 514/44 A; 514/1.1; 530/389.1; 514/16.7

(57) ABSTRACT

The present invention is directed to a method for detecting abnormal lymphocytes said method comprising detecting an amount or expression of the FOXP2 gene in lymphocytes in a sample, wherein an increased amount or expression of the FOXP2 gene in said lymphocytes indicates the presence of abnormal lymphocytes. Additionally, the invention concerns a method for detecting or assessing a condition associated with the presence of abnormal lymphocytes. The methods of the invention may also be useful for diagnosing myeloma or MGUS or for determining the prognosis for patients with lymphoma, myeloma or MGUS. The severity of bone disease or bone colonisation of tumours may also be able to be predicted. Further, treatment of conditions associated with the presence of abnormal lymphocytes using an agent which inhibits FOXP2 expression and/or FOXP2 activity is provided. An antibody which binds to the N-terminus of FOXP2 has also been developed.

The present invention is concerned with FOXP2 and its use as a marker to detect abnormal e.g. malignant or pre-malignant lymphocytes. Such abnormal lymphocytes may occur in a variety of conditions, namely certain haematological disorders or malignancies and accordingly their detection may be used to assist in the diagnosis and/or prognosis of such conditions. In particular, FOXP2 expression in lymphocytes may be used as a marker for malignancy. Further, the invention relates to the use of agents capable of reducing or inhibiting FOXP2 expression or activity for the treatment or prevention of conditions or disorders associated with abnormal lymphocytes, or abnormal FOXP2 expression in lymphocytes, for example myeloma or lymphoma. The invention also concerns a monoclonal antibody which binds specifically to FOXP2.

The forkhead box (FOX) proteins are a family of transcription factors defined by a common DNA-binding domain termed the forkhead box or winged helix domain. Many different transcription factors have been identified in this family and FOX protein family members are important for a wide range of biological processes, including development, metabolism, proliferation, differentiation, migration and longevity. There are at least 17 FOX gene subfamilies (FOXA-R) with at least 41 genes identified in humans.

The FOXP family of transcription factors is a subfamily of the FOX protein family, with four currently identified members, namely FOXP1, FOXP2, FOXP3 and FOXP4. These forkhead proteins have an atypical C-terminal forkhead domain and an N-terminal C2H2 zinc finger motif. The FOXP transcription factors are able to bind to a common consensus DNA sequence and their homo and/or heterodimerisation is required for DNA binding.

FOX transcription factors, including members of the FOXP sub-family, are increasingly being implicated in cancer and suggestions have been made that they may be useful as biomarkers and potential targets for therapy. The picture as regards different specific factors is however not entirely consistent.

FOXP1 has been identified as a candidate tumour suppressor gene (Banham et al, 2001, Cancer Res., 61, 8820-8829). Additionally and in contrast, it has also been reported that FOXP 1 expression can be de-regulated by recurrent chromosome translocations in B-cell lymphoma patients and that high levels of protein expression are associated with a poor prognosis (Banham et al, 2005, Clin. Cancer Res., 11 (3), 1065-1072), although this may reflect the expression of potentially smaller oncogenic isoforms of the FOXP1 protein (Brown et al, 2008, Blood, 111 (5), 2816-2824). Retroviral insertion into the 5′ coding sequence of the foxP1 gene has been reported as an oncogenic event in an avian nephroblastoma model (Pajer et al, 2006, Cancer Res., 66 (1), 78-86).

FOXP3 has a role in regulating the development of B cells, T cells and CD25⁺CD4⁺ regulatory T cells. It is expressed in adult T-cell leukaemia/lymphoma and is widely used as a marker of a population of regulatory T cells (Tregs) that control immunotolerance and enable tumour cells to evade the host response (Bignone and Banham, 2008, EOBT, 8 (12), 1897-1920).

The role of FOXP4 in malignancy has not been extensively studied. It has been reported that FOXP4 mRNA is down regulated in kidney tumours and a balanced chromosome translocation involving FOXP4 in a human breast cancer cell line has been identified (Teufel et al, 2003, Biochim Biophy Acta, 1627, 147-152; Howarth et al, 2008, Oncogene, 27 (23), 3345-3359).

Unlike certain other FOX proteins, FOXP2 has not been extensively studied in cancer and the principal focus of research has been on the role of FOXP2 in neuronal systems. Most notably, the primary association of FOXP2 in the art is with speech and language disorders. Mutation of FOXP2 has been linked to an inherited speech and language disorder (Lai et al, 2001, Nature, 413, 519-523). In one of the cases, a three generation family referred to as “KE” has a single amino acid substitution in the forkhead domain which compromises FOXP2 DNA binding activity and thus its ability to regulate gene expression. This has prompted extensive study of the role of FOXP2 in neuronal development and human language, and comparison with Neanderthal DNA has suggested a role in the evolution of human speech (Krause et al, 2007, Curr Biol, 17 (21), 1908-1912).

The FOXP2 gene is highly conserved between different species, has several splice variants, at least four different transcriptional start sites and more highly conserved regions within its introns than virtually any known gene.

The FOXP2 protein has been shown to bind to the SMAD3/MADH3 promoter in both human fetal brain and lung tissues, using chromatin immunoprecipitation (Spiteri et al, 2007, Am J Hum Genet, 81(6), 1144-1157). SMAD proteins are a small family of proteins that bind DNA and regulate transcription. They can propagate the TGF-β signal downstream of the cell surface receptors, which negatively regulates the cellular proliferation and differentiation of normal B lymphocytes, together with their ability to undergo apoptosis.

A practical utility based on an association of FOXP2 expression with cancer has not yet emerged. However, in WO 2004/022104 a possible use of FOXP2 in treating or identifying patients with cancer was discussed. It was reported that the levels of FOXP2 mRNA were found to be markedly diminished in cancers such as kidney, colon, uterus, prostate, breast or stomach cancer. Methods for treating such cancers by increasing levels of FOXP2 were proposed, by administering enhancers of FOXP2 expression to a patient. Thus WO 2004/022104 teaches that FOXP2 expression is decreased in cancer and should be upregulated in therapy.

Surprisingly, the present inventors have now found that in the case of malignant lymphocytes, in contrast to the results showing cancer-associated reduced expression reported in WO 2004/022104, FOXP2 expression is increased and that such increased expression allows FOXP2 to be used as a marker for lymphoid malignancies, or more generally for abnormal lymphocytes, which may occur in various disorders. The present inventors have thus discovered that, in contrast to the teaching of the prior art, increased expression of FOXP2 can identify abnormal e.g. malignant or pre-malignant lymphocytes from their normal counterparts. Thus, whilst FOXP2 is normally expressed in most normal tissues, it is notably not expressed in normal haematopoietic cells, irrespective of lineage or stage of differentiation. More particularly, FOXP2 is not expressed in normal lymphocytes. However, in abnormal (e.g. neoplastic or malignant) lymphocytes, expression of FOXP2 may be seen, and FOXP2 is thus proposed herein as a novel marker for abnormal lymphocytes. FOXP2 expression has now been shown to be detectable in a variety of cell lines derived from various haematological malignancies, and notably such malignancies are associated with lymphocytes, including various lymphomas, e.g. diffuse large B-cell lymphoma (DLBCL), Hodgkin lymphoma (HL), and myeloma. Furthermore, in tissue samples obtained from patients with such malignancies increased FOXP2 expression in lymphocytes may be seen (for example in bone marrow samples or in other biopsy samples). In particular, in the case of myeloma, a consistent increase in FOXP2 expression in bone marrow plasma cells may be seen; the expression of FOXP2 in plasma cells has been detected in more than 95% of bone marrow samples studied from patients with myeloma (as compared with 71% expressing CD56, a currently used marker for myeloma). Also significantly, expression of FOXP2 has been detected in plasma cells from patients with monoclonal gammopathy of undetermined significance (MGUS), a benign condition characterised by an increase in plasma cells, which in certain patients can progress to myeloma. Thus, FOXP2 expression has particularly been shown to be increased in conditions exhibiting plasma cell abnormalities, and in a specific aspect the inventors have accordingly identified a novel marker for abnormal plasma cells.

The discovery of FOXP2 as a novel disease marker is an extremely important development for the identification of patients with abnormal lymphocytes and in particular of patients with abnormal plasma cells, as may occur most notably in myeloma, and related conditions such as MGUS.

Myeloma, commonly referred to as multiple myeloma (MM), and known also as plasma cell myeloma or symptomatic myeloma, is a cancer of the plasma cells caused by proliferation and clonal expansion of terminally differentiated malignant post-germinal centre B lymphocytes (plasma cells) in the bone marrow. Myeloma interferes with normal plasma cell production of immunoglobulins and myeloma cells produce an abnormal immunoglobulin called M protein or paraprotein which does not function. Myeloma cells are addicted to an aberrant IRF4 regulatory network that fuses the gene expression programmes of activated B cells and normal plasma cells. Inhibition of this pathway is toxic to the tumour cells (Shaffer et al, 2008, Nature, 454 (7201), 226-231). Myeloma is the second most common blood cancer in the United States and accounts for approximately 1% of all cancers. Although treatments have improved over the years, currently the prognosis for myeloma sufferers is not good; patients presently have only a five year relative survival rate of approximately 35%, although it is hoped that the upcoming promising new array of novel therapies for this malignancy may improve matters (Piazza et al, 2007, Ann Hematol, 86 (3), 159-172; Harousseau, 2008, Ann Oncol, 19 Suppl 5, v68-70; San-Miguel et al, 2008, J Clin Oncol, 26 (16), 2761-2766). The clinical and economic significance of this disease is therefore high. Whilst most cases of myeloma arise de novo, a minority arise from the pre-malignant condition monoclonal gammopathy of undetermined significance (MGUS), which affects approximately 3% of individuals over 50 and 5% of those over 70 (Kyle et al, 2006). The rate of disease progression in MGUS patients is approximately 1% per year, with their risk of developing myeloma being increased by 25 fold.

MGUS is a non-cancerous (benign) condition in which plasma cell numbers are increased and an abnormally high amount of a single antibody is produced (monoclonal gammopathy or paraproteinaemia), but with no evidence of cancer (either myeloma or lymphoma). The levels of plasma cells and antibody (monoclonal/para- protein) are raised over normal, but are not as high as in myeloma (MM). MGUS and myeloma may thus be seen as lying sequentially along a disease spectrum of plasma cell disorders, characterised by increased plasma cells, with the diagnosis depending on the number of plasma cells and/or clinical features. This disease spectrum includes also smouldering myeloma (also known as indolent or asymptomatic myeloma), in which the increase in plasma cell number and monoclonal protein is increased relative to MGUS but in which the patient is not showing overt signs of disease in terms of organ damage or tissue impairment (typically referred to as “end-organ damage”). In general, a level of less than 10% clonal plasma cells on bone marrow biopsy and less than 30 g/L of monoclonal protein (paraprotein) in serum is classified as MGUS. In asymptomatic myeloma, clonal plasma cells are >10% on bone marrow biopsy and/or serum monoclonal protein is >30 g/L and there is no myeloma-related organ or tissue impairment. In (symptomatic) myeloma, clonal plasma cells are >10% on bone marrow biopsy, there is usually monoclonal protein in either serum or urine and there is evidence of related organ or tissue impairment (e.g. hypercalcaemia, renal insufficiency, anaemia, bone lesions (lytic lesions or osteoporosis with compression fractures), frequent severe infections (more than 2 a year), amyloidosis of other organs or hyperviscosity syndrome).

Diagnosis of myeloma is currently based on the demonstration of a monoclonal protein (M-protein/paraprotein) in the serum or urine and/or the presence of lytic lesions on X-ray, together with the quantification of the clonal plasma cell infiltration (more than 10%) in bone marrow aspirate smears or trephines. Neoplastic plasma cells from myeloma patients also display an aberrant phenotype that can be used to distinguish them from their normal counterparts in bone marrow. This is particularly important because a high ratio of abnormal to normal plasma cells at presentation is one of the most significant predictors of MGUS and asymptomatic myeloma patients with a greatly increased likelihood of disease progression. Whilst CD138 is a universal marker of both normal and malignant plasma cells, markers which are particularly useful for distinguishing normal from abnormal plasma cells include the B-cell marker CD19, which is expressed in more than 70% of normal plasma cells and is absent from 95% of MM cases and the natural killer cell marker CD56 which is expressed in less than 15% of normal plasma cells and is strongly expressed in more than 75% of MM cases. A panel based on CD19 and CD56 will identify approximately 90% of MM cases while the inclusion of CD20, CD117, CD28 and CD27 has been predicted to increase this to more than 95% of MM cases. However, to date, no single marker has been reported systematically to differentiate neoplastic plasma cells from their normal counterparts.

This need has now been met by the present invention. The increased expression of FOXP2 in abnormal lymphocytes and in particular in abnormal plasma cells, allows the use of FOXP2 as a single marker for the detection of abnormal lymphocytes, particularly abnormal plasma cells and accordingly for the detection or assessment of disease associated with abnormal lymphocytes, for example lymphoma or myeloma, and particularly abnormal plasma cells, for detection or assessment of plasma cell disorders, including myeloma. Whether used as a single marker, or in conjunction with other markers (such as CD138 and CD56), the use of FOXP2 as a marker may improve or facilitate the detection of abnormal lymphocytes, and thereby aid the diagnosis or prognosis of conditions associated with abnormal lymphocytes e.g. lymphocyte malignancies. FOXP2 may thus be used for the detection of abnormal lymphocytes and, advantageously in conjunction with other parameters such as lymphocyte (for example plasma cell) number (count) or clinical parameters such as indicators of end-organ or tissue damage, in the detection and assessment of conditions associated with abnormal lymphocytes. Expression of FOXP2 in lymphocytes is thus associated with abnormality or malignancy. As discussed above, the detection of FOXP2 mRNA expression in 96% of myeloma samples at diagnosis (26/27, including both purified plasma cells and total bone marrow) and FOXP2 protein in 95% of samples from MM patients (originally in 82% of samples but this increased to 95% after re-examination of clinical notes) and also in plasma cell samples from patients with MGUS allows the use of FOXP2, whether as FOXP2 protein or as FOXP2 nucleic acid, e.g. mRNA, as a marker and possibly a single or independent marker for detecting abnormal (e.g. malignant or pre-malignant) plasma cells and as a tool in the diagnosis or prognosis of plasma cell disorders, for example in the diagnosis of myeloma. The use of FOXP2 as a marker is particularly advantageous over other myeloma markers previously used in the art e.g. CD56 which only identifies 75% of MM cases and which is also expressed in natural killer cells. The FOXP2 marker is thus more specific and may not require the detection of a combination of different molecules, particularly where FOXP2 mRNA is detected. FOXP2 may accordingly be used as the sole marker for abnormality or malignancy. It may be beneficial in some cases however to use FOXP2 as a marker for example in combination with other markers for cell type, such as for example in conjunction with CD138 as a plasma cell marker. By using CD138 labelling it can be established whether the FOXP2 positive cell is a plasma cell and it is also possible to visualise weak staining without the use of a nuclear counterstaining which increases the number of FOXP2 positive cases identified.

Further, the inventors have found that FOXP2 mRNA is expressed in osteoblasts, as is the RUNX2 gene. Thus, according to our findings, plasma cells in myeloma express two genes, namely FOXP2 and RUNX2 (Colla et al, 2005, Leukemia, 19, 2166-2176) Which are normally expressed in osteoblasts. The expression of these osteoblast transcription factors in plasma cells in myeloma may be functionally associated with the bone disease phenotype observed in myeloma. Further, the level of FOXP2 expression in plasma cells in myeloma may correlate to the severity of bone disease seen, with a high level of expression indicating an increased severity of bone disease. In other malignancies, the expression of FOXP2 in lymphocytes (or indeed as discussed in more detail later on, in tumours in general, including tumours involving other cell types) may identify patients with tumours that are likely to colonise the bone marrow.

Hence, the inventors have firstly surprisingly identified that FOXP2 can be used as a marker for abnormal lymphocytes, in view of its increased expression in such cells, a surprising and unpredictable observation given the reports in the prior art of decreased FOXP2 expression in cancer. Secondly, it has been identified that FOXP2 expression in lymphocytes e.g. plasma cells and in other tumours may be associated with bone disease development and may accordingly be targeted in the treatment of bone disease, particularly malignant bone disease, both primary and secondary (i.e. primary bone tumours and bone metastases).

In a first aspect, the present invention accordingly provides a method for detecting abnormal lymphocytes, said method comprising detecting an amount or expression of the FOXP2 gene in lymphocytes in a sample, wherein an increased amount or expression of the FOXP2 gene in said lymphocytes indicates the presence of abnormal lymphocytes.

The FOXP2 gene is defined herein as any gene or any DNA molecule encoding a FOXP2. protein. FOXP2 gene expression may be determined or detected by measuring or detecting expression of a FOXP2 protein or a FOXP2 mRNA, and as discussed below this may be achieved by a variety of methods known in the art. An increase in expression of the FOXP2 gene may however occur by a variety of mechanisms and the exact nature of the mechanism is not critical. Thus in addition to “switching” or “turning” on, or increasing, transcription of the FOXP2 gene, or effects on translation of FOXP2 mRNA into protein, or stabilising either mRNA and/or protein, an increase in expression may also occur as a result of an increase in gene dosage, or other effects at the DNA or gene level e.g. gene or chromosomal translocations or rearrangements which result in gene expression, or increased gene expression. The method of the invention thus encompasses detecting such translocations or rearrangements or determining the amount of the FOXP2 gene, namely gene copy number or dosage, as well as detecting or determining the level of gene expression.

The sample may be any sample which contains lymphocytes. Generally this will be a clinical sample, e.g. a sample of body tissue or fluid, which may be taken from a subject or patient under study or investigation. The precise nature of the body or tissue fluid sample may depend on the subject and/or condition under investigation but generally will be a sample of haematological tissue or fluid, or put another way a sample containing haematological cells, particularly lymphoid tissue or cells or plasma cells. In practice the sample will commonly be taken, obtained or derived from bone marrow, blood or tumour material, and may for example be a bone marrow biopsy sample (e.g. a bone marrow trephine) or a bone marrow aspirate, a tumour biopsy sample, a lymph node biopsy sample, peripheral blood mononuclear cells or a blood-derived sample such as serum or plasma. Other clinical samples may include cerebrospinal fluid or pleural effusions, or other tissues in which abnormal lymphocytes may occur.

In addition to clinical samples, other samples containing lymphocytes may be used, for example any biological sample containing lymphocytes, e.g. for laboratory investigation (for example a sample derived or obtained from a test animal) or lymphocyte or other cell or tissue cultures or cell lines. Thus although commonly the sample will be from a human subject, it may be from any non-human animal subject.

The amount or expression of the FOXP2 gene may be increased relative to the amount or expression in normal lymphocytes, more particularly corresponding or equivalent normal lymphocytes e.g. a sample or population of lymphocytes from the same species, or from a lymphocyte cell line derived from the same species, including a disease-derived cell line such as a myeloma or lymphoma cell line (e.g. the myeloma cell line JJN3). Thus, the amount or expression may be increased in comparison to a control or reference sample of lymphocytes, e.g. corresponding or equivalent lymphocytes taken from a healthy subject. The amount or expression may also be determined in a sample from a subject relative to a sample from the same subject, taken at a different e.g. earlier time point, e.g. prior to the onset of disease or prior to treatment or earlier in treatment, for example in the context of monitoring or determining disease progression or treatment response or for the detection of minimal residual disease. Thus the amount or expression of FOXP2 in the test sample may be determined or detected, both in absolute terms, or relative to another sample, depending on the use to which the method is put. As explained in more detail below, in terms of detecting expression of FOXP2, a comparison to a control or reference or any other sample may not be necessary, since normal lymphocytes rarely express FOXP2 and hence expression of FOXP2 in more than a few cells e.g. in more than 10% of lymphocytes, particularly, CD138⁺ plasma cells is indicative of abnormal lymphocytes. In this context, an increase in FOXP2 expression can be seen as any expression or any significant expression (e.g. any expression over zero, or any detectable expression, or any expression over a minimum number of cells, which may occasionally occur in samples containing “normal” lymphocytes). Thus a very small number, or low or very low incidence, of cells expressing FOXP2 detected may not be indicative of abnormality. However, if such a small number of FOXP2-expressing cells is seen, this may be accounted for using appropriate controls, or discounting de minimis expression etc. For example, a cut off value may be set e.g. for the number or proportion of cells expressing FOXP2 below which the sample is not scored as positive e.g. less than 5%, 8%, 10% or 12%.

The human FOXP2 gene has a nucleic acid sequence as set forth in SEQ ID NO.1. The sequence of human FOXP2 protein as well as variants generated by alternative splicing are known in the art and are shown in SEQ ID NOs 2, 4, 6, 8, 10, 12, 14, 16, 18, 20, 22, 24, 26, 28, 30, 32, 34 and 37. Reference to the “FOXP2 gene” or to “FOXP2” as used herein hence encompasses the nucleotide sequence set forth in SEQ ID NO.1 or any nucleotide sequence encoding FOXP2 having an amino acid sequence as set out in SEQ ID NOs 2, 4, 6, 8, 10, 12, 14, 16, 18, 20, 22, 24, 26, 28, 30, 32, 34 or 37. Additionally, reference to “FOXP2” also includes substantially homologous variants of the nucleotide sequence set forth in SEQ ID NO.1 or a nucleotide sequence which encodes substantially homologous variants of the FOXP2 amino acid sequence as set out in SEQ ID NOs 2, 4, 6, 8, 10, 12, 14, 16, 18, 20, 22, 24, 26, 28, 30, 32, 34 or 37, since for example the method can be used to detect abnormal lymphocytes in other non-human animals, for example the method can be used on a mouse, rat, rabbit, monkey, cat or dog. Thus homologous or orthologous FOXP2 or FOXP2 sequences from other species are covered. Further, the invention can be used to detect polymorphic forms of FOXP2, naturally occurring allelic variants of FOXP2 or mutant forms of FOXP2 which contain mutations compared to the sequence set out in SEQ ID NO.1. or which may encode an amino acid sequence which contains variations (mutations) as compared with SEQ ID NOs 2, 4, 6, 8, 10, 12, 14, 16, 18, 20, 22, 24, 26, 28, 30, 32, 34 or 37. A mutation can consist of an addition or deletion or substitution of any one or more nucleotides e.g. 1, 2, 3, 4, 5, 6, 7, 8, 9 or 10 nucleotides. Such a mutation may be a substitution, for example which results in a conservative amino acid substitution in the encoded amino acid sequence. Such mutations can hence result in amino acid substitutions of D by E or vice versa, N by Q or L; I by V of vice versa etc. Substantially homologous variants may have at least 80, 85, 90, 95, 96, 97, 98 or 99% sequence identity to the nucleotide sequence as set out in SEQ ID NO. 1 or may encode an amino acid sequence having at least 80, 85, 90, 95, 96, 97, 98 or 99% sequence identity to the amino acid sequence as set out in SEQ ID NOs 2, 4, 6, 8, 10, 12, 14, 16, 18, 20, 22, 24, 26, 28, 30, 32, 34 or 37 (FOXP2). Identity may be determined using the BestFit program of the Genetics Computer Group Version 10 software package from the University of Wisconsin. The program uses the local hand algorithm of Smith and Waterman with the default values: Gap creation penalty=8, Gap extension penalty=2, Average match =2.912, Average mismatch=2.003.

The detection of the amount of the FOXP2 gene in lymphocytes in a sample includes the determination of the number of copies of FOXP2 within said lymphocytes. An increase in the amount of FOXP2 hence can refer to an increase in the number of copies of FOXP2 present in a lymphocyte compared to the number of copies of FOXP2 which occur in a normal lymphocyte. Preferably, therefore, an increased amount of FOXP2 means the presence of more than 2 copies of the FOXP2 gene, for example, 3, 4, 5 or more copies. Thus, the determination of the amount of FOXP2 includes the determination of the number of copies of the sequence of SEQ ID NO.1 or the number of copies of a sequence which encodes the FOXP2 protein present in a lymphocyte cell.

The amount of FOXP2 can be determined using an oligonucleotide sequence as a probe or primer comprising at least 10 consecutive nucleotides from FOXP2, for example, comprising at least 10 nucleotides from SEQ ID NO. 1 or from a sequence encoding FOXP2 e.g. encoding a sequence as set out in SEQ ID NOs 2, 4, 6, 8, 10, 12, 14, 16, 18, 20, 22, 24, 26, 28, 30, 32, 34 or 37. Said oligonucleotide sequence may thus comprise a sequence of at least 20, 30, 40, 50, 60 or 70 consecutive nucleotides from FOXP2. The oligonucleotides may be produced according to techniques well known in the art e.g. by synthetic or recombinant means. The oligonucleotides may be used as primers to initiate replication for example in PCR or as probes which are contacted with the sample under hybridising conditions where the presence of any duplex or triplex formation between the probe and any nucleic acid in the sample is detected.

The probes used for determination of the amount of FOXP2 may be anchored to a solid support, for example the probes may be spotted or synthesised in situ on an array. In this way, the copy number or amount of FOXP2 may be measured by for example comparative genomic hybridisation, where sample and reference DNAs are differentially fluorescently labelled and hybridised together to the array. The resulting fluorescent ratio can be measured and an increased copy number can be detected by a stronger fluorescent signal from the sample DNA as compared to the fluorescent signal from the reference DNA i.e. from normal lymphocytes.

Copy number gains in lymphocytes from fresh, frozen or routinely formalin fixed paraffin embedded tissues can also be detected by fluorescent in situ hybridisation (FISH). Bacterial artificial chromosome (BAC) clones containing large regions of DNA flanking and/or across the FOXP2 gene can be labelled with fluorescent conjugates that enable copy number visualisation using a microscope. These FISH probes can also be used to simultaneously detect both copy number gains and chromosome rearrangements targeting the FOXP2 locus via labelling probes upstream and downstream of the FOXP2 gene with different fluorochromes. Fused signals (usually red+green, which show as yellow) indicate an intact gene, while break apart signals (separate red and green) detect chromosomal translocation.

In such an embodiment, the method may therefore comprise a comparison step wherein the amount of FOXP2 detected in the lymphocytes from the sample (i.e. in the test sample) may be compared to the amount of FOXP2 present in a normal equivalent or corresponding lymphocyte sample or population (i.e. in a control or reference sample). Thus if the test sample is from a subject individual e.g. a patient, it may be compared to a reference sample from a healthy subject of the same species.

The detection of expression of the FOXP2 gene includes the detection of FOXP2 mRNA present in lymphocytes and/or the amount of FOXP2 protein produced. Hence, since normal lymphocytes do not usually express FOXP2, the detection of FOXP2 mRNA or FOXP2 protein in a lymphocyte sample is sufficient to detect abnormal lymphocytes i.e. the actual level of expression does not essentially need to be determined. Therefore, the detection of FOXP2 mRNA or FOXP2 protein in lymphocytes generally indicates an increase in the level of expression of FOXP2. Hence, any detection of expression generally evidences an increase in the level of expression as compared to normal non-expressing lymphocytes.

The detection of expression of FOXP2 can however additionally involve a step of determining the level of expression of FOXP2 in lymphocytes in the sample i.e. the level of FOXP2 mRNA or FOXP2 protein can be assessed. An increased level of expression may encompass an increase of for example, 10, 20, 30, 40, 50, 60, 70, 80, 90, 100, 200 or 300% in the amount of FOXP2 mRNA or FOXP2 protein present within a lymphocyte or lymphocyte sample. Alternatively viewed, an increased level of expression is seen where an increase of up to 2-fold in the amount of FOXP2 mRNA or FOXP2 protein is detected relative to a normal or reference lymphocyte. In other embodiments 2, 3, 4, 5, 6, 7, 8, 9 or 10 fold increase in the amount of FOXP2 mRNA or FOXP2 protein is present in a lymphocyte as compared with a normal counterpart lymphocyte or a reference cell. It will be appreciated, however, for reasons explained above, that simply the presence of FOXP2 mRNA or FOXP2 protein may be scored as increased FOXP2 expression, particularly if specific thresholds are set for detection.

Reference to a FOXP2 protein as used herein includes any known FOXP2 protein and homologues or orthologues thereof. Amino acid sequences for FOXP2 proteins from various species are known in the art, as indeed are various isoforms or variant sequences e.g. splice variants. As noted above, particularly included are human FOXP2 sequences, Thus, specifically, the FOXP2 protein may have an amino acid sequence as set out in SEQ ID NOs 2, 4, 6, 8, 10, 12, 14, 16, 18, 20, 22, 24, 26, 28, 30, 32, 34 or 37 or any alternative spliced isoform of the protein. Hence, detection of FOXP2 encompasses the detection of any FOXP2 protein or any of its alternatively spliced isoforms. Substantially homologous variants of FOXP2 e.g. non-human Foxp2 from different species e.g. mouse, rat, rabbit, monkey, cat, dog etc can be detected in the method and the method is hence not limited to use on human samples. The term “FOXP2” encompasses all such substantially homologous variants. Substantially homologous variants may include those which have at least 80, 85, 90, 95, 96, 97, 98 or 99% similarity or preferably identity to the amino acid sequences set out in SEQ ID NOs 2, 4, 6, 8, 10, 12, 14, 16, 18, 20, 22, 24, 26, 28, 30, 32, 34 or 37. Amino acid sequence identity or similarity may be determined using the BestFit program of the Genetics Computer Group Version 10 software package from the University of Wisconsin. The program uses the local hand algorithm of Smith and Waterman with the default values: Gap creation penalty=8, Gap extension penalty=2, Average match=2.912, Average mismatch=2.003. Thus, a naturally occurring variant of FOXP2 may differ by 1 to 10, 1 to 6, 1 to 4, 1 to 3 or 1 to 2 amino acid substitutions, insertions, and/or deletions which may be contiguous or non-contiguous as compared to SEQ ID NOs 2, 4, 6, 8, 10, 12, 14, 16, 18, 20, 22, 24, 26, 28, 30, 32, 34 or 37. The substituted amino acid may be one of the well known 20 conventional amino acids, although typically it may be a conservative amino acid substitutions. Thus an amino acid may be replaced by another which preserves the physiochemical character of FOXP2 (e.g. D may be replaced by E or vice versa, N by Q or L; I by V or vice versa).

The reference to “detecting the expression of FOXP2” may also include the detection of specific mutations or chromosomal translocations in the FOXP2 gene which increase the level of expression of FOXP2. Hence, the direct detection of any mutations or translocations may indirectly detect expression of FOXP2 and increased expression of FOXP2. It is possible to detect chromosomal translocations by FISH (fluorescence in situ hybridisation) or by PCR using primers than flank the breakpoints in FOXP2 and the partner gene.

FOXP2 mRNA can be detected and/or levels of mRNA determined using many different techniques which are well known in the art, for example by Northern blotting or using microarrays. Further, real time PCR can be used to determine mRNA levels, where total RNA can be extracted from a cell and reverse transcribed. Real time PCR can then be carried out on the reverse transcribed sample and the mRNA expression level determined.

FOXP2 protein (and its variants produced by alternative splicing) can be detected and/or levels determined using many well known techniques. The assays to detect FOXP2 may be qualitative, quantitative or semi-quantitative.

Such an assay may for example simply involve contacting a sample with an antibody which can detect FOXP2 and detecting whether or not it binds. Such antibodies are described in greater detail below. Such an assay may be carried out using any of the well known immunoassay techniques which are widespread in the art e.g. sandwich assays, competitive assays, immunometric assays etc. Other assay formats may also be used, e.g. assays based on flow cytometry. Western blotting may also be used where proteins are resolved by SDS-PAGE and transferred to nitrocellulose membranes. FOXP2 protein can then be detected using an anti-FOXP2 antibody. Antibodies such as FOXP2 N-16 sc-21069 (Santa Cruz, Calif., USA), or FOXP2-73A/8 (deposited under Accession Number 08101410 on 14 Oct. 2008 at ECACC (European Collection of Cell Cultures), Centre for Emergency Preparedness and Response, The Health Protection Agency, Porton Down, Salisbury, SP4 0JG) may preferably be used to identify FOXP2.

The antibody may be labelled and may be detected and/or measured by means of the label. Labelling may be by any convenient means and a wide variety of labels and labelling techniques for antibodies are well known in the art.

Such labels may include for example, fluorochromes, radioisotopes, coloured dyes, quantum dots, or other chromogenic agents, enzymes, colloidal metals, chemi- and bio-luminescent compounds. The labels may be directly detectable or signal giving such as those listed above, or they may be labels which take part in a signal giving or detectable reaction, for example by binding to another molecule e.g. they may be an indirectly detectable label. Thus a label may be a small molecule such as a hapten or a tag e.g. biotin, which may be bound by a binding partner therefor (e.g. streptavidin/avidin for biotin).

Further, FOXP2 protein may be detected and/or its levels assessed by immunohistochemistry, preferably using the antibody FOXP2-73A/8.

It will be appreciated that the amount or level of expression of FOXP2 may not be increased in all lymphocytes present in the sample. Hence, it is possible that a sample may comprise a heterogeneous population of lymphocytes, some of which may have an increased amount or level of expression of FOXP2 and some of which may not. The present method can be used to detect the abnormal lymphocytes in such a sample which show an increased expression level or amount of FOXP2.

Although as described previously, an increased level of expression of FOXP2 can be determined by merely detecting any expression of FOXP2 since normal lymphocytes do not usually express FOXP2, in one embodiment of the invention, the method can include a step of comparing the level of expression of FOXP2 (and also the amount of FOXP2) with the level of expression of FOXP2 (or amount of FOXP2) in normal lymphocytes, or any desired or appropriate control or reference sample. Hence, the invention can provide a method for detecting abnormal lymphocytes comprising determining the amount or level of expression of the FOXP2 gene in lymphocytes in a test sample and comparing said amount or expression level of the FOXP2 gene with the amount or expression level of the FOXP2 gene detected in normal lymphocytes (or in a control or reference sample), wherein an increased amount or level of expression of FOXP2 in lymphocytes in the test sample indicates the presence of abnormal lymphocytes in said test sample.

However, as noted above, such a comparison step is not an essential feature of the invention. Accordingly, alternatively viewed the present invention can also be seen to provide a method for detecting abnormal lymphocytes, said method comprising detecting expression of the FOXP2 gene in lymphocytes in a sample, wherein expression of the FOXP2 gene in said lymphocytes indicates the presence of abnormal lymphocytes. In particular, such a method may include determining the amount or proportion or percentage of lymphocytes in the sample which express FOXP2 (or any other such indication of the amount or relative amount of FOXP2-expressing lymphocytes in the sample). As described previously, a sample may be considered to be FOXP2 positive if FOXP2 expression is detected in more than 10% of cells, particularly in more than 10% of CD138⁺ plasma cells.

The term “lymphocyte” as used herein refers to a cell which is derived from the so-called common lymphoid progenitor. A lymphocyte is thus a cell of the lymphoid lineage. Hence a lymphocyte may be a T-cell, a B-cell or a natural killer cell, or any cell which may differentiate therefrom. B and T cells and cells derived therefrom may be at various stages of differentiation or activation and all such stages are included. T and B cells and their derivative cells are particular lymphocytes where abnormalities may occur and which may give rise to pathological or clinical conditions associated with such abnormalities, particularly neoplastic disorders or malignancies. Thus a type of lymphocyte of particular interest according to the present invention is a plasma cell.

“Plasma cells” (or plasma B cells or plasmocytes) are differentiated from B cells upon stimulation by CD4+ lymphocytes and usually secrete antibodies. Plasma cells are hence generally considered to be terminally differentiated B cells. Plasma cells do not express common pan B cell markers such as CD19 and CD20 and instead are usually identified by their expression of CD38, CD78 and the Interleukin-6 receptor.

“T cells” can be distinguished from other lymphocytes by the presence of the T-cell receptor on their cell surface and include several different subsets e.g. cytotoxic T cells and helper T cells.

B cells are lymphocytes that play a large role in the humoral immune response and their principal functions are the production of antibody, their role as antigen presenting cells and eventually their development into memory B cells.

The method of the invention can be used to detect abnormal T cells, B cells and/or plasma cells in a sample. In a preferred embodiment of the invention, the abnormal lymphocytes detected are abnormal plasma cells. Hence, a method for detecting abnormal plasma cells comprises detecting the amount or expression of FOXP2 in plasma cells in a sample wherein an increased amount or level of expression of FOXP2 in said plasma cells indicates the presence of abnormal plasma cells.

As noted above, plasma cells may be identified or characterised by the expression of the plasma cell marker CD138. Thus, in an advantageous embodiment the method of the invention for detecting abnormal plasma cells combines the detection of FOXP2 amount or expression with the detection of CD138. Thus, in particular, the FOXP2 protein and CD138 protein may be detected, e.g. the presence or absence, or amount of FOXP2 and CD138. This may be achieved for example using antibody labelling techniques, as discussed further herein.

The results reported herein show particularly that detection of FOXP2 mRNA may be used reliably and efficiently for detection of FOXP2 expression. This represents a preferred embodiment of the invention, and in particular in such an embodiment FOXP2 mRNA may be detected as a single marker for abnormal plasma cells. As further described herein, FOXP2 mRNA may conveniently be detected using PCR or PCR-based techniques.

The term “abnormal” as used herein refers to cells e.g. to lymphocytes which may be differentiated or distinguished from a corresponding normal lymphocyte (i.e. from their normal counterpart), in terms of phenotypic characteristics and/or behaviour. An abnormal lymphocyte is thus different from a normal lymphocyte. Typically this may be manifested by altered phenotypic characteristics which may include altered cellular morphology (e.g. altered appearance), but most commonly include the altered expression of proteins, including cell surface proteins. Thus, an abnormal cell e.g. an abnormal lymphocyte shows an altered or aberrant phenotype (a phenotype different from that expected of it). Thus the expression of markers, e.g. markers used to identify or classify lymphocytes may be altered e.g. usual cell markers may be absent and different cell markers may be present, and/or may exhibit an altered gene expression profile compared to normal lymphocytes under the same conditions.

Such abnormal cells e.g. lymphocytes may contain one or more gene mutations, alterations or chromosome translocations and may demonstrate a spectrum of abnormality.

An abnormal cell e.g. an abnormal lymphocyte may also show abnormal characteristics in terms of abnormal proliferation compared to its normal counterpart. Thus proliferation (or the proliferative capacity) of the cell may be increased. Such increased or abnormal proliferation may result in the production of a clonal population of cells. Proliferation may be increased by more than 1%, for example by more than 2, 3, 4, 5, 6, 7, 8, 9, or 10% or possibly in some cases by more than 20, 30, 40 or 50%. In terms of plasma cells, for example, it is believed that in normal conditions the majority of these are not actively proliferating. In disease conditions such as myeloma the proportion or amount of proliferating cells may increase. This may be assessed by techniques known in the art, for example immunohistochemistry with ki67/MIB1 staining or flow cytometry and staining with propidium iodide or Hoescht dye.

An abnormal lymphocyte may thus be a neoplastic lymphocyte. More particularly, the abnormal lymphocyte may be malignant or pre-malignant. A pre-malignant lymphocyte, or a pre-cancerous lymphocyte, is one which is not malignant but which shows changes which are associated with progression to malignancy e.g. which is at risk of or likely to become malignant i.e. is potentially malignant. In one aspect, pre-malignant cells do not exhibit malignant behaviour, (for example invade tissues or cause overt symptoms of disease or tumour development, or exhibit increased proliferation etc) but have a risk of developing into cells which will. Malignant lymphocytes are lymphocytes which exhibit abnormal behaviour, particularly in terms of growth, death and/or differentiation. Malignant lymphocytes may not be self-limited in their growth, they may fail to die at the appropriate time, and/or they may be arrested at a particular stage of development or differentiation or may exhibit altered differentiation. Alternatively viewed, a malignant lymphocyte is a cancerous lymphocyte.

As discussed above, reference to an abnormal lymphocyte includes reference to an abnormal (e.g. malignant or pre-malignant) plasma cell, B-cell or T-cell. Abnormal plasma cells are of particular interest in the present invention. An abnormal plasma cell may be identified by an aberrant phenotype, for example as reported in the art with reference to expression of cell markers. Thus by way of example an abnormal plasma cell may be identified or classified as CD19⁻ CD56⁺, and optionally CD117⁻, CD20⁺, CD28⁺⁺ and CD27⁻ or weak (normal cells tend to be strongly CD27⁺). Further markers which may be included in a marker panel for identification of an abnormal plasma cell include CD81 (weak or negative) and CD200 (strongly positive).

Markers for identifying other abnormal e.g. malignant lymphocytes are reported in the art, for example see Cheshire P, “Identification of B-cell lymphoma”, Biomedical Scientist, November 2001, 1194-1197 and http://www.nhlcyberfamily.org/tests/cdmarkers.htm.

Abnormal, e.g. neoplastic, pre-malignant or malignant lymphocytes can be found in patients with a variety of different conditions, including abnormal plasma cells in plasma cell disorders which may include MGUS and myeloma and which may thus be non-malignant (benign) or malignant, and in various lymphomas, for example, Hodgkin Lymphoma (HL) and diffuse large B-cell lymphoma (DLBCL). As noted above abnormal plasma cells in myeloma may lack the B-cell marker CD19, and express the natural killer cell marker CD56. As will be described in more detail in the examples below, the methods of the present invention have been used to detect abnormal lymphocytes expressing the FOXP2 gene in subjects with MGUS, MM, Hodgkin lymphoma, ABC-like DLBCL and T-cell lymphomas.

Hence, in a preferred embodiment, the method of the invention can be used to detect abnormal lymphocytes, particularly malignant and/or pre-malignant lymphocytes in the diagnosis and/or assessment of such disorders or conditions or their treatment e.g. diagnosis and/or prognosis of such conditions, or to monitor treatment response or detect minimal residual disease etc.

In a preferred embodiment, the method of the invention can be used to detect malignant and/or pre-malignant lymphocytes. In this aspect, the methods of the invention can hence detect malignant or pre-malignant T cells, B cells or plasma cells. Preferably, the invention provides a method as previously defined for detecting malignant or pre-malignant plasma cells.

As discussed above, abnormal and particularly malignant and pre-malignant lymphocytes are associated with many different disorders or disease conditions. It is possible to use the FOXP2 marker of the invention to indicate or identify the presence of such a disease condition associated with the presence of abnormal lymphocytes, optionally in association with other measures or parameters.

Hence, the invention extends to a method for detecting or assessing in a subject a condition associated with the presence of abnormal lymphocytes, said method comprising detecting the amount or expression of the FOXP2 gene in lymphocytes in a sample from said subject, wherein an increased amount or level of expression of the FOXP2 gene indicates or suggests the presence or status of said condition.

Detecting a condition may be seen as identifying a subject with the condition or identifying the condition in a subject. The term “assessing” broadly includes assessing the status of the condition, in terms of for example severity of disease, risk of or potential for progression, early detection of relapse, monitoring of response to treatment (i.e. any therapeutic intervention) or the presence of disease following treatment e.g. monitoring for the presence of minimal residual disease.

Such a method may thus be viewed as a method of diagnosis and/or prognosis. Accordingly, the invention provides a method for diagnosis and/or prognosis of a condition associated with the presence of abnormal lymphocytes in a subject, said method including the step of detecting an increased amount of the FOXP2 gene or expression of the FOXP2 gene in lymphocytes in a sample from said subject.

Particularly, with regard to prognosis, a sample of lymphocytes obtained from a subject in which a high percentage of lymphocytes express the FOXP2 gene and/or in which a high amount or expression of the FOXP2 gene is detected, may be associated with a poor prognosis.

A “poor prognosis” may relate to an increased severity of disease, a shorter life expectancy, a more rapid progression of disease and/or a reduced efficacy of treatment by therapeutics, e.g. as compared to usual or average disease progression/severity. For example, a poor prognosis may indicate a life expectancy of less than two years, more particularly of less than one year. Further, a poor prognosis may relate to an increased severity of bone disease or colonisation.

A high percentage of lymphocytes expressing FOXP2 may refer to more than 50, 60, 70, 80 or 90% of lymphocytes in a sample being FOXP2 positive i.e. expressing the FOXP2 gene. A high level of expression of the FOXP2 gene may be the result of the FOXP2 positive cells strongly expressing FOXP2 and/or an increased percentage of lymphocytes expressing the FOXP2 gene e.g. more than 50, 60, 70, 80 or 90% of lymphocytes in the sample expressing the FOXP2 gene. Thus, a high expression level includes a sample in which more, than 50% e.g. more than 60, 70, 80 or 90% of lymphocytes, particularly plasma cells, express the FOXP2 gene. Samples with a high level of expression of the FOXP2 gene include those which have FOXP2 mRNA or FOXP2 protein levels more than 50, 100, 200, 300, 400 or 500% higher than that seen in a normal sample or a sample taken from a patient with MGUS.

Thus, the invention provides a method of determining the prognosis of a condition associated with the presence of abnormal lymphocytes in a subject, said method including the step of detecting an amount or expression of the FOXP2 gene in lymphocytes in a sample from said patient, wherein the detection of a high amount or expression level of FOXP2 is indicative of a poor prognosis.

Conditions associated with the presence of abnormal lymphocytes include conditions associated with the presence of abnormal plasma cells, T cells or B cells e.g. malignant or pre-malignant plasma cells, T cells or B cells. The term “associated with” includes conditions which are characterised by the presence of abnormal lymphocytes, caused by the presence of abnormal lymphocytes, which exhibit abnormal lymphocytes or in which abnormal lymphocytes occur or exist.

A condition associated with abnormal lymphocytes may thus include a lymphocytic neoplastic disorder or a lymphocyte malignancy. Particularly included are plasma cell disorders which may or may not be malignant, specifically MGUS and myeloma, which may include smouldering myeloma. Thus, in a preferred aspect the present invention includes detecting or assessing a condition associated with abnormal plasma cells and particularly detecting or assessing a plasma cell disorder

Plasma cell disorders may be included under the general term “plasma cell dycrasia” and such dyscrasias are included within the scope of the present invention. At its broadest, a plasma cell dyscrasia has been defined as a plasma cell disorder. More particularly, a plasma cell dyscrasia may be seen as a diverse group of diseases characterised by the proliferation of a single clone of cells producing a monoclonal immunoglobulin or immunoglobulin fragment (a serum M component). The cells usually have plasma cell morphology, but may have lymphocytic or lymphoplasmacytic morphology. This group includes multiple myeloma, Waldenstrom's macroglobulinaemia, the heavy chain disease, benign monoclonal gammopathy, immunocytic amyloidosis, primary amyloidosis, plasmacytoma, plasma cell leukaemia, and POEMS syndrome.

Specifically a plasma cell disorder includes MGUS and myeloma, and more specifically multiple myeloma and asymptomatic myeloma.

Other lymphocyte disorders include lymphomas, which may include both Hodgkin and non-Hodgkin's lymphomas. The latter may include various lymphoma sub-types including both T- and B-cell lymphomas, for example diffuse large B cell lymphoma, small lymphocytic lymphoma, lymphoplasmacytic lymphoma, splenic marginal zone lymphoma, extranodal marginal B-cell lymphoma, follicular lymphoma, mantle cell lymphoma, Adult T-cell leukaemia/lymphoma, Enteropathy-type T-cell lymphoma, hepatosplenic T-cell lymphoma and angioimmunoblastic T-cell lymphoma. As will be described in more detail below, the method of the invention may be used to detect abnormal lymphocytes, which may occur in certain lymphomas and thus may used to aid the diagnosis of such lymphomas or in the prognosis of lymphoma.

The term “lymphoma” as used herein refers to a tumour of lymphoid tissue or any malignant disease that usually start in the lymph nodes or lymphoid tissue. Lymphoid malignancies include the following conditions which are classified by the WHO as (i) Precursor Lymphoid Malignancies e.g. B lymphomoblastic leukaemia/lymphoma and T lymphomoblastic lymphoma, (ii) Mature B-cell neoplasms e.g. Chronic lympocytic leukaemia/small lymphocytic lymphoma, B-cell prolymphocytic leukaemia, Splenic B-cell marginal zone lymphoma, Hairy cell leukaemia, Lymphoplasmacytic lymphoma, Extranodal marginal zone lymphoma of mucosa associated tissue, Nodal marginal zone lymphoma, Follicular lymphoma, Primary cutaneous follicle centre lymphoma, Mantle cell lymphoma, Diffuse large B-cell lymphoma, Lymphotoid granulomatosis, Primary mediastinal large B-cell lymphoma, Intravascular large B-cell lymphoma, ALK positive large B-cell lymphoma, Plasmablastic lymphoma, Large B-cell lymphoma arising in HHV8-associated multicentric Castleman disease, Primary effusion lymphoma, Burkitt lymphoma, B-cell lymphoma, unclassifiable, with features intermediate between diffuse large B-cell lymphoma and Burkitt lymphoma, B-cell lymphoma, unclassifiable, with features intermediate between diffuse large B-cell lymphoma and classical Hodgkin lymphoma, (iii) Mature T-cell and NK-cell Neoplasms e.g. T-cell prolymphocytic leukaemia, T-cell large granular lymphocytic leukaemia, Aggressive NK cell leukaemia, Systemic EBV positive T-cell lymphoproliferative disease of childhood, Hydroa vacciniforme-like lymphoma, Adult T-cell leukaemia/lymphoma, Extranodal NK/T cell lymphoma, nasal type, Enteropathy-associated T-cell lymphoma, Hepatosplenic T-cell lymphoma, Subcutaneous panniculitis-like T-cell lymphoma, Mycosis fungoides, Sezary syndrome, Primary cutanoeus CD30 positive T-cell lymphoproliferative disorders, Primary cutaneous gamma-delta T-cell lymphoma, Peripheral T-cell lymphoma, NOS, Angioimmunoblastic T-cell lymphoma, Anaplastic large cell lymphoma, ALK positive and negative, (iv) Hodgkin lymphoma e.g. Nodular lymphocyte predominant Hodgkin lymphoma, Classical Hodgkin lymphoma, (v) Post Transplant Lymphoproliferative Disorders e.g. Early lesions, Polymorphic PTLD, Monomorphic PTLD and Classical Hodgkin lymphoma type PTLD.

Further, it may be possible to detect tumours which are likely to colonise the bone marrow by detecting an amount or the expression of the FOXP2 gene in lymphocytes in a sample from a subject and the invention extends to the detection of such tumours. Thus, the detection of an amount or the expression of the FOXP2 gene in a sample of lymphocytes may be indicative not only of the presence of abnormal lymphocytes but also of a disease e.g. a malignancy which is likely to colonise the bone marrow and/or lead to bone disease.

In conditions such as myeloma which are usually associated with the bone marrow, a high amount or a high level of expression of the FOXP2 gene may be associated with an increased severity of bone disease. Thus, a high amount or level of expression of the FOXP2 gene in plasma cells in a myeloma sample may indicate a patient who is likely to have an increased severity of bone disease. The high level of expression of the FOXP2 gene may be the result of the presence of plasma cells strongly expressing the FOXP2 gene and/or an increased percentage of plasma cells expressing FOXP2 e.g more than 50, 60, 70, 80 or 90% of CD138⁺ cells in the sample expressing the FOXP2 gene. Thus, a high expression level includes a sample in which more than 50%, e.g. more than 60, 70, 80 or 90% of plasma cells express the FOXP2 gene. Samples with a high expression of FOXP2 include those which have FOXP2 mRNA or FOXP2 protein levels more than 50, 100, 200, 300, 400 or 500% higher than that seen in a normal sample or seen in a sample (e.g. comprising plasma cells) obtained from a patient with a low level of bone disease or in an MGUS patient. A method of predicting the severity of bone disease in myeloma is therefore encompassed comprising the step of detecting the amount or expression level of the FOXP2 gene (e.g. the frequency or intensity of FOXP2 protein expression) in a sample of plasma cells from a patient with myeloma wherein a high amount or expression of the FOXP2 gene is indicative of an increased severity of bone disease. A step of comparing the expression level measured with a normal sample or sample obtained from a myeloma patient with a low level of bone disease or an MGUS patient may also be carried out.

In a particularly preferred aspect, the methods of the invention may be used in the diagnosis or identification of MGUS or myeloma in a subject, including particularly multiple myeloma (MM). In such aspects the invention can be seen to provide a method of diagnosis of MGUS or myeloma which includes the step of detecting abnormal plasma cells as described herein.

As noted above symptomatic or smouldering myeloma and MGUS are currently diagnosed in the art by determining the percentage of clonal plasma cells present in the bone marrow, the presence or amount of paraprotein in either serum or urine, and end-organ damage. The detection of increased FOXP2 expression levels in more than 95% of MM samples as shown by the present inventors, allows FOXP2 to be used in the diagnosis of MM, together with a determination or measurement of the percentage of clonal plasma cells present in the bone marrow to enable distinction between MM and MGUS.

Thus in one embodiment, the invention provides a method for diagnosing myeloma in a subject having >10% clonal plasma cells in bone marrow, said method comprising detecting an amount or expression of the FOXP2 gene in said plasma cells, wherein an increased amount or expression of the FOXP2 gene indicates the presence of myeloma. Further, a method of diagnosing myeloma is provided, said method comprising the steps of determining the percentage of clonal plasma cells in a bone marrow sample and detecting an amount or expression of FOXP2 in said plasma cells, wherein the detection of >10% clonal plasma cells in said sample and an increased amount or expression of FOXP2 in said plasma cells is indicative of myeloma.

Multiple myeloma may further be distinguished from asymptomatic myeloma by one or more parameters of end-organ damage, as indicated above.

In another embodiment, the invention provides a method for diagnosing MGUS in a subject having <10% clonal plasma cells in bone marrow, said method comprising detecting an amount-or expression of the FOXP2 gene in said plasma cells, wherein an increased amount or expression'of the FOXP2gene indicates the presence of MGUS. Further, a method of diagnosing MGUS is provided, said method comprising the steps of determining the percentage of clonal plasma cells in a bone marrow sample and detecting an amount or expression of FOXP2 in said plasma cells, wherein the detection of <10% clonal plasma cells in said sample and an increased amount or expression of FOXP2 in said plasma cells is indicative of MGUS.

The determination of the amount or expression of the FOXP2 gene in abnormal lymphocytes may also be utilised in the prognosis for patients with conditions associated with abnormal lymphocytes, including determining or identifying a risk or potential for disease progression. In particular, the methods of the invention may be used in the prognosis of certain types of lymphoma. Thus, results obtained by the present inventors suggest that increased expression of FOXP2 or increased amounts of FOXP2 may indicate a poor prognosis. Particularly, in relation to this aspect, the present inventors have found that FOXP2 expression may be increased in aggressive lymphomas e.g. in lymphomas which progress rapidly or are resistant to therapy. Hence, the detection of FOXP2 expression in a lymphoma patient may be indicative of a poor prognosis. As discussed above, the detection of FOXP2 expression in lymphocytes in a lymphoma patient may also be indicative of a tumour which is likely to colonise the bone marrow.

A further embodiment of the invention is therefore a method for determining the prognosis of a subject having a lymphoma, said method comprising detecting an amount or expression of the FOXP2 gene in a sample of said lymphoma, wherein an increased amount or expression of FOXP2 is indicative of a poor prognosis. Said amount or expression of FOXP2 may be detected in a sample of lymphocytes from said subject, which may be for example a sample of lymphocytes taken from a tumour or from a site of disease.

In particular, in the case of DLBCL, abnormal lymphocytes expressing the FOXP2 gene may be detected in ABC (activated B-cell-like)-type DLBCL but not GC (germinal centre)-type. ABC-type patients have a poorer prognosis than GC-type. Thus, the methods of the present invention may be used to identify high or increased-risk DLBCL patients.

Additionally, the detection of an increase in FOXP2 expression or the amount of FOXP2 can be used to assess the prognosis of a patient with MGUS or asymptomatic myeloma. A high ratio of abnormal to normal plasma cells in patients with MGUS and asymptomatic myeloma is considered to be a significant predictor of patients with a greatly increased likelihood of disease progression. Hence, in MGUS and asymptomatic myeloma patients a high ratio of plasma cells with expression of FOXP2 or an increased amount of FOXP2 may be indicative of a poor prognosis and of disease progression to multiple myeloma. The invention thus provides a method for determining the prognosis of a subject having MGUS or asymptomatic myeloma, said method comprising determining the ratio of FOXP2-expressing plasma cells to non-FOXP2 expressing plasma cells in a bone marrow sample from said subject, wherein a high ratio is indicative of a poor prognosis and/or of disease progression.

Additionally, a method is provided for determining the prognosis of a subject having MM, comprising determining the ratio of FOXP2 expressing plasma cells to non-FOXP2 expressing plasma cells in a bone marrow sample from said subject wherein a high ratio is indicative of a poor prognosis.

The term “high ratio” as used herein refers to a ratio of FOXP2 expressing cells:non-FOXP2 expressing cells of at least 1:1, for example 2:1, 3:1, 4:1, 5:1 or more. Alternatively viewed, a poor prognosis could be indicated where more than 10% of the plasma cells present in a sample express FOXP2 or have an increased amount of FOXP2. For example, more than 20, 30, 40, 50, 60, 70, 80, or 90% or all of the plasma cells present in a sample expressing FOXP2 may be indicative of a poor prognosis.

It has been described in the art that detection of the proportion of plasma cells in S-phase (as a measure of the proliferative activity of the cells) is prognostically significant in myeloma and that, combined with CD 138-based detection of plasma cells, it may be used as an important staging system in the prognosis of MM. Accordingly, in an embodiment of the present invention detection of FOXP2 amount or expression may be combined with detecting the number or proportion of cells in a sample in S-phase, or more particularly with detecting the number or proportion of plasma cells in S-phase. Such a method may be used as the basis of an improved method of prognosis of a plasma cell disorder, and particularly myeloma, for example to predict or assess the risk or likelihood of relapse. This may be achieved using flow cytometric techniques known and described in the art, for example using DNA/CD138 double-staining techniques as described in San Miguel et al. 1995 Blood 85: 448-465. Alternatively to determining cells in S-phase, the proliferative activity or capacity of the cells may be assessed by other means, such as for example the use of Ki-67 as mentioned above.

The methods of the invention described above may be performed on cells or tissues removed from a human subject or patient. However, as indicated above it is also within the scope of the invention to perform the method on cells or tissues removed from a non-human mammal, such as a laboratory, livestock or domestic animal e.g. a mouse, rat, rabbit, monkey, dog or cat.

Samples may be obtained from subjects by methods known in the art, for example by biopsy. The samples may be used directly in the methods, or they may be treated in any desired or convenient way. For example, lymphocytes may be isolated or purified therefrom.

It will be appreciated from the discussions above that the invention may be viewed as providing a use of the FOXP2 gene as a marker for abnormal lymphocytes, wherein detection of FOXP2 expression or of an increased amount of FOXP2 indicates the presence of abnormal lymphocytes, particularly abnormal plasma cells. Preferably, as set out above, FOXP2 may be used as a marker for pre-malignant or malignant lymphocytes. Additionally, in line with the discussion above, the invention includes the use of the FOXP2 gene as a marker for detecting or assessing a condition associated with the presence of abnormal lymphocytes. The FOXP2 gene (or indeed the FOXP2 protein) may thus be used a prognostic and/or diagnostic marker for such a condition, and in particular plasma cell disorders or lymphomas.

In addition to a utility as a prognostic and/or diagnostic marker, the association of FOXP2 expression with abnormal lymphocytes identifies it as a potential therapeutic target in the treatment of conditions associated with the presence of abnormal lymphocytes. Further, as discussed above, since FOXP2 expression may be indicative of tumours or diseases (including malignancies and tumours) which are likely to colonise bone or which may have an increased severity of accompanying or associated bone disease, targeting FOXP2 expression may reduce the severity of the bone disease and/or reduce colonisation of bone.

Accordingly, a further aspect of the invention provides a method for treating, in a subject suffering therefrom, a condition associated with abnormal lymphocytes, said method comprising administering to said subject an agent which inhibits FOXP2 expression and/or FOXP2 activity.

This aspect of the invention also provides an agent which inhibits FOXP2 expression and/or FOXP2 activity for use in the treatment of a condition associated with abnormal lymphocytes. Further, the invention encompasses the use of an agent which inhibits FOXP2 expression and/or FOXP2 activity in the manufacture of a medicament for treating a condition associated with abnormal lymphocytes.

Said condition associated with abnormal lymphocytes may include a primary disease (including particularly a malignancy) or a secondary disease (e.g. metastases) or indeed any accompanying or associated disease which may accompany or be associated with a primary disorder, such as bone disease as discussed above.

Thus, additionally, a method of reducing the severity of bone disease and/or colonisation of the bone in conditions associated with abnormal lymphocytes is provided, said method comprising administering an agent which inhibits FOXP2 expression and/or FOXP2 activity. This aspect also provides an agent which inhibits FOXP2 expression and/or FOXP2 activity for use in reducing the severity of bone disease and/or colonisation of the bone in conditions associated with abnormal lymphocytes.

As discussed further below in the Examples, it has been shown by the present inventors that silencing FOXP2 expression results in upregulation of SMAD-3 expression. The SMAD proteins are involved in the propagation of the TGF-β signal which negatively regulates cellular proliferation. SMAD3 also negatively regulates both Runx2 expression and Runx2 activity (Borton Hjelmeland et al, 2005, Mol. Cell Biol., 25 (21), 9460-9468), thus FOXP2 regulation of SMAD3 may be necessary to activate Runx2. Hence, upregulating the expression of SMAD3 (by inhibiting FOXP2 expression) may present a method for preventing cellular proliferation and for treating conditions associated with the presence of abnormal lymphocytes. Further, the inventors have shown that IRF-4 expression and cyclin D1 expression are repressed when FOXP2 expression is silenced. Inhibition of the IRF-4 pathway is toxic to myeloma tumour cells and cyclin D1 has a role in cell proliferation and hence these results also show that inhibition of FOXP2 expression can be used to treat conditions associated with the presence of abnormal lymphocytes.

Additionally, a key feature of myeloma is that in vivo the cells localise to the bone marrow and interact with stromal cells. This is a particularly important aspect of myeloma biology because cell adhesion mediated drug resistance is an intrinsic mechanism of myeloma resistance to chemotherapeutic drugs. The inventors have shown that FOXP2 silencing reduces the number of myeloma cells adhering to the stromal cell monolayer and thus targeting FOXP2 expression or function may be able to overcome cell adhesion mediated drug resistance and allow the effective treatment of myeloma.

Conditions which may be treated according to this aspect of the invention may be any condition associated with the presence of abnormal lymphocytes, as discussed above, including particularly plasma cell disorders, and particularly plasma cell malignancies, notably myeloma, and lymphomas, including both Hodgkin lymphoma and non-Hodgkin's lymphomas, which may include various T-cell lymphomas, and B-cell lymphomas, e.g. diffuse large B-cell lymphoma. T-cell lymphomas include Adult T-cell lymphoma, Enteropathy-type T-cell lymphoma, hepatosplenic T-cell lymphoma and angioimmunoblastic T-cell lymphoma. Other lymphomas are listed above. Advantageously, the condition is multiple myeloma, asymptomatic myeloma or MGUS.

The term “treatment” is used herein to broadly include both therapeutic and prophylactic treatment i.e. both therapy and prevention. Hence, in prophylactic applications, the agent or more particularly a pharmaceutical composition comprising the agent may be administered to a patient who may not yet have developed a condition associated with the presence of abnormal lymphocytes. Such a patient may for example be at risk of developing the condition.

The agent which inhibits FOXP2 expression and/or activity may be any agent (e.g. inhibitor or antagonist) which can prevent or reduce the transcription or translation of FOXP2 mRNA or FOXP2 protein, can degrade FOXP2 mRNA or protein or can prevent or reduce the activity of FOXP2 protein e.g. can alter its binding or functionality. Such an agent can for example reduce the expression of FOXP2 by 10, 20, 30, 40, 50, 60, 70, 80, 90 or 100%. A reduction in the level of expression of FOXP2 can be determined or detected by any of the methods described previously above.

An agent which inhibits FOXP2 expression includes an antisense molecule (e.g. an antisense oligonucleotide) of FOXP2 or a nucleic acid molecule capable of hybridising to FOXP2 to inhibit expression thereof. Antisense technology can be used to control gene expression through triple-helix formation of antisense DNA or RNA, both of which methods are based on binding of a polynucleotide to DNA or RNA. For example, the 5′ coding portion or the mature protein sequence which encodes for the FOXP2 protein can be used to design an antisense RNA oligonucleotide of from 10 to 40 base pairs in length. The antisense RNA oligonucleotide hybridises to the mRNA and blocks translation of an mRNA molecule into protein. A DNA oligonucleotide is designed to be complementary to a region of the gene involved in transcription thereby preventing transcription and protein production. Hence, an antisense nucleic acid molecule comprising at least 10, 15, 20, 25, 30, 35, 40, 45 or 50 nucleotides which is capable of hybridising to FOXP2 can be used to treat a condition associated with the presence of abnormal lymphocytes. Absolute complementarity is not required and any oligonucleotide having sufficient complementarity to form a stable duplex with the target is suitable.

However, antisense sequence are usually designed to complement the mRNA target (FOXP2) and form RNA:antisense duplex. This duplex formation can prevent processing, splicing, transport, or translation of FOXP2 mRNA. Certain antisense sequences can also elicit cellular RNase H activity when hybridised with the mRNA, resulting in mRNA degradation. In that case, RNase H will cleave the RNA of the duplex and can potentially release the antisense molecule to hybridise further with additional molecules of FOXP2 mRNA. As described above, an additional mode of action results from the interaction of antisense with genomic DNA to form a triple helix which may be transcriptionally inactive. The sequence target segment of FOXP2 for the antisense oligonucleotide is selected such that the sequence exhibits suitable energy related characteristics important for oligonucleotide duplex formation with their complementary templates, and shows a low potential for self-dimerisation or self-complementation. It is possible to use the OLIGO computer program (primer analysis software version 3.4) to determine the antisense sequence melting temperature, free energy properties and to estimate potential self-dimer formation and self-complementarity properties. The program allows the determination of a qualitative estimation of these two parameters and provides an indication of “no potential” or “some potential” or “essentially complete potential”.

An antisense oligonucleotide may be delivered to cells by procedures well known in the art, for example, in vectors e.g. in plasmid vectors or viruses.

Small nucleic acid molecules such as siNA, siRNA, dsRNA, miRNA and shRNA which can be used to mediate RNA interference can be used as inhibitors of FOXP2 expression. shRNA is a sequence of RNA that makes a tight hairpin turn that can be used to silence gene expression via RNAi. siRNA are a class of double-stranded RNA molecules that may be from 18 to 25 (e.g. 19, 20, 21, 22, 23, 24) nucleotides in length. In a particularly preferred embodiment of the invention, a duplex siRNA of ATGGAAGACAATGGCATTAAA may be used as an inhibitor of FOXP2.

Further, the following siRNAs (Invitrogen) may be used:

2580-double stranded made from
5′-GACAGGCAGUUAACACUUAAUGAAA-3′
plus
5′-UUUCAUUAAGUGUUAACUGCCUGUC-3′
0274-double stranded made from
5′-CAGUUUAGGCUAUGGAGCAGCUCUU-3′
plus
5′-AAGAGCUGCUCCAUAGCCUAAACUG-3′
0275-double stranded made from
5′-CAGAGAGAUUGAAGAAGAGCCUUUA-3′
plus
5′-UAAAGGCUCUUCUUCAAUCUCUCUG-3′

Aptamers are nucleic acid molecules that can bind to proteins, nucleotides and complexes and can be use to inhibit FOXP2 expression and/or FOXP2 activity. Aptamers can be modified for stability or other desired qualities and modifications can be introduced anywhere in the molecule such as at the 5′ or 3′ termini or at any internally defined modification site. For example RNA aptamers can be stabilised with 2′-fluoro or 2′-amino modified pyrimidines. Aptamers can also be linked to reporter molecules or linkers. An aptamer inhibitor of FOXP2 may be from 10 to 50 nucleotides (e.g. from 15, 20, 30, 40 nucleotides) in length.

Ribozymes may further be used as inhibitors of FOXP2 expression in accordance with the present invention. Ribozymes possess RNA catalytic ability and can cleave a specific site in RNA. Ribozymes catalyse the phosphodiester bond cleavage of RNA and several ribozyme structural families have been identified including Group I introns, RNase P, the hepatitis delta virus ribozyme, hammerhead ribozymes and the hairpin ribozyme originally derived from the negative strand of the tobacco ringspot virus satellite RNA. In general, the ribozyme has a length of from 30 to 100 nucleotides.

Additionally, decoy oligonucleotides that prevent DNA binding, i.e. the binding of FOXP2 to DNA, may be used as inhibitors of FOXP2 activity, since FOXP2 is a transcription factor. Further, any molecules which prevent or disrupt FOXP2 interaction with a coregulatory protein which is necessary for transcription may be used to inhibit FOXP2. Dimerisation of FOXP2 may be required for DNA binding and hence disruption of dimerisation can be used to inhibit FOXP2.

In a further embodiment of the invention, the agent may be an anti-FOXP2 antibody. An antibody to FOXP2 may be raised according to standard techniques well known to those skilled in.the art by using a FOXP2 protein as previously described herein, or a fragment thereof as an antigen/immunogen in a host animal e.g. the FOXP2 protein of SEQ ID NO 2, 4, 6, 8, 10, 12, 14, 16, 18, 20, 22, 24, 26, 28, 30, 32, 34 or 37 or a fragment or epitope from the protein

The antibody may be monoclonal or polyclonal and may be of any convenient or desired species, class or sub-type. Furthermore, the antibody may be natural, derivatised or synthetic.

The “anti-FOXP2 antibody” which can be used as an inhibitory agent thus includes:

(a) any of the various classes or subclasses of immunoglobulin e.g. IgG, IgA, IgM, IgD or IgE derived from any animal e.g. any of the animals conventionally used e.g. sheep, rabbits, goats, or mice or egg yolk;

(b) monoclonal or polyclonal antibodies;

(c) intact antibodies or fragments of antibodies, monoclonal or polyclonal, the fragments being those which contain the binding region of the antibody e.g. fragments devoid of the Fc portion (e.g. Fab, Fab′, F(ab′)2, Fv), the so called “half molecule” fragments obtained by reductive cleavage of the disulphide bonds connecting the heavy chain components in the intact antibody. Fv may be defined as a fragment containing the variable region of the light chain and the variable region of the heavy chain expressed as two chains

(d) antibodies produced or modified by recombinant DNA or other synthetic techniques, including monoclonal antibodies, fragments of antibodies, humanised antibodies, chimeric antibodies, or synthetically made or altered antibody-like structures.

Also included are functional derivatives or “equivalents” of antibodies e.g. single chain antibodies. A single chain antibody may be defined as a genetically engineered molecule containing the variable region of the light chain, the variable region of the heavy chain, linked by a suitable polypeptide linker as a fused single chain molecule. Methods for producing antibodies and the fragments and derivatives of the antibodies are described in greater detail below.

Anti-FOXP2 antibodies known in the art include FOXP2 N-16 sc-21069 (Santa Cruz, Calif., USA). Such antibodies may form the basis for preparing derivatives or synthetic constructs which might be used as therapeutic antibodies according to this aspect of the invention.

In order to enable or facilitate the use of antibodies as therapeutic agents, single chain Sv intrabodies may be engineered, to target the FOXP2 protein. Such an approach has been described for p53 and EGFR in Caron de Fromentel et al. 1999 Oncogene 18: 551-557 and Beerli et al. 1996 Breast Cancer Res. Treat. 38: 11-17.

As will be described further below, in work leading up to this invention a novel anti-FOXP2 antibody has been developed, and this antibody identified herein as FOXP2-73A18 antibody, or a derivative thereof may be used as a therapeutic agent according to this aspect of the invention.

Other inhibitory agents which can be used to inhibit FOXP2 activity may be agents which can inhibit dimerisation of FOXP2. Additionally, it is possible to inhibit FOXP2 activity by administering a binding partner for FOXP2, which would have the effect of “mopping up” or sequestering FOXP2 in a cell and hence preventing it from exerting its effect in the cell. It will be appreciated that such a binding partner may be an antibody, but it may also be any binding partner for FOXP2 and such a binding partner may be identified by a variety of techniques known in the art. By way of example, such agents may include CtBP1 and NFAT, or FOXP2 binding fragments thereof, or other proteins or protein fragments, peptides etc. which have been reported to, or are capable of, binding FOXP2. Molecules which alter (increase or decrease) the phosphorylation, ubiquitination, acetylation or SUMOlation of FOXP2 can also be used as agents in the present invention.

Small molecules may also be used as agents, for example non-biological molecules such as organic compounds, organometallic compounds, salts of organic and organometallic compounds, saccharides, and amino acids. Additionally, other small molecules can include biological molecules of low molecular weight including lipids, oligosaccharides, oligopeptides or their derivatives. Such inhibitory small molecules may be identified by screening for ability to inhibit FOXP2 expression or FOXP2 activity according to principles or techniques known and described in the art.

An agent which inhibits FOXP2 expression or activity can be a known agent or novel agents can be identified by screening for their ability to inhibit FOXP2 expression or activity. Test agents for screening may be obtained from a variety of sources e.g. compound libraries, for example combinatorial libraries or peptide libraries such as phage display libraries, which may be generated according to procedures or principles well known in the art, or from libraries of natural compounds e.g. in the form of bacterial, plant, fungal and animal extracts which can be obtained from commercial sources or collected in the field. The agent which inhibits FOXP2 expression or activity may also be obtained by rational design, for example based on structures of known agents which inhibit FOXP2 expression or activity. Known agents which inhibit FOXP2 expression or activity may be subjected to directed or random chemical modification to produce structural analogues.

The agent may be formulated in a pharmaceutical composition together with any suitable pharmaceutically acceptable carrier, diluent or excipient. The agents may be encapsulated and/or combined with suitable carriers in solid dosage forms for oral administration or alternatively with suitable carriers for administration in aerosol form. The inhibitors may further be combined with any other carrier for administration by injection e.g. by subcutaneous or intramuscular injection.

The pharmaceutical compositions may include pharmaceutically acceptable carriers including for example non-toxic salts, sterile water or the like. A suitable buffer may also be present allowing the compositions to be lyophilised and stored in sterile conditions prior to reconstitution by the addition of sterile water for subsequent administration. The carrier can also contain other pharmaceutically acceptable excipients for modifying other conditions such as pH, osmolarity, viscosity, sterility, lipophilicity, somobility or the like. Pharmaceutical compositions which permit sustained or delayed release following administration may also be used.

Furthermore, the dosage regime may be determined according to the nature and weight of the patient, and may depend on the particular route of administration to be used. The dosage administered may readily be determined by a medical practitioner based on the circumstances pertaining to the disorder to be treated, such as the severity of the symptoms, the age, weight, and response of the individual etc.

Thus, the amount of agent in the composition may vary considerably e.g. from less than 0.5%, usually 1% to as much as 15 or 20% by weight. This can also be dependent on the fluid volumes, viscosities, etc., preferable for the particular mode of administration selected.

Single or multiple administrations of the compositions can be carried out with dose levels and patterns being selected by the treating physician.

The agents may also be administered in combination together, separately, sequentially, or simultaneously, with other therapeutic agents effective against, or used in the treatment of the condition in question. In this respect, since FOXP2 silencing reduces the number of myeloma cells adhering to the stromal cell monolayer, targeting FOXP2 expression or function in this way may be able to overcome cell adhesion mediated drug resistance and allow the effective treatment of myeloma. Thus, the invention encompasses the treatment of a condition associated with abnormal lymphocytes using an agent which is capable of inhibiting FOXP2 expression or FOXP2 activity, in combination with a further therapeutic agent which can treat the condition. A product is therefore provided which contains an agent which inhibits FOXP2 expression and/or FOXP2 activity and a therapeutic agent effective against or used in the treatment of a condition associated with abnormal lymphocytes as a combined preparation for simultaneous, separate or sequential use in treating a condition associated with abnormal lymphocytes.

Particularly, the therapeutic agent may be a chemotherapeutic agent. Chemotherapy is the use of drugs to destroy or control cancer cells and thus a chemotherapeutic agent may have a cytotoxic or cytostatic or other controlling effect on cancer cells. Many different drugs or chemotherapeutic agents are commonly use to treat MM, for example: melphalan, vincristine, cyclophosphamide, carmustine, doxorubicin, Thalidomid® (thalidomide), Velcade® (bortezomib), Lenalidomide or Revlimid®. These drugs may be given in combination and may be combined with other drugs such as immunomodulating agents or corticosteroids, immunotherapies or other treatments such as radiotherapy and autologous or allogeneic peripheral blood stem cell transplants. Combinations are determined by many factors and include melphalan and prednisone (MP), with or without thalidomide or bortezomib; vincristine, doxorubicin and dexamethasone (VAD); thalidomide and dexamethasone; bortezomib, thalidomide, plus dexamethasone; and liposomal doxorubicin, vincristine, dexamethasone. Thus, any one or more of these chemotherapeutic agents, drugs or treatments may be administered simultaneously, separately or sequentially to the agent which inhibits FOXP2 expression and/or FOXP2 activity.

In this aspect, the product may comprise an antisense RNA e.g. a siRNA molecule as described previously or an antibody as described herein. Thus, particularly, the invention provides a method of treating a condition associated with abnormal lymphocytes in a subject suffering therefrom said method comprising administering to said subject an agent which inhibits FOXP2 expression and/or FOXP2 activity, together with a therapeutic agent, wherein said agent and therapeutic agent are administered either simultaneously, sequentially or separately. As discussed above, the therapeutic agent may be a chemotherapeutic agent.

Additionally, the inventors have found that an increased expression of FOXP2 occurs in other tumours which have a bone disease phenotype, for example in Ewing's sarcoma. This data, together with the bone disease phenotype observed in myeloma where an increase of FOXP2 expression in lymphocytes is also seen, indicates that inhibition of the activity of FOXP2 or expression of the FOXP2 gene could be used for the treatment of bone disease associated with FOXP2-expressing tumours. The inhibition of FOXP2 activity or FOXP2 expression may prevent such tumours from homing to the bone marrow e.g. colonising the bone, or may reduce the severity of bone disease seen.

Thus, the invention provides a method of treating bone disease associated with a tumour having an increased amount or expression of FOXP2 gene, said method comprising administering to a subject having or at risk of said bone disease, an agent which inhibits FOXP2 expression and/or FOXP2 activity.

This aspect of the invention also provides an agent which inhibits FOXP2 expression and/or FOXP2 activity for use in the treatment of bone disease associated with tumours having an increased amount or expression of the FOXP2 gene. This may include primary tumours localised in the bone marrow, such as myeloma and some lymphomas together with those where the cancer metastasizes to the bone marrow e.g. lymphoma and some carcinomas e.g. thyroid and kidney.

“Bone disease” which may be associated with a FOXP2-expressing tumour encompasses osteoporosis, bone lesions, bone pain, fractures, immunosuppression or other such phenotypes which can occur as the result of the presence of a tumour. The treatment of bone disease may reduce the severity of the bone disease which occurs e.g. the number of lesions in the bones may be reduced by at least 10, 20, 30, 40 or 50% or may prevent bone disease from occurring.

“A tumour having an increased amount or expression of FOXP2” refers to an abnormal mass of tissue (e.g. comprising abnormal cells which may have an increased size, increased proliferation, loss of characteristics e.g. cell markers, may be less differentiated etc than their normal cell counterparts), which results from excessive cell division. The tumour may comprise malignant cells and the tumour (or a sample thereof) has an increased amount or expression of FOXP2 compared to normal or non-malignant cells of the same type. The tumour may be heterogeneous and may therefore comprise both abnormal (e.g. malignant) cells and normal cells. Not all cells in the tumour may therefore have an increased amount or expression of FOXP2, but generally, at least 5%, preferably 10% of the tumour cells will express FOXP2 for the tumour to be considered as FOXP2 expressing. As discussed above, the tumour may include primary tumours localised in the bone marrow, such as myeloma and some lymphomas together with those where the cancer metastasizes to the bone marrow e.g. lymphoma and some carcinomas e.g. thyroid and kidney.

If the non-malignant or normal cell counterparts do not express the FOXP2 gene, an increased expression of the FOXP2 gene in a tumour may be the detection of any expression of the FOXP2 gene, as discussed previously for abnormal lymphocytes (and the comments regarding the expression of FOXP2 made previously with respect to abnormal lymphocytes apply equally here). However, preferably, as discussed above, at least 5% or 10% of cells in the tumour should express FOXP2 for the tumour to be considered as FOXP2 expressing, to rule out de minimis expression.

If the non-malignant or normal cells usually express FOXP2, then a tumour which has an increased amount or expression of FOXP2, has an increased or higher amount or expression of the FOXP2 gene compared to the amount or expression of FOXP2 in the normal or non-malignant cells. Thus, the tumour cells may contain more copies of the FOXP2 gene than the normal counterpart cells, or may have an increased level of FOXP2 mRNA or FOXP2 protein than the normal counterpart cells e.g. more than 10, 20, 30, 40, 50, 60, 70, 80 or 90%. Examples of tumours having an increased amount or expression of FOXP2 include Ewing's sarcoma and bone-metastatic lesions of other mesenchymal tumours eg rhabdomyosarcoma.

The term “malignant” as used above and as discussed previously in terms of lymphocytes, refers to cells which exhibit abnormal behaviour, particularly in terms of growth, death or differentiation. Malignant cells may not be self limited in their growth, they may fail to die at the appropriate time and/or they may be arrested at a particular stage of development or differentiation. For example, malignant cells may have increased proliferation compared to their normal counterparts e.g. increased by more than 5, 10, 15, 20, 25, 30, 40 or 50%. A malignant cell may be considered as a cancerous cell.

An agent which inhibits FOXP2 expression and/or FOXP2 activity to treat bone disease is as defined previously for the treatment of conditions associated with abnormal lymphocytes. Further, as described previously, such an agent can be combined with a therapeutic agent which is active against the tumour having an increased amount or expression of FOXP2 e.g. a chemotherapeutic agent as defined above.

As described previously, the detection of FOXP2 in the method of detecting abnormal lymphocytes can be achieved by using an antibody. However, during the studies which were carried out in association with the present invention, it was surprisingly discovered that the antibodies of the prior art were not suitable for detecting endogenous FOXP2 by immunohistochemistry, although they were reactive with recombinantly expressed FOXP2 protein. The inventors therefore sought to develop antibodies which could reliably be used to detect FOXP2 protein using any method. It was surprisingly discovered that antibodies directed to the N-terminus of FOXP2 could be used to detect endogenous FOXP2.

Hence, a further aspect of the present invention is directed to an antibody which binds specifically to the N-terminus of FOXP2.

Thus, the antibody may bind specifically to the N-terminus of FOXP2 and not to other FOXP proteins. Particularly, the antibody can bind to both recombinant FOXP2 and to native endogenous FOXP2. Thus, a preferred and advantageous feature of the antibodies of the present invention is that may bind to native FOXP2 protein as expressed by a native cell, i.e. in situ in a native cell (namely a cell which has not been recombinantly or genetically engineered or manipulated in any way).

By “binding specifically” is meant that the antibody is capable of binding to the FOXP2 protein in a manner which distinguishes it from the binding to non-target molecules. Thus, the antibody either does not bind to non-target molecules or exhibits negligible or substantially reduced (as compared to FOXP2) e.g. background, binding to non-target molecules. Thus the antibody specifically recognises FOXP2, in particular specifically recognises or binds to the N-terminus (also referred to herein as the N-terminal domain of FOXP2). The antibody does not therefore bind or exhibits negligible binding to FOXP1, FOXP3 or FOXP4 proteins.

The antibody of the present invention is capable of binding to the N-terminal domain of the FOXP2 protein sequence defined above and hence may be capable of recognising any FOXP2 variants or isoforms comprising this region or an epitope therefrom. The N-terminal domain of FOXP2 may consist of the region from position 1 to position 250 from a FOXP2 protein, and particularly FOXP2 as defined above. Hence, an antibody of the invention may bind to any epitope or at any point within this N-terminal domain. Preferably, the N-terminal domain is from position 1 to position 200, from position 1 to position 150, from position 1 to position 100 or from position 1 to position 90. In a particularly preferred embodiment, the antibody binds to the N-terminal region as defined by position 1 to position 86 for example in SEQ ID NO. 2.

Whilst the antibody of the invention may be of any type, including both polyclonal and monoclonal antibodies, it is advantageous that the antibody is of a single specificity.

The antibody may be of any convenient or desired species, class or sub-type. Furthermore, the antibody may be natural, derivatised or synthetic. The term antibody as used herein thus includes all types of antibody molecules and antibody fragments.

More particularly the “antibody” according to the present invention includes:

(a) any of the various classes or subclasses of immunoglobulin e.g. IgG, IgA, IgM, IgD or IgE derived from any animal e.g. any of the animals conventionally Used e.g. sheep, rabbits, goats, or mice or egg yolk

(b) monoclonal or polyclonal antibodies

(c) intact antibodies or fragments of antibodies, monoclonal or polyclonal, the fragments being those which contain the binding region of the antibody e.g. fragments devoid of the Fc portion (e.g. Fab, Fab′, F(ab′)2, Fv), the so called “half molecule” fragments obtained by reductive cleavage of the disulphide bonds connecting the heavy chain components in the intact antibody. Fv may be defined as a fragment containing the variable region of the light chain and the variable region of the heavy chain expressed as two chains

(d) antibodies produced or modified by recombinant DNA or other synthetic techniques, including monoclonal antibodies, fragments of antibodies, humanised antibodies, chimeric antibodies, or synthetically made or altered antibody-like structures. Also included are functional derivatives or “equivalents” of antibodies e.g. single chain antibodies. A single chain antibody may be defined as a genetically engineered molecule containing the variable region of the light chain, the variable region of the heavy chain, linked by a suitable polypeptide linker as a fused single chain molecule. Also included are single chain (Sv) intrabodies as discussed above.

Methods of making such antibody fragments and synthetic and derivatised antibodies are well known in the art. Also included are antibody fragments containing the complementarity-determining regions (CDRs) or hypervariable regions of the antibodies. These may be defined as the region comprising the amino acid sequences on the light and heavy chains of an antibody which form the three dimensional loop structure that contributes to the formation of the antigen binding site. CDRs may be used to generate CDR-grafted antibodies. As used herein “CDR grafted” defines an antibody having an amino acid sequence in which at least parts of one or more sequences in the light and/or variable domains have been replaced by analogous parts of CDR sequences from an antibody having a different binding specificity for a given antigen. One of skill in the art can readily produce such CDR grafted antibodies using methods well known in the art.

A chimeric antibody may be prepared by combining the variable domain of an anti-FOXP2 antibody of one species with the constant regions of an antibody derived from a different species.

Monoclonal antibodies and their fragments and derivatives are preferred antibodies according to the present invention. A further aspect of the invention is thus a hybridoma or cell line producing an antibody of the invention as defined above.

In particular, a novel antibody has been obtained having the properties defined above. This antibody is defined herein as FOXP2-73A/8 and the hybridoma producing this antibody has been deposited under the terms of the Budapest Treaty at the ECACC (Porton Down, Salisbury) on 14 Oct. 2008 under the Accession Number 08101410.

Accordingly, a preferred embodiment of this aspect of the invention provides a monoclonal antibody produced by or obtainable from the hybridoma cell line of ECACC deposit Accession Number 08101410.

In a further related aspect, the invention provides a hybridoma (or hybrid cell line) being that on deposit at the ECACC under. Accession Number 08101410.

Also part of the invention are antibodies and hybridomas or cell lines having the identifying characteristics of antibody FOXP2-73A/8 or the hybridoma of ECACC Accession Number 08101410.

By “identifying characteristics” is meant for example the properties and characteristics e.g. functional effects of the antibodies of the invention (e.g. the ability to bind to the N-terminus of FOXP2). In particular, included under this aspect of the invention are antibodies having the same binding specificities and/or binding properties as antibody FOXP2-73A/8 produced by hybridoma ECACC Accession No. 08101410.

Thus, for example, the genes encoding antibody FOXP2-73A/8 may be identified, cloned and examined. These may be utilised to create an antibody construct (e.g. a synthetic antibody chain, a CDR grafted antibody or a chimeric antibody) having a binding pattern and/or specificity identical to that of antibody FOXP2-73A/8. An antibody having the identifying characteristics or binding specificity of antibody FOXP2-73A/8 may thus be a derivative of the antibody FOXP2-73A/8 (the antibody produced by hybridoma ECACC Deposit Accession No.) 08101410.

An antibody of the invention can be obtained or made according to techniques standard and well known in the art and widely described in the literature. Thus, a host animal may be immunised with a FOXP2 immunogen from the N-terminal region and used to generate a polyclonal or more preferably a monoclonal antibody using well known standard techniques. Likewise, techniques for generating fragments of antibodies or antibody derivatives are also well known in the art. Thus for example, phage display methods may be used to make antibodies.

The antibody of the invention may also be labelled e.g. with a small molecule such as hapten, biotin etc, or with a fluorochrome, radioisotope, coloured dye, enzyme, colloidal metal, chemi or bio-luminescent compound. Further, the antibody of the invention may be bound to a carrier or immobilised on a solid support. For example, the antibody may be immobilised on glass, polystyrene, polyethylene etc, dextran, nylon, amyloses, celluloses, polyacrylamides, agaroses, or solid surfaces such as particles e.g. magnetic or non-magnetic beads, the surfaces of plates, wells and tubes or strips. Methods for coupling or immobilising antibodies are well known in the art.

The antibody of the invention can be used to detect the FOXP2 protein and hence can be used in the methods described above e.g. in the methods of detecting abnormal lymphocytes. Further, the antibody of the invention can be used in the methods of treating conditions associated with the presence of abnormal lymphocytes as described above. Thus, also provided herein as further aspects of the present invention are an antibody of the invention as defined herein for use in therapy, and particularly for use in treating a condition associated with abnormal lymphocytes, a pharmaceutical composition containing an antibody of the invention and the use of antibody of the invention in the manufacture of a medicament for use in treating a condition associated with abnormal lymphoctyes.

A kit comprising the antibody of the invention is also encompassed where the kit is for use in detecting FOXP2. Other components of such a kit may include secondary antibodies.

The invention will now be described in more detail in the following non-limiting Examples with reference to the drawings in which:

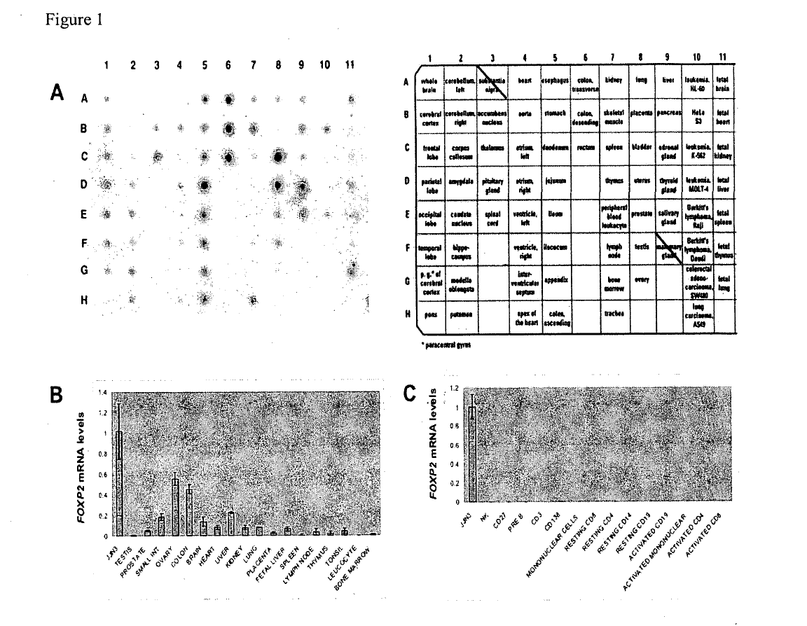

FIG. 1 shows expression of FOXP2 mRNA in normal human tissues. FIG. 1 A shows results obtained from hybridising the FOXP2 cDNA to an MTE array (Clontech Takara BioEurope, France). The top right panel shows the identity and location of tissues on the MTE array. qRT-PCR analysis of FOXP2 expression in normal human tissues is shown in FIG. 1B and in purified mononuclear cell populations is shown in FIG. 1C. FOXP2 levels are relative to those in the MM cell line JJN3.

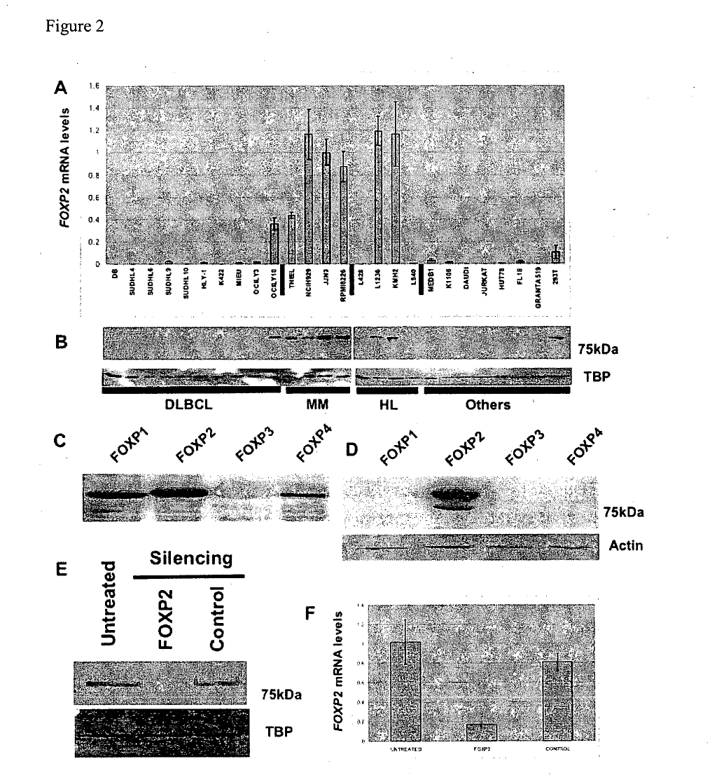

FIG. 2 shows FOXP2 mRNA and protein is expressed in lymphoma and myeloma cell lines. FIG. 2A shows qRT-PCR analysis of FOXP2 mRNA expression in cell lines derived from haematological malignancies. FIG. 2B) shows Western blotting analysis of FOXP2 protein expression in the same panel of cell lines, using the Santa Cruz polyclonal anti-FOXP2 antibody, with the lower panel showing a TBP loading control. Validation of the specificity of commercial FOXP2 antibodies for FOXP2 showing the additional cross reactivity of the Abcam antibody with FOXP1 and FOXP4 (FIG. 2C) while the Santa Cruz antibody was specific for FOXP2 (as shown in FIG. 2D). Silencing FOXP2 expression using RNAi confirmed the knock down of both endogenous FOXP2 mRNA expression (FIG. 2F) and protein expression (FIG. 2E) in the JJN3 myeloma cell line.

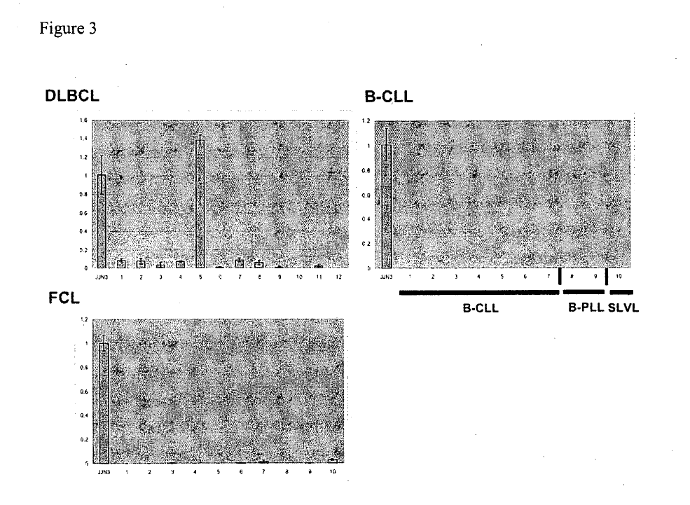

FIG. 3 shows an investigation of FOXP2 mRNA expression in B-CLL tumour cells and biopsies from DLBCL, and FL patients. qRT-PCR analysis of FOXP2 mRNA expression showed expression in a subgroup of DLBCL biopsies but not in FL (FCL) biopsies. There was no FOXP2 expression in B-CLL patients or those with B-PLL (B-cell prolymphocytic leukaemia) or SLVL (splenic lymphoma with villous lymphocytes).

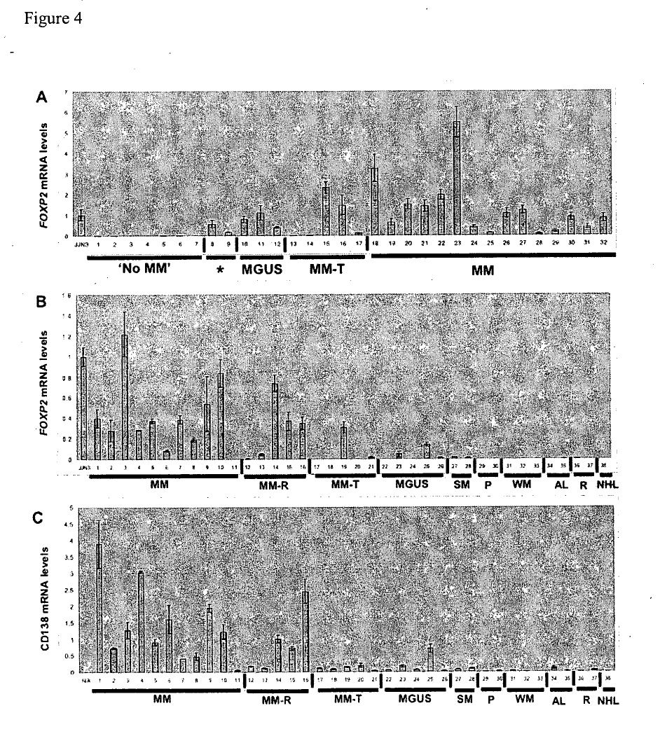

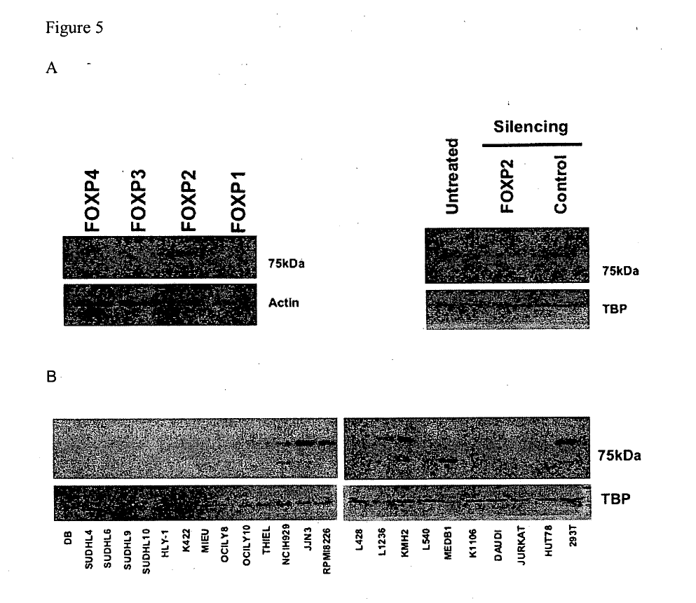

FIG. 4 shows an investigation of FOXP2 mRNA expression in purified CD138⁺ plasma cells and total bone marrow aspirates. FIG. 4A shows expression in CD138+ purified bone marrow plasma cells. Those indicated with a * represent two non-MM patients with elevated FOXP2 expression; one patient had a trephine reported as either LPL or myeloma, although clinical data suggested the more likely diagnosis was a low grade lymphoma and the other patient had a bone plasmacytoma with insufficient bone marrow trephine to define a malignant infiltrate. qRT-PCR analysis of both FOXP2 expression (FIG. 4B) and CD138 expression (FIG. 4C) was also performed using 38 aspirate samples from total bone marrow. MM=multiple myeloma at diagnosis; no MM includes a heterogenous group of patients whose plasma cells should not be malignant or pre-malignant; MM-T refers to samples taken from patients post-treatment; MM-R=MM patients at relapse; SM=smoldering myeloma; P=plasmacytoma, WM=Waldenstroms Macroglobulinaemia; AL=amyloid; R=reactive marrow; NHL=non-Hodgkin's lymphoma.

FIG. 5 shows validation of the FOXP2-73A/8 monoclonal antibody. Western blotting (FIG. 5A) showed that 73A/8 specifically recognised FOXP2 transfected cells and not the other FOXP proteins (top left). Silencing using FOXP2-targeted siRNA confirmed the identity of the endogenous protein in the JJN3 cell line as FOXP2. Western blotting of nuclear extracts from lymphoma and MM cell lines confirms the pattern of reactivity observed using the polyclonal FOXP2 antibody from Santa Cruz. However, the 73A/8 antibody also detects two additional lower molecular weight bands that might represent additional FOXP2 isoforms (possibly generated via the known alternative splicing of this gene). Although the full length FOXP2 protein was not expressed in the MEDB1 cell line, although it did express some FOXP2 mRNA, 73A/8 detected significant amounts of the smallest protein. The differences between the two antibodies may reflect their differential ability to recognise epitopes in individual FOXP2 isoforms.

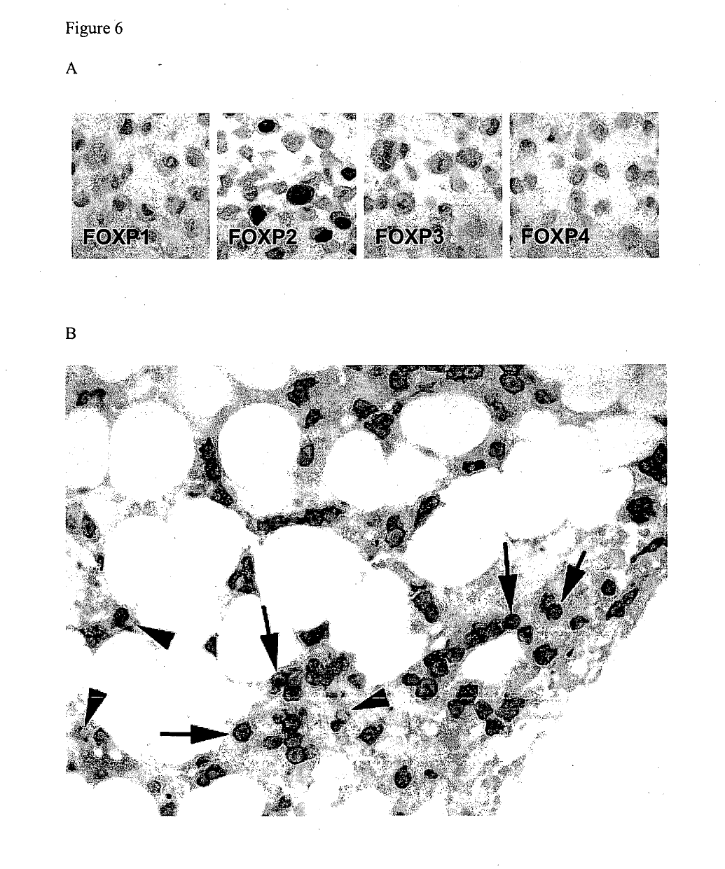

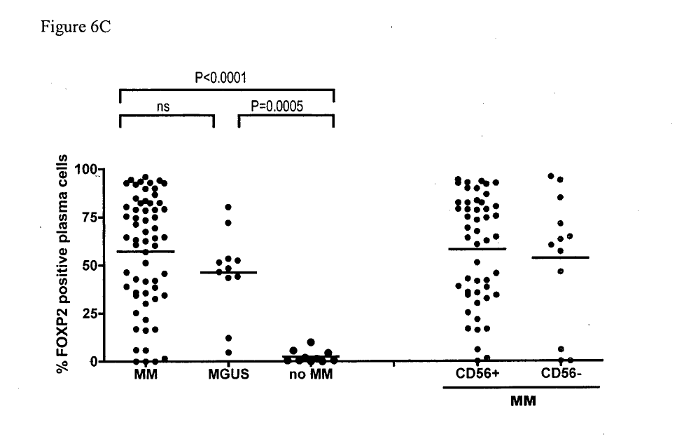

FIG. 6 shows immunolabelling with antibody FOXP2-73A/8 and analysis of the data obtained using immunohistochemistry. The top panel of FIG. 6A illustrates FOXP2-73A/8 peroxidase immunolabelling of FOXP transfectants (detection using the Dako Envision System). The strong nuclear labelling of cells transfected with the FOXP2 cDNA, and not those transfected with the related FOXP proteins (FOXP1, FOXP3 and FOXP4), confirms the ability of this antibody to specifically detect FOXP2 by immunohistochemistry. The bottom panel of FIG. 6B illustrates a FOXP2-positive MM case stained for CD138 (cell surface labelling of plasma cells) and FOXP2 (nuclear labelling), confirming the expression of FOXP2 in malignant plasma cells. Arrowheads indicate the occasional CD138-positive cells that lack FOXP2 expression, while the arrows indicate examples of the majority of the CD 138-positive cells that express nuclear FOXP2. FIG. 6C shows the percentage of FOXP2+ plasma cells in MM, MGUS and reactive BM (no MM) in the CD138+ population. Both MGUS and MM patients exhibit a significantly higher frequency of FOXP2 plasma cell expression than reactive BM plasma cells. To the right of this panel, the MM patients are grouped by CD56 expression, CD56 positive cases on the left and CD56 negative on the right.

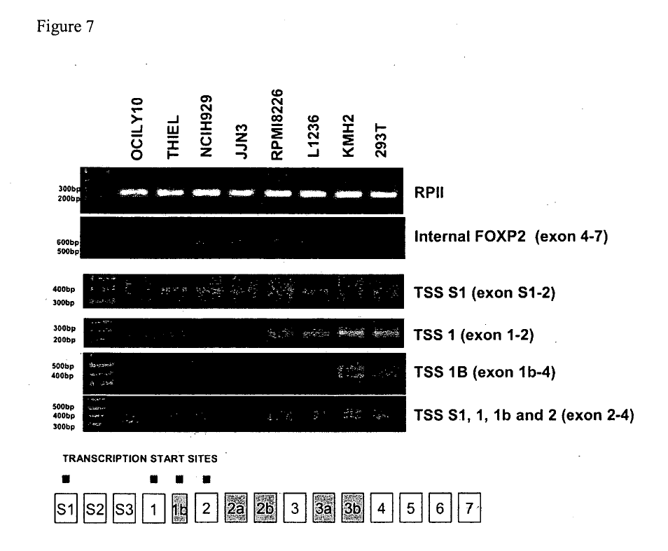

FIG. 7 shows RT-PCR analysis of FOXP2 transcription start sites in MM and lymphoma cell lines. Names to the right of each PCR panel indicate the transcription start site assayed and the location of the PCR primers within individual exons is indicated in brackets. TSS=transcription start site. RPII=RNA polymerase II (RNA quality control), Internal FOXP2 confirms the expression of FOXP2 mRNA independently of the TSS. The diagram at the bottom shows the exon structure of the FOXP2 gene in the region analysed using RT-PCR. Alternatively spliced exons are shaded grey and the transcriptional start sites are indicated above the exons as small black squares.

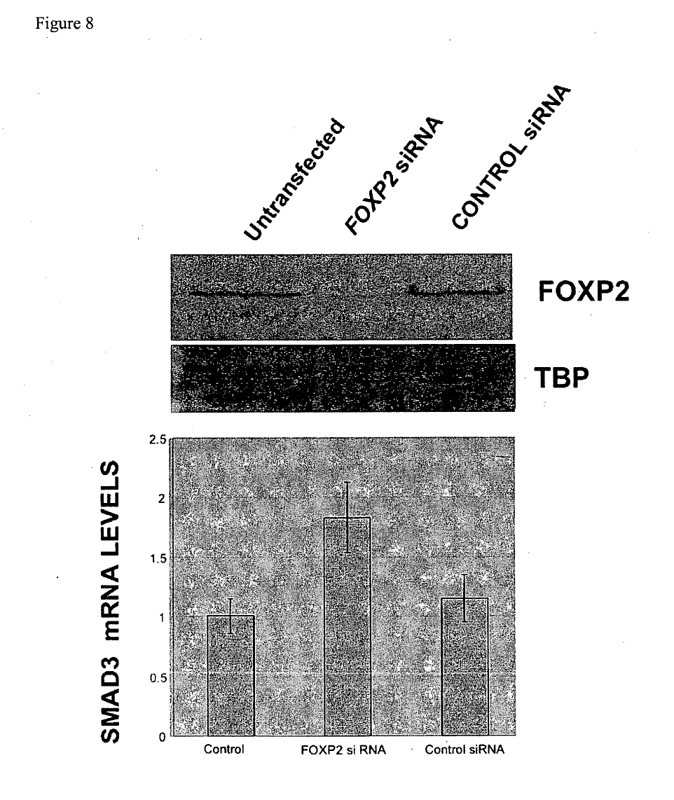

FIG. 8 shows silencing FOXP2 expression up-regulates SMAD3 expression in the JJN3 MM cell line. The top panels show Western blotting with FOXP2-73A/8 demonstrating the effective silencing of FOXP2 at the protein level using siRNA. FOXP2 silencing was not observed using a universal control duplex, and equal sample loading was confirmed using TBP protein expression. The bottom panel illustrates qRT-PCR analysis of SMAD3 mRNA expression in RNA prepared from the same experiment, showing specific up-regulation of SMAD3 expression on silencing FOXP2 (FOXP2 siRNA) compared to untransfected cells or those transfected with a control siRNA.

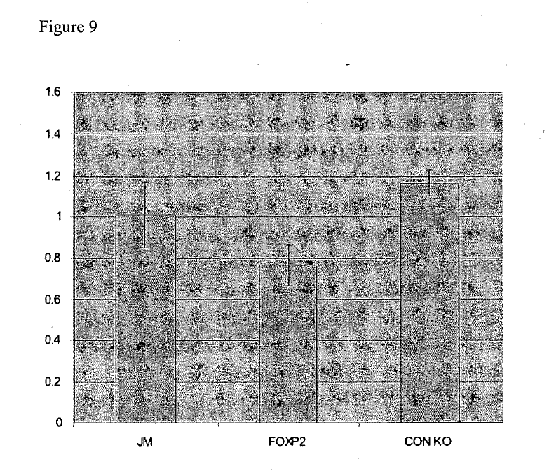

FIG. 9 shows silencing FOXP2 expression down-regulates IRF4 expression in the JJN3 MM cell line. qRT-PCR showing specific down-regulation of IRF4 expression on silencing FOXP2 (FOXP2) compared to cells treated with a universal siRNA (CON KO) or cells just grown normally in culture medium (JM).

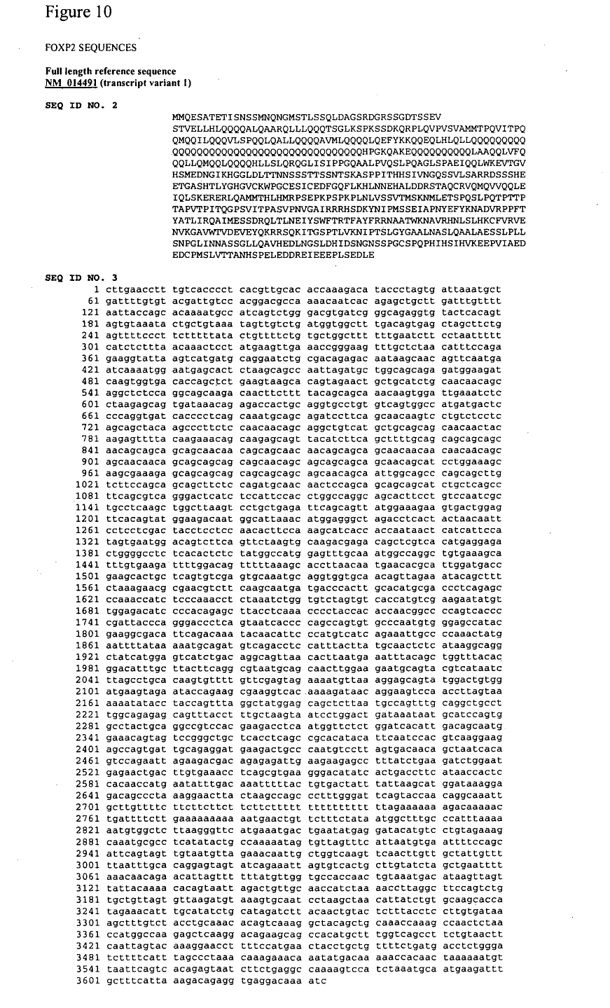

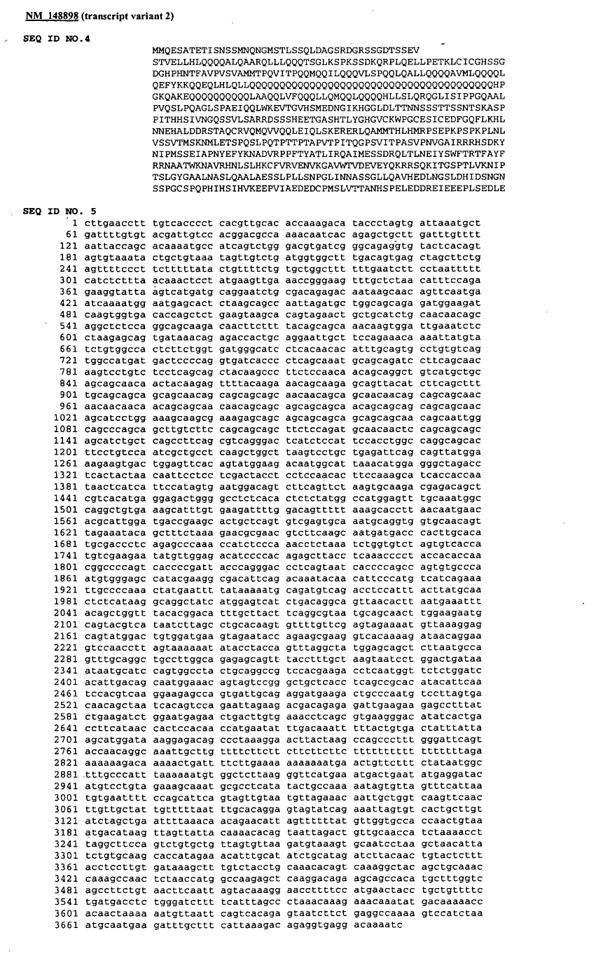

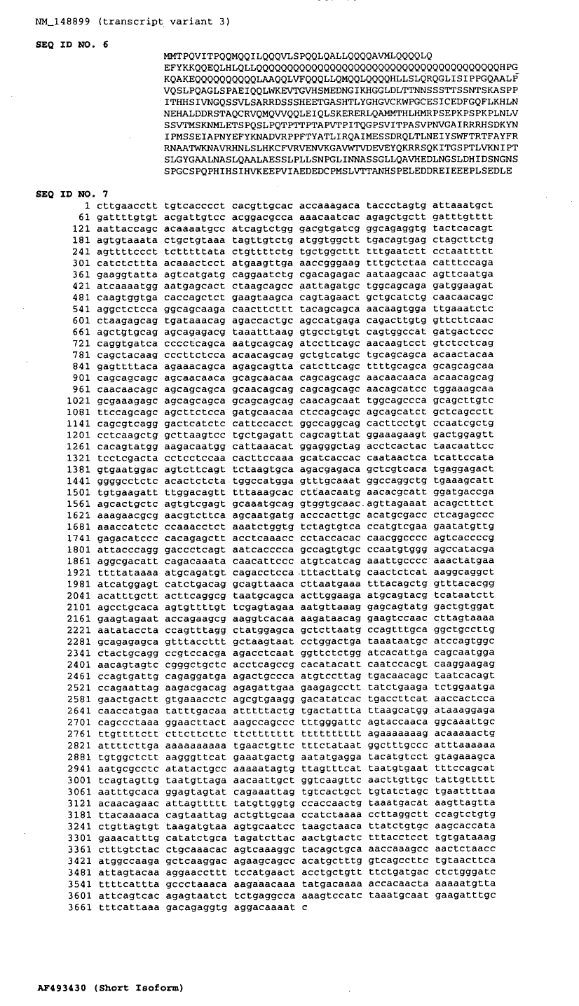

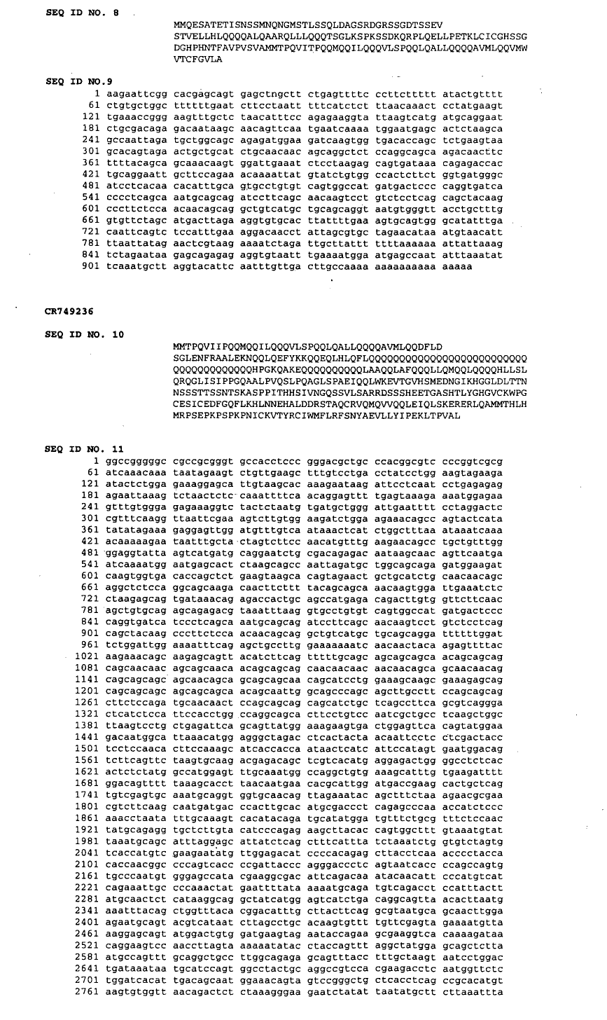

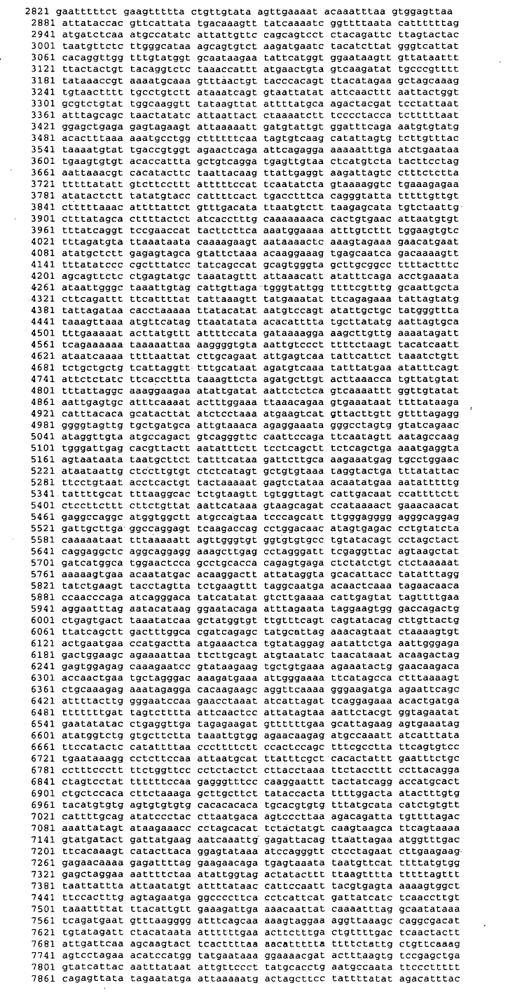

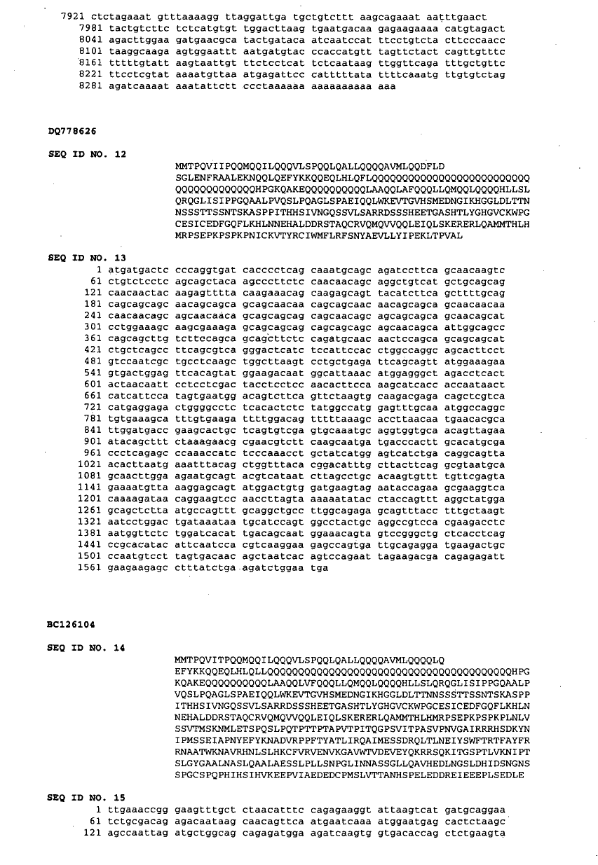

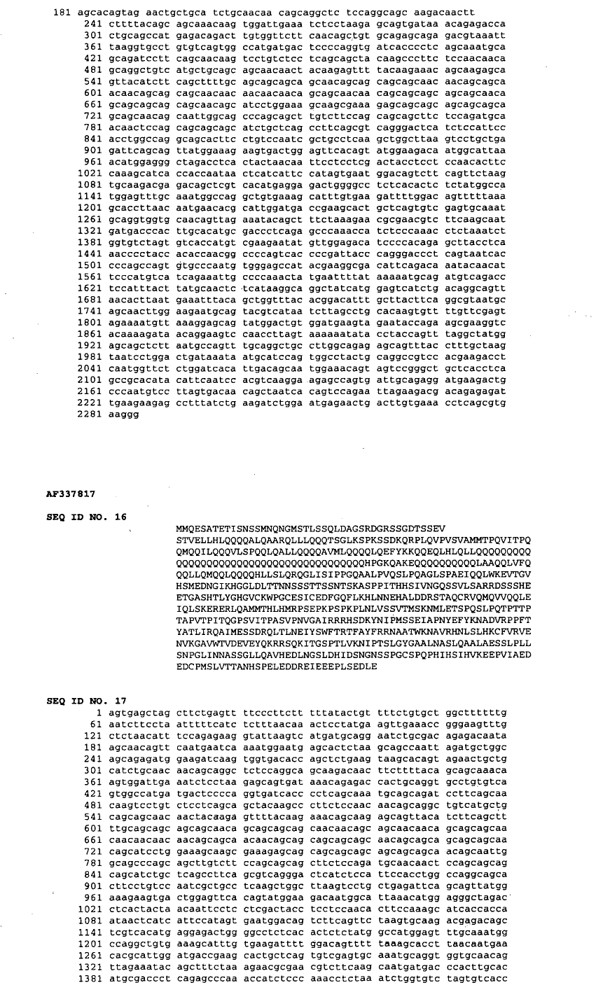

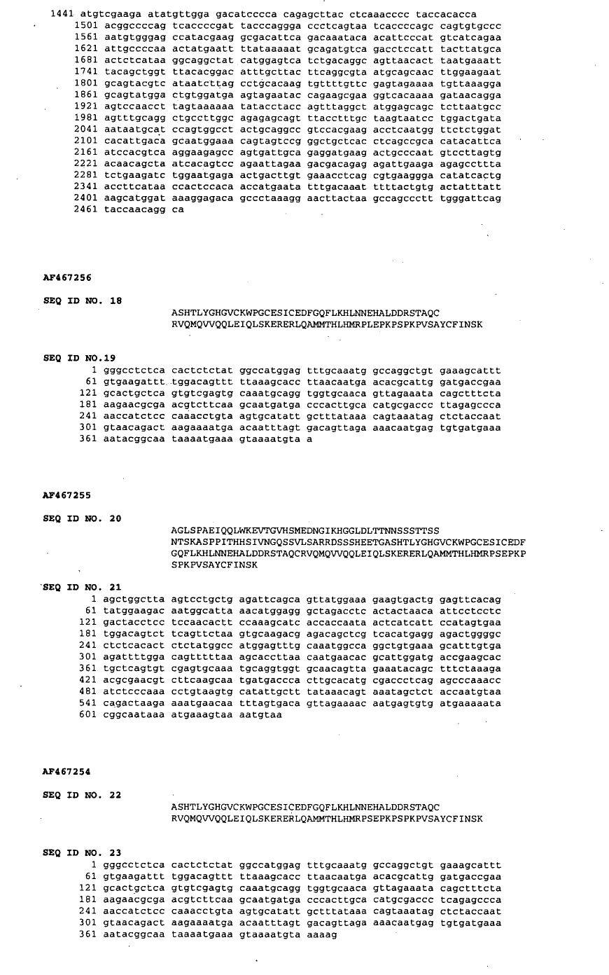

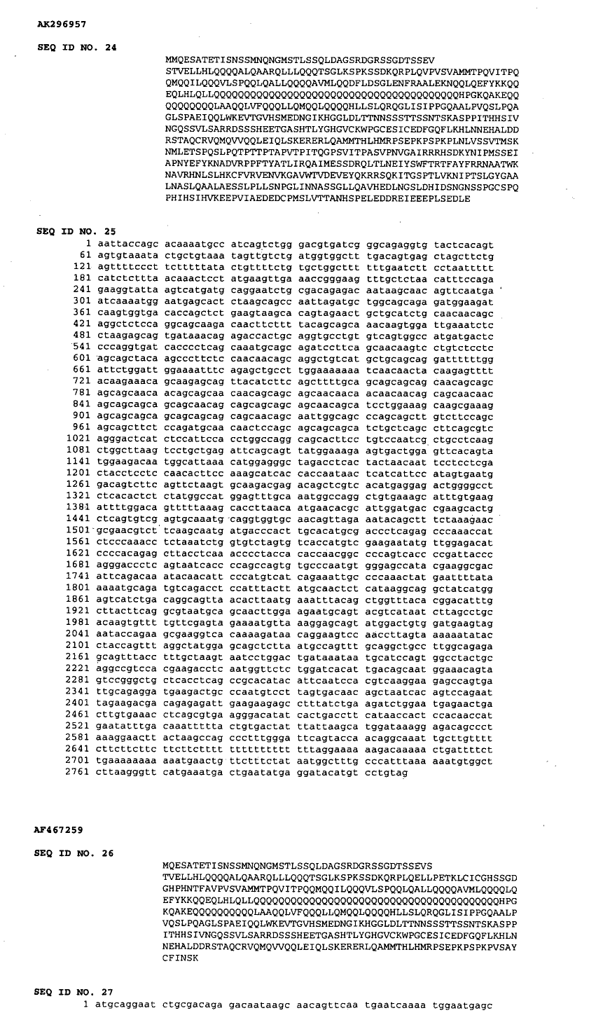

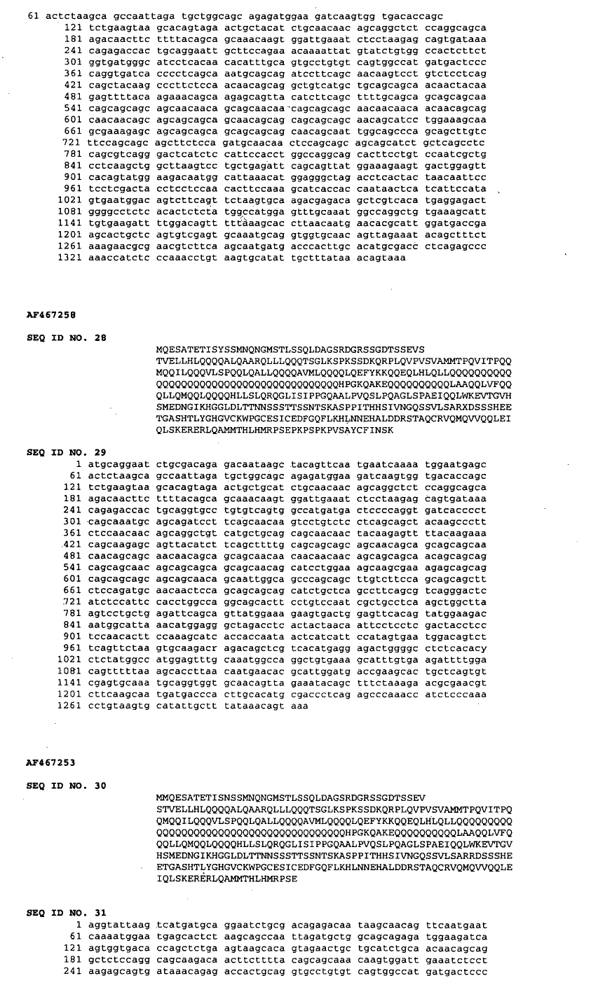

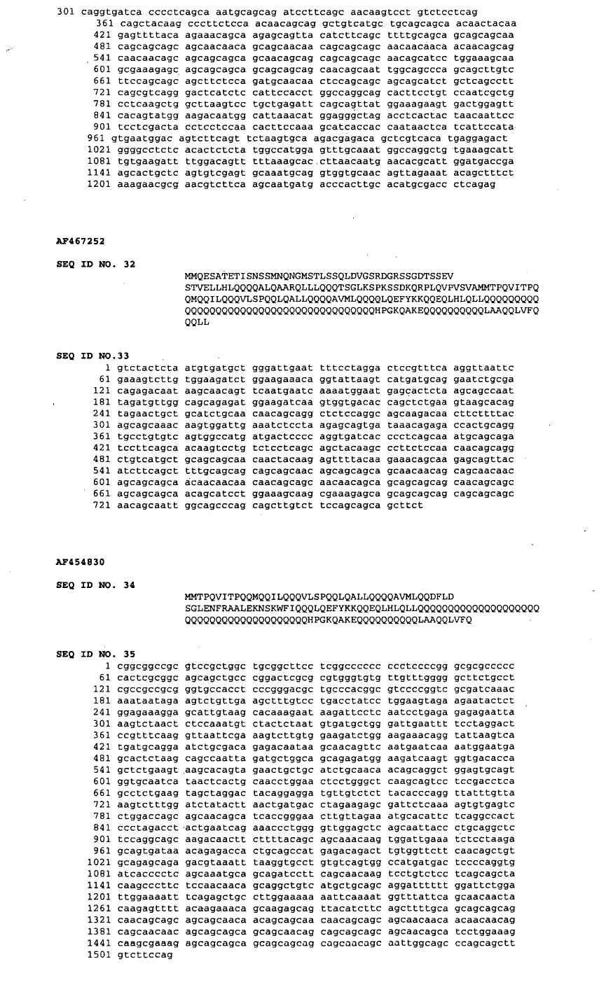

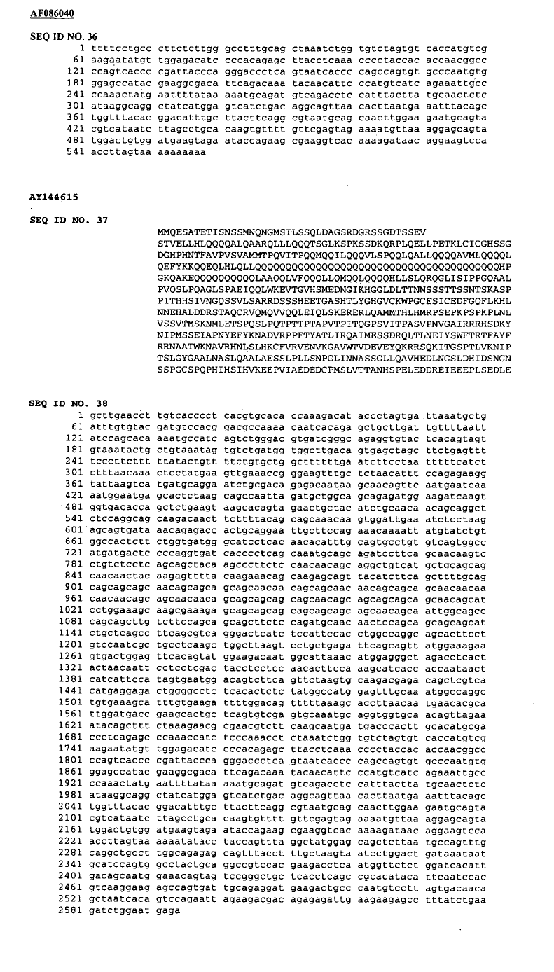

FIG. 10 shows all 19 mRNA sequences from the Unigene folder for FOXP2, including all the known variant transcripts. Their encoded protein products are also shown.

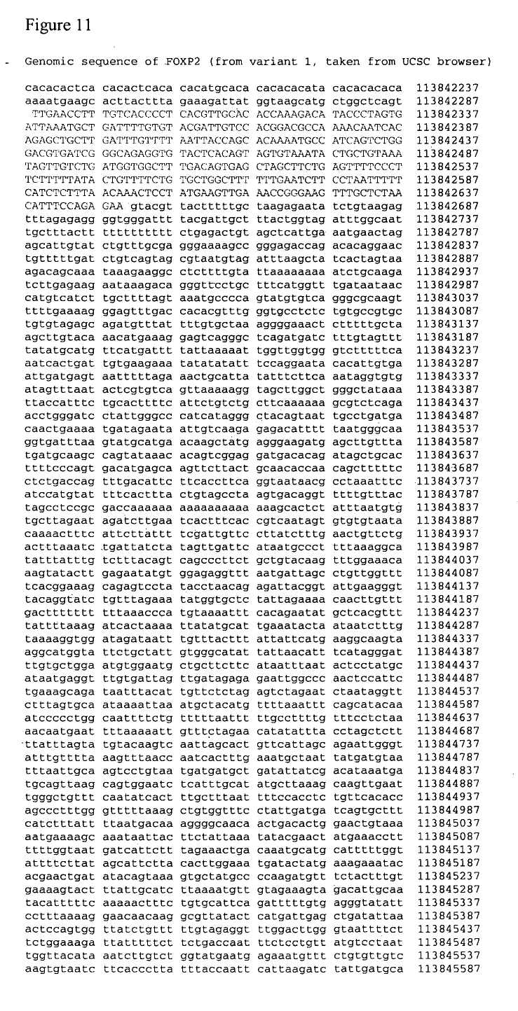

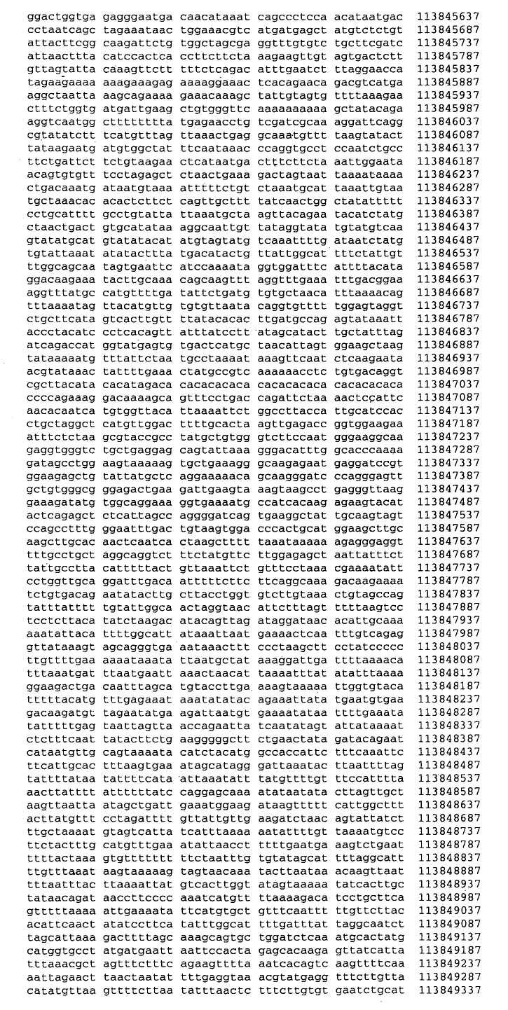

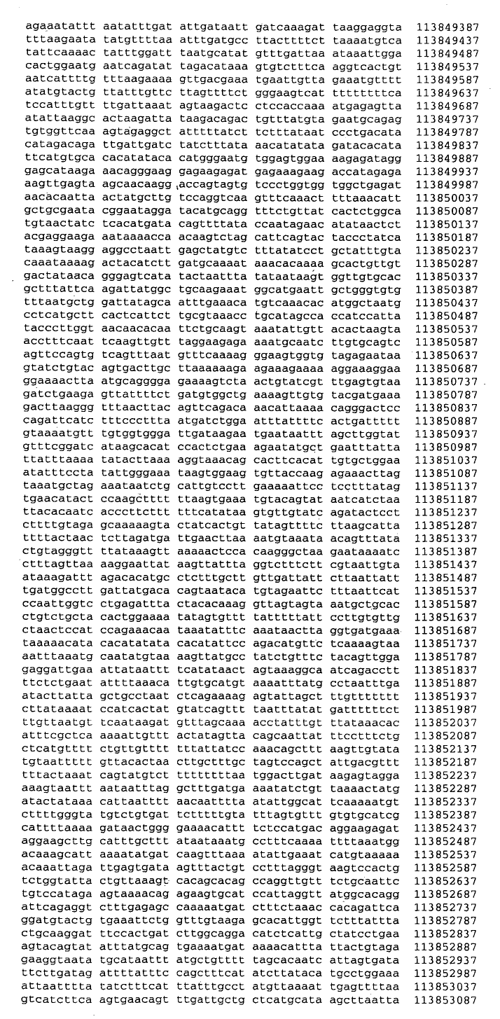

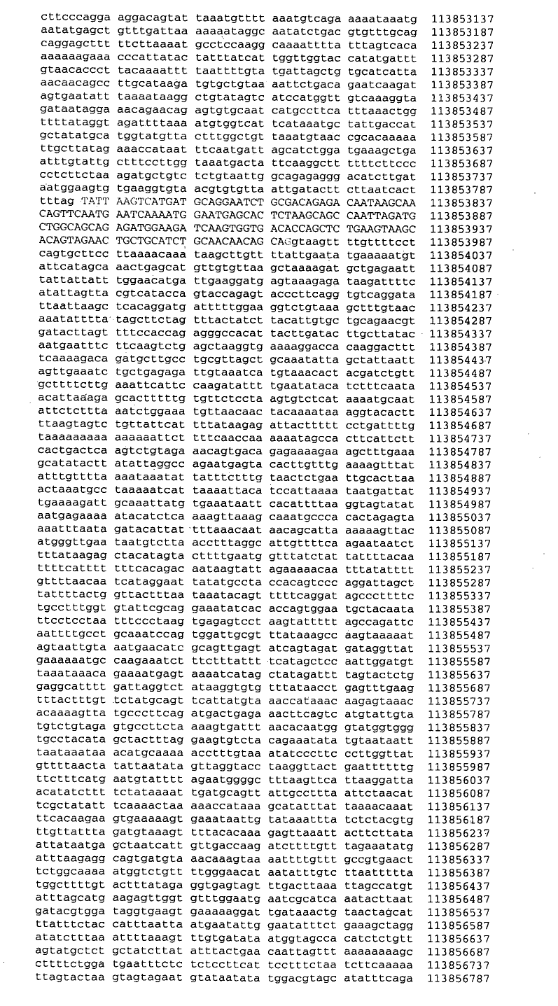

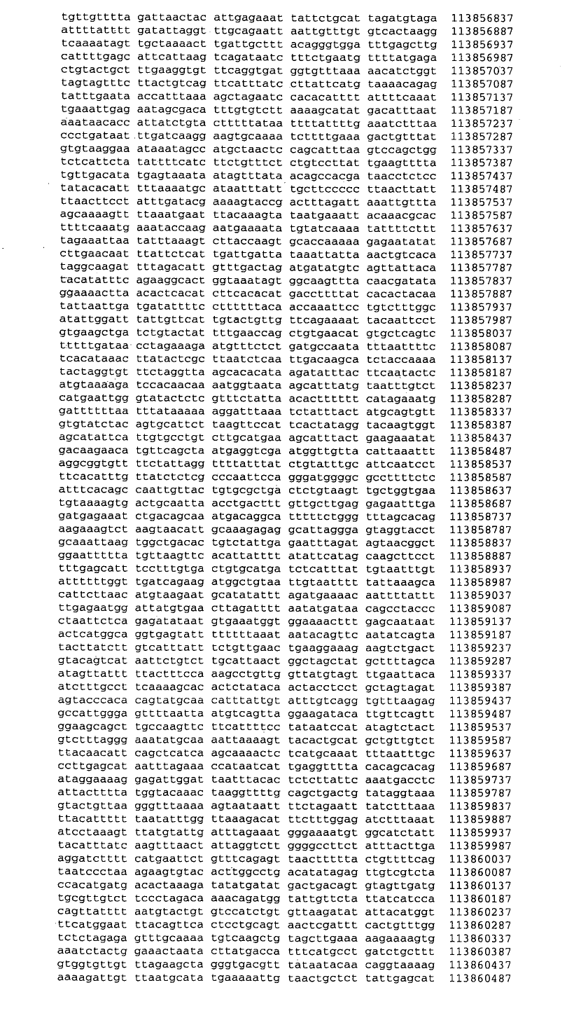

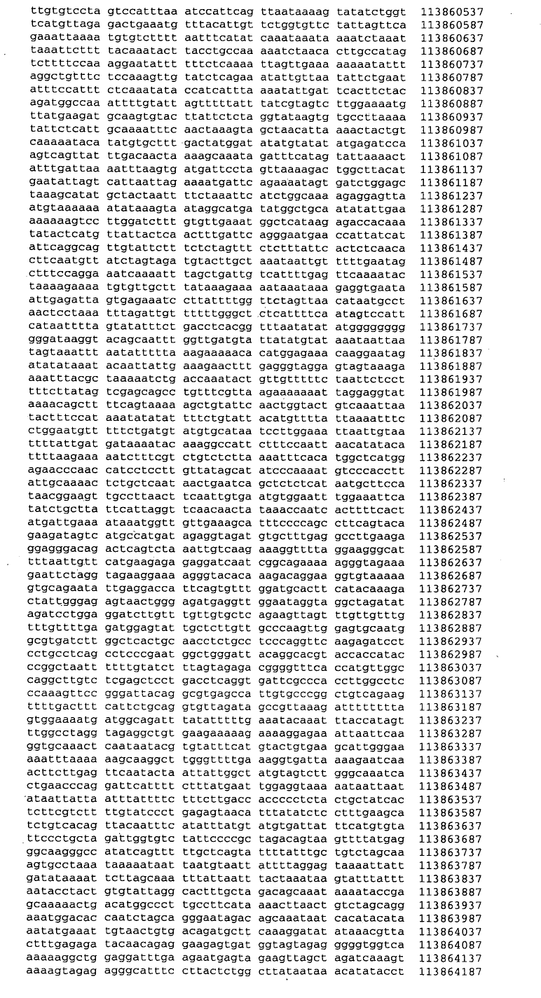

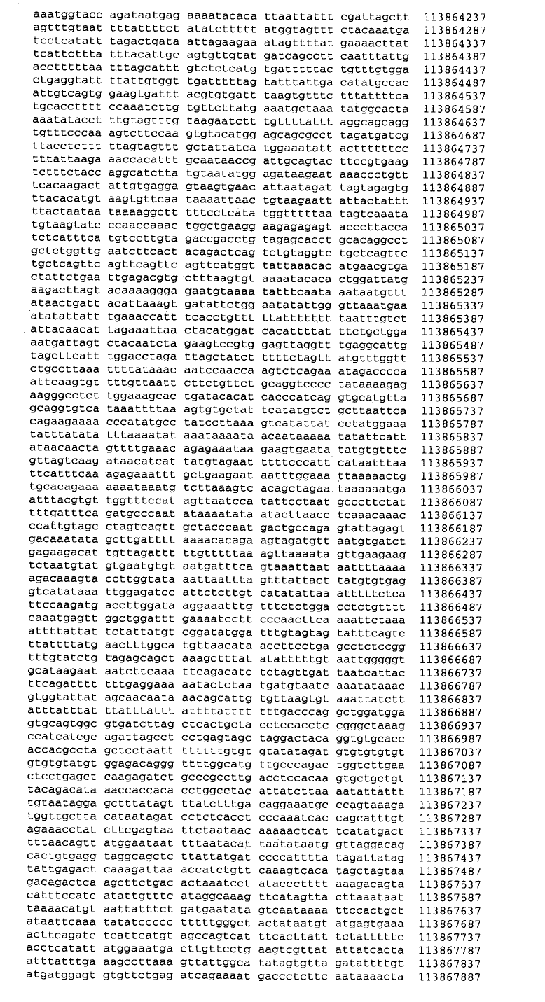

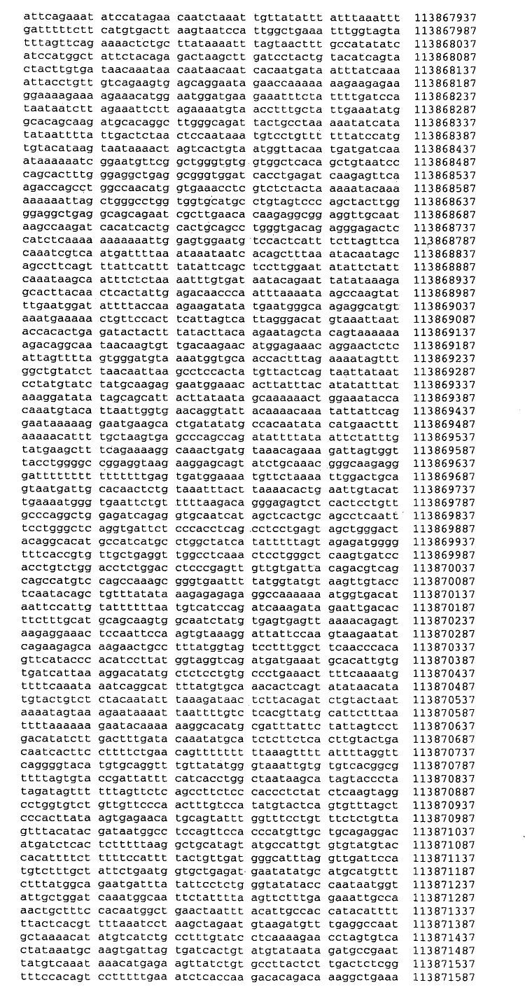

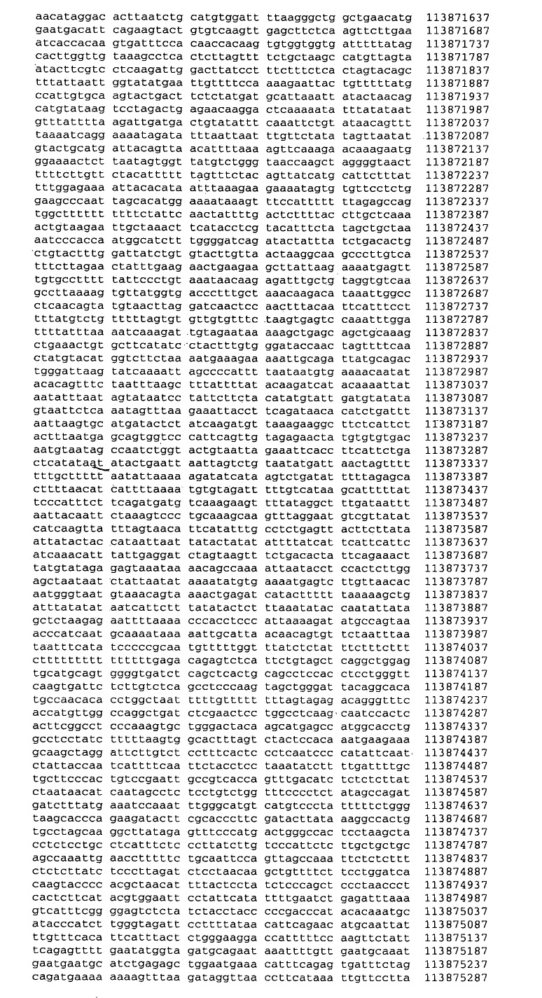

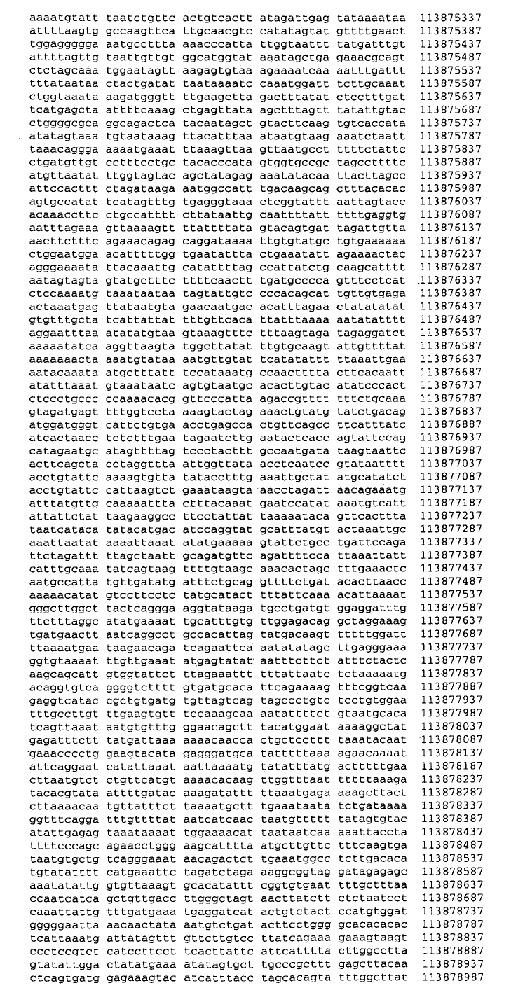

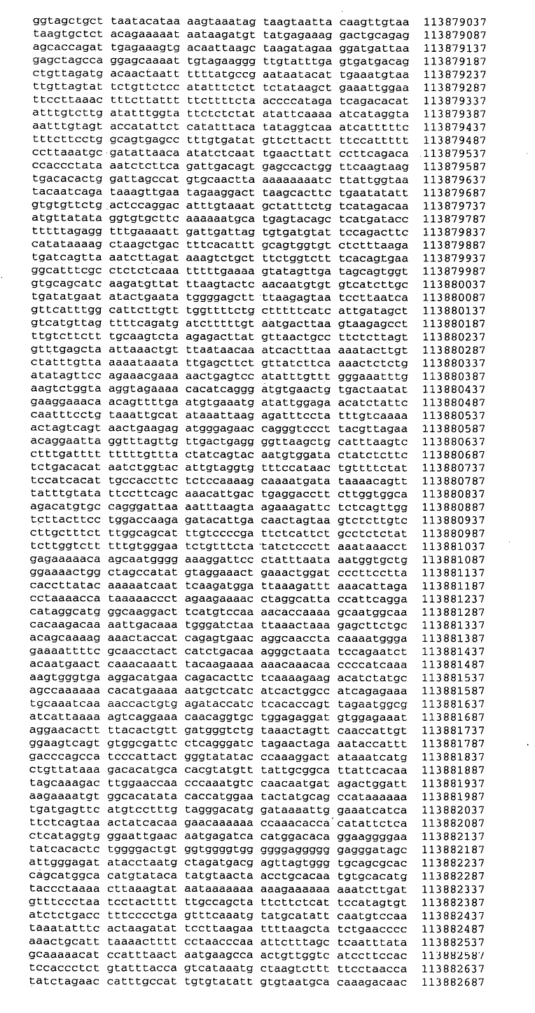

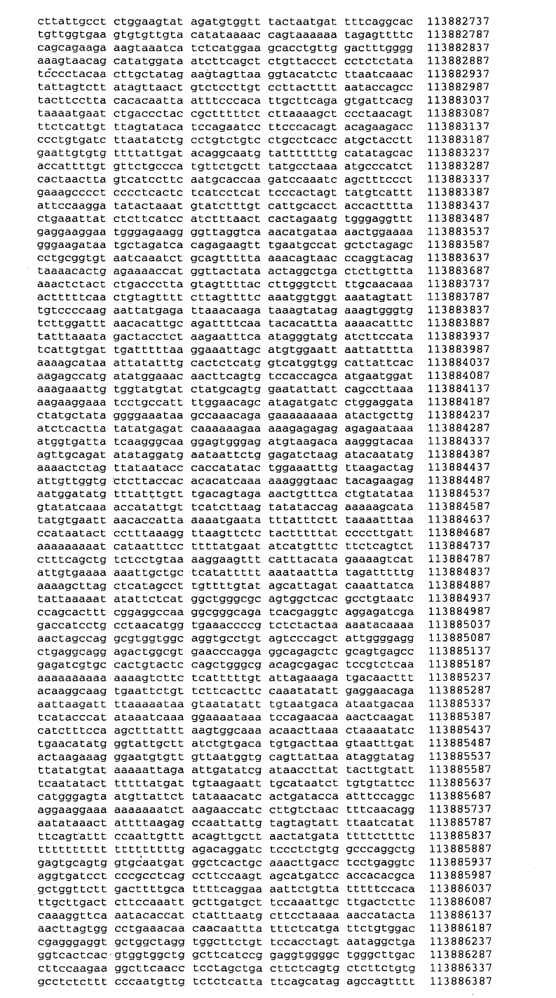

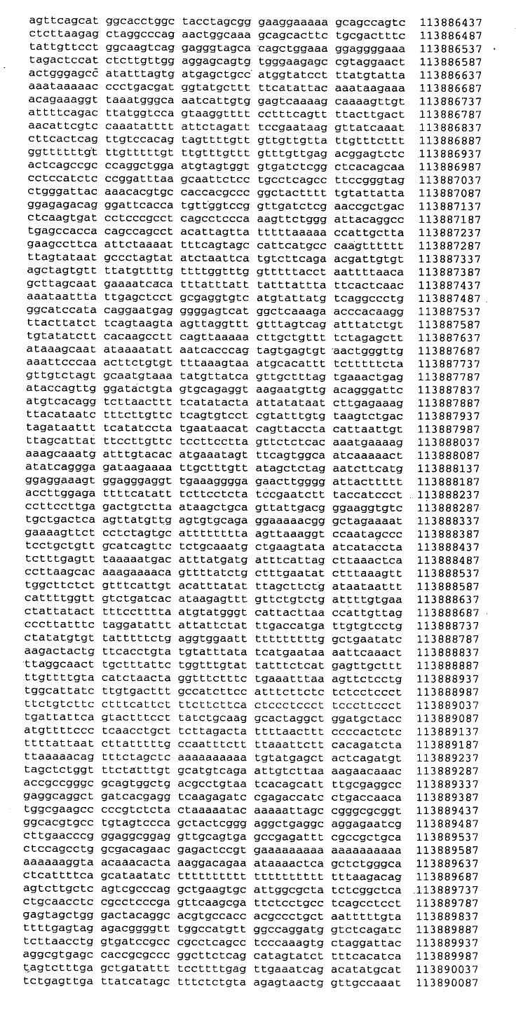

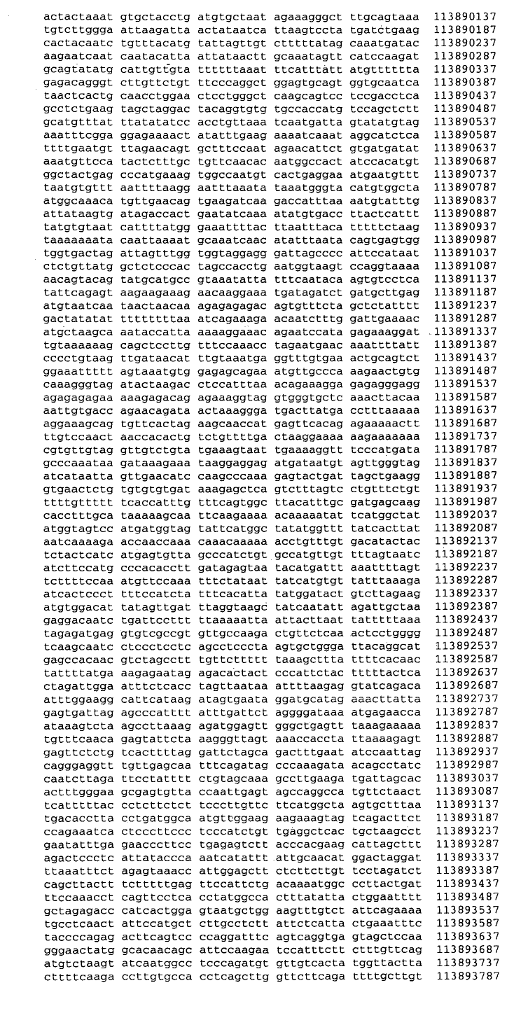

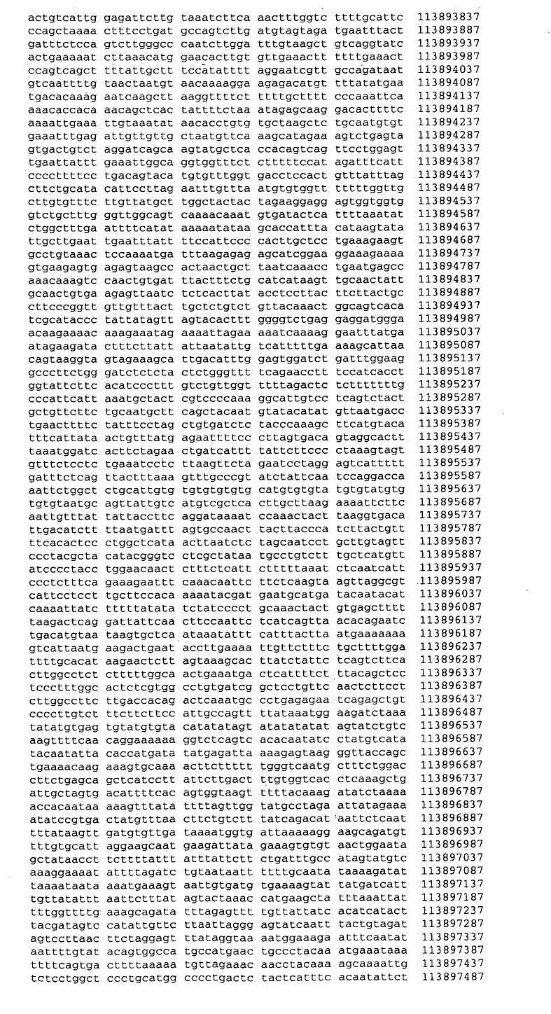

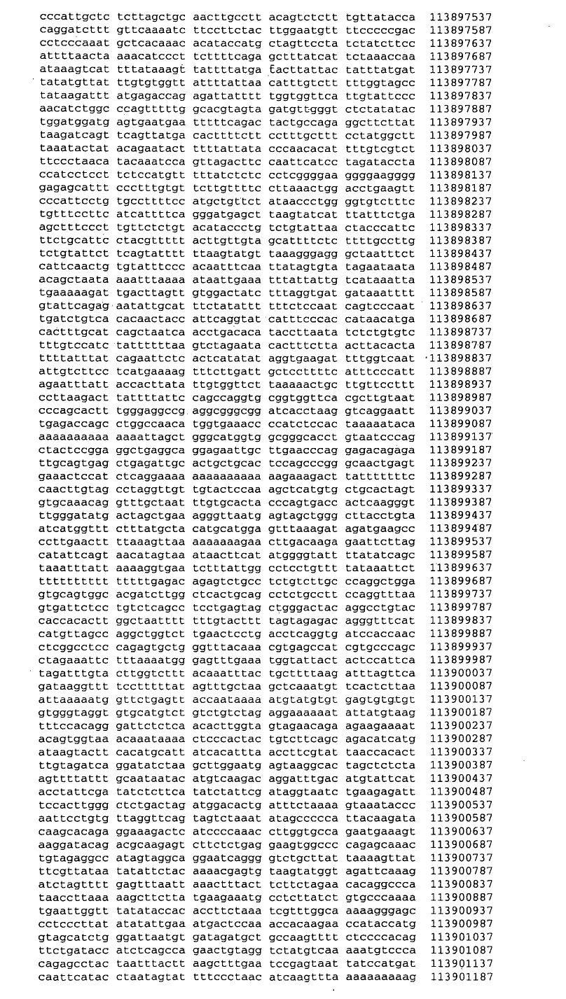

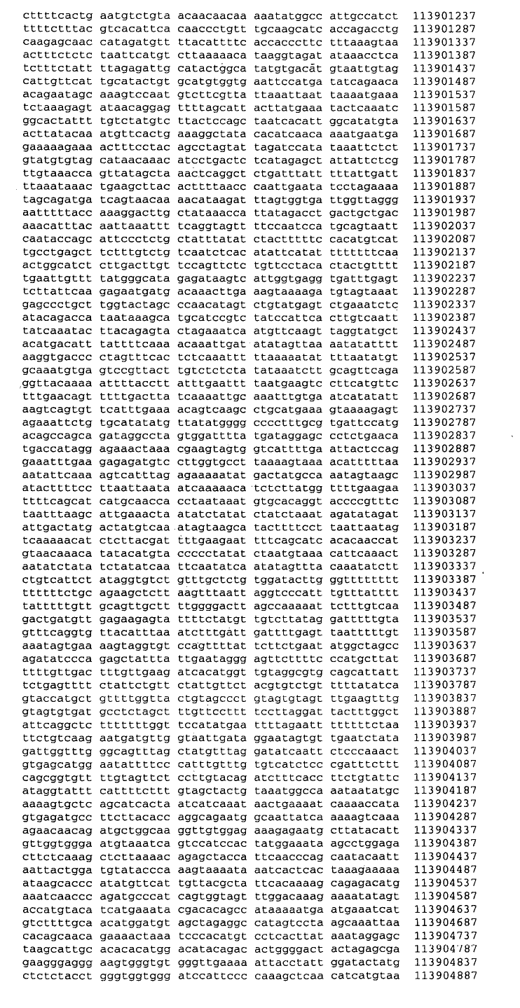

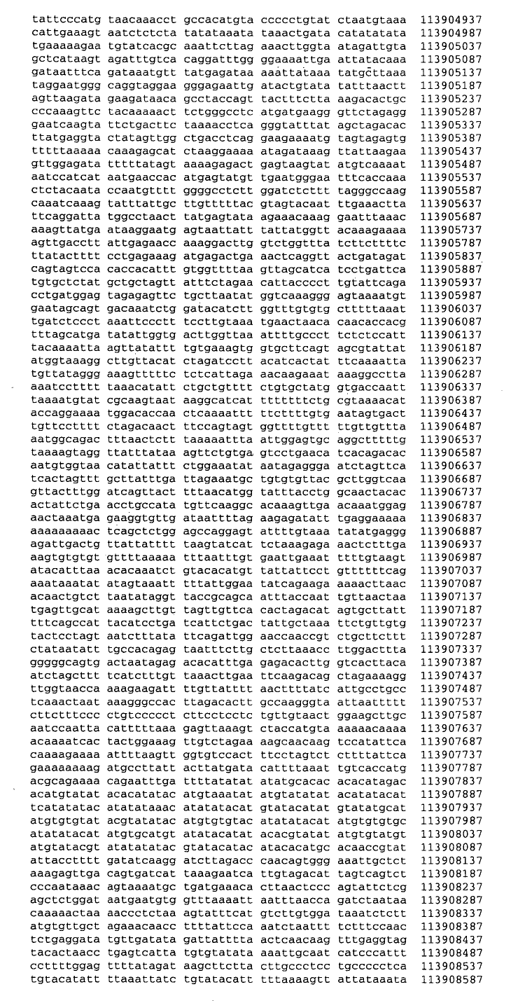

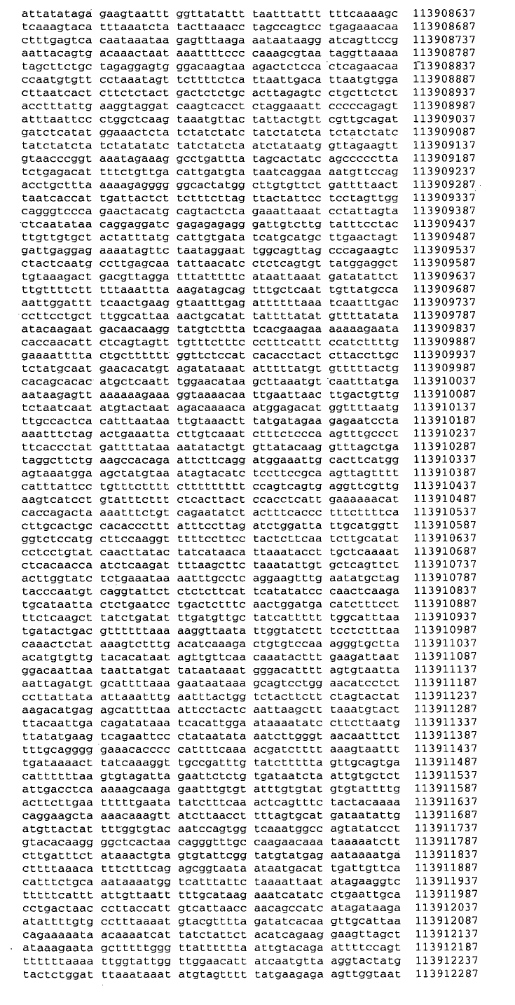

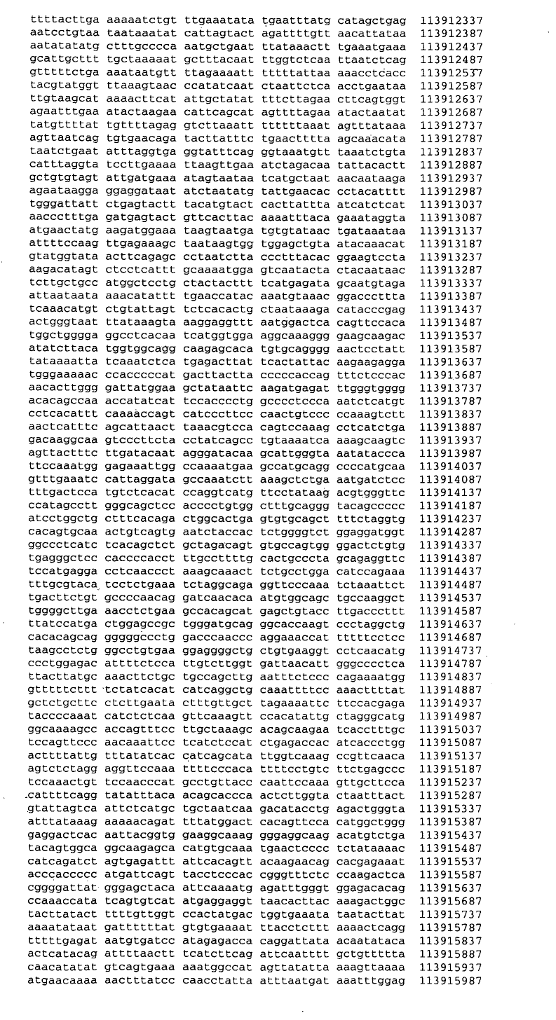

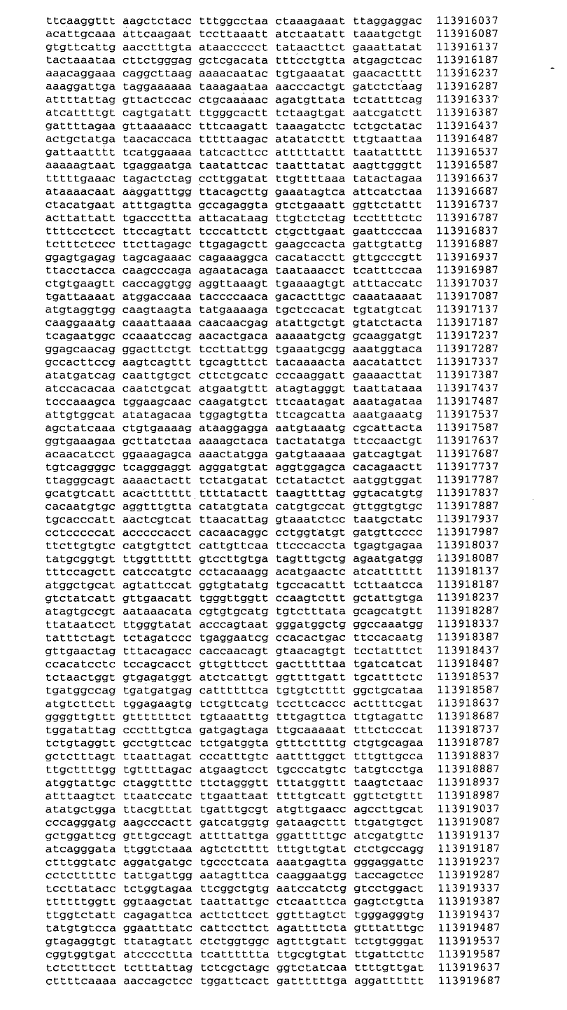

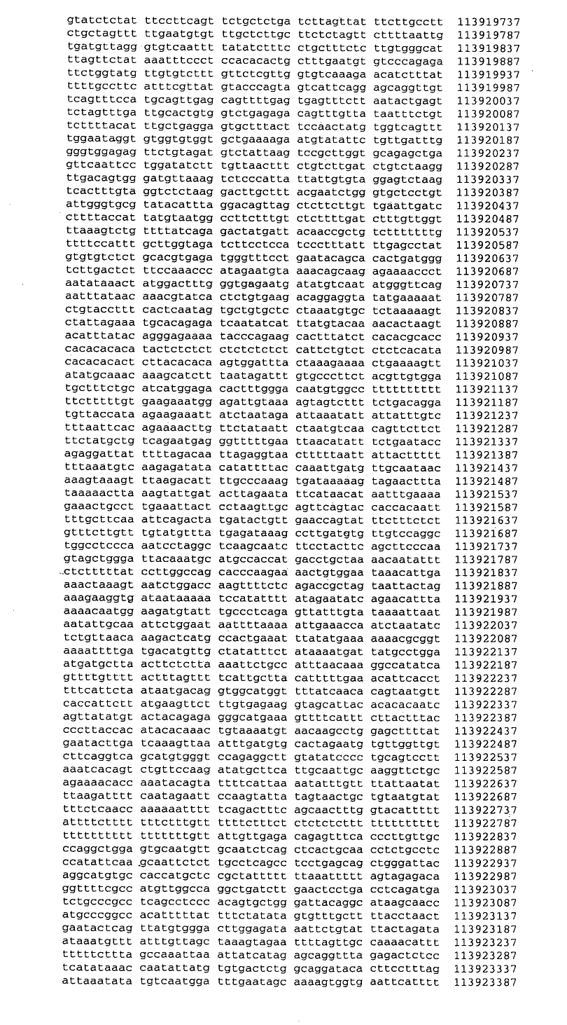

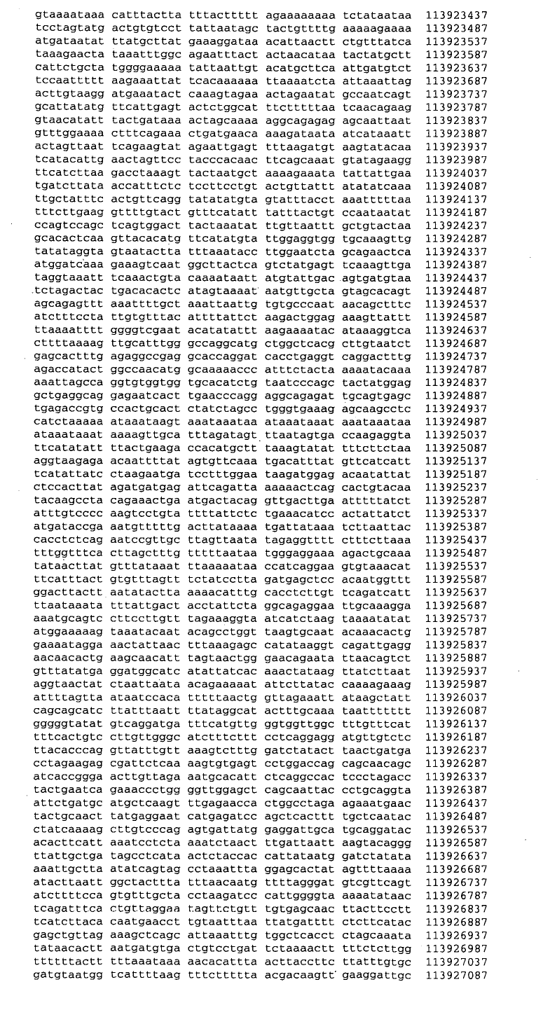

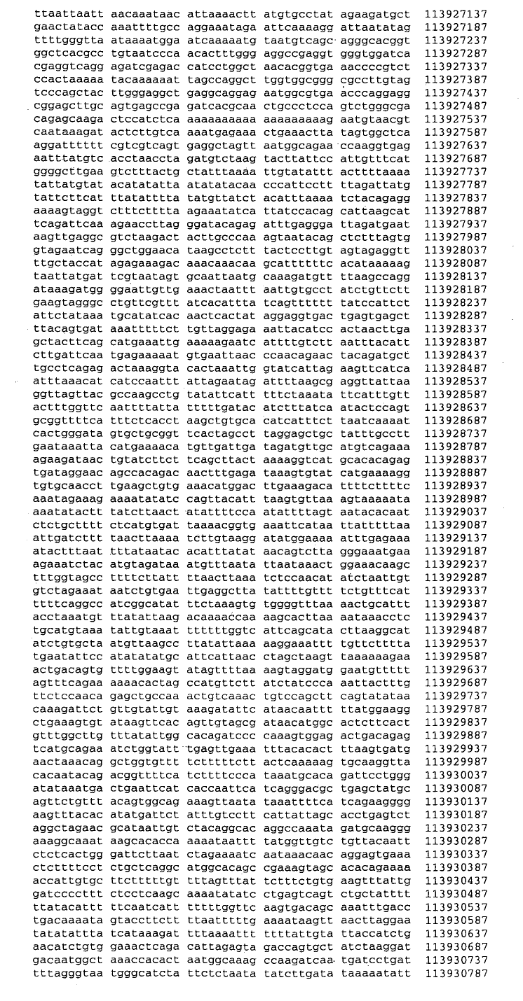

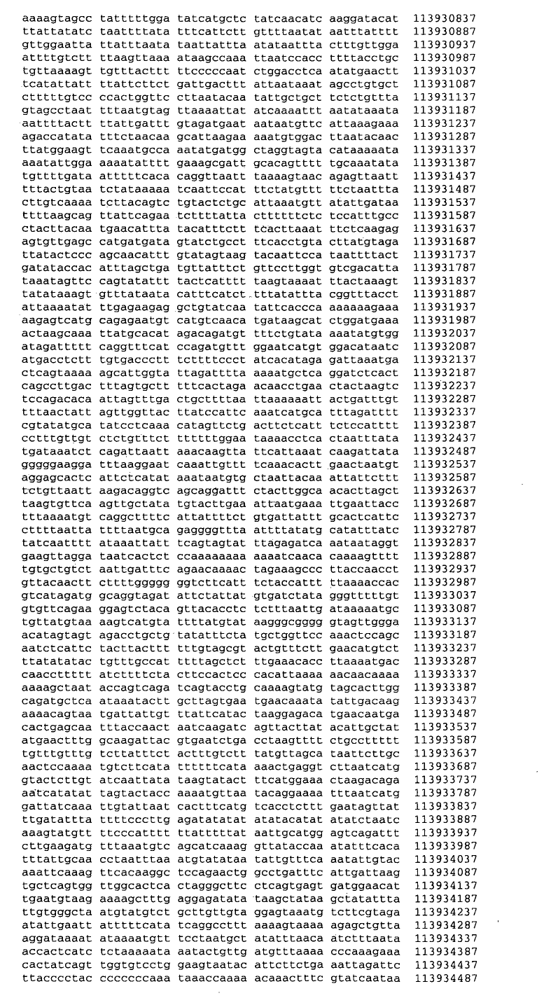

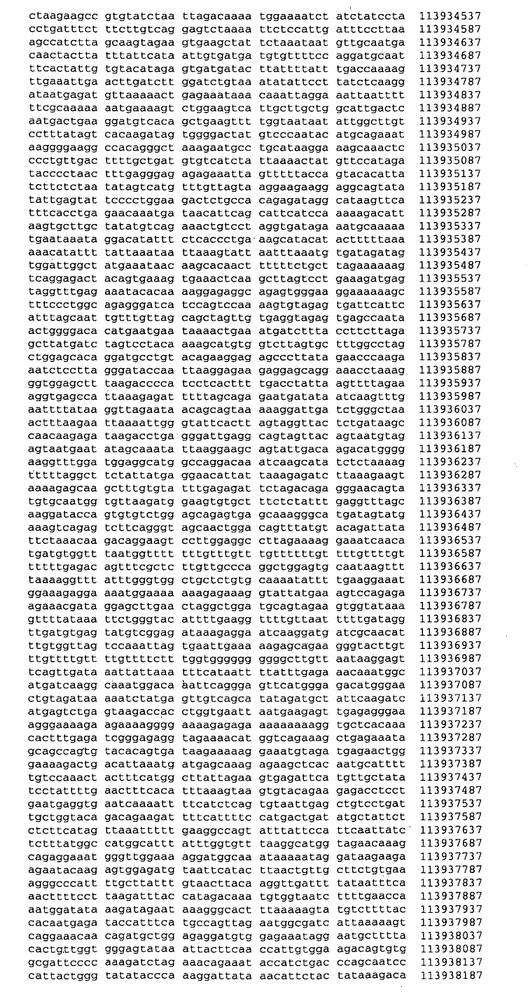

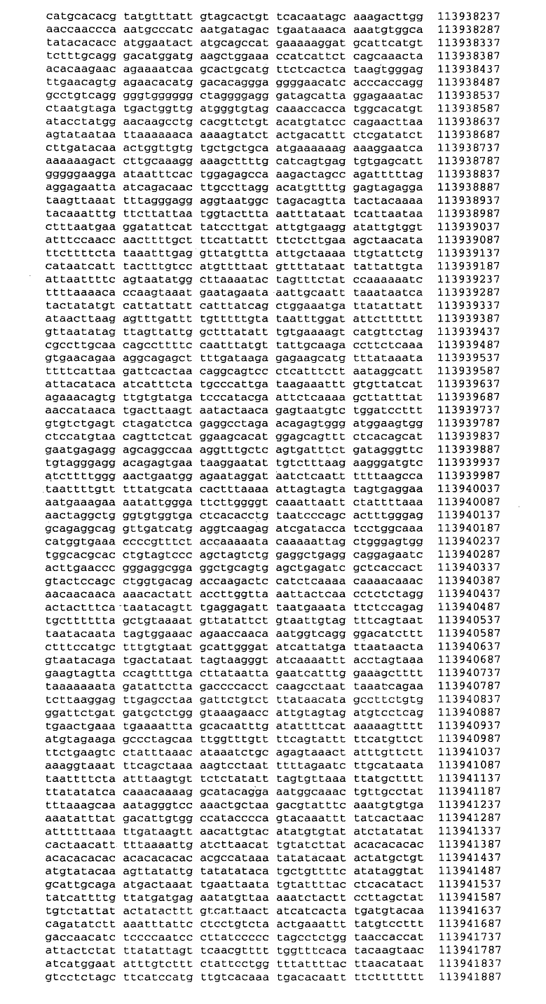

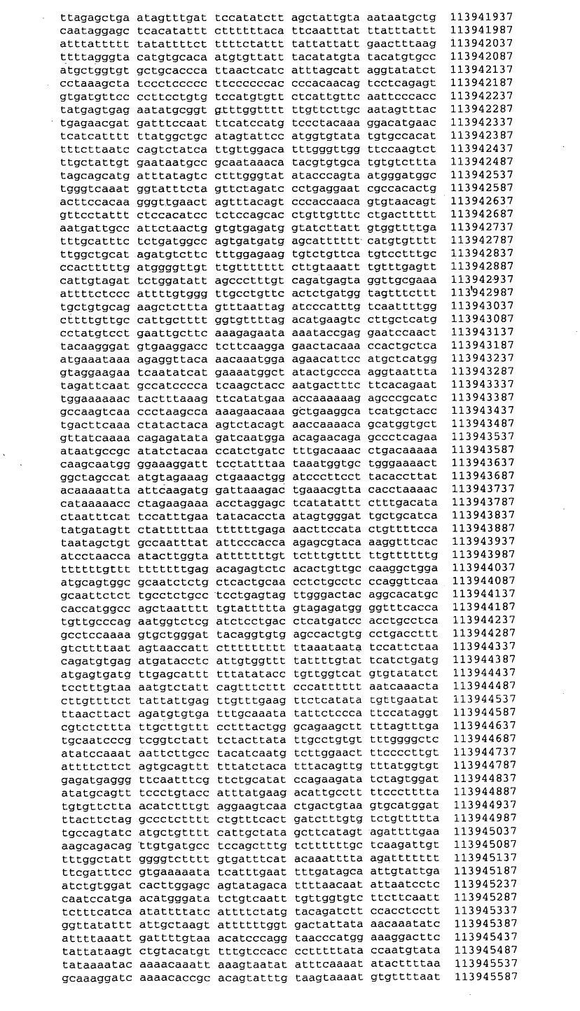

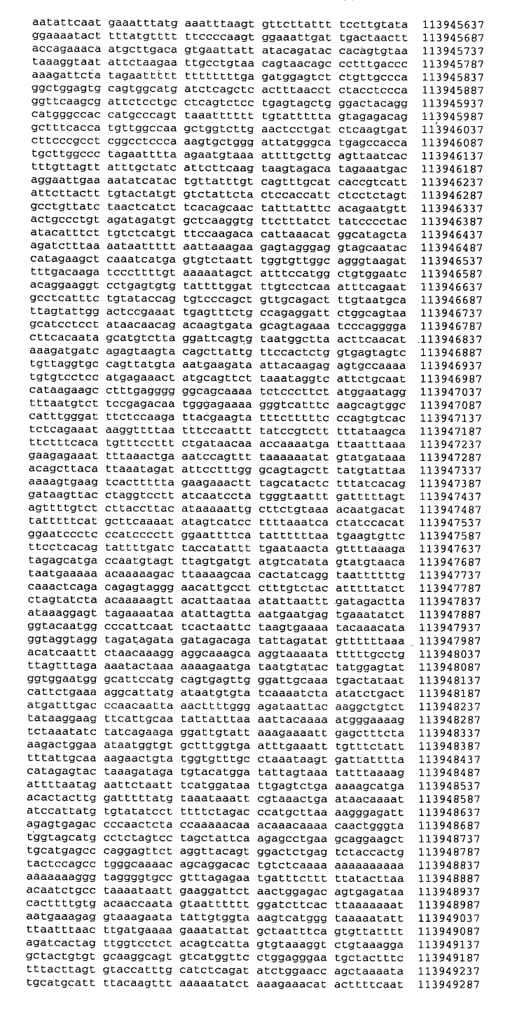

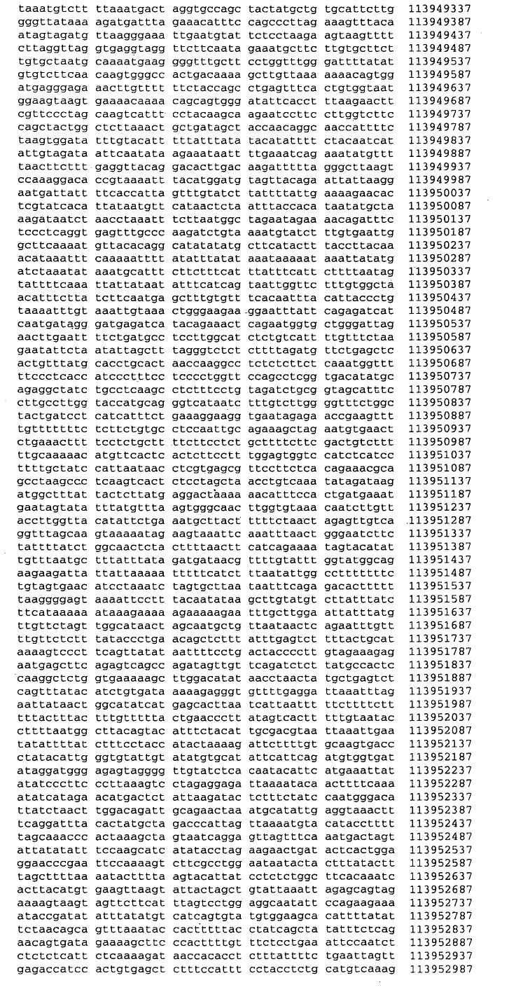

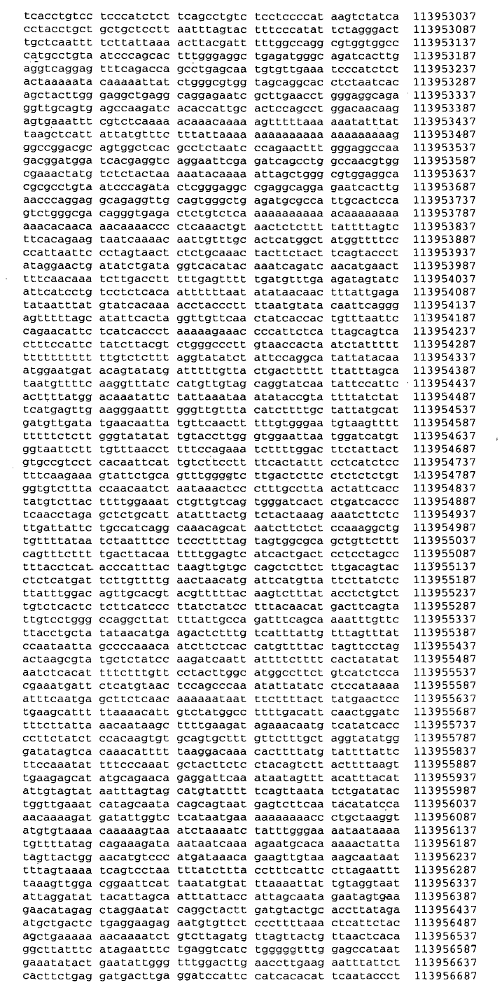

FIG. 11 shows the genomic sequence of the FOXP2 gene (SEQ ID NO.1).

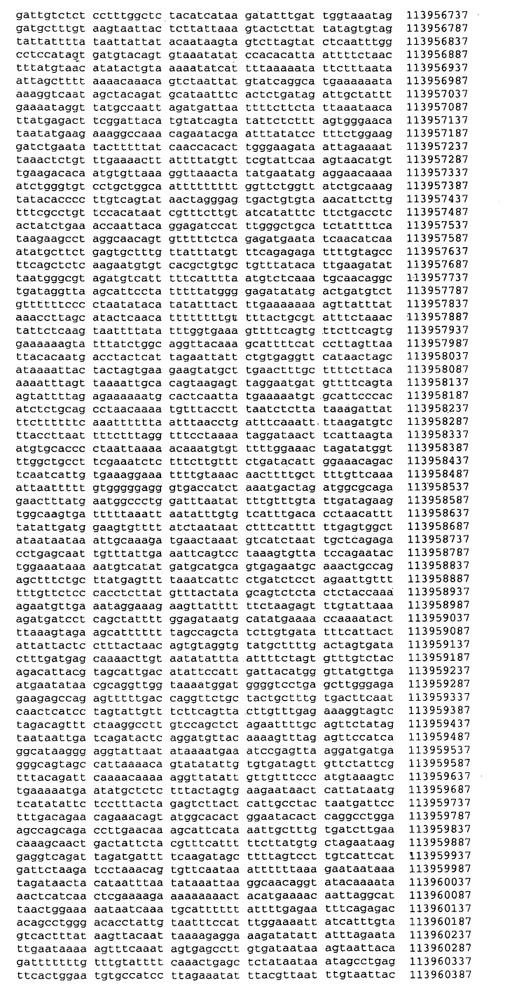

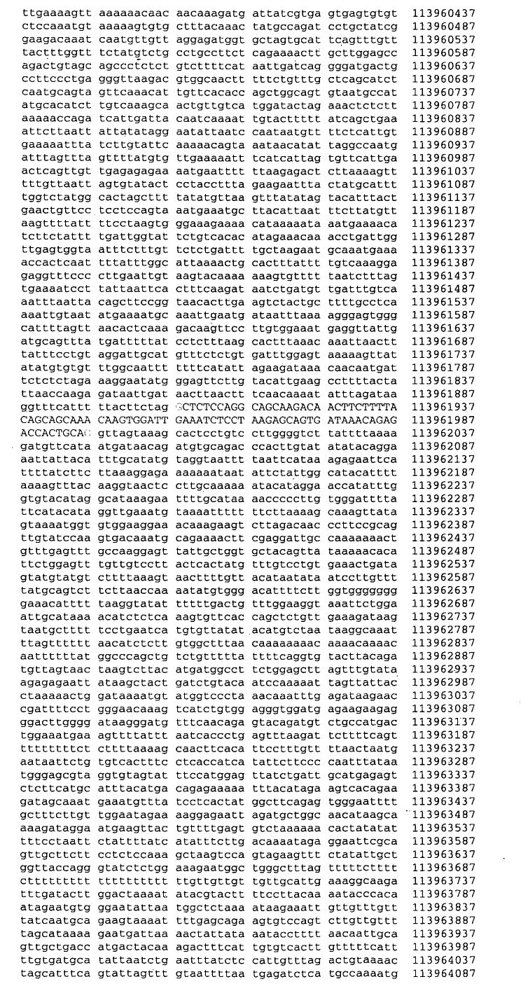

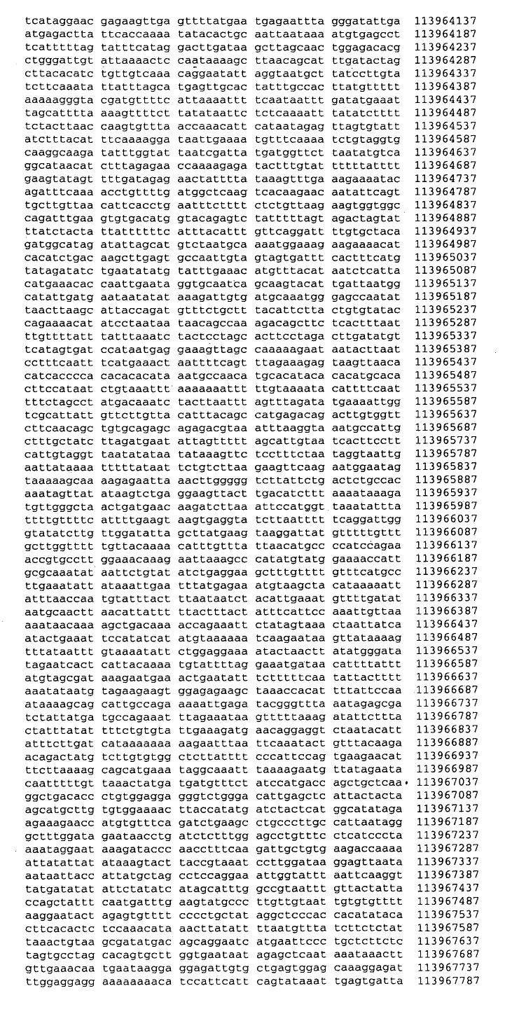

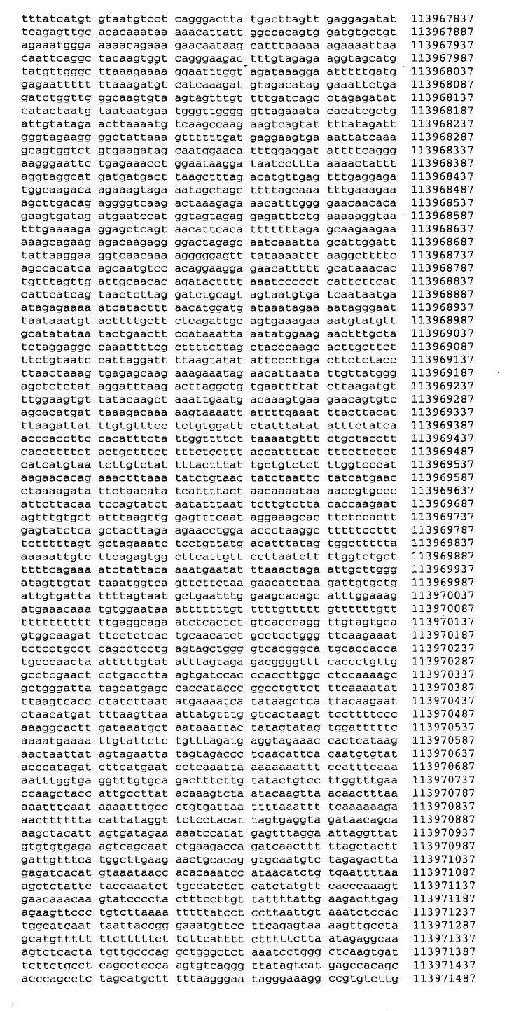

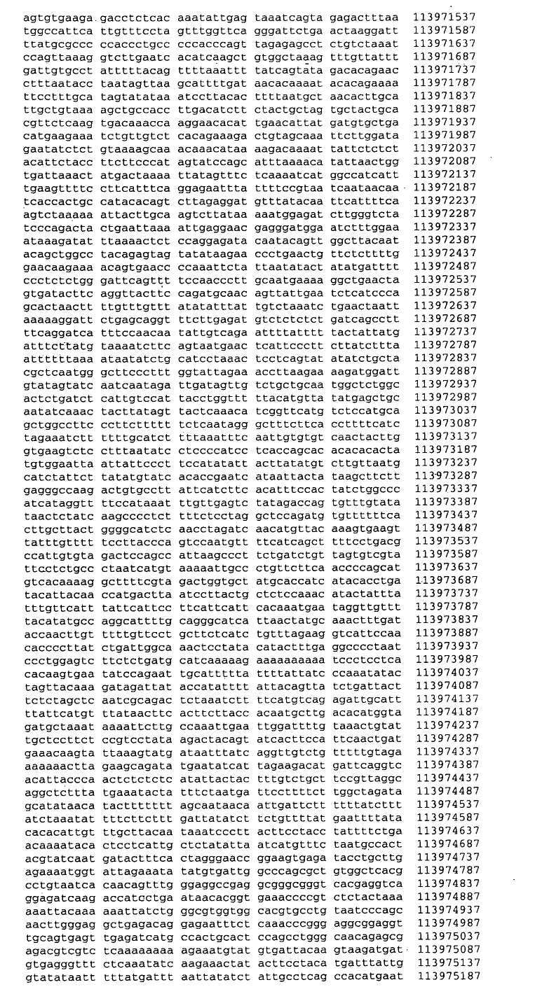

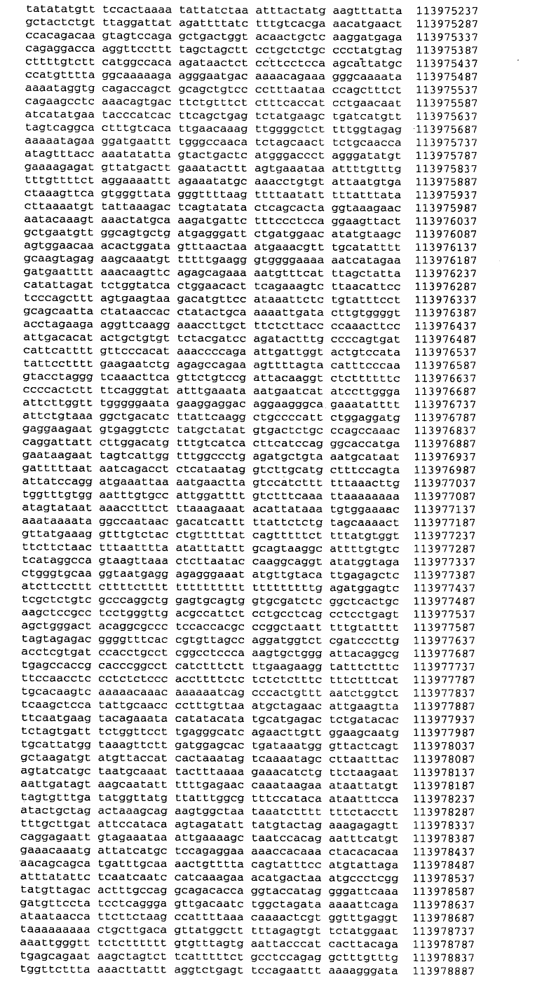

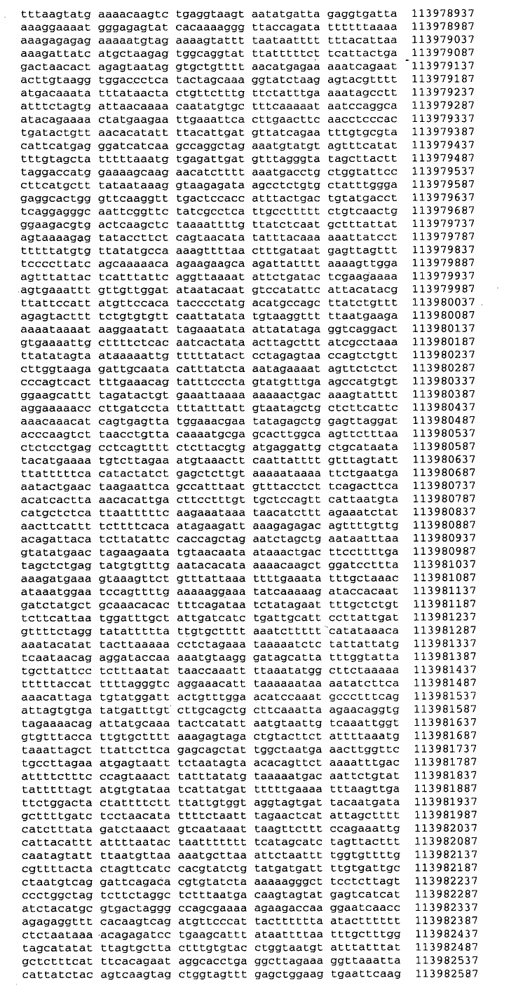

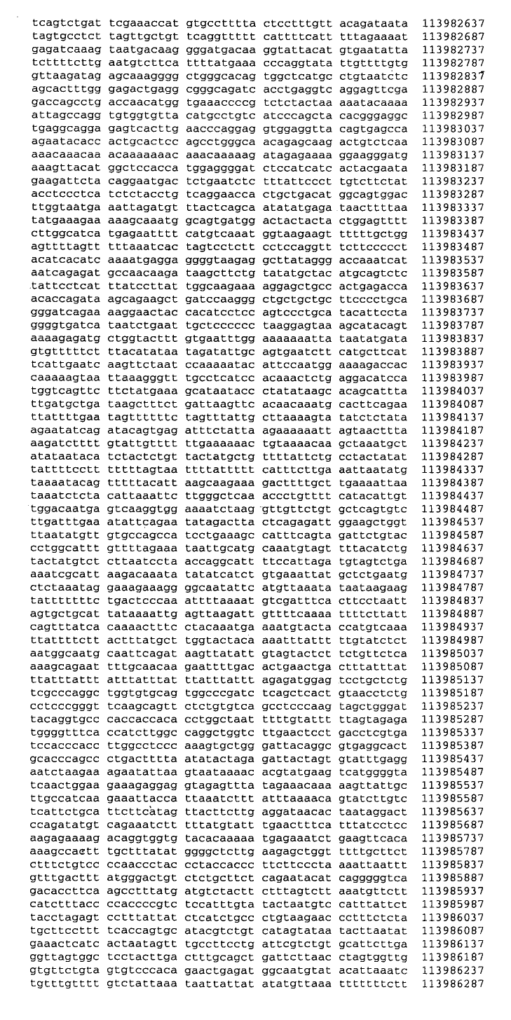

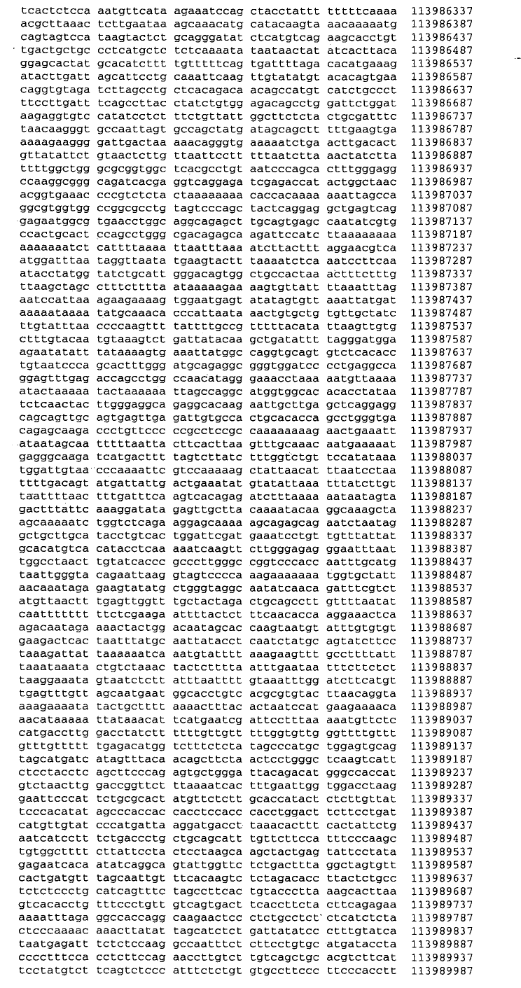

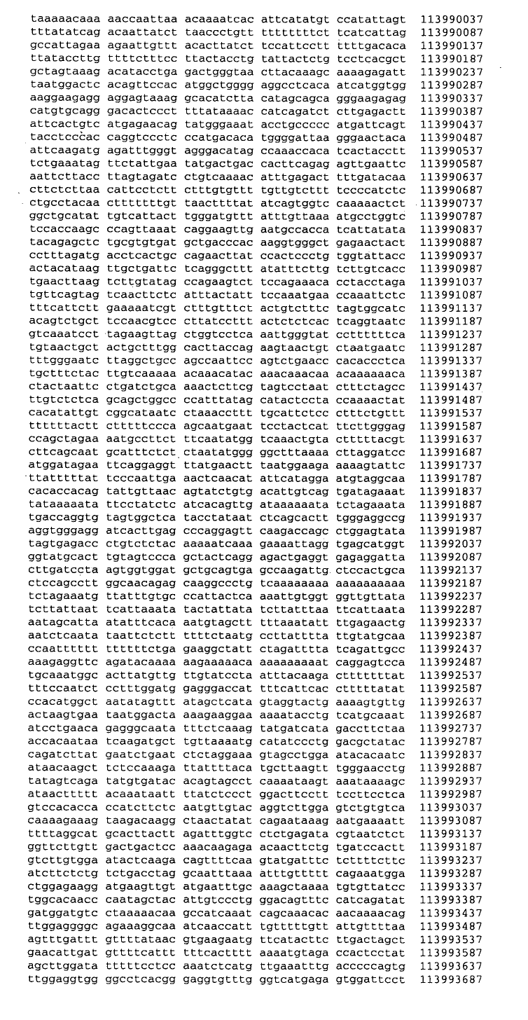

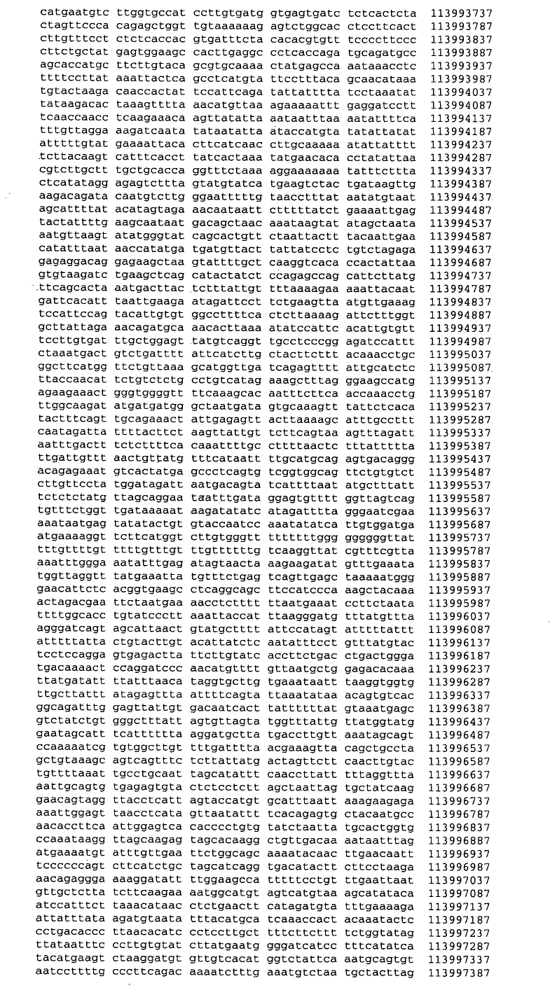

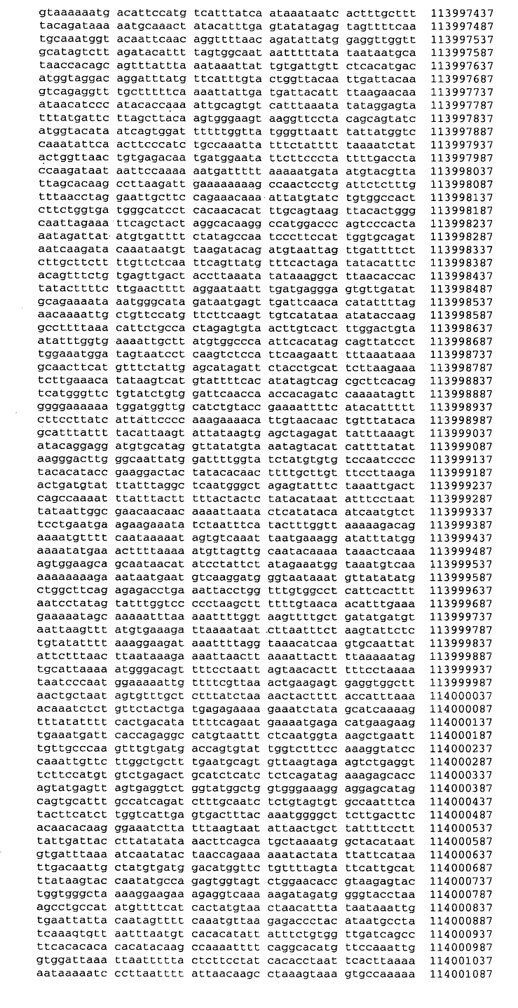

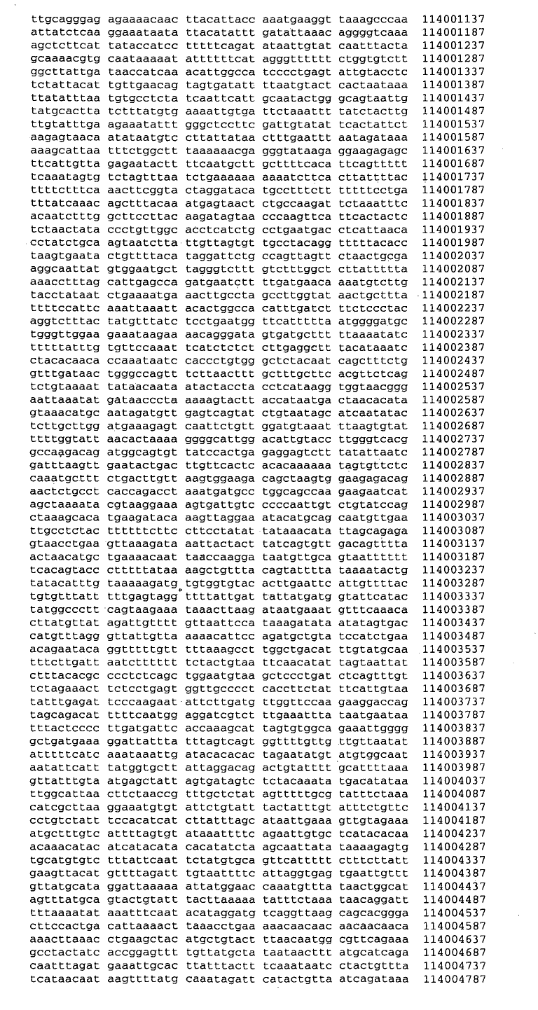

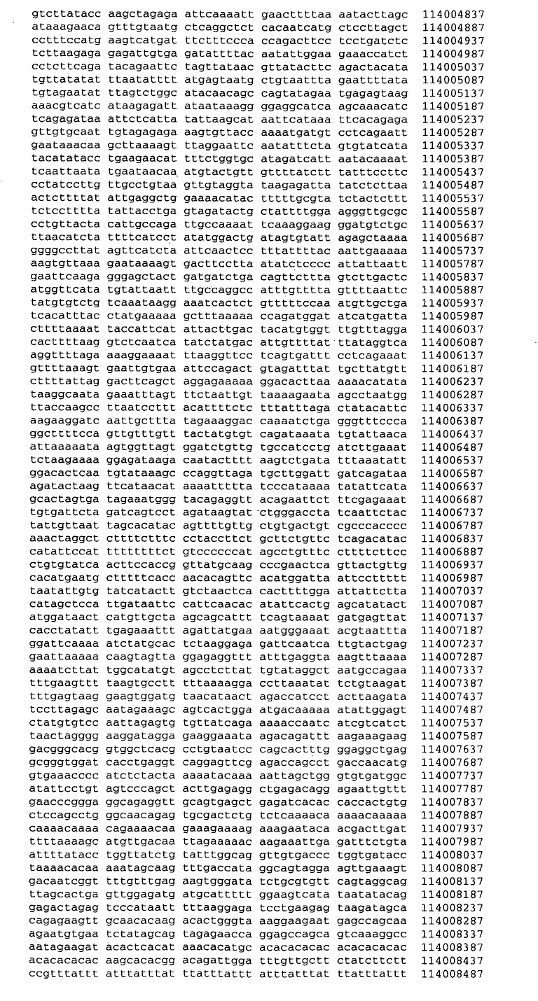

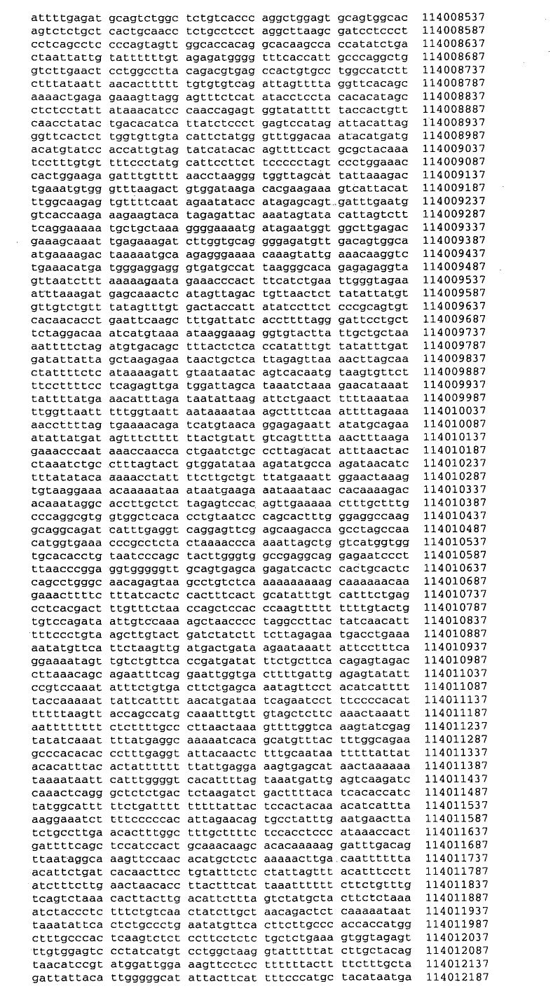

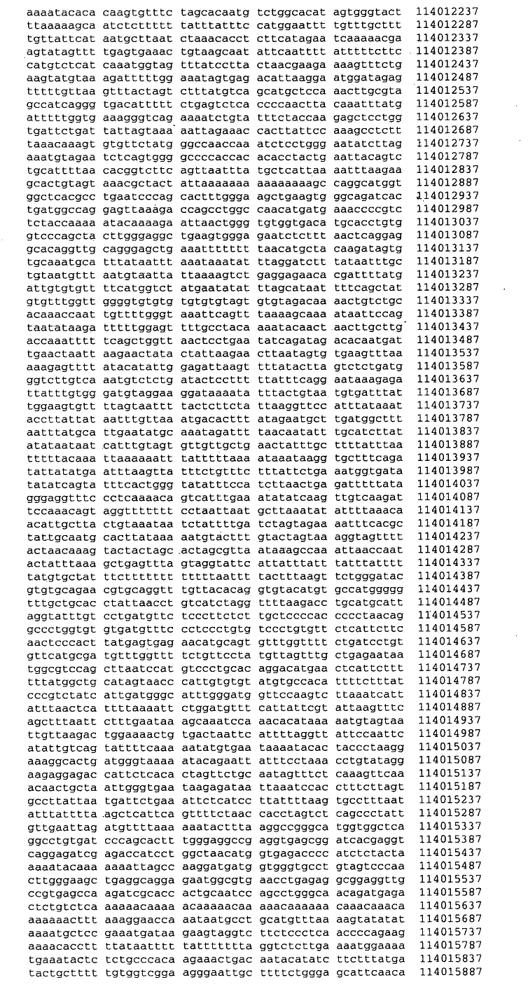

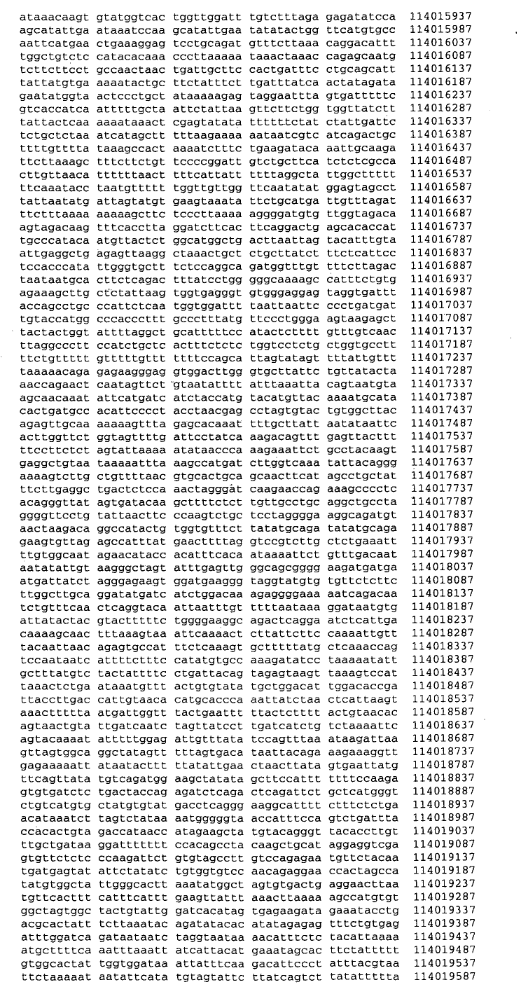

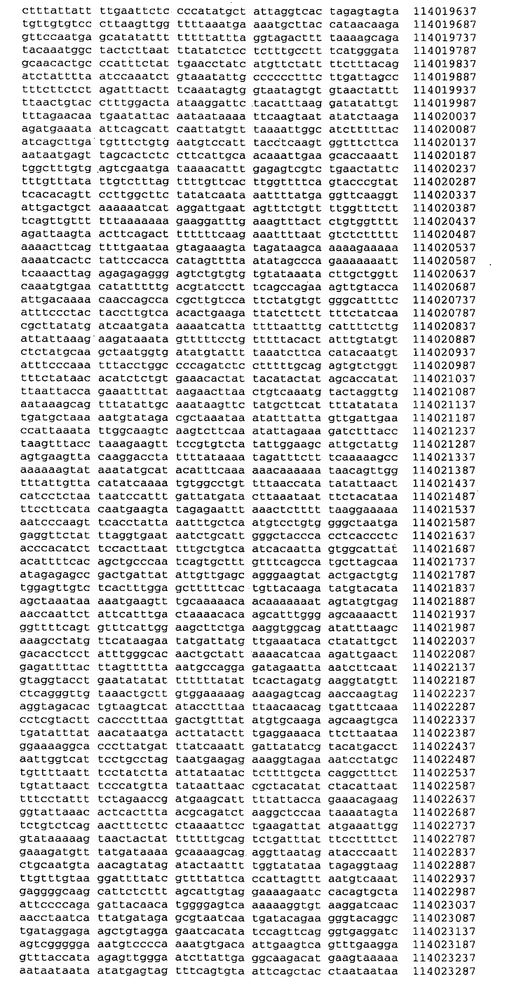

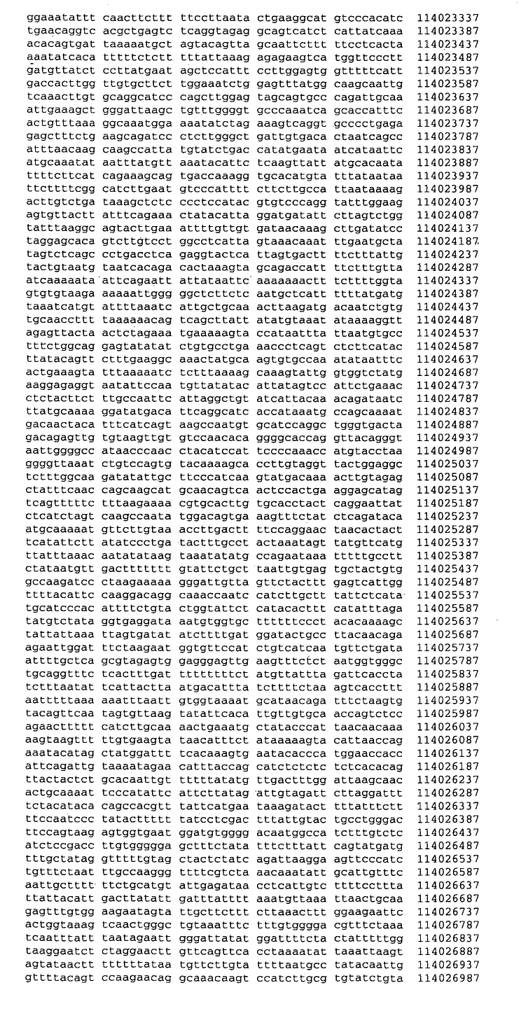

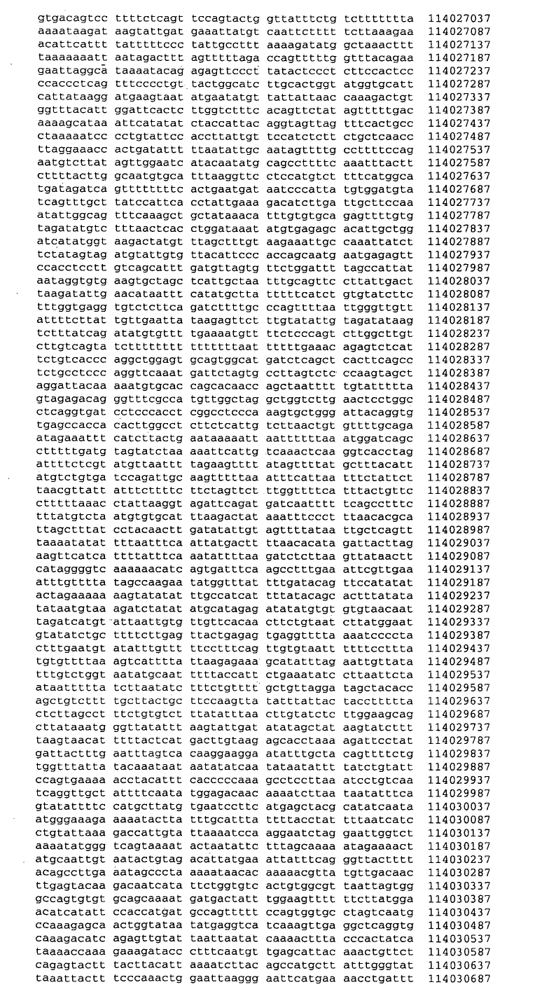

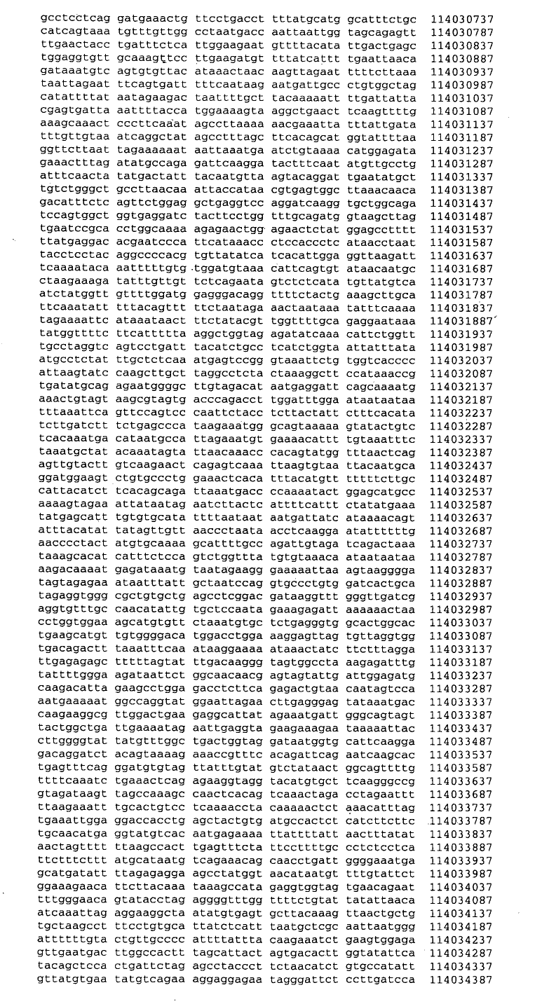

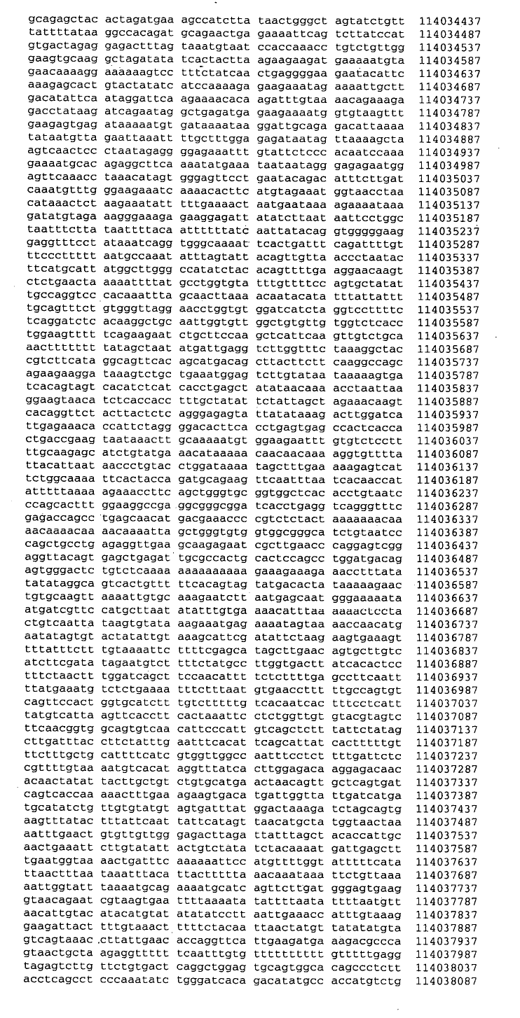

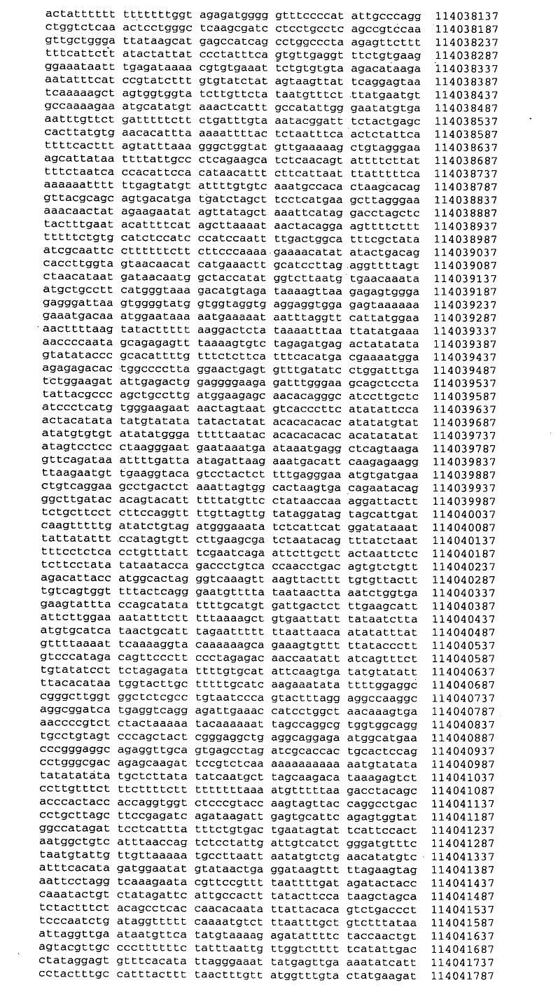

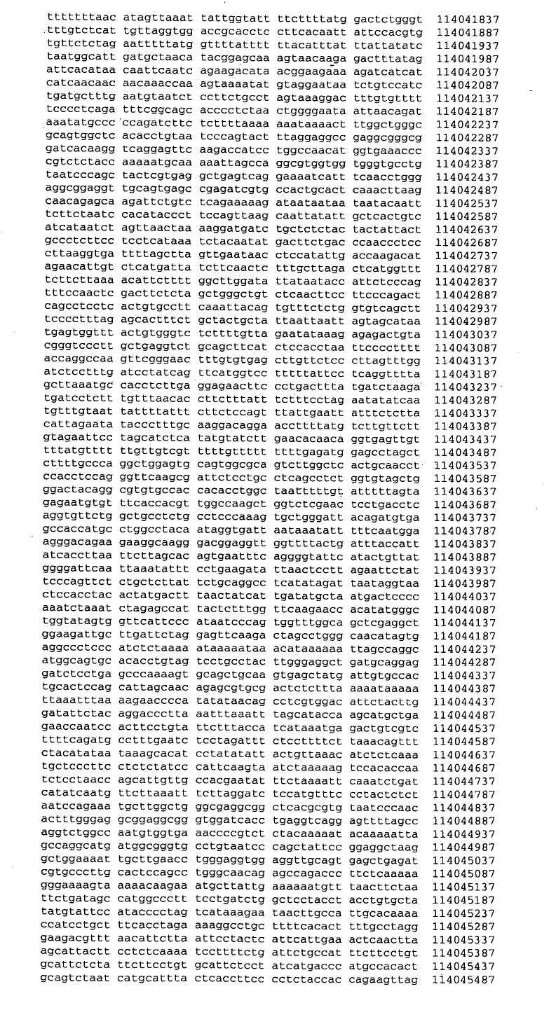

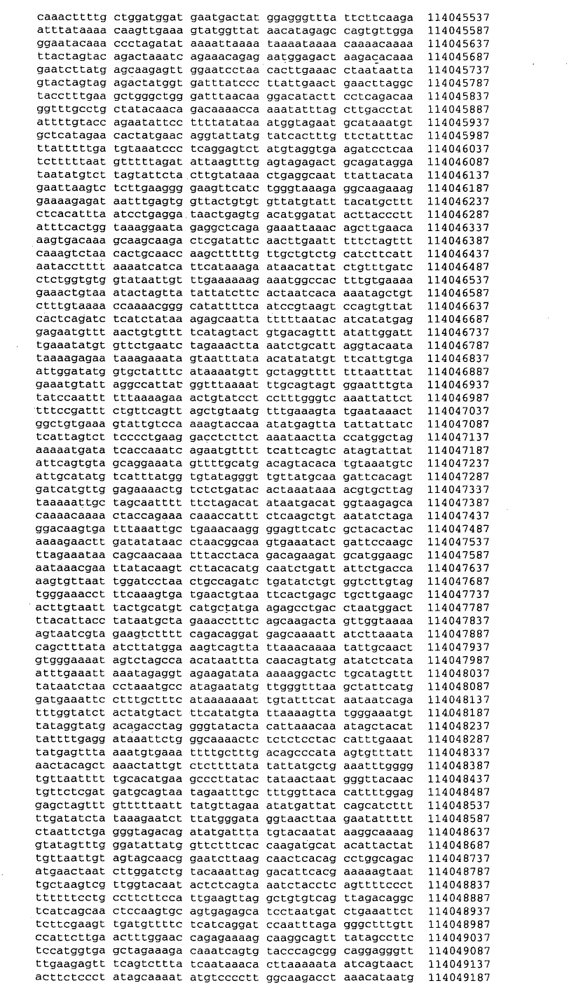

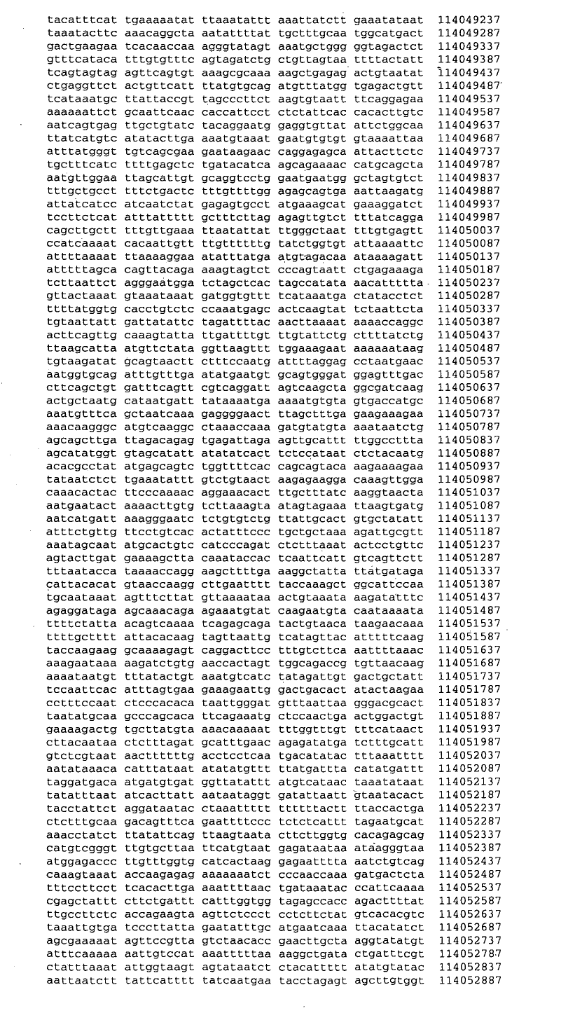

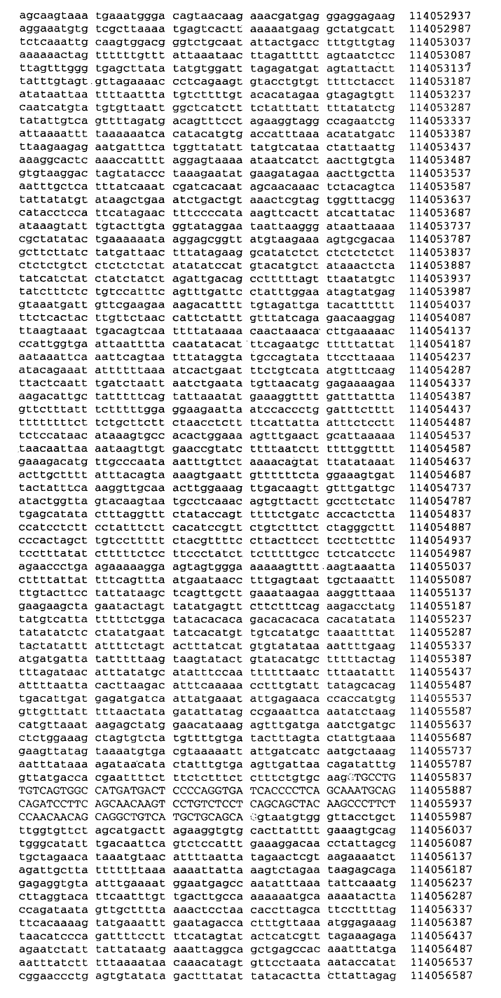

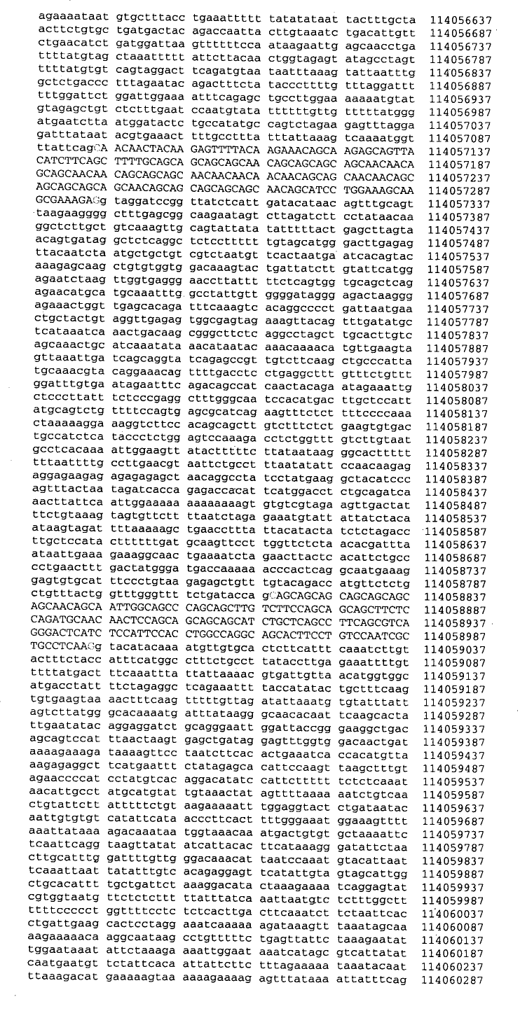

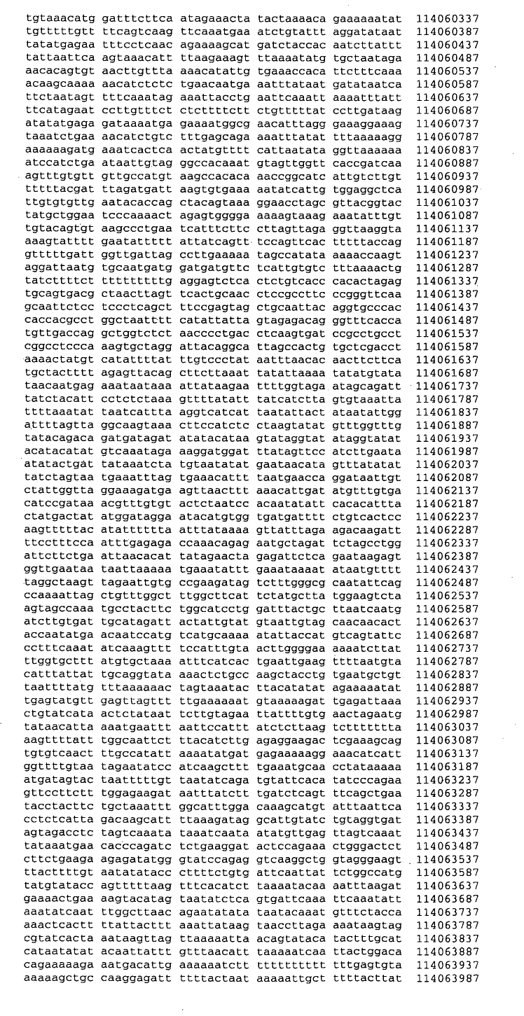

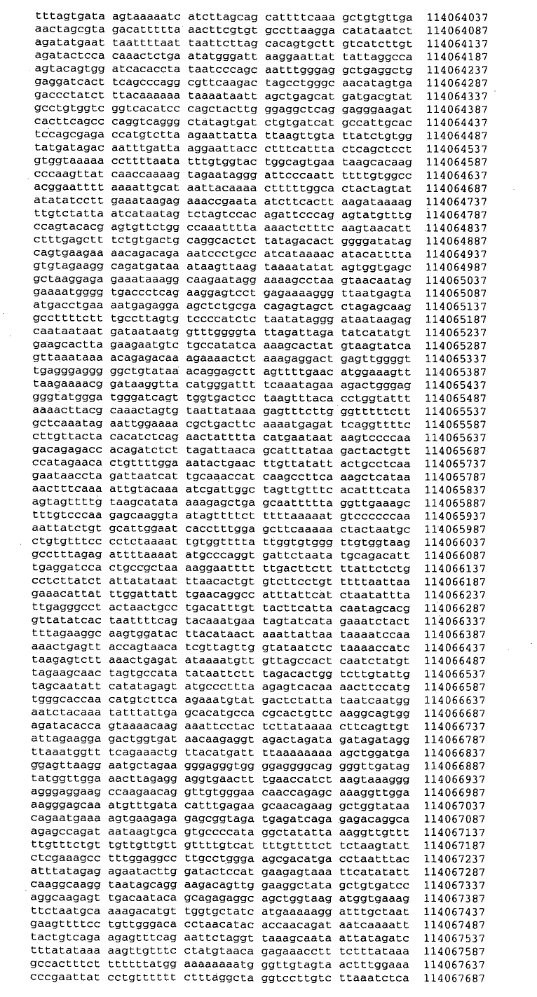

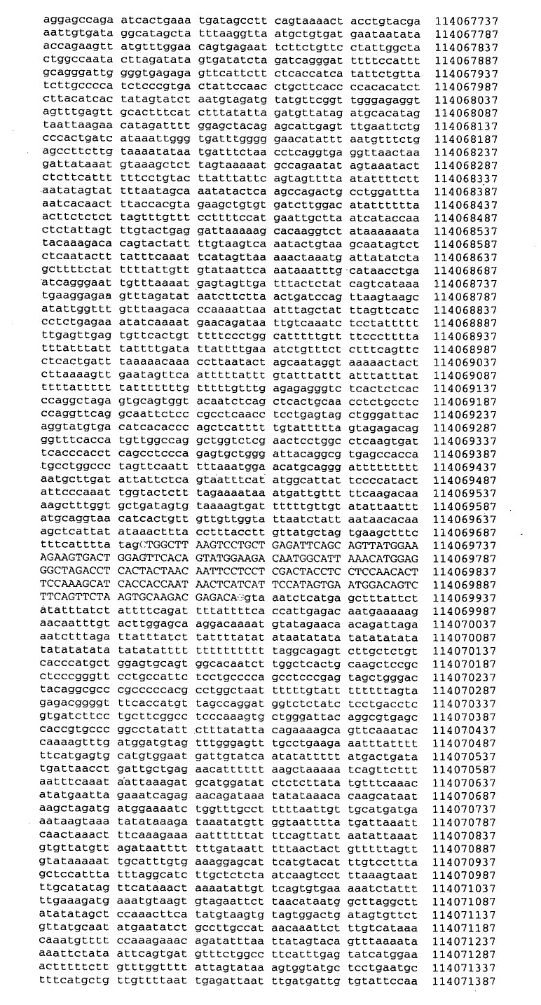

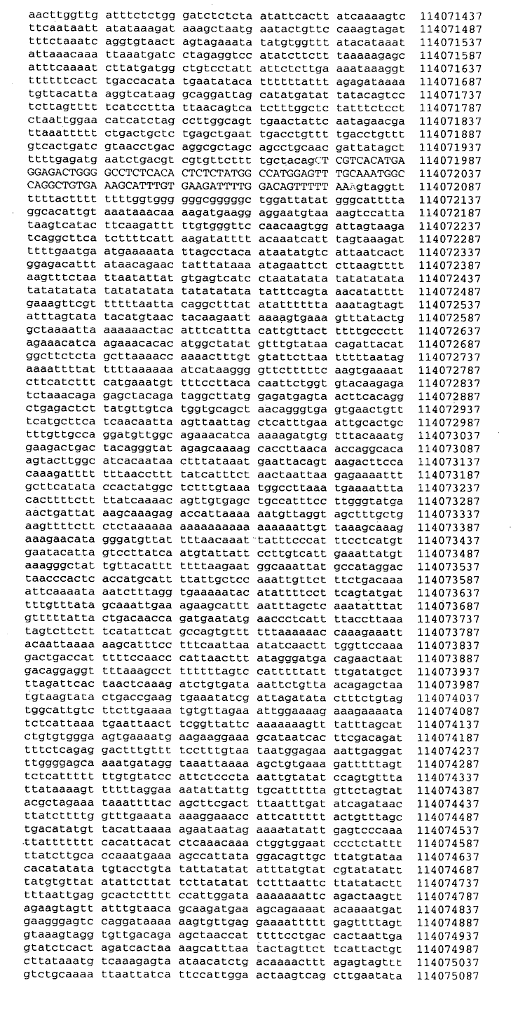

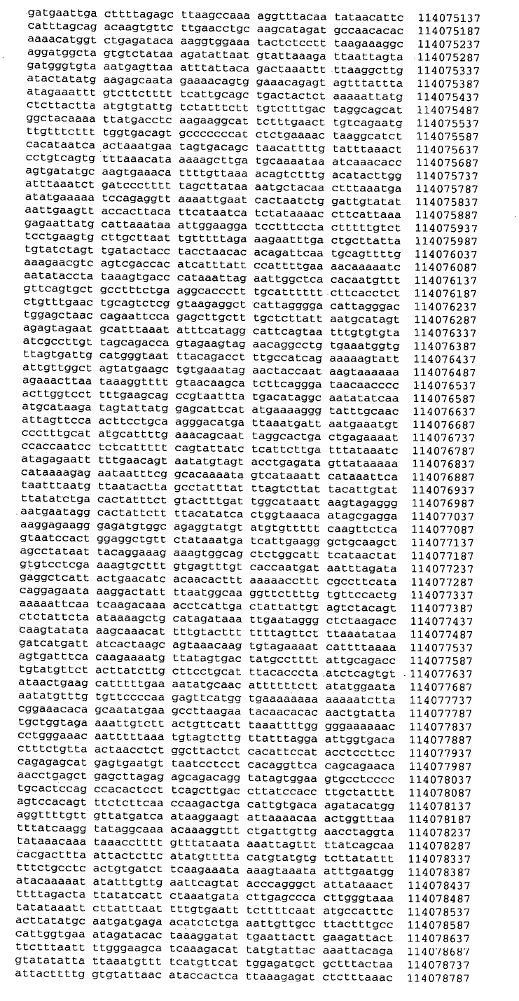

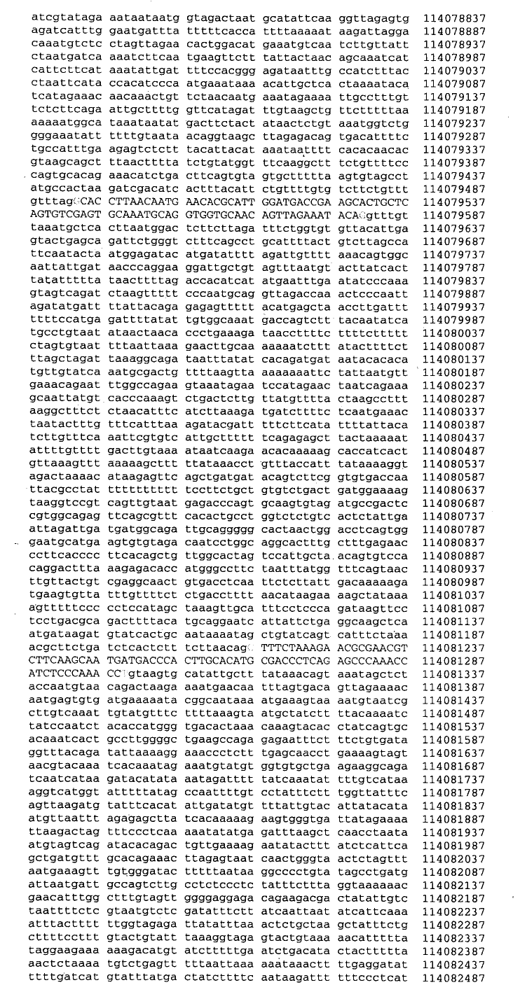

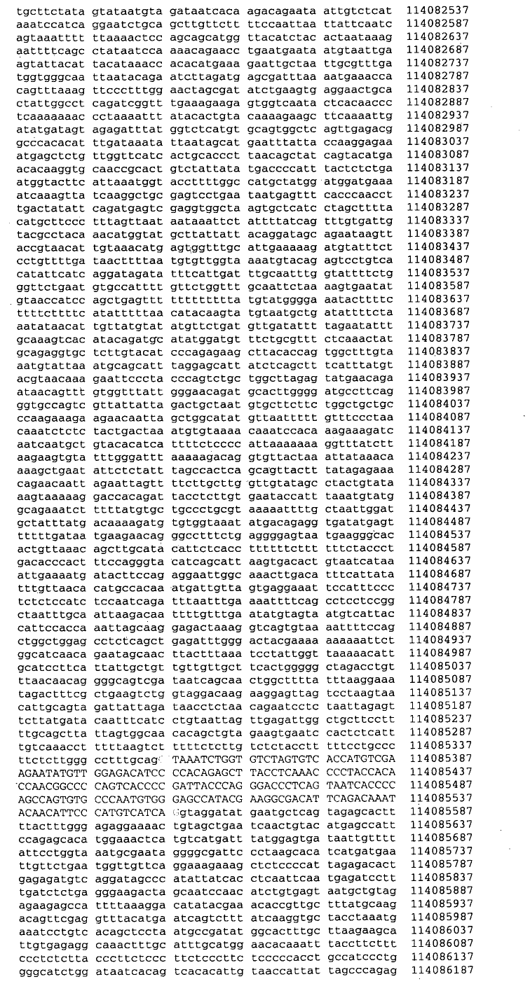

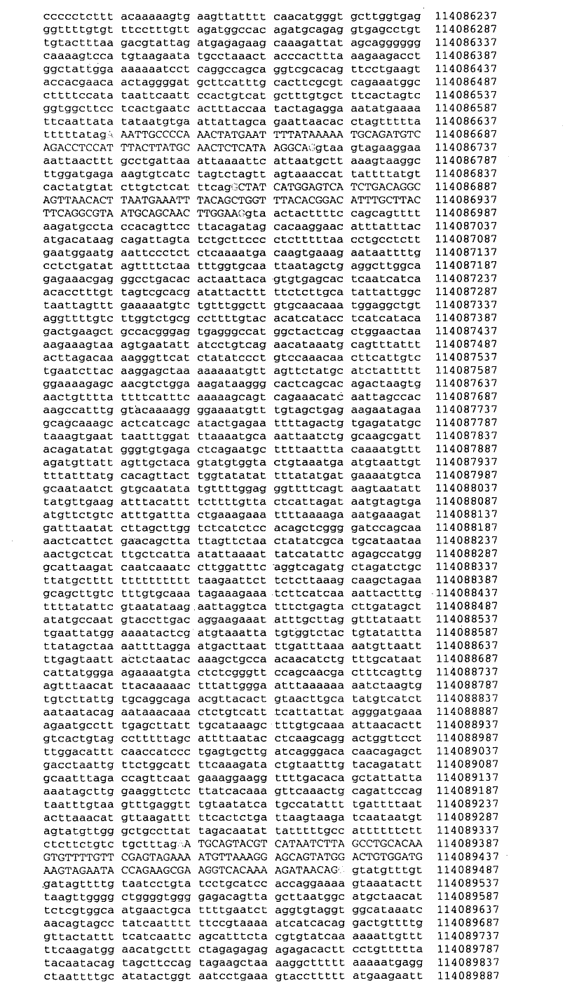

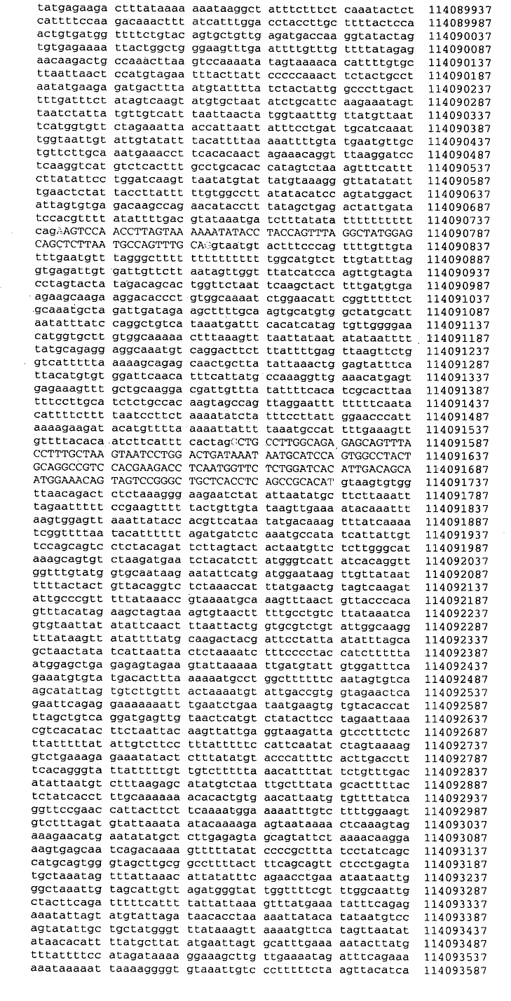

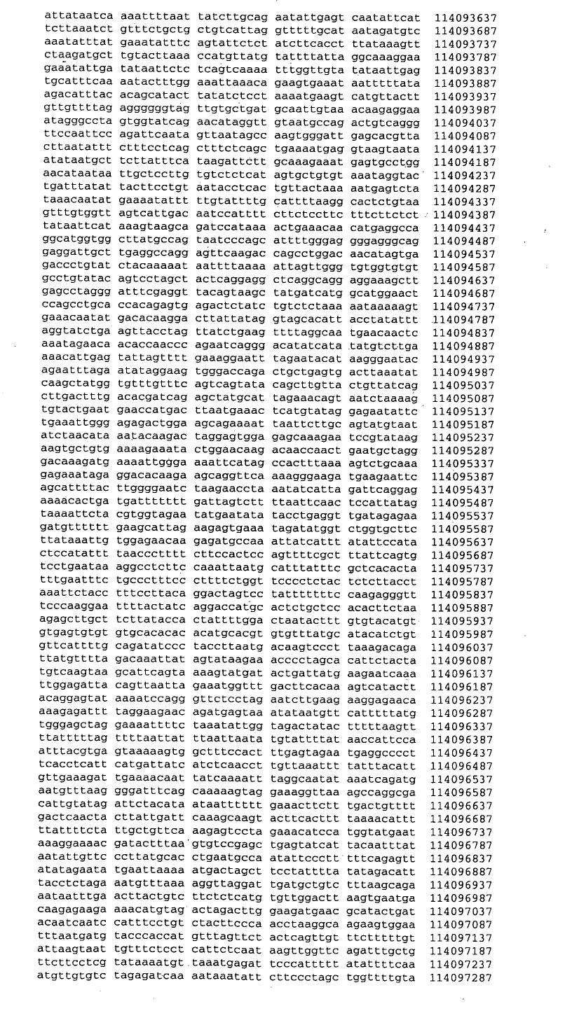

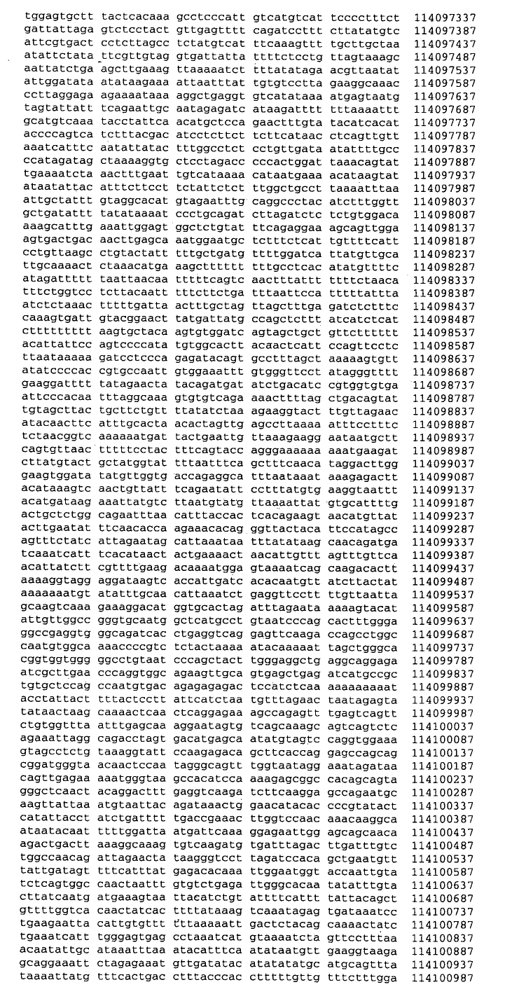

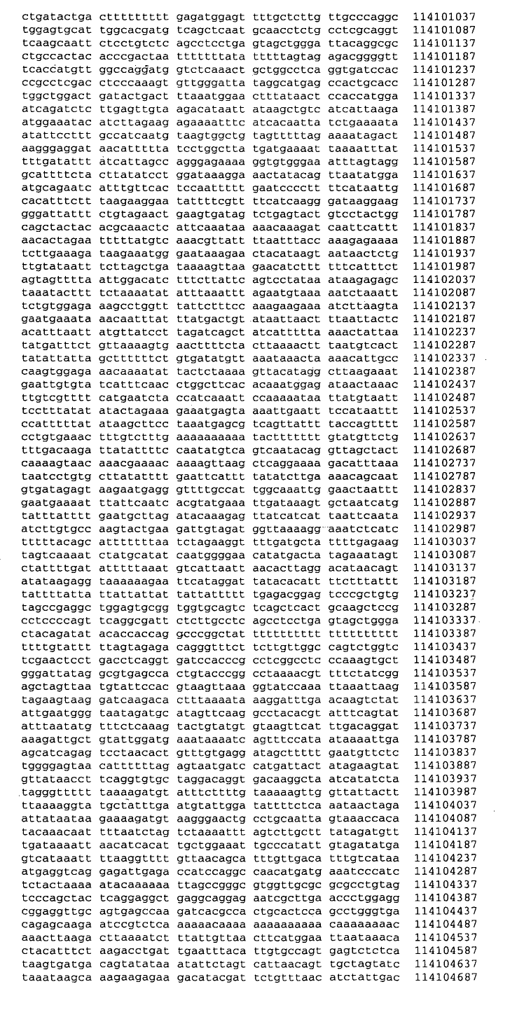

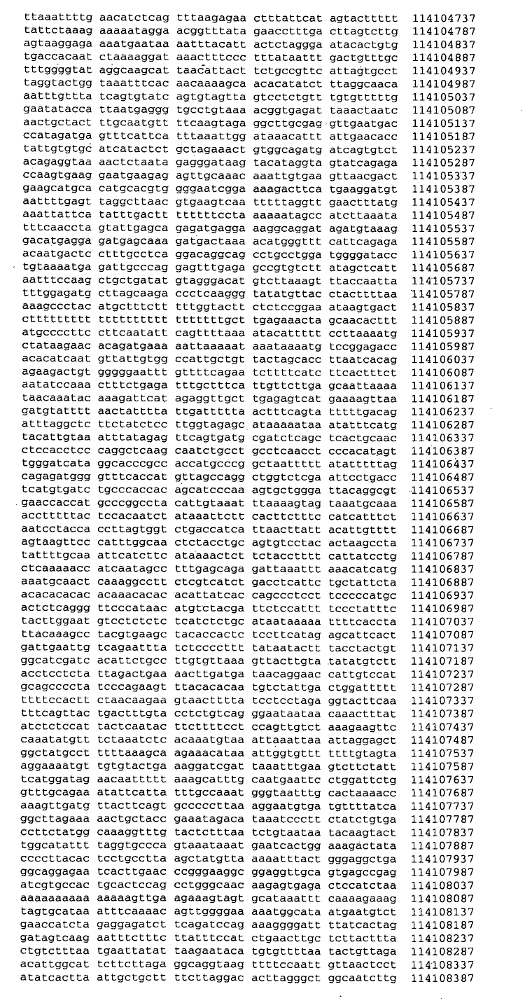

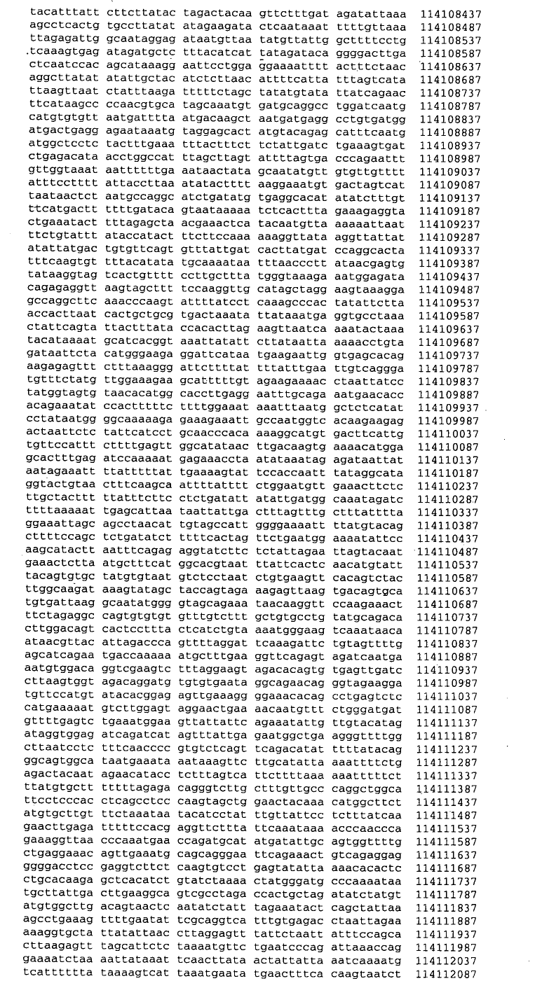

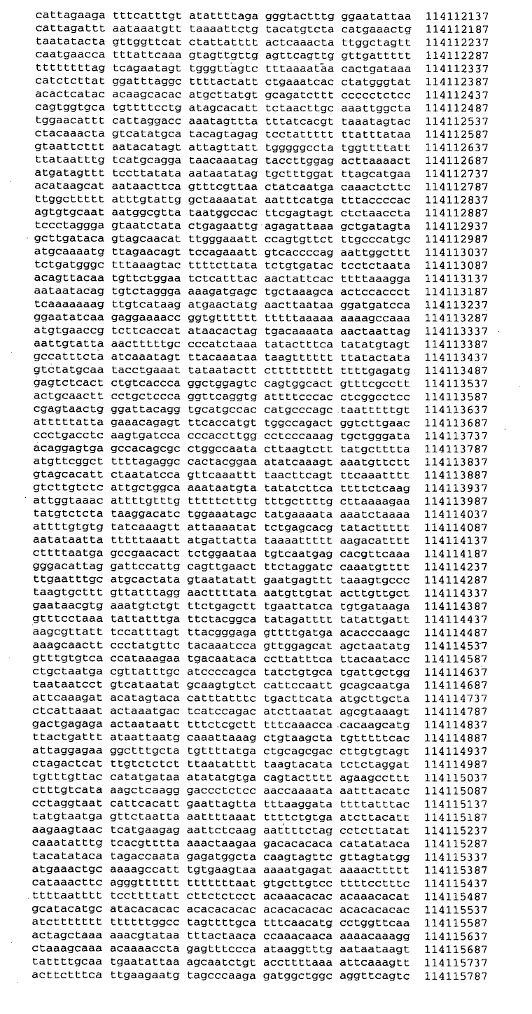

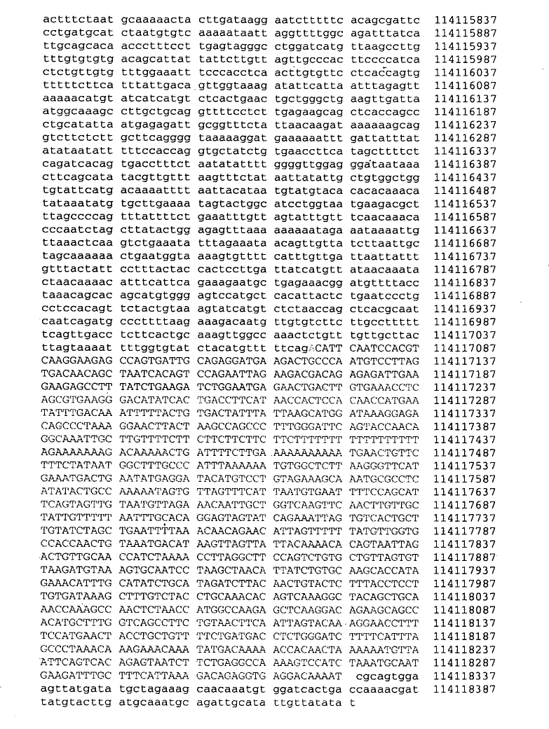

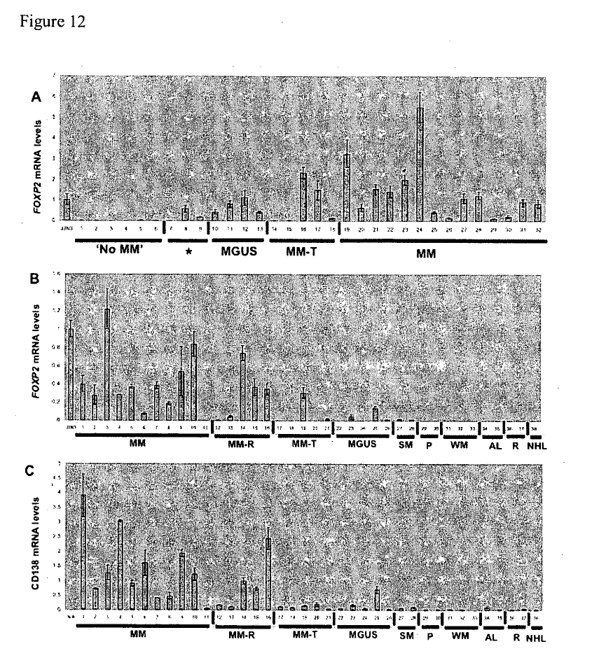

FIG. 12 shows FOXP2 mRNA expression in purified CD 138⁺ plasma cells and total bone marrow aspirates. This figure corresponds to FIG. 4 but has reclassified patient 7 in panel A. FIG. 12 A) shows FOXP2 expression in CD138⁺ purified bone marrow plasma cells. Those indicated with a * represent three patients that were difficult to classify; two patients had a trephine reported as either LPL or myeloma, although clinical data suggested the more likely diagnosis was a low grade lymphoma and the other patient (number 9) had a bone plasmacytoma with insufficient bone marrow trephine to define a malignant infiltrate. qRT-PCR analysis of both FOXP2 expression (B) and CD138 expression (C) was also performed using 38 aspirate samples from total bone marrow. MM=multiple myeloma at diagnosis; no MM includes a heterogeneous group of patients whose plasma cells should not be malignant or pre-malignant; MM-T refers to samples taken from patients post-treatment; MM-R=MM patients at relapse; SM=smoldering myeloma; P=plasmacytoma, WM=Waldenstrom's Macroglobulinaemia; AL=amyloid; R=reactive marrow; NHL=non-Hodgkin's lymphoma.

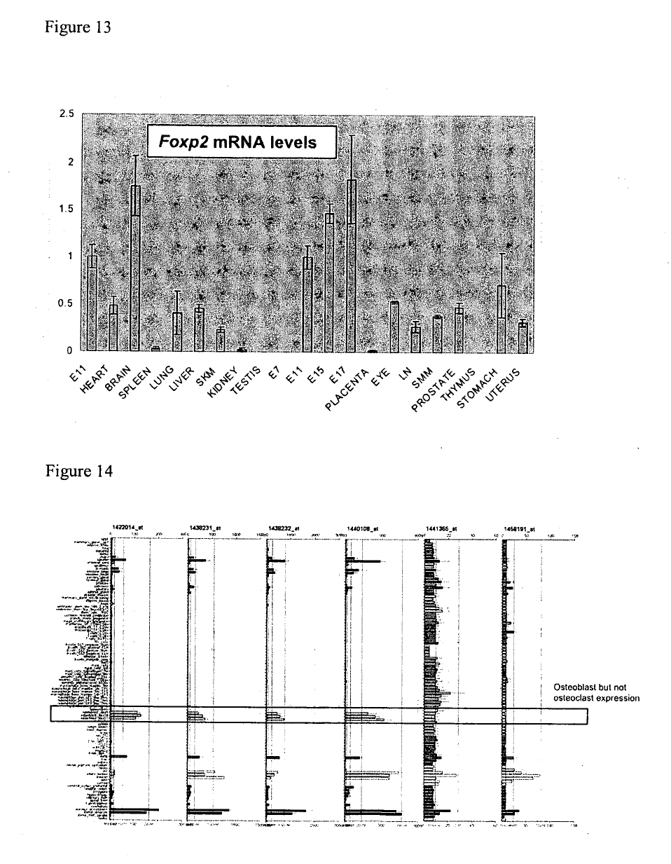

FIG. 13 shows qRT-PCR of murine FOXP2 expression in a panel of adult murine tissues and in embryonic tissues. E11, 7, 15 and 17 refer to days of embryonic development, LN:lymph node, SKM: skeletal muscle, SMM: smooth muscle. FOXP2 expression levels were normalised using murine Tbp expression and are expressed relative to those in the Ell embryo.

FIG. 14 shows an investigation of murine Foxp2 mRNA expression in normal tissues. The boxed section illustrates lack of osteoclast expression in the top sample, while high levels of Foxp2 expression were observed in the three osteoblast samples below.

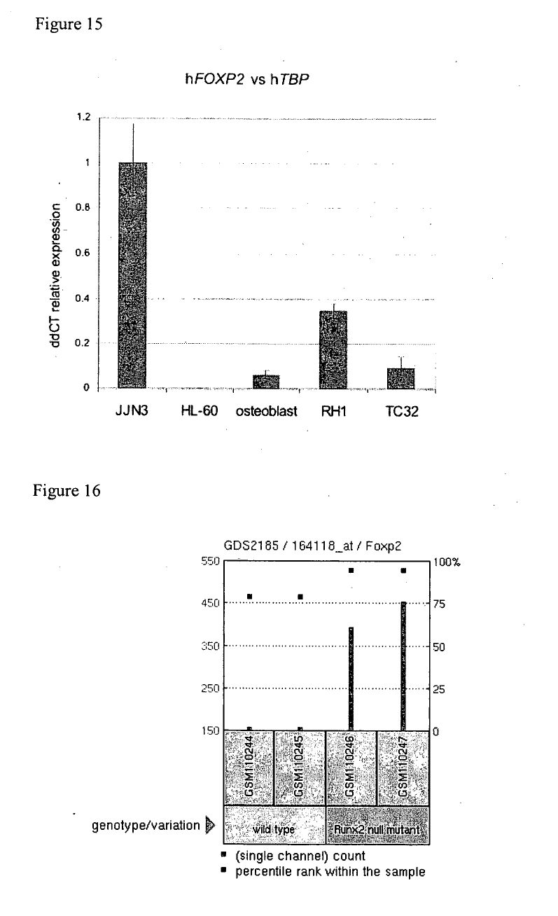

FIG. 15 shows FOXP2 mRNA expression in human osteoblasts and Ewing's sarcoma cells lines. FOXP2 expression was normalised using TBP and expressed relative to levels in the JJN3 myeloma cell line. RH1 and TC 32 are Ewing's sarcoma cell lines.

FIG. 16 shows Foxp2 mRNA expression is elevated in the bones of Runx2 null mice during embryonic development.

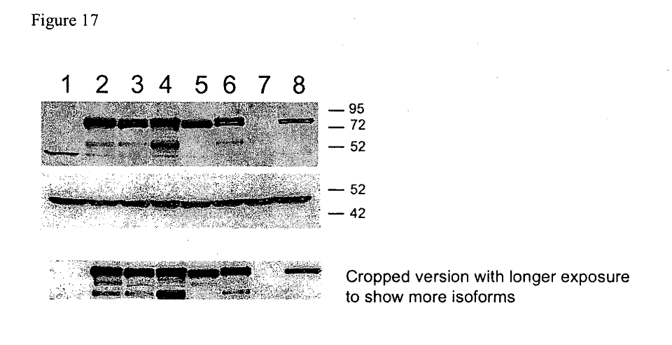

FIG. 17 shows Western blotting using antibody FOXP2-73A/8 of Ewing's sarcoma cell lines. Strong expression of two proteins with a molecular weight comparable to that of the full length FOXP2 protein, and additional smaller proteins that may represent FOXP2 isoforms, was observed in ES-derived cell lines. 1 ND7 cells, a murine neuroblastoma/primary sensory neuron hybrid; 2 RH1 cells, EWS/FLI1 Ewing's sarcoma cells human; 3 TC32 cells, EWS/FLI1 Ewing's sarcoma cells human; 4 SKNMC cells, EWS/FLI1 Ewing's sarcoma cells human; 5 RDES cells, EWS/FLI1 Ewing's sarcoma cells human; 6 RH30 cells, PAX37FKHR alveolar rhabdomyosarcoma human; 7 RD cells, embryonal rhabdomyosarcoma human; 8 SHSY5Y cells, neuroblastoma human.

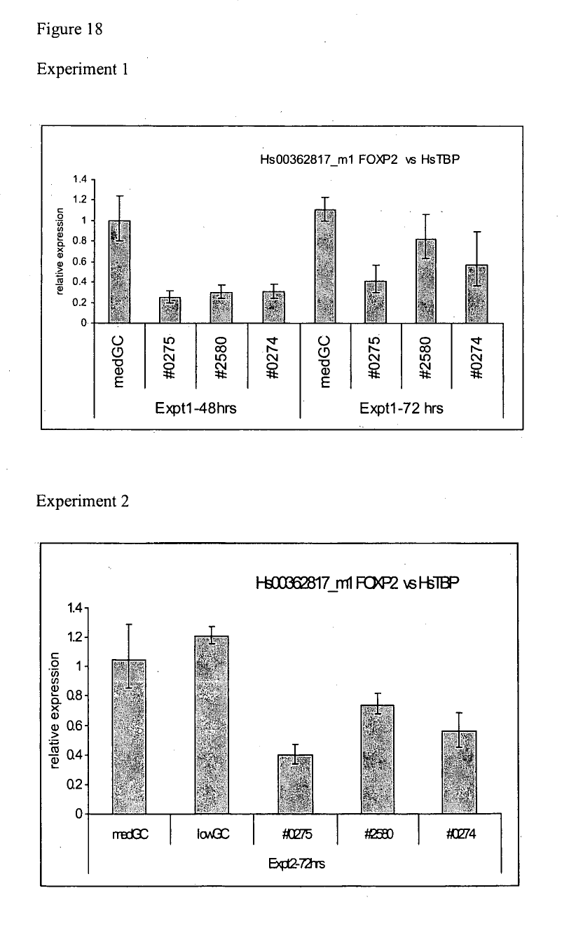

FIG. 18 shows FOXP2 silencing in the JJN3 MM cell line. Silencing was performed using three individual stealth siRNAs from Invitrogen (#0275, #2580, #0274). Silencing was compared to universal control siRNAs. FOXP2 expression levels were normalised using TBP expression and are expressed relative to those in the medGC control siRNA treated cells.

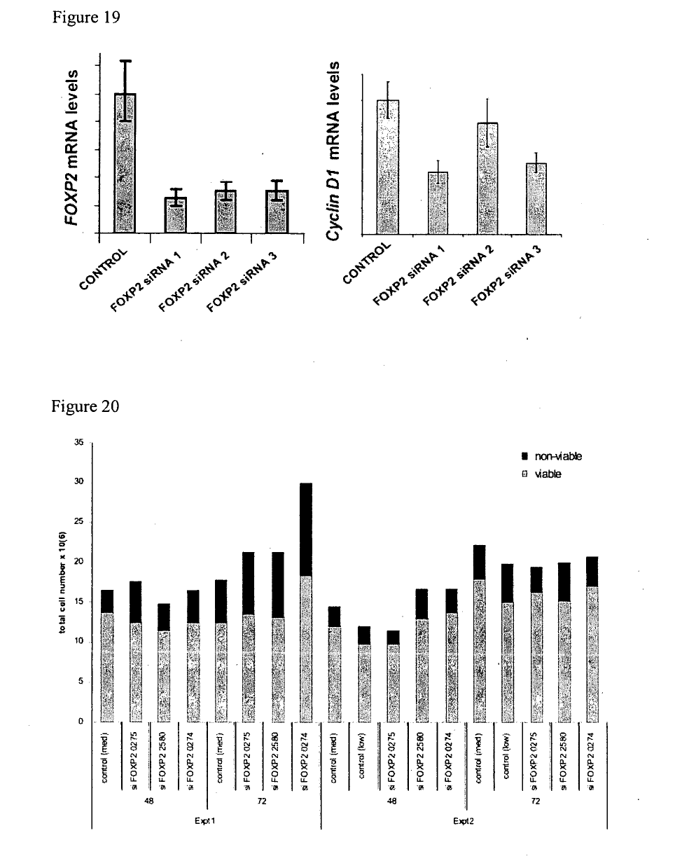

FIG. 19 shows CCND1 expression is reduced on FOXP2 silencing in the JJN3 cell line. FOXP2 silencing was performed for 48h using three individual stealth siRNAs from Invitrogen (1=#0275, 2=#2580, 3=#0274); the data in the left panel is reproduced from FIG. 2, experiment 1. Silencing was compared to a universal medGC control siRNA. FOXP2 and CCND1 expression levels were normalised using TBP expression and expressed relative to those in the medGC control siRNA treated cells.

FIG. 20 shows that cell viability is not dramatically affected by FOXP2 silencing. Silencing was performed in duplicate experiments using three individual stealth siRNAs from Invitrogen (#0275, #2580, #0274). Silencing was compared to universal control siRNAs. Total cell numbers were counted after the time indicated (48 h or 72 h). The number of non-viable cells, that were stained with trypan blue, were subtracted from the total to yield the number of viable cells.

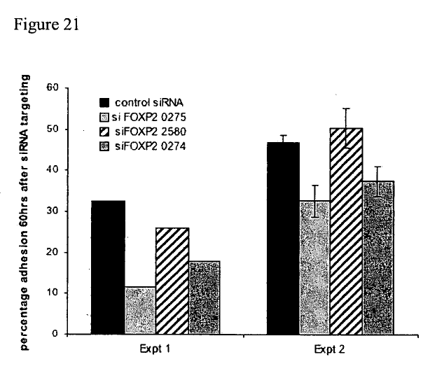

FIG. 21 shows that FOXP2 silencing reduces JJN3 cell adhesion to stromal cells. In the first experiment only the number of adherent JJN3 cells were determined, thus the percentage of adherent cells was calculated relative to the 200,000 that were plated. In the second experiment both adherent and non-adherent populations were counted and the percentage adherence was determined in relation to the combined number of adherent and non-adherent myeloma cells.

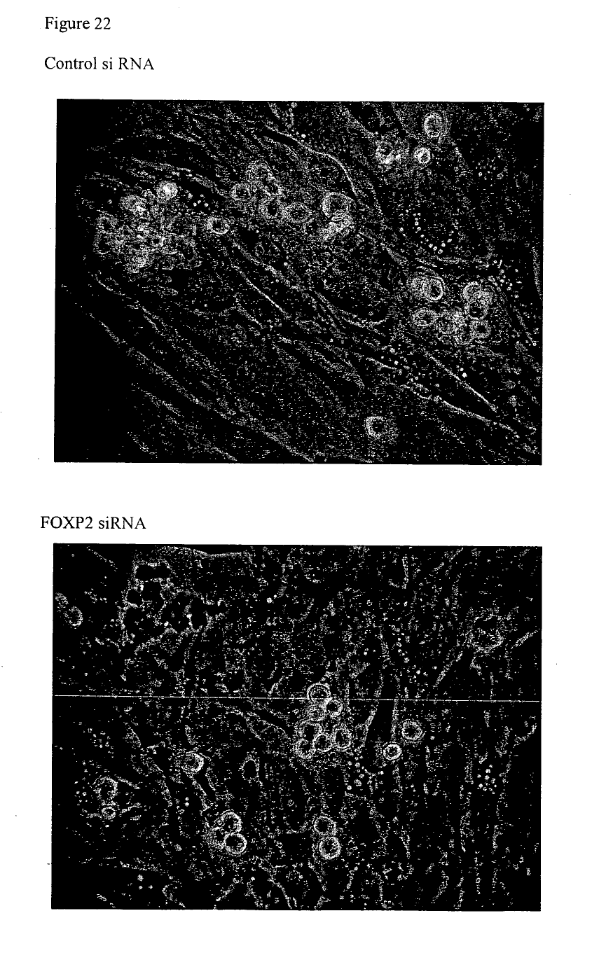

FIG. 22 shows JJN3 cell adhesion to stromal cells after FOXP2 silencing. Clumps of small round JJN3 myeloma cells are shown adhering to the OP9-GFP stromal cell layer after washing. Larger clumps are present in the cells treated with the control siRNA when compared to those treated with FOXP2 siRNA. This is consistent with the reduction in the number of FOXP2 siRNA treated cells adhering to the stromal cell monolayer.

### EXAMPLE 1

Patients

Patients' samples were obtained with informed consent from the John Radcliffe Hospital, Oxford, the Dubrava University hospital, Zagreb, Croatia and the Centro Nacional de Investigaciones Oncológicas (CNIO), Madrid, Spain. This study was conducted under ethical approval from the Oxfordshire Clinical Research Ethics Commitee.

Cell lines and Culture Conditions

SUDHL9 was a kind gift from Professor Martin Dyer, Leicester; the OCI-Ly3, OCI-Ly10 (ABC-derived), SUDHL6, SUDHL10 and DB (GC-derived) DLBCL cell lines were a kind gift from Dr Eric Davis, Bethesda USA; the MIEU and HLY-1 DLBCL cell lines were generously provided by Dr Talal Al Saati, Toulouse, France. MEDB1 was kindly provided by Drs S. Briiderlein, P. Möller and T. Barth (Institute of Pathology, Ulm, Germany). Cells were maintained in RPMI 1640 media supplemented with 10% FCS, 2 mM glutamine and antibiotics [streptomycin (50 μg/ml) and penicillin (50 U/ml)] at 37 oC and 5% CO2. Normocin was added to culture media for nucleofection experiments (100 μg/ml).

Quantitative Real-time PCR

Total RNA was extracted from cell lines using an RNeasy kit according to the manufacturers instructions (Qiagen, Crawley West, Sussex, UK). 100 ng of total RNA was reverse transcribed using random primers (Promega, Southampton, UK) and Superscript III reverse transcriptase (Invitrogen, Paisley, UK). CD138 cells were isolated from bone marrow aspirates using MACS microbeads (Miltenyi Biotec, Bergisch Gladbach, Germany). Normal tissue and lymphoid cDNAs were obtained from Clontech, CA, USA and specific lymphoid subsets were isolated from pooled buffy coat samples or tonsil, as previously described.(Lawrie et al., 2007, Int J Cancer, 121 (5), 1156-1161) RNA from BM aspirates was isolated using QIAamp RNA Blood Mini Kit (QIAGEN, Hilden, Germany) according to the manufacturer's protocol. One microgram of total RNA was used for cDNA synthesis using GeneAmp Gold RNA PCR Core Kit (Applied BioSystems, New Jersey, USA) according to the manufacturer's protocol. Real-Time PCR amplification was performed with a Chromo 4 continuous fluorescence detector (MJ Research, Waltham, Mass.). A TaqMan inventoried pre-verified assay reagent was used to amplify FOXP2, (Hs00362817_l ml), CD138 (Hs00896423_ml) and SMAD3 (Hs00706299_s1); Applied Biosystems. Experiments were performed in triplicate for each data point. The relative gene expression level was normalized on the basis of the expression of a reference gene, TATA Box binding protein (TBP; 4326322E; Applied Biosystems). The formula 2 (cycle threshold FOXP2-cycle threshold TBP) was used to calculate normalized expression.

Western Blotting

Nuclear extracts were prepared using the Panomics Nuclear extraction kit according to the manufacturer's instructions (Panomics, CA, USA). Proteins were resolved by SDS-PAGE and transferred to Hybond nitrocellulose membrane (GE Healthcare, Amersham, UK). The membrane was blocked (1×PBS, 5% Marvel) for 1 hour followed by incubation with primary antibody overnight at 4oC. Primary antibodies used were FOXP2 N-16 sc-21069 at 1/2000 (Santa Cruz, Calif., USA), FOXP2 ab16046 at 1/1000 (AbCam, Cambridge, UK), TATA binding protein (TBP) 1TBP18 at 1/2000 (AbCam) and Actin ab6276 at 1/10,000 (AbCam). The membrane was washed and incubated with 1/5,000 horseradish peroxidase (HRP) linked secondary antibody (Dako). Labelling was detected using the enhanced chemiluminescence (ECL) reagent (Amersham Biosciences, Amersham, UK). Blots were re-probed with antibodies to TBP or actin to confirm adequate sample loading.

FOXP2 Silencing Using siRNA

JJN3 cells were transfected using the siRNA duplex FOXP2-ATGGAAGACAATGGCATTAAA or standard non-silencing control (Qiagen, Crawley, UK). Nucleofection was used to introduce the duplex into cells according to manufacturer's protocols (Amaxa Inc, Gaithersburg, Md.). Cells were harvested at 48 hours and underwent either nuclear extraction (for Western Blotting) or total RNA extraction (for qRT-PCR). The duplex concentration used for nucleofection was 3.3 μM.

Monoclonal Antibody Production

Balb-c mice were immunised with a bacterially expressed recombinant protein comprising the N-terminal region of FOXP2 (aa 1-86) fused to glutathione-S-transferase. The FOXP2-73A/8 antibody was found to have specific reactivity against FOXP2 by screening for ELISA reactivity with the recombinant protein, validation on FOXP2, FOXP1, FOXP3 and FOXP4 transfectants and staining of the JJN3 cell line.

Immunohistochemistry

Fresh frozen sections and cytospins were stored at −20° C. and fixed for 10 minutes in acetone and air-dried for 30 minutes. Paraffin embedded slides were dewaxed in Citroclear (HD Supplies, Aylesbury, UK) followed by antigen retrieval using microwaving in 50mM Tris/2mM EDTA (pH 9.0). The Xpress antibody (R910-25 1/2000; Invitrogen, Paisley, UK) or the CD56 antibody (CD56-1B6-L-CE 1/80; Leica Microsystems, Wetzlar, Germany) was applied for 30 minutes at room temperature, while the FOXP2-73A/8 antibody was applied at a 1/1000 dilution overnight at 4 oC. Immunostaining was performed using the Envision system (Dako) for Xpress and CD56, while FOXP2 staining was performed using the NovoLink polymer detection system according to the manufacturer's instructions (Novocastra, Newcastle upon Tyne, UK). Counterstaining was with hematoxylin (Gill's No. 2; Sigma-Aldrich, St Louis, MO) and slides were mounted in Aquamount (VWR International, Lutterworth, UK). For double immunoenzymatic labelling the FOXP2 staining was performed as described above, without applying the counterstain, followed by the second antibody that was detected by means of a Vector SG blue horseradish peroxidase substrate kit (Vector Laboratories, Peterborough, UK). Second antibodies included CD138 (M7228; dilution 1/50; Dako); CD3 (M7254; dilution 1/100; Dako) and hybridoma supernatants Ki67, JC70, PGM1 (all used neat), PD7+2B11 (CD45, dilution 1/5), KP1, L26 (both diluted 1/20) and VS38C (diluted 1/50). Slides were washed in water, dehydrated in xylene and mounted in VectorMount (Vector Laboratories). The cases were scored without information on the diagnosis.

Results

Northern blotting and RT-PCR studies have reported widespread expression of the FOXP2 mRNA in adult murine and human tissues (Lai et al., 2001, Nature, 413, 519-523; Shu et al., 2001,1 Biol. Chem., 276, 27488-27497; Schroeder and Myers, 2008, Gene, 413(1-2), 42-48).

Analysis of FOXP2 Transcript Levels in Normal Human Tissues

Probing a normal tissue-derived MTE cDNA array with a FOXP2 cDNA fragment confirmed its widespread expression in normal tissues (FIG. 1A). Only very low-level expression was observed in spleen and lymph node, while no expression was detected in bone marrow, peripheral blood leukocytes and thymus. Analysis using qRT-PCR confirmed the absence or only very low-level expression of FOXP2 in hematological tissues (FIG. 1B). Lymphoid tissues contain a variety of different cell types, including epithelial and endothelial cells. Therefore, FOXP2 mRNA expression was also investigated in purified cell populations derived from both resting and activated mononuclear cell populations (FIG. 1C). No expression of FOXP2 was observed in monocytes, T cells, or in B cells at different stages of development, including terminally differentiated CD138⁺ plasma cells.

FOXP2 is Expressed at High Levels in Myeloma and Lymphoma Cell Lines

Analysis of FOXP2 mRNA expression in a panel of cell lines derived from hematological malignancies (FIG. 2) identified particularly high-level expression in all four MM cell lines, two Hodgkin lymphoma (HL) cell lines (n=4) and one diffuse large B-cell lymphoma (DLBCL) cell line (n=10). The expression of FOXP2 in five of the seven strongly positive lymphoma/MM cell lines was higher than observed in any normal human tissue.

Two commercial polyclonal antibodies were validated for their ability to specifically detect the FOXP2 protein using Western blotting and immunohistochemistry. The antibody from Abcam was excluded because it also recognized both the FOXP1 and FOXP4 proteins (FIG. 2C). The Santa Cruz antibody was FOXP2 specific but could only be used to detect FOXP2 by Western blotting (FIG. 2D). A good correlation was observed between the presence of FOXP2 mRNA and nuclear protein expression in the cell lines (FIGS. 2A and B). Silencing FOXP2 expression in the JJN3 cell line (FIG. 2E) using RNA interference (RNAi) confirmed the identity of the endogenous protein as FOXP2.

FOXP2 Transcript Levels in Patients' Samples

Expression studies were extended to patients' samples. FOXP2 mRNA was undetectable in the circulating malignant population in B-CLL (n=10) and biopsies from follicular lymphoma patients (n=8) also contained little if any FOXP2 mRNA (FIG. 3). One DLBCL biopsy (n=10) contained high levels of FOXP2 and a further three cases had more expression than observed in normal lymph node or FL biopsies (FIG. 3). Hodgkin lymphomas were not studied by qRT-PCR due to the scarcity of the malignant Reed Sternberg cells.

Normal plasma cells lacked FOXP2 mRNA expression, and FOXP2 expression was also absent, or expressed at very low levels, in CD138 purified bone marrow plasma cells from a heterogeneous patient group whose plasma cells should not be malignant or pre-malignant (n=7 originally (FIG. 4), and then one patient re-classified in view of additional clinical data; thus n=6 (FIG. 12)). All the CD138 purified samples from patients with MGUS (n=3) or active MM (n=15) exhibited FOXP2 expression (FIG. 4). High-level FOXP2 mRNA expression was also present in two of the five treated MM patients (15 & 16). Notably these were the only two treated cases having residual populations of plasma cells visible in the post-treatment trephine.

FOXP2 mRNA expression was also studied in total bone marrow aspirates from an independent patient cohort of 38 marrows (FIG. 4). Ten patients with active MM (n=11) and four MM patients in relapse (n=5) had detectable FOXP2 mRNA in their aspirate, with levels generally being consistent with the CD138 mRNA expression that was used to assess plasma cell infiltration. The negative MM patient (#11) also had exceptionally low CD138 expression, suggesting that there were very few plasma cells present in the sample. Of the five MM patients who had undergone treatment, only the one with refractory disease (#19) retained significant FOXP2 mRNA expression. Interestingly, only the two MGUS patients (n=5) with the highest levels of CD138 mRNA also expressed FOXP2.

Production of a Novel Anti-FOXP2 Monoclonal Antibody

The commercially available anti-FOXP2 polyclonal antibodies were unsuitable for detecting FOXP2 by immunohistochemistry. Therefore, a novel murine monoclonal antibody (FOXP2-73A/8) was raised to a bacterially expressed GST-fusion protein comprising the N-terminus of the human FOXP2 protein. Western blotting studies confirmed the specificity of this antibody for FOXP2, demonstrated the loss of endogenous protein after FOXP2 targeted siRNA in the JJN3 cell line and produced the same pattern of full length FOXP2 expression in the cell line panel as observed using the commercial polyclonal antibody (FIG. 5). However, the FOXP2-73A/8 antibody was also effective in immunohistochemistry applications and specifically recognized a formalin resistant FOXP2 epitope that was not cross-reactive with the related FOXP1, 3 and 4 proteins (FIG. 6). Immunolabelling studies confirmed the expression levels of FOXP2 and its nuclear localization in MM and lymphoma cell lines and demonstrated heterogeneity in FOXP2 levels at the single cell level (FIG. 6).

FOXP2 Protein Expression in Normal Human Tissues

FOXP2 protein expression was assessed by immunohistochemistry with antibody FOXP2-73A/8 on whole sections of tonsil and a normal tissue microarray containing 39 different human tissues. FOX2 was widely but not ubiquitously expressed in normal human tissues with FOXP2-73A/8 exhibiting primarily nuclear but also cytoplasmic labelling patterns (FIG. 6). FOXP2 expression in tonsil was restricted to epithelial and endothelial cell nuclei and to scattered nuclei in the interfollicular areas. Double labelling studies confirmed the absence of FOXP2 protein expression in haematopoietic cells including CD20⁺ B-cells, CD3 ⁺T cells and CD138+ plasma cells. However, occasional CD138⁺ plasma cells in reactive tonsil and bone marrow were noted to exhibit nuclear FOXP2 labelling. Double labelling with an antibody to CD45 (leucocyte common antigen) indicated that the scattered FOXP2⁺ cells in the interfollicular areas were not leucocytes. Importantly there was no indication that FOXP2 was expressed in a significant population of lymphocytes (including plasma cells) at other sites, including reactive lymph node, bone marrow, spleen and gut.

FOXP2 Protein Expression in MGUS and MM

FOXP2 protein expression in routinely fixed bone marrow trephines from non-malignant reactive marrows (n=7), patients with MGUS (n=9) or MM (n=73) was assessed by immunohistochemistry with antibody 73A/8 (FIG. 6). Double labelling with CD138 was performed to confirm FOXP2 expression in the plasma cell infiltrate and cases were also labelled for expression of the natural killer (NK) cell marker CD56, which is expressed in more than 75% of MM. None of the reactive bone marrows were scored as FOXP2⁺, although it was noted that several did contain occasional FOXP2+CD138+ cells. Eight of the nine MGUS samples were. FOXP2⁺ (only one also expressed CD56) and heterogeneity in FOXP2 expression levels was commonly observed, with not all CD138+ cells expressing FOXP2. Of the MM samples FOXP2 expression was generally less heterogenous and 82% were FOXP2⁺ compared to 71% that expressed CD56. Significantly, 55% ( 12/22) of the 22 CD56-negative cases were FOXP2⁺.

Some additional analysis was carried out on samples using antibody 73A/8 by immunohistochemistry. Thus, in this instance, FOXP2 protein expression in routinely fixed bone marrow trephines from non-malignant reactive marrows (n=10), patients with MGUS (n=11), MM (n=61), Waldenstrom's macroglobulinemia (WM) (n=2), plasma cell leukemia (PCL) (n=1), primary amyloid (n=3) or lymphoplasmacytoid lymphoma LPL (n=1) was assessed by immunohistochemistry with antibody 73A/8. FOXP2 expression was predominantly nuclear, cytoplasmic labelling lacked reproducibility, and showed heterogeneity of nuclear expression in terms of both the intensity and frequency of labelling. Double labeling with CD138 confirmed FOXP2 expression in the plasma cell infiltrate, demonstrated the existence of a small CD138⁻FOXP2⁺ population in some cases and enabled the percentage of FOXP2⁺ plasma cells to be assessed. Double labeling was used for scoring FOXP2 expression as it resulted in a higher frequency of FOXP2⁺ cases (55 versus 44 FOXP2⁺ MM within 61 cases) due to the improved visibility of weak nuclear labeling in the absence of a nuclear counterstain and the detection of low frequency FOXP2 expression in plasma cells.

FOXP2 protein expression was detectable in plasma cells in 8/10 of the reactive marrows, 11/11 MGUS and 58/61 MM. The frequency of plasma cell FOXP2 expression (FIG. 6C) was significantly higher in MGUS (p=0.0005; mean 46.4%, range 4.7-80.3%) and MM patients (p=<0.0001; mean 57.3%, range 0-96%) than in reactive marrows (mean 2.5%, range 0-10%). There was no significant difference between the frequency of FOXP2 expression in MGUS and MM. One uninvolved marrow from a Non-Hodgkin's lymphoma patient contained 10% FOXP2⁺ plasma cells. Double labelling with anti-CD20 confirmed FOXP2-negativity in B cells and excluded the possibility of marrow infiltration with a FOXP2⁺CD138⁺ B-cell lymphoma. A cut-off of >10% plasma cell FOXP2-positivity excluded all reactive marrows, WM, PCL, and LPL cases, while including 90.2% of MM (55/61), 90.9% (10/11) of MGUS and 66.7% (2/3) of primary amyloid patients.

FOXP2 expression was compared to that of the natural killer (NK) cell marker CD56, which is expressed in more than 75% of MM. In this series, 80% (49/61) of the MM and 18.2% of MGUS (2/11) expressed CD56. Greater than 10% FOXP2 plasma cell positivity (range 46.5-94.1%) was detectable in 75% (9/12) of MM that lacked CD56 expression, while only 50% (3/6) of the FOXP2-negative cases were CD56⁺ (FIG. 6C). Combining FOXP2 (>10% positivity) and CD56 expression detected 95.1% of MM (58/61).

FOXP2 Abnormalities in MM and Analysis of Transcriptional Start Sites in MM and Lymphoma Cell Lines

There are 368 MM, including the RPMI 8226 MM cell line (having seven copies of chromosome 7), 225 cases of DLBCL, and 18 cases of HL with gains of the FOXP2 locus (7q31) in the NCBI Cancer Chromosomes database (http://www.ncbi.nlm.nih.gov/sites/entrez?db=cancerchromosomes). Of particular interest were two MM cases that exhibit a t(7;14)(q31;32) translocation. Translocations involving the immunoglobulin heavy chain gene (IGH) locus at 14q32 are a relatively common mechanism of oncogene activation in both B-cell lymphomas and MM and may act to up-regulate FOXP2 expression. As the MM and lymphoma cell lines consistently expressed the full-length FOXP2 protein the usage of known, mutually exclusive, alternative transcriptional start sites for the human FOXP2 transcripts was also investigated (FIG. 7). Consistent with a previous study of carcinoma cell lines,(Schroeder and Myers, 2008) most cell lines initiated basal FOXP2 transcription from the S1 exon start site. In addition, differential usage of the transcriptional start sites in exons 1 and 1b, which lie in an area of particularly high sequence conservation, was observed among the lymphoma and MM cell lines. Interestingly, only the two HL cell lines and not the MM cell lines initiated transcription from exon 1b. Furthermore, only two of the four MM cell lines utilized the transcriptional start site in exon 1. There was also evidence of multiple FOXP2 transcriptional start sites in two of the MM cell lines, THIEL and RPMI8226 (exons S1 and 1). However, these expressed less FOXP2 mRNA than the JJN3 and NCIH929 MM cell lines, suggesting that this may not necessarily contribute to increased gene dosage as previously proposed.(Schroeder and Myers, 2008, supra)

Regulation of SMAD3 and IRF4 Expression by FOXP2

The transforming growth factor-β (TGF-β) signalling pathway negatively regulates the cellular proliferation and differentiation of normal B lymphocytes, together with their ability to undergo apoptosis (Dong and Blobe, 2006, Blood, 107(12), 4589-4596; Isufi et al., 2007, J Interferon Cytokine Res, 27 (7), 543-552). Alterations in this pathway that result in the resistance of the growth inhibitory affects of TGF-β have been described in many haematological malignancies, including lymphomas and in MM. Downstream of the cell surface receptors, the TGF-β signal is propagated by a small family of SMAD proteins that bind DNA and regulate transcription. Binding of the FOXP2 protein to the SMAD3/MADH3 promoter was identified in both human foetal brain and lung tissues, using chromatin immunoprecipitation (Spiteri et al., 2007, supra). Silencing FOXP2 expression in the JJN3 MM cell line, using RNAi, is shown to up-regulate the expression of Smad3 (see FIG. 8) providing one potential mechanism for increasing the resistance of MM cells to TGF-β signalling.

MM are addicted to an aberrant IRF4 regulatory network that fuses the gene expression programmes of activated B cells and normal plasma cells and inhibition of this pathway is toxic to the tumour cells (Shaffer et al., 2008, Nature, 454(7201), 226-231). Data is also provided demonstrating the repression of IRF-4 expression on silencing FOXP2 expression in the JJN3 cell line (FIG. 9).

### EXAMPLE 2

Immunohistochemical Evaluation of Nuclear FOXP2 Expression in Reactive Bone Marrow Trephines and Trephines from Patients with MGUS and MM Using FOXP2-73A/8

Samples from a series of patients with multiple myeloma or MGUS were double labelled for FOXP2 and CD138 expression to evaluate FOXP2 expression in plasma cells, using the immunohistochemical methods described previously. Cases where there were less than approximately 10% nuclear FOXP2-positive cells were scored as negative. The results are shown in Table 1 below.

| | | | | Percentage |
|---|---|---|---|---|
| | | Number of | Percentage | of strongly |
| | | FOXP2 | of FOXP2- | FOXP2- |
| | Number of | positive | positive | positive |
| Diagnosis | cases | cases | cases | cases |
| MM | 72 | 59 | 82% | 22% |
| MGUS | 9 | 8 | 89% | 33% |
| CD56 negative | 22 | 12 | 55% | |
| MM (within | | | | |
| the 72 MM | | | | |
| patients) | | | | |
| Histologically | 2 | 0 | 0% | 0% |
| LPL or MM | | | | |
| Bone marrow | 9 | 0 | 0% | 0% |
| from patients | | | | |
| with no | | | | |
| histological | | | | |
| evidence of | | | | |
| MM or MGUS | | | | |
| * | | | | |

* includes cases from patients with MPD, reactive thrombocytosis, clear DLBCL staging marrow, reactive HIV+, previous treated MM in remission etc

### EXAMPLE 3

Immunohistochemical Evaluation of Nuclear FOXP2 Expression in Lymphoma Biopsies Using FOXP2-73A/8

Nuclear expression of FOXP2 in several lymphoma samples was investigated using the immunohistochemical methods described previously. The results are shown in Table 2 below.

| | | Number of | |
|---|---|---|---|
| | | cases with | |
| | | nuclear | Percentage of |
| | Number of | FOXP2 | FOXP2- |
| Diagnosis | cases | positivity | positive cases |
| CLL | 9 | 0 | 0% |
| FL | 8 | 1 (high-grade) | 12.5% |
| Mantle cell lymphoma (MCL) | 7 | 0 | 0% |
| DLBCL | 9 | 3 | 33% |
| Burkitt's lymphoma (BL) | 7 | 0 | 0% |
| Classical Hodgkin's lymphoma | 10 | 1 | 10% |
| (cHL) | | | |
| LPHL | 3 | 1 | 33% |
| Peripheral T-cell lymphoma | 6 | 2 | 33% |
| (PTCL) | | | |
| Angioimmunoblastic | 6 | 2 | 33% |
| lymphadenopathy (AILD)-type | | | |
| T-cell lymphoma | | | |
| ALK+ anaplastic large cell | 1 | 1 | 100% |
| lymphoma (ALCL) | | | |
| | Total no. | Total no. | Percentage |
| | cases | FOXP2 | positive |
| | 66 | positive cases | lymphomas |
| | | 11 | 16.7% |

### EXAMPLE 4

Comparison of FOXP2 mRNA and FOXP2 Protein Expression in Whole Bone Marrow Samples

Routinely fixed bone marrow trephines were available for some of the samples analysed for FOXP2 mRNA expression in FIG. 4B. These were investigated for FOXP2 protein expression using single FOXP2-73A/8 labelling (haematoxylin counterstained) and double labelling with FOXP2-73A/8 and CD138 (no haematoxylin).

As shown in Table 3 below, a good correlation can be seen between FOXP2 mRNA and protein expression. Double labelling was more effective at detecting FOXP2 protein expression than single labelling and the results show that mRNA detection of FOXP2 alone is a sensitive method for identifying FOXP2-positive samples. FOXP2 mRNA expression was categorised as high (>0.2 relative to the JJN3 cell line) or low (<0.2).

| | | | FOXP2/ | FOXP2 | |
|---|---|---|---|---|---|
| Case | | FOXP2 | CD138 double | single | |
| number | Diagnosis | mRNA | labelling | labelling | CD56 |
| 1 | MM | High | Weak-moderate | Negative | Positive |
| | | | majority positive | | |
| 2 | MM | High | Positive, strong, | Positive, | Positive |
| | | | majority | strong | |
| 3 | MM | High | Positive, | Positive, | Positive |
| | | | moderate-strong, | moderate- | |
| | | | majority | strong | |
| 4 | MM | High | Positive, strong, | Positive | Positive |
| | | | majority | | |
| 12 | MM-R | Low | Positive cluster, | Scattered | Negative |
| | | | most plasma cells | positive | |
| | | | negative | cells | |
| 22 | MGUS | Negative | Positive clusters, | Negative | Negative |
| | | | most plasma cells | | |
| | | | negative | | |
| 23 | MGUS | Low | Negative | Negative | Negative |
| 27 | SM | Low | Positive, weak, | Negative | Negative |
| | | | <50% plasma cells | | |
| 31 | WM | Negative | Majority negative, | Negative | Negative |
| | | | occasional positive | | |
| 32 | WM | Negative | Negative | Negative | Negative |

### EXAMPLE 5

Foxp2 Expression in Normal Murine Tissues

FOXP2 expression was not detected in haematological cell populations in human tissues. However, a report of high level Foxp2 expression in murine spleen (Shu et al., 2001, J. Biol Chem, 276, 27488-27497) raised the possibility that there might be differences between species, which could become relevant when studying Foxp2 function in murine models.

Foxp2 mRNA expression was investigated in a commercially available panel of murine cDNAs (Clontech) by real-time PCR using a pre-designed Foxp2 Taqman probe (Mm00475030_ml; Applied Biosystems).

The results obtained were similar to those from human tissues, showing particularly low expression in tissues such as testis, spleen, placenta and thymus (FIG. 13). The low Foxp2 expression in murine spleen observed here thus differs from the previously reported data (Shu et al., 2001, supra). The highest Foxp2 expression in adult murine tissues was observed in brain and FOXP2 expression was shown to increase during embryonic development. The slightly more abundant expression of Foxp2 in murine lymph node, when compared to other lymphoid tissues, was also seen in the human tissue panel and is likely to reflect expression by epithelium, endothelium and scattered interfollicular cells.

### EXAMPLE 6

FOXP2 mRNA is Highly Expressed in Osteoblasts

Microarray expression data (GeneAtlas MOE420) for murine FOXP2 was also analysed using the BioGPS website (http://biogps.gnf.org/). Four array probes indicated that FOXP2 was particularly highly expressed in osteoblasts (FIG. 14). Two further probes did not detect the osteoblast expression but did still identify the high level FOXP2 mRNA expression in brain and eye; indicating that FOXP2 splice variants might be differentially expressed.

### EXAMPLE 7

FOXP2 mRNA is Expressed in Primary Human Osteoblasts

Primary human NHOst osteoblasts (obtained from Lonza) were analysed for FOXP2 mRNA expression by real time PCR using the commercial Taqman probe (Applied Biosystems), as previously described. These data identified the expression of FOXP2 mRNA in primary human osteoblasts (FIG. 15) that had been through six passages in culture.

These findings have the potential to be significant in relation to the capacity of multiple myeloma to induce osteolytic bone lesions. Osteoblasts have an established role in new bone formation (Giuliani et al., 2006, Blood, 108, 3992-3996), and in myeloma bone remodelling is unbalanced, with bone reabsorption being increased while bone formation is either decreased or absent. There is considerable interest in identifying therapeutic targets (proposed examples including Runx2 and Wnt pathways) for the treatment of multiple myeloma bone disease that counterbalance the block of osteogenic differentiation in the bone marrow microenvironment induced by multiple myeloma cells (Giuliani et al., 2009, Exp. Hematol., 37 (8), 879-886).

Interestingly the related FOXP3 transcription factor is able to interact with Runx family members, and the interaction with Runx1 is essential for both FOXP3 function and optimal FOXP3 expression (Ono et al., 2007, Nature, 446 (7136), 685-689; Kitoh et al., 2009, Immunity, 31 (4), 609-620). Thus, it is possible that FOXP2 may interact with RUNX2 in myeloma cells. Furthermore, Runx2 is a key osteoblast transcription factor that is also expressed in myeloma plasma cells (as is Runx1), where it regulates osteopontin (OPN) production that is involved in the pathophysiology of myeloma-induced angiogenesis (Colla et al., 2005, Leukemia, 19, 2166-2176).

The finding that two osteoblast genes, RUNX2 and FOXP2, are expressed in myeloma plasma cells indicate that malignant plasma cells aberrantly express multiple transcription factors from this lineage. Thus it is possible that myeloma cells may have adopted enough of the osteoblast gene expression phenotype to reduce the ratio of normal osteoblasts:osteoclasts (perhaps by mimicking osteoblast cross-talk with osteoclasts) thus promoting bone resorption and the release of growth factors that benefit myeloma cell growth. Therefore the expression of osteoblast transcription factors may be functionally associated with the bone disease phenotype observed in myeloma and these may also be expressed in other tumours associated with bone disease either as primary tumours or as bone metastases.

There is an existing literature indicating that RUNX2 has a regulatory role in metastatic tumour and cancer cell interactions with bone, which in part is mediated via SMAD interactions (Pratap et al., 2006, Cancer Metastasis Rev., 25 (4), 589-600). Thus the ability of FOXP2 silencing to regulate SMAD3 expression in myeloma may also affect RUNX2 function. Furthermore, microarray data from the Geo database (murine) demonstrate that at embryonic day 14.5 humeri of Runx2 deficient mice show elevated expression of Foxp2 (FIG. 16) thus demonstrating a developmental association between the expression of these transcription factors. While the two genes exhibit a reciprocal relationship during development this does not preclude their context dependent co-expression in other normal or malignant tissues.

### EXAMPLE 8

FOXP2 is Expressed in the Bone Tumour Ewing's Sarcoma

Ewing's sarcoma (ES) arises in mesenchymal tissue and is the second most common primary malignant bone tumour. It was therefore investigated whether the FOXP2 protein was highly expressed in a panel of ES-derived cell lines by Western blotting (FIG. 17).

Good levels of FOXP2 protein expression were observed in all four ES lines (lanes 2-5 of FIG. 17). FOXP2 was also detectable in the two neuroblastoma cell lines (1 and 8). Interestingly the RH30 cells, which are derived from a bone metastasis of alveolar rhabdomyosarcoma, were also strongly FOXP2-positive.

Initial data identify FOXP2 expression in several types of tumours associated with bone. Further studies will be needed to determine whether the presence and/or level of FOXP2 expression in tumours, such as myeloma, lymphoma, Ewing's sarcoma and bone metastases of other tumours has a relationship with the presence and/or severity of bone disease. FOXP2 expression might represent a marker whose high level expression promotes tumour cell interaction with the bone marrow environment and/or promotes bone loss. Thus FOXP2 expression in some malignancies (e.g. lymphoma where only a proportion of patients are FOXP2-positive) may help to identify patients with tumours that are likely to colonise the bone marrow. Additionally, high-level FOXP2 expression in malignancies that normally home to the bone marrow, such as myeloma, may have an association with the severity of bone disease.

### EXAMPLE 9

FOXP2 Silencing Using Stealth siRNAs (Invitrogen)

Three commercial siRNAs were purchased from Invitrogen for silencing FOXP2 expression in the JJN3 myeloma cells using Amaxa-mediated electroporation. Universal medium GC-content or low GC-content scrambled control siRNAs were used (Invitrogen) as controls in siRNA experiments. A\tilde{m}axa-mediated electroporation of JJN3 was performed under standard conditions, Program X-005, Solution C, 2×10⁶ cells in 100 μl, 1 μM oligo, plated into 3 mls RPMI culture medium.

Samples were taken at 48 and/or 72 hrs for RNA isolation (Trizol method, Invitrogen), cDNA production (Superscript II, Invitrogen), and real-time PCR (TBP as control and human FOXP2 as test, both commercial TaqMan probes). Relative expression, compared with untreated or control-siRNA treated samples, was determined by ddCT method (as per Applied Biosystems).

These experiments show that after 48 hours the #0275 siRNA most effectively silenced FOXP2 expression and that the #0274 siRNA was also reasonably effective (FIG. 18). Less effective silencing was observed with the #2580 siRNA and after 72 hours FOXP2 silencing was less effective with all three siRNAs than at 48 hours.

### EXAMPLE 10

Silencing FOXP2 Expression Reduced Cyclin DI Expression

Foxp2 knockout mice show early post-natal lethality with defects in lung development. These defects are more severe in mice that also lack one Foxp1 allele, indicating that these molecules cooperatively regulate lung development (Shu et al., 2007, Development, 134, 1991-2000). The reduced proliferation in lung epithelial cells was accompanied by a reduction in the expression of cyclin D1 and an increase in p57 expression in the Foxp2−/−; Foxp1+/−mutants. Thus these transcription factors are able to affect the expression of cell cycle regulators.

Cyclin D dysregulation has been proposed to represent an early and unifying pathogenic event in multiple myeloma and recurrent translocations target the cyclin D1 (CCND1) gene (Bergsagel et al., 2005, Blood, 106 (1), 296-303). The FOXP2 48 hour siRNA treated samples from the previous experiment 1 were analysed for CCND1 expression to investigate whether FOXP2 had the ability to upregulate the expression of CCND1 in the myeloma cell line JJN3 (FIG. 19).

The data indicate that silencing FOXP2 expression in the myeloma cell line JJN3 reduced CCND1 expression and that this was mediated most strongly by those siRNAs that most effectively silenced FOXP2 expression. The prediction of FOXP binding sites within the CCND1 promoter suggest that this molecule may be a direct FOXP2 target gene, although CCND1 expression can also affected by the Wnt pathway. Thus FOXP2 expression in MM may contribute to the overexpression of CCND1 in this malignancy.

### EXAMPLE 11

Biological Affects Mediated by FOXP2 Silencing in the MM Cell Line JJN3

To investigate the affects of FOXP2 silencing on MM cell line viability, the total number of cells and the proportion that were viable after FOXP2 silencing was determined. FOXP2 silencing did not affect the viability of the JJN3 cells and the lack of significant change in cell numbers suggests that their proliferation and/or survival in vitro were not affected by the loss of FOXP2 expression (FIG. 20).

### EXAMPLE 12

FOXP2 Silencing Reduced Myeloma Adhesion to Stromal Cells

A key feature of myeloma is that in vivo the cells localise to the bone marrow and interact with stromal cells; this initiates the production of proteins that stimulate or support tumour survival (Dalton, 2003, Cancer Treatment Reviews, 29, 11-19). This is a particularly important aspect of myeloma biology because cell adhesion-mediated drug resistance (CAM-DR) is an intrinsic mechanism of myeloma resistance to chemotherapeutic drugs (Dalton, 2003, supra).

It was therefore investigated whether FOXP2 silencing had an affect on the ability of JJN3 cells to adhere to a stromal cell monolayer. JJN3 cell samples were taken (200,000 cell per condition) 48 hours after siRNA and were plated onto confluent monolayers of adherent OP9-GFP stromal cells. After an overnight incubation, to enable cell:cell interactions to take place, the co-cultures were washed vigorously twice with fresh medium and then the medium was replaced. In the second experiment non-attached cells were also retained for counting. Photographs of multiple fields were then taken. Co-cultures were then vigorously resuspended with p1000 Gilson pipette and cells were passed through a 40 μM cell strainer to separate myeloma cells (<40 μM) and stromal sheets (>40 μM). The myeloma cells that passed through the cell strainer were then counted to quantify the number of adherent cells.

FOXP2 silencing reduced the number of myeloma cells adhering to the stromal cell monolayer (FIGS. 21 and 22). Cells transfected with those siRNAs with the greatest ability to silence FOXP2 expression were the least able to adhere to the stromal monolayer. The data therefore suggest that FOXP2 has a role in mediating the ability of myeloma cells to bind to cells in their microenvironment. Thus targeting FOXP2 expression and/or function may be able to overcome cell adhesion-mediated drug resistance.

1. A method for detecting abnormal lymphocytes said method comprising
detecting an amount or expression of the FOXP2 gene in lymphocytes in a sample
wherein an increased amount or expression of the FOXP2 gene in said lymphocytes indicates the presence of abnormal lymphocytes.

2. The method of claim 1 wherein said abnormal lymphocytes are abnormal plasma cells.

3. The method of claim 1 wherein said abnormal lymphocytes are malignant or pre-malignant.

4. The method of claim 1 wherein said detecting step comprises determining the number of copies of the FOXP2 gene, detecting FOXP2 mRNA and/or FOXP2 protein and/or detecting a mutation or chromosomal translocation which results in FOXP2 gene expression.

5. The method of claim 1 wherein said method comprises determining the amount or level of expression of the FOXP2 gene and comparing said amount or level of expression with the amount or level of expression of the FOXP2 gene in a normal lymphocyte sample.

6. The method of claim 1, wherein detecting further comprises diagnosing, prognosing or monitoring of a condition associated with abnormal lymphocytes or its treatment.

7. The method of claim 1, wherein an increased amount or expression of the FOXP2 gene indicates or suggests the presence or status of a condition associated with the presence of abnormal lymphocytes.

8. (canceled)

9. The method of claim 7 or g wherein said condition is a plasma cell disorder or a lymphoma.

10. The method of claim 9 wherein said plasma cell disorder is myeloma or monocolonal gammopathy of undetermined significance (MGUS).

11. (canceled)

12. (canceled)

13. The method of claim 20 wherein said condition is a plasma cell disorder or is lymphoma.

14. The method of claim 13 wherein said plasma cell disorder is myeloma or MGUS.

15. The method of claim 20 wherein said agent is an antisense sequence, siRNA, a FOXP2 binding protein, small molecule inhibitor, FOXP2 consensus DNA target sequence or an antibody.

16. The method of claim 15 wherein said antibody is an antibody which binds the N-terminus of FOXP2.

17. The method of claim 16 wherein said antibody is FOXP2-73A/8 produced by the hybridoma cell line of ECACC deposit Accession No. 08101410 or an antibody being a derivative of FOXP2-73A/8 or having the identifying characteristics of FOXP2-73A/8.

18. The method of claim 20 further comprising administering a therapeutic agent effective against or used in the treatment of a condition associated with abnormal lymphocytes, as a combined preparation for simultaneous, separate or sequential use in treating a condition associated with abnormal lymphocytes.

19. The composition of claim 18 wherein said therapeutic agent is a chemotherapeutic agent.

20. A method of treating a condition associated with the presence of abnormal lymphocytes in a subject suffering therefrom and/or for reducing the severity of bone disease associated with said condition, comprising administering to said subject an agent which inhibits FOXP2 expression and/or FOXP2 activity.

21. (canceled)

22. An antibody that specifically binds the N-terminus of FOXP2.

23. The antibody of claim 22 wherein said antibody i) does not bind FOXP1, FOXP3 or FOXP4 and ii) binds FOXP2 in its native form.

24. The antibody of claim 22 wherein said antibody is FOXP2-73A/8 produced by the hybridoma cell line of ECACC deposit Accession No. 08101410.

25. A hybridoma being that of ECACC deposit Accession No. 08101410.

26. (canceled)

27. A pharmaceutical composition comprising the antibody of claim 22 and a pharmaceutically acceptable carrier.


SEQUENCE LISTING (45 sequences)

SEQ ID NO: 1 - DNA, 276241 bp, Homo sapiens
cacacactcacacactcacacacatgcacacacacacatacacacacacaaaaatgaagcacttactttagaaagattatggtaagcatgctggctcagtcttgaacctttgtcacccctcacgttgcacaccaaagacataccctagtgattaaatgctgattttgtgtacgattgtccacggacgccaaaacaatcacagagctgcttgatttgttttaattaccagcacaaaatgccatcagtctgggacgtgatcgggcagaggtgtactcacagtagtgtaaatactgctgtaaatagttgtctgatggtggctttgacagtgagctagcttctgagttttcccttctttttatactgttttctgtgctggcttttttgaatcttcctaatttttcatctctttaacaaactcctatgaagttgaaaccgggaagtttgctctaacatttccagagaaggtacgttactttttgctaagagaatatctgtaagagtttagagaggggtgggattttacgattgctttactggtagatttggcaattgctttacttttttttttttctgagactgtagctcattgaaatgaactagagcattgtatctgtttgcgagggaaaagccgggagaccagacacaggaactgtttttgatctgtcagtagcgtaatgtagatttaagctatcactagtaaagacagcaaataaagaaggcctcttttgtattaaaaaaaaatctgcaagatcttgagaagaataaagacagggttcctgctttcatggtttgataataaccatgtcatcttgcttttagtaaatgccccagtatgtgtcagggcgcaagtttttgaaaagggagtttgaccacacgtttgggtgcctctctgtgccgtgctgtgtagagcagatgtttattttgtgctaaaggggaaactctttttgctaagcttgtacaaacatgaaaggagtcagggctcagatgatctttgtagttttatatgcatgttcatgattttattaaaaattggttggtgggtctttttcaaatcactgattgtgaagaaatatatatatttccaggaatacacattgtgaattgatgagtaatttttagaaactgcattatatttcttcaaataggtgtgatagtttaatactcgtgtcagttaaaaaggtagcttggctgggctataaattaccatttctgcacttttcattctgtctgcttcaaaaaagcgtctcagaacctgggatcctattgggcccatcatagggctacagtaattgcctgatgacaactgaaaatgatagaataattgtcaagagagacatttttaatgggcaaggtgatttaagtatgcatgaacaagctatgagggaagatgagcttgtttatgatgcaagccagtataaacacagtcggaggatgacacagatagctgcacttttcccagtgacatgagcaagttcttactgcaacaccaacagctttttcctctgaccagtttgacattcttcaccttcaggtaataacgcctaaatttcatccatgtattttcactttactgtagcctaagtgacaggtttttgtttactagcctccgcgaccaaaaaaaaaaaaaaaaaaagcactctatttaatgtgtgcttagaatagatcttgaatcactttcaccgtcaatagtgtgtgtaatacaaaactttcattcttattttcgattgttccttatctttgaactgttctgactttaaatctgattatctatagttgattcataatgcccttttaaaggcatatttatttgtctttacagtcagcccttctgctgtacaagtttggaaacaaagtatacttgagaatatgtggagaggtttaatgattagcctgttggttttcacggaaagcagagtcctatacctaacagagattacggtattgaagggttacaggtatctgtttagaaatatggtgctctattagaaaacaacttgtttgacttttttttttaaacccatgtaaaatttcacagaatatgctcacgttttattttaaagatcactaaaattatatgcattgaaatactaataatctttgtaaaaggtggatagataatttgtttactttattattcatgaaggcaagtaaggcatggtattctgctattgtgggcatattattaacatttcatagggatttgtgctggaatgtggaatgctgcttcttcataatttaatactcctatgcataatgaggtttgtgattagttgatagagagaattggcccaactccattctgaaagcagataatttacattgttctctagagtctagaatctaataggttctttagtgcaataaaattaaatgctacatgttttaaatttcagcatacaaatccccctggcaattttctgtttttaatttttgccttttgtttcctctaaaacaatgaattttaaaaattgtttctagaacatatatttacctagctcttttatttagtatgtacaagtcaattagcactgttcattagcagaattgggtatttgttttaaagtttaaccaatcactttgaaatgctaattatgatgtaatttaattgcaagtcctgtaatgatgatgctgatattatcgacataaatgatgcagttaagcagtggaatctcatttgcatatgcttaaagcaagttgaattgggctgtttcaatatcactttgctttaattttccacctctgttcacaccagccctttgggtttttaaagctgtggtttcctattgatgatcagtgctttcatctttattttaatgacaaaggggcaacaactgacactggaactgtaaaaatgaaaagcaaataattacttctattaaatatacgaactatgaaaccttttttggtaatgatcattctttagaaactgacaaatgcatgcatttttggtattttcttatagcattcttacacttggaaatgatactatgaaagaaatacacgaactgatatacagtaaagtgctatgccccaagatgtttctactttgtgaaaagtactttattgcatcttaaaatgttgtagaaagtagacattgcaatacatttttcaaaaactttctgtgcattcagatttttgtgagggtatattcctttaaaaggaacaacaaggcgttatactcatgattgagctgatattaaactccagtggttatctgtttttgtagaggtttggacttgggtaattttcttctggaaagattatttttcttctgaccaatttctcctgttatgtcctaattggttacataaatcttgtctggtatgaatgagaaatgtttctgtgttgtcaagtgtaatcttcacccttatttaccaattcattaagatctattgatgcaggactggtgagagggaatgacaacataaatcagccctccaacataatgaccctaatcagctagaaataactggaaacgtcatgatgagctatgtctctgtattacttcggcaagattctgtggctagcgaggtttgtgtctgcttcgatcattaactttacatccactcaccttcttctaaagaagttgtagtgactcttgttagtattacaaagttctttttctcagacatttgaatctttaggaaccatagaagaaaaaaagaaagagaaaaggaaactcacagaacagacgtcatgaaggctaattaaagcagaaaagaaacaaagctattgtagtgttttaaagaactttctggtgatgattgaagctgtgggttcaaaaaaaaaagctatacagaaggtcaatggcttttttttatgagaacctgtcgatcgcaaaggattcaggcgtatatctttcatgtttagttaaactgaggcaaatgttttaagtatacttataagaatgatgtggctatttcaataaacccaggtgcctccaatctgccttctgattcttctgtaagaactcataatgactttcttctaaattggaataacagtgtgtttcctagagctctaactgaaagactagtaattaaaataaaactgacaaatgataatgtaaaatttttctgtctaaatgcattaaattgtaatgctaaacacacactcttctcagttgcttttatcaactggctatatttttcctgcattttgcctgtattattaaatgctaagttacagaatacatctatgctaactgactgtgcatataaaggcaattgttataggtatatgtatgtcaagtatatgcatgtatatacatatgtagtatgtcaaattttgataatctatgtgtattaaatatatactttatgacatactgttattggcattttctattgtttggcagcaatagtgaattcatccaaaataggtggatttcattttacataggacaagaaatacttgcaaacagcaagtttaggtttgaaatttgacggaaaggtttatgccatgttttgatattctgatgtgtgctaacatttaaaacagtttaaaatagttacatgttgtgtgttaatacaggtgtttttggagtaggtctgcttcatagtcacttgttttatacacacttgatgccagagtataaattaccctacatccctcacagttatttatccttatagcatacttgctatttagatcagaccatggtatgagtgtgactcatgctaacattagtggaagctaagtataaaaatgtttattctaatgcctaaaataaagttcaatctcaagaataacgtataaactattttgaaactatgccgtcaaaaaacctctgtgacaggtcgcttacatacacatagacacacacacacacacacacacacacacacacaccccagaaaggacaaaagcagtttcctgaccagattctaaaactccattcaacacaatcatgtggttacattaaaattctggccttaccattgcatccacctgctaggctcatgttggacttttgcactaagttgagaccggtggaagaaatttctctaagcgtaccgcctatgctgtgggtcttccaatgggaaggcaagaggtgggtctgctgaggagcagtattaaagggacatttggcacccaaaagatagcctggaagtaaaaagtgctgaaagggcaagagaatgaggatccgtggaagagctgtattatgctcaggaaaaacagcaagggatcccagggagttgctgtgggcgggagactgaagattgaagtaaagtaagcctgagggttaaggaaagatatgtggcaggaaaggtgaaaatgccatcacaagagaagtacatactcagagctctcattagccaggggatcagtgaaggctattgcaagtagtccagcctttgggaatttgactgtaagtggacccactgcatggaagcttgcaagcttgcacaactcaatcactaagctttttaaataaaaaagagggaggttttgcctgctaggcaggtctttctatgttcttggagagctaattatttcttattgccttacatttttactgttaaattctgtttcctaaacgaaaatattcctggttgcaggatttgacaatttttcttcttcaggcaaagacaagaaaatctgtgacagaatatacttgcttacctggtgtcttgtaaactgtagccagtatttatttttgtattggcaactaggtaacattctttagtttttaagtcctcctcttacatatctaagacatacagttagataggataacacattgcaaaaaatattacattttggcattataaattaatgaaaactcaatttgtcagaggttataaagtagcagggtgaaataaactttccctaagcttcctatcccccttgttttgaaaaaataaatattaatgctataaaggattgattttaaaacatttaaatgatttaatgaattaaactaacattaaaatttatatatttaaaaggaagactgacaatttagcatgtaccttgaaaagtaaaaattggtgtacatttttacatgtttgagaaataaatatatacagaaattatatgaatgtgaagacaagatgttagaatatgaagattaatgtgaaaatataattttgaaatatatttttgagtaattagttaaccagaattatcaatatagtattataaaatctctttcaattatacttctgaagggggcttctgaactatagatacagaatcataatgttgcagtaaaatacatctacatggccaccattctttcaaattcttcattgcactttaagtgaaatagcatagggattaaatacttaattttagtattttataatattttcataattaaatatttatgttttgtttccattttaaacttattttattttttatccaggagcaaaatataatatacttagttgctaagttaattaatagctgattgaaatggaagataagtttttcattggctttacttatgtttcctagattttgttattgttgaagatctaacagtattatctttgctaaaatgtagtcattatcatttaaaaaatattttgttaaaatgtccttctactttgcatgtttgaaatattaacctttttgaatgaaagtctgaatttttactaaagtgtttttttttctaatttgtgtatagcattttaggcattttgtttaaataagtaaaaagtagtaacaaatacttaataaacaagttaattttaatttacttaaaattatgtcacttggtatagtaaaaatatcacttgctataacagataaccttccccaaatcatgttttaaaagacatcctgcttcagtttttaaaaattgaaaatattcatgtgctgtttcaatttttgttcttacacattcaactatatccttcatatttggcattttgatttattaggcaatcttagcattaaagacttttagcaaagcagtgctggatctcaaatgcactatgcatggtgcctatgatgaattaattccactagagcacaagagttatcattatttaaacgctagtttctttcagaagttttaaatcacagtcaagttttcaaaattagaacttaactaatattttgaggtaaacgtatgaggtttcttgttacatatgttaagttttcttaatatttaactctttcttgtgtgaatctgcatagaaatatttaatatttgatattgataattgatcaaagattaaggaggtatttaagaatatatgttttaaatttgatgccttacttttcttaaaatgtcatattcaaaactatttggatttaatgcatatgtttgattaaataaattggacactggaatgaatcagatattagacataaagtgtctttcaaggtcactgtaatcattttgtttaagaaaagttgacgaaatgaattgttagaaatgttttatatgtactgttatttgttcttagttttctgggaagtcatttttttttcatccatttgttttgattaaatagtaagactcctccaccaaaatgagagttaatattaaggcactaagattataagacagactgtttatgtagaatgcagagtgtggttcaaagtagaggctatttttatcttctttataatccctgacatacatagacagattgattgatctatctttataaacatatatagatacacatattcatgtgcacacatatacacatgggaatgtggagtggaaaagagatagggagcataagaaacagggaaggagaagagatgagaaagaagaccatagagaaagttgagtaagcaacaaggaccagtagtgtccctggtggtggctgagataacacaattaactatgcttgtccaggtcaagtttcaaacttttaaacattgctgcgaatacggaataggatacatgcaggtttctgttatcactctggcatgtaactatctcacatgatacagttttataccaatagaacatataactctacgaggaagaaataaaaccaacaagtctagcattcagtactaccctatcataaagtaaggaggcctaattgagctatgtctttatatcctgctatttgtacaaataaaagactacatcttgatgcaaaataaacacaaaagcactgttgtgactataacagggagtcatatactaatttatataataagtggttgtgcacgctttattcaagattatggctgcaagaaatggcatgaattgctgggtgtgtttaatgctggattatagcaatttgaaacatgtcaaacacatggctaatgcctcatgcttcactcattcttgcgtaaacctgcatagccaccatccattatacccttggtaacaacacaattctgcaagtaaatattgttacactaagtaacctttcaattcaagttgtttaggaagagaaaatgcaatcttgtgcagtcagttccagtgtcagtttaatgtttcaaaagggaagtggtgtagagaataagtatctgtacagtgacttgcttaaaaaagaagaaagaaaaaggaaaggaaggaaaacttaatgcaggggagaaaagtctaactgtatcgtttgagtgtaagatctgaagagttattttctgatgtggctgaaaagttgtgtacgatgaaagacttaagggtttaacttacagttcagacaaacattaaaacagggactcccagattcatctttccctttaatgatctggaatttattttcactgatttttgtaaaatgtttgtggtgggattgataagaatgaataatttagcttggtatgtttcggatcataagcacatccactctgaaagaatatgctgaatttattattatttaaaatatacttaaaaggtaaacagcacttcacattgtgctggaaatatttcctatattgggaaataagtggaagtgttaccaagagaaacttagtaaatgctagaaataatctgcattgtccttgaaaaattcctcctttatagtgaacatactccaagcttttttaagtgaaatgtacagtataatcatctaattacacaatcacccttcttttttcatataagtgttgtatcagatactcctcttttgtagagcaaaaagtactatcactgttatagttttcttaagcattattttactaactcttagatgattgaacttaaaatgtaaataacagtttatactgtagggttttataaagttaaaaactccacaagggctaagaataaaatcctttagttaaaaggaattataagttatttaggtctttcttcgtaattgtaataaagatttagacacatgcctctttgcttgttgattattcttaattatttgatggccttgattatgacacagtaatacatgtagaattctttaattcatccaattggtcctgagatttactacacaaaggttagtagtaaatgctgcacctgtctgctacactggaaaatatagtgttttatttttattccttgtgttgctaactccatccagaaacaataaatatttcaaataacttaggtgatgaaataaaaacatacacatatatacacatattccagacatgttctcaaaagtaaaatttaaatgcaatatgtaaaagttatgcctatctgtttctacagttggagaggattgaaattataattttcatataactagtaaaggcaatcagaccttttctctgaatattttaaacattgtgcatgtaaaatttatgcctaatttgaatacttattagctgcctaatctcagaaaagagtattagctttgtttttttcttataaaatccatcactatgtatcagttttaatttatatgattttttctttgttaatgttcaataagatgtttagcaaaacctatttgtttataaacacatttcgctcaaaaattgtttactatagttacagcaattatttcctttctgctcatgttttctgttgtttttttattatccaaacagctttaagttgtatatgtaatttttgttacactaacttgctttgctagtccagctattgacgttttttactaaatcagtatgtctttttttttaatggacttgataagagtaggaaaagtaatttaataatttaggctttgatgaaaatatctgttaaaactatgatactataaacattaattttaacaattttaatattggcattcaaaaatgtcttttgggtatgtctgtgattctttttgtatttagtgtttgtgtgcatcgcattttaaaagataactggggaaaacattttctccatgacaggaagagataggaagcttgcatttgctttataataaatgcctttcaaaattttaaatggacaaagcattaaaatatgatcaagtttaaaatattgaaatcatgtaaaaaacaaattagattgagtgataagtttactgtcctttagggtaagtccactgtctggtattactgttaaagtcacagcacagccaggttgtttctgcaattctgtccatagaagtaaaacagagaagtgcatccattaggttatggcacaggattcagaggtctttgagagccaaaaatgatctttctaaaccacagattcaggatgtactgtgaaattctggtttgtaagagcacattggttctttatttactgcaaggatttccactgatcttggcaggacatctcattgctatcctgaaagtacagtatatttatgcagtgaaaatgataaaacatttattactgtagagaaggtaatatgcataatttatgctgtttttagcacaatcattagtgatattcttgatagattttatttccagctttcatatcttatacatgcctggaaaattaattttatatctttcatttatttgcctatgttaaaattgagttttaagtcatcttcaagtgaacagtttgattgctgctcatgcataagcttaattacttcccaggaaggacagtattaaatgttttaaatgtcagaaaaataaatgaatatgagctgtttgattaaaaaaataggcaatatctgacgtgtttgcagcaggagctttttcttaaaatgcctccaaggcaaaattttatttagtcacaaaaaaagaaacccattatactatttatcattggttggtaccatatgatttgtaacacccttacaaaattttaattttgtatgattagctgtgcatcattaaacaacagccttgcataagatgtgctgtaaaattctgacagaatcaagatagtgaatatttaaaataaggctgtatagtcatccatggttgtcaaaggtagataataggaaacagaacagagtgtgcaatcatgccttcatttaaactggttttataggtagattttaaaatgtggtcattcataaatgctattgaccatgctatatgcatggtatgttactttggctgttaaatgtaaccgcacaaaaattgcttatagaaaccataatttcaatgattagcatctggatgaaagctgaatttgtattgctttccttggtaaatgactattcaaggcttttttcttccccctcttctaaagatgctgtctctgtaattggcagagagggacatcttgataatggaagtgtgaaggtgtaacgtgtgttaattgatacttcttaatcacttttaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggtaagttttgttttcctcagtgcttccttaaaacaaataagcttgttttattgaatatgaaaaatgtattcatagcaaactgagcatgttgtgttaagctaaaagatgctgagaatttattattatttggaacatgattgaaggatgagtaaagagataagattttcatattagttacgtcataccagtaccagagtacccttcaggtgtcaggatattaattaagctcacaggatgatttttggaaggtctgtaaagctttgtaacaaatattttatagcttctagtttactatcttacattgtgctgcagaacgtgatacttagttttccaccagagggccacattacttgatacttgcttatacaatgaatttcttcaagtctgagctaaggtgaaaaggaccacaaggacttttcaaaagacagatgcttgcctgcgttagctgcaaatattagctattaattagttgaaatctgctgagagattgtaaatcatgtaaacactacgatctgttgcttttcttgaaattcattccaagatattttgaatatacatctttcaataacattaaagagcactttttgtgttctcctaagtgtctcataaaatgcaatattctctttaaatctggaaatgttaacaactacaaaataaaggtacacttttaagtagtctgttattcattttataagagattactttttcctgattttgtaaaaaaaaaaaaaaattcttttcaaccaaaaaatagccacttcattcttcactgactcaagtctgtagaaacagtgacagagaaaagaaagctttgaaagcatatacttatattaggccagaatgagtacacttgtttgaaaagtttatatttgttttaaaataaatattatttctttgtaactctgaattgcacttaaactaaatgcctaaaaatcattaaaattacatccattaaaataatgattattgaaaagattgcaaattatgtgaaataattcacattttaaggtagtatataatgagaaaaatacatctcaaaagttaaagcaaatgcccacactagagtaaaatttaatagatacattattttaaacaataacagcattaaaaaagttacatgggttgaataatgtcttaacctttaggcattgttttcaagaataatcttttataagagctacatagtacttttgaatggtttatctattattttacaattttcatttttttcacagacaataagtattagaaaaacaatttatattttgttttaacaatcataggaattatatgcctaccacagtcccaggattagcttattttactggttactttaataaatacagtttttcaggatagcccttttctgcctttggtgtattcgcaggaaatatcacaccagtggaatgctacaatattcctcctaatttccctaagtgagagtcctaagtatttttagccagattcaattttgcctgcaaatccagtggattgcgtttataaagccaagtaaaaatagtaattgtaaatgaacatcgcagttgagtatcagtagatgataggttatgaaaaaatgccaagaaatctttctttattttcatagctccaattggatgttaaataaacagaaaatgagtaaaatcatagctatagattttagtactctggaggcattttgattaggtctataaggtgtgtttataacctgagtttgaagtttactttgttctatgcagttcattatgtaaaccataaacaagagtaaacacaaaagttatgcccttcagatgactgagaaacttcagtcatgtattgtatgtctgtagagtgccttctaaaagtgatttaacacaatgggtatggtgggtgcctacatagctactttaggaagtgtctacagaaatatatgtaataatttaataaataaacatgcaaaaacctttgtaaatatcccttcccttggttatgttttaactatattaatatagttaggtacctaaggttactgaattttttgttctttcatgaatgtattttagaatggggctttaagttcattaaggattaacatatcttttctataaaattgatgcagttattgcctttaattctaacattcgctatatttcaaaactaaaaaccataaagcatatttattaaaacaaatttcacaagaagtgaaaaagtgaaataattgtataaatttatctctacgtgttgttatttagatgtaaagttttacacaaagagttaaattacttcttataattataatgagctaatcattgttgaccaagatcttttgtttagaaatatgatttaagaggcagtgatgtaaacaaagtaaaattttgtttgccgtgaacttctggcaaaaatggtctgttttgggaacataatatttgtcttaatttttatggcttttgtactttatagaggtgagtagtttgacttaaattagccatgtatttagcatgaagagttggtgtttggaatgaatcgcatcaaatacttaatgatacgtggataggtgaagtgaaaaaggattgataaactgtaactagcatttatttctaccatttaattaatgaatattggaatatttctgaaagctaggatatctttaaattttaaagtttgtgatataatggtagccacatctctgttagtatgctctgctatcttatatttactgaacaattagtttaaaaaaaagccttttctggatgaatttctctctccttcattcctttctaatcttcaaaaattagtactaagtagtagaatgtataatatatggacgtagcatatttcagatgttgttttagattaactacattgagaaattattctgcattagatgtagaattttattttgatattaggtttgcagaattaattgtttgtgtcactaaggtcaaaatagttgctaaaacttgattgctttacagggtggatttgagcttgcattttgagcattcattaagtcagataatctttctgaatgttttatgagactgtactgctttgaaggtgtttcaggtgatggtgtttaaaaacatctggttagtagtttcttactgtcagttcatttatccttattcatgtaaaacagagtatttgaataaccatttaaaagctagaatccacacattttattttcaaattgaaattgagaatagcgacatttgtgtcttaaaagcatatgacatttaataaataacaccattatctgtactttttataattttattttgaaatctttaaccctgataatttgatcaaggaagtgcaaaatcttttgaaagactgtttatgtgtaaggaaataaatagccatgctaactccagcatttaagtccagctggtctcattctatattttcatcttctgtttctctgtccttattgaagttttatgttgacatatgagtaaataatagtttataacagccacgataacctctcctatacacatttttaaaatgcataatttatttgcttcccccttaacttattttaacttcctatttgatacgaaaagtaccgactttagattaaattgtttaagcaaaagttttaaatgaatttacaaagtataatgaaattacaaacgcacttttcaaatgaaataccaagaatgaaaatatgtatcaaaatattttcttttagaaattaatatttaaagtcttaccaagtgcaccaaaaagagaatatatcttgaacaatttattctcattgattgattataaattattaaactgtcacataggcaagattttagacattgtttgactagatgatatgtcagttattacatacatatttcagaaggcactggtaaatagtggcaagtttacaacgatataggaaaacttaacactcacatcttcacacatgaccttttatcacactacaatattaattgatgatattttccttttttacaaccaaattcctgtctttggcatattggatttattgttcattgtactgttgttcagaaaattacaattcctgtgaagctgatctgtactattttgaaccagctgtgaacatgtgctcagtctttttgataacctagaaagaatgtttctctgatgccaatatttaattttctcacataaacttatactcgcttaatctcaattgacaagcatctaccaaaatactaggtgtttctaggttaagcacacataagatatttacttcaatactcatgtaaaagatccacaacaaaatggtaataagcatttatgtaatttgtctcatgaattgggtatactctcgtttctattaacacttttttcatagaaatggattttttaatttataaaaaaggatttaaatctatttactatgcagtgttgtgtatctacagtgcattcttaagttccattcactataggtacaagtggtagcatattcattgtgcctgtcttgcatgaaagcatttactgaagaaatatgacaagaacatgttcagctaatgaggtcgaatggttgttacattaaatttaggcggtgttttctattaggttttatttatctgtatttgcattcaatcctttcacatttgttatctctcgcccaattccagggatggggcgccttttctcatttcacagccaattgttactgtgcgctgactctgtaagttgctggtgaatgtaaaagtgactgcaattaacctgactttgttgcttgaggagaatttgagatgagaaatctgacagcaaatgacaggcatttttctgggtttagcacagaagaaagtctaagtaacattgcaaagagaggcattagggagtaggtacctgcaaattaagtggctgacactgtctattgagaatttagatagtaacggctggaatttttatgttaagttcacattattttatattcatagcaagcttccttttgagcatttcctttgtgactgtgcatgatctcatttattgtaatttgtattttttggttgatcagaagatggctgtaattgtaatttttattaaagcacattcttaacatgtaagaatgcatatatttagatgaaaacaattttatttttgagaatggattatgtgaacttagattttaatatgataacagcctacccctaattctcagagatataatgtgaaatggtggaaaactttgagcaataatactcatggcaggtgagtattttttttaaataatacagttcaatatcagtatacttatcttgtcatttatttctgttgaactgaaggaaagaagtctgactgtacagtcataattctgtcttgcattaactggctagctatgcttttagcaatagttatttttactttccaaagcctgttggttatgtagtttgaattacaatctttgccttcaaaagcacactctatacaactacctcctgctagtagatagtacccacacagtatgcaacatttattgtatttgtcaggtgtttaagaggccattgggagttttaattaatgtcagttaggaagatacattgttcagttggaagcagcttgccaagttcttcattttcctataatccatatagtctactgtctttagggaaatatgcaaaattaaaagttacactgcatgctgttgtctttacaacattcagctcatcaagcaaaactctcatgcaaattttaatttgcccttgagcataatttagaaaccataatcattgaggttttacacagcacagataggaaaaggagattggattaatttacactctcttattcaaatgacctcattacttttatggtacaaactaaggttttgcagctgactgtataggtaaagtactgttaagggtttaaaaagtaataattttctagaatttatctttaaattacattttttaatatttggttaaagacatttctttggagatctttaaatatcctaaagtttatgtattgatttagaaatgggaaaatgtggcatctatttacatttatcaagtttaactattaggtcttggggccttctatttacttgaaggatcttttcatgaattctgtttcagagttaactttttactgttttcagtaatccctaaagaagtgtacacttggcctgacatatagagttgtcgtctaccacatgatgacactaaagatatatgatatgactgacagtgtagttgatgtgcgttgtcttccctagacaaaacagatggtattgttctattatcatccacagttattttaatgtactgtgtccatctgtgttaagatatattacatggtttcatggaatttacagttcactcctgcagtaactcgatttcactgtttggtctctagagagtttgcaaaatgtcaagctgtagcttgaaaaagaaaagtgaaatctactggaaactaatacttatgaccatttcatgcctgatctgctttgtggtgttgtttagaagctagggtgacgtttataatacaacaggtaaaagaaaagattgtttaatgcatatgaaaaattgtaactgctcttattgagcatttgtgtcctagtccatttaaatccattcagttaataaaagtatatctggttcatgttagagactgaaatgtttacattgttctggtgttctattagttcagaaattaaaatgtgtcttttaatttcatatcaaataaataaaatctaaattaaattcttttacaaatacttacctgccaaaaatctaacacttgccatagtcttttccaaaggaatattttttctcaaaattagttgaaaaaaaatatttaggctgtttctccaaagttgtatctcagaaatattgttaatattctgaatatttccatttctcaaatataccatcatttaaaatattgattcacttctacagatggccaaattttgtattagtttttatttatcgtagtcttggaaaatgttatgaagatgcaagtgtacttattctctaggtataagtgtgccttaaaatattctcattgcaaaatttcaactaaagtagctaacattaaaactactgtcaaaaatacatatgtgctttgactatggatatatgtatatatgagatccaagtcagttatttgacaactaaaagcaaatagatttcatagtattaaaactatttgattaaaatttaagtgatgattcctagttaaaagactggcttacatgaatattagtcattaattagaaaatgattcagaaaatagtgatctggagctaaagcatatgctactaattttctaaattcatctggcaaaagaggagttaatgtaaaaaaatataaagtaataggcatgatatggctgcaatatattgaaaaaaaagtccttggatctttgtgttgaaatggctcataagagaccacaaatatactcatgttattactcaactttgattcagggaatgaaccattatcatattcaggcagttgtattctttctctagtttctctttattcactctcaacacttcaatgttatctagtagatgtacttgctaaataattgtttttgaatagctttccaggaaatcaaaatttagctgattgtcattttgagttcaaaatactaaaagaaaatgtgttgctttataaagaaaaataaataaagaggtgaataattgagattagtgagaaatccttattttggttctagttaacataatgcctaactcctaaatttagattgttttttgggctctcattttcaatagtccattcataattttagtatatttctgacctcacggtttaatatatatgggggggggggataaggtacagcaatttggttgatgtattatatgtataaataattaatagtaaatttaatatttttaaagaaaaacacatggagaaacaaggaatagatatataaatacaattattgaaagaactttgagggtaggagtagtaaagaaaatttacgctaaaaatctgaccaaatactgttgtttttctaattctccttttcttatagtcgagcagcctgtttcgttaagaaaaaaattaggaggtataaaacagcttttcagtaaaaagctgtattcaactggtactgtcaaattaatactttccataaatatatattttctgtattacatgttttattaaaatttcctggaatgtttttctgatgtatgtgcataatccttggaaattaattgtaattttattgatgataaaatacaaaggccattctttccaattaacatatacattttaagaaaaatctttcgtctgtctcttaaaatttcacatggctcatggagaacccaaccatcctccttgttatagcatatcccaaaatgtcccaccttattgcaaaactctgctcaataactgaatcagctctctcataatgcttccataacggaagttgccttaacttcaattgtgaatgtggaatttggaaattcatatctgcttattcattaggttcaacaactataaaccaatcacttttcactatgattgaaaataaatggttgttgaaagcatttccccagccttcagtacagaagatagtcatgccatgatagaggtagatgtgctttgaggccttgaagaggagggacagactcagtctaaattgtcaagaaaggttttaggaagggcattttaattgttcatgaagagagaggatcaatcggcagaaaaagggtagaaagaattctaggtagaaggaaaagggtacacaaagacaggaaggtgtaaaaagtgcagaatattgaggaccattcagtgtttggatgcacttcatacaaagactattgggagagtaactgggagatgaggttggaataggtaggctagatatagatcctggaggatcttgtttgttgtgctcagaagttagtttgttgtttgtttgttttgagatggagtattgctcttgttgcccaagttggagtgcaatggcgtgatcttggctcactgcaacctctgcctcccaggttcaagagatcctcctgcctcagcctcccgaatggctgggattacaggcacgtaccaccatacccggctaattttttgtatctttagtagagacggggtttcaccatgttggccaggcttgtctcgagctcctgacctcaggtgattcgcccaccttggcctcccaaagttccgggattacaggcgtgagccattgtgcccggctgtcagaagttttgactttcattctgcaggtgttagatagccgttaaagattttttttagtggaaaatgatggcagatttatatttttgaaatacaaatttaccatagtttggcctaggtagaggctgtgaagaaaaagaaaaggagaaattaattcaaggtgcaaactcaataatacgtgtatttcatgtactgtgaagcattgggaaaaatttaaaaaagcaaggcttgggttttgaaaggtgattaaaagaatcaaacttcttgagttcaatactaattattggctatgtagtcttgggcaaatcactgaacccaggattcattttctttatgaattggaggtaaaaataattaatataattattaatttattttctttcttgaccaccccctctactgctatcactcttcgtcttttgtatccctgagagtaacatttatatctcctttgaagcatctgtcacagttacaatttcatatttatgtatgtgattatttcatgtgtattccctgctagattggtgtctattccccgctagacagtaagttttatgagggcaagggccatatcagtttttgctcagtattttatttgctgtctagcaaagtgcctaaataaaaataattaatgtaattattttaggagtaaaattattgatataaaattcttagcaaatttattaatttactaaataagtatttatttaatacctactgtgtattaggcactttgctagacagcaaataaaataccgagcaaaaactgacatggcccttgccttcataaaacttaactgtctagcaggaaatggacaccaatctagcagggaatagacagcaaataatcacatacataaatatgaaattgtaactgtgacagatgcttcaaaggatatataaacgttactttgagagatacaacagaggaagagtgatggtagtagagggggtggtcaaaaaaggctggaggatttgaagaatgagtagaagttagctagatcaaagtaaaagtagagagggcatttccttactctggcttataataaacatatacctaaatggtaccagataatgagaaaatacacattaattatttcgattagcttagtttgtaattttattttctatatctttttatggtagtttctacaaatgatcctcatatttagactgataattagaagaaatagttttatgaaaacttattcattctttatttacattgcagtgttgtatgatcagccttcaatttattgacctttttaatttagcatttgtctctcatgtgatttttactgtttgtggactgaggtattttattgtggttgatttttagtatttattgacatatgccacattgtcagtggaagtgatttacgtgtgatttaagtgtttctttattttcatgcaccttttccaaatcttgtgttcttatgaaatgctaaatatggcactaaaatatacctttgtagtttgtaagaatctttgttttatttaggcagcaggtgtttcccaaagtcttccaagtgtacatggagcagcgccttagatgatcgttacctctttttagtagtttgctattatcatggaaatattacttttttcctttattaagaaaccacatttgcaataaccgattgcagtacttccgtgaagtctttctaccaggcatcttatgtaatatggagataagaataaaccctgtttcacaagactattgtgaggagtaagtgaacattaatagattagtagagtgttacacatgtaagtgttcaataaaattaactgtaagaattattactatttttactaataataaaaggctttttcctcatatggtttttaatagtcaaatatgtaagtatcccaaccaaactggctgaaggaagagagagtacccttaccatctcatttcatgtccttgtagaccgacctgtagagcacctgcacaggcctgctctggttgaatcttcactacagactcagtctgtaggtctgctcagttctgctcagttcagttcagttcagttcatggttattaaacacatgaacgtgactattctgaattgagacgtgctttaagtgtaaaatacacactggattatgaagacttagtacaaaagggagaatgtaaaatatttcaataaataatgtttataactgattacattaaagtgatattctggaatatattgggttaaatgaaatatattatttgaaaccatttcacctgtttttattttttttaatttgtctattacaacattagaaattaactacatggatcacattttatttctgctggaaatgattagtctacaatctagaagtccgtggagttaggtttgaggcattgtagcttcatttggacctagattagctatctttttctagttatgtttggttctgccttaaattttataaacaatccaaccaagtctcagaaatagaccccaattcaagtgttttgttaattcttctgttctgcaggtcccctataaaagagaagggcctcttggaaagcactgatacacatcacccatcaggtgcatgttagcaggtgtcataaattttaaagtgtgctattcatatgtctgcttaattcacagaagaaaacccatatgcctatccttaaagtcatattatcctatggaaatatttatatatttaaaatataaataaaataacaataaaaatatattcattataacaactagttttgaaacagagaaataagaagtgaatatatgtgtttcgttagtcaagataacatcattatgtagaatttttcccattcataatttaattcatttcaaagagaaatttgctgaagaataatttggaaattaaaaactgtgcacagaaaaaaataaatgtcttaaagtcacagctagaataaaaaatgaatttacgtgttggtttccatagttaatccatattcctaatgcccttctattttgatttcagatgcccaatataaaatataatacttaacctcaaacaaacccattgtagcctagtcagttgctacccaatgactgccagagtattagagtgacaaatatagcttgattttaaaacacagaagtagatgttaatgtgatctgagaagacattgttagatttttgtttttaaagttaaaatagttgaagaagtctaatgtatgtgaatgtgtaatgatttcagtaaattaataattttaaaaagacaaagtaccttggtataaattaatttagtttattacttatgtgtgaggtcatataaattggagatccattctcttgtcatatattaaatttttctcattccaagatgaccttggataaggaaatttgtttctctggacctctgttttcaaatgagttggctggatttgaaaatccttcccaacttcaaaattctaaaattttattattctattatgtcggatatggatttgtagtagtatttcagtcttattttatgaactttggcatgttaacataaccttcctgagcctctccggtttgtatctgtagagcagctaaagctttatatatttttgtaattgggggtgcataagaataatcttcaaattcagacatctctagttgattaatcattacttcagatttttttgaggaaaaatactctaatgatgtaatcaaatataaacgtggtattatagcaacaataaacagcattgtgttaagtgtaaattatcttatttatttatttatttatttattttatttttttgacccaggctggatggagtgcagtggcgtgatcttagctcactgctacctccacctccgggctaaagccatcatcgcagattagcctcctgagtagctaggactacaggtgtgcaccaccacgcctagctcctaattttttttgtgtgtatatagatgtgtgtgtgtgtgtgtatgtggagacagggttttggcatgttgcccagactggtcttgaactcctgagctcaagagatctgcccgccttgacctccacaagtgctgctgttacagacataaaccaccacacctggcctacattatcttaaaatattattttgtaataggagctttatagtttatctttgacaggaaatgcccagtaaagatggttgcttacataatagatcctctcacctcccaaatcaccagcatttgtagaaacctatcttcgagtaattctaataacaaaaactcattcatatgacttttaacagttatggaataattttaatacattaatataatggttaggacagcactgtgaggtaggcagctcttattatgatccccattttatagattatagtattgagactcaaagattaaaccatctgttcaaagtcacatagctagtaagacagactcaagcttctgacactaaatcctatacccttttaaagacagtacatttccatcatattgtttcataggcaaagttcatagttacttaaataattaaaacatgtaattatttctgatgaatatagtcaataaaattccactgctataattcaaatatatccccctttttgggctactataatgtatgagtgaaaacttcagatctcattcatgtagccagtcatttcacttatttctatttttcacctcatattatggaaatgacttgttcctgaagtcgttatattatcactaatttatttgaaagccttaaagttattggcatatagtgttagatattttgtatgatggagtgtgttctgagatcagaaaatgaccctcttcaataaaactaattcagaaatatccatagaacaatctaaattgttatatttatttaaatttgatttttcttcatgtgacttaagtaatccattggctgaaatttggtagtatttagttcagaaaactctgcttataaaatttagtaactttgccatatatcatccatggctattctacagagactaagcttgatcctactgtacatcagtactacttgtgataacaaataacaataacaatcacaatgataatttatcaaaattacctgttgtcagaagtgagcaggaatagaaccaaaaaaagaagagaaggaaaagaaaagaaacatggaatggatgaagaaatttctattttgatccataataatcttagaaattcttagaaaatgtaacctttgctattgaaatatggcacagcaagatgcacaggcttgggcagattactgcctaaaaatatcatatataattttattgactctaactccaataaatgtcctgttttttatccatgtgtacataagtaataaaactagtcactgtaatggttacaatgatgatcaaataaaaaatcggaatgttcggctgggtgtggtggctcacagctgtaatcccagcactttgggaggctgaggcgggtggatcacctgagatcaagagttcaagaccagcctggccaacatggtgaaacctcgtctctactaaaaatacaaaaaaaaattagctgggcctggtggtgcatgcctgtagtcccagctacttggggaggctgaggcagcagaatcgcttgaacacaagaggcggaggttgcaataagccaagatcacatcactgcactgcagcctgggtgacagagggagactccatctcaaaaaaaaaaattggagtggaatgtccactcatttcttagttcacaaatcgtcaatgattttaaataaataatcacagctttaaatacaatagcagccttcagtttattcattttatattcagctccttggaatatattctattcaaataagcaatttctctaaaatttgtgataatacagaattatataaagagcacttacaactcactattgagacaacccaatttaaaataagccaagtatttgaatggatattttaccaaagaagatatatgaatgggcaagaggcatgtaaatgaaaaactgttccacttcattagtcattagggacatgtaaattaataccacactgagatactactttatacttacaagaatagctacagtaaaaaaagacaggcaataacaagtgttgacaagaacatggagaaacaggaactctcattagttttagtgggatgtaaaatggtgcaaccactttagaaaatagtttggctgtatcttaacaattaagcctccactatgttactcagtaattataatcctatgtatctatgcaagaggaatggaaacacttatttacatatatttataaaggatatatagcagcattacttataatagcaaaaaactggaaataccacaaatgtacattaattggtgaacaggtattacaaaacaaatattattcaggaataaaaaggaatgaagcactgatatatgccacaatatacatgaactttaaaaacattttgctaagtgagcccagccagatattttataattctatttgtatgaagctttcagaaaaggcaaactgatgtaaacagaaagattagtggttacctggggccggaggtaagaaggagcagtatctgcaaacgggcaagagggatttttttttttttttgagtgatggaaaatgttctaaaattggactgcagtaatgattgcacaactctgtaaatttacttaaaacactgaattgtacattgaaaatgggtgaattctgtttttaagacagggagagtctcactcctgttgcccaggctggagatcagaggtgcaatcatagctcactgcagcctcaatttcctgggctcaggtgattctcccacctcagcctcctgagtagctgggactacaggcacatgccatcatgcctggctatcatatttttagtagagatggggtttcaccgtgttgctgaggttggcctcaaactcctgggctcaagtgatccacctgtctggacctctggacctcccgagttgttgtgattacagacgtcagcagccatgtccagccaaagcgggtgaattttatggtatgtaagttgtacctcaatacagctgtttatataaagagagagaggccaaaaaaatggtgacataattccattgtattttttaatgtcatccagatcaaagatagaattgacacttctttgcatgcagcaagtggcaatctatgtgagtgagttaaaacagagtaagaggaaactccaattccaagtgtaaaggattattccaagtaagaatatcagaagagcaaagaactgcctttatggtagtcctttggcttcaacccacagttcatacccacatccttatggtaggtcagatgatgaaatgcacattgtgtgatcattaaaggacatatgctctcctgtgccctgaaacttttcaaaatgttttcaaataaatcaggcattttatgtgcaaacactcagtatataacatatgtactgtctctacaatatttaaagataactcttacagatctgtactaataaaatagtaaagaataaaattaattttgtctcacgttatgcattctttaattttaaaaaagaatacaaaaaaggcacatgcgatttattctattagtcctgacatatcttgactttgatacaaatatgcatctcttctcacttgtactgacaatcacttccttttctgaacagtttttttttaaagttttattttaggttcaggggtacatgtgcaggtttgttatatgggtaaattgtgtgtcacggcgttttagtgtaccgattatttcatcacctggctaataagcatagtaccctatagatagttttttagttctcagccttctcccaccctctatctcaagtaggcctggtgtctgttgttcccaactttgtccatatgtactcagtgtttagctcccacttataagtgagaacatgcagtatttggtttcctgtttctctgttagtttacatacgataatggcctccagttccacccatgttgctgcagaggacatgatctcactctttttaaggctgcatagtatgccattgtgtgtatgtaccacattttctttttccattttactgttgatgggcatttaggttgattccatgtctttgctattctgaatggtgctgagatgaatatatgcatgcatgtttctttatggcagaatgatttatattcctctgggtatatacccaataatggtattgctggatcaaatggcaattctattttaagttctttgagaaattgccaaactgctttccacaatggctgaactaatttacattgccaccatacattttttactcacgttttaaatcctaagctagaatgtaagatgtttgaggccaatgctaaaacatatgtcatctgcctttgtatcctcaaaagaacctagtgtcactataaatgcaagtgattagtgatcactgtatgtataatagatgccgaattatgtcaaataaacatgagaagttatctgtgccttactcttgactctcggtttccacagtcctttttgaaatctcaccaagacacagacaaaggctgaaaaacataggacacttaatctgcatgtggattttaagggctggctgaacatggaatgacattcagaagtactgtgtcaagttgagcttctcaagttcttgaaatcaccacaagtgatttccacaaccacaagtgtggtggtgatttttatagcacttggttgtaaagcctcactcttagttttctgctaagccatgttagtaatacttcgtcctcaagattggacttatcctttctttctcactagtacagctttattaattggtatatgaattgttttccaaaagaattactgtttttatgccattgtgcaagtactgacttctctatgatgcattaaattatactaacagcatgtataagtcctagactgagaacaaggactcaaaaatatttatataatgtttattttaagattgatgactgtatatttcaaattctgtataacagttttaaaatcaggaaaatagatatttaattaatttgttctatatagttaatatgtactgcatgattacagttaacattttaaaagttcaaagaacaaagaatgggaaaactcttaatagtggttatgtctgggtaaccaagctaggggtaactttttcttgttctacattttttagtttctacagttatcatgcattctttattttggagaaaattacacataatttaaagaagaaaatagtgtgttcctctggaagcccaattagcacatggaaaataaagtttccatttttttagagccagtggcttttttttttctattcaactattttgactcttttaccttgctcaaaactgtaagaattgctaaacttcatacctcgtacatttctatagctgctaaaatcccaccaatggcatctttggggatcagatactatttatctgacactgctgtactttggattatctgtgtacttgttaactaaggcaagcccttgtcatttcttagaactatttgaagaactgaagaagcttattaagaaaatgagtttgtgcctttttattccctgtaaataacaagagatttgctgtaggtgtcaagccttaaaagtgttatggtgaccctttgctaaacaagacataaattggccctcaacagtatgtaacttaggatcaactccaactttacaattcatttccttttatgtctgtttttagtgtgttgtgtttctaagtgagtccaaatttggattttatttaaaatcaaagattgtagaataaaaagctgagcagctgcaaagctgaaactgtgcttcatatcctactttgtgggataccaactagttttcaactatgtacatggtcttctaaaatgaaagaaaaattgcagattatgcagactgggattaagtatcaaaattagccccattttaataatgtgaaaacaatatacacagtttctaatttaagctttattttatacaagatcatacaaaattataatatttaatagtataatcctattcttctacatatgtattgatgtatatagtaattctcaaatagtttaagaaattaccttcagataacacatctgatttaattaagtgcatgatactctatcaagatgttaaagaaggcttctcattctactttaatgagcagtggtcccattcagttgtagagaactatgtgtgtgacaatgtaatagccaatctggtactgtaattagaaattcaccttcattctgactcatataatatactgaattaattagtctgtaatatgattaactagtttttttgctttttaatattaaaaagatatcataagtctgatatttttagagcacttttaacatcattttaaaatgtgtagatttttgtcataagcatttttattcccatttcttcagatgatgtcaaagaagttttataggctttgataatttaattacaattctaaagtccctgcaaagcaagtttaggaatgtcgttatatcatcaagttatttagtaacattcatatttgcctctgagttacttcttataattatactaccataattaattatactatatattttatcattcattcattcatcaaacatttattgaggatctagtaagtttctgacactattcagaaacttatgtatagagagtaaataaaacagccaaaattaatacctccactcttggagctaataatctattaatataaaatatgtgaaaatgagtcttgttaacacaatgggtaatgtaaacagtaaaactgagatcatacttttttaaaaagctgatttatatataatcattctttatatactctttaaatataccaatattatagctctaagagaattttaaaacccacctcccattaaaagatatgccagtaaacccatcaatgcaaaataaaaattgcattaacaacagtgttctaatttaataatttcatatcccccgcaatgtttttggtttatctctatttctttctttctttttttttttttttgagacagagtctcattctgtagctcaggctggagtgcatgcagtggggtgatctcagctcactgcagcctccacctcctgggttcaagtgattctcttgtctcagcctcccaagtagctgggattacaggcacatgccaacacacctggctaatttttgttttttttagtagagacagggtttcaccatgttggccaggctgatctcgaactcctggcctcaagcaatccactcacttcggcctcccaaagtgctgggactacaagcatgagccatggcacctggcctcctatctttttaagtggcactttagtctactccacaaatgaagaaagcaagctaggattcttgtctcctttcactccctcaatccccatattcaatctattaccaatcattttcaattctacctcctaaatatcttttgattttgctgcttcccactgtccgaattgccgtcaccagtttgacatctctctcttatctaataacatcaatagcctctcctgtctggtttcccctctatagccagatgatctttatgaaatccaaatttgggcatgtcatgtccctatttttctgggtaagcacccagaagatacttcgcacccttcgatacttataaaggccactgtgcctagcaaggcttatagagtttcccatgactgggccactcctaagctacctctcctgcctcatttctcccttatcttgtcccattctcttgctgctgcagccaaattgaacctttttctgcaattccagttagccaaattctctctttctctcttatctcccttagatctcctaacaagctgttttcttcctggatcacaagtaccccacgctaacattttactcctatctcccagctccctaaccctcactcttcatacgtggaattcctattcatattttgaatctgagatttaaagtcatttcggggagtctctatctacctacccccgacccatacacaaatgcatacccatcttgggtagattccttttataacattcagaacatgcaattatttgtttcacattcatttactctgggaaggaccatttttccaagttctatttcagagttttgaatatggtagatgcagaataaattttgttgaatgcaaatgaatgaatgcatctgagagctggaatgaaacatttcagagtgatttctagcagatgaaaaaaaagtttaagataggttaaccttcataaattgttccttaaaaatgtatttaatctgttcactgtcacttatagattgagtataaaataaattttaagtggccaagttcattgcaacgtccatatagtatgttttgaacttggagggggaaatgcctttaaaacccattattggtaattttatgatttgtattttagttgtaattgttgtggcatggtataaatagctgagaaacgcagtctctagcaaatggaatagttaagagtgtaaagaaaatcaaaatttgattttttataataactactgatattaataaaatccaaatggatttcttgcaaatctggtaaataaagatgggttttgaagcttagactttatatctcctttgattcatgagctaattttcaaagctgagttataagctttagtttatattgtacctggggcgcaggcagactcatacaatagctgtacttcaagtgtcaccataatatagtaaatgtaataaagttacatttaaataatgtaagaaatctaatttaaacagggaaaaatgaaatttaaagttaagttaatgcctttttctattcctgatgttgtcctttcctgctacacccatagtggtgccgctagccttttcatgttaatatttggtagtacagctatagagaaatatacaattacttagccattccactttctagataagaaatggccatttgacaagcagctttacacacagtgccatattcatagtttgtgagggtaaactcggtatttaattagtaccacaaaccttcctgccattttcttataattgcaattttattttttgaggtgaatttagaaagttaaaagttttattttatagtacagtgattagattgttaaacttctttcagaaacagagcaggataaaattgtgtatgctgtgaaaaaactggaatggaacatttttggtgaatatttactgaaatattagaaaactacagggaaaatattacaaattgcatattttagccattatctgcaagcattttaatagtagtagtatgctttcttttcaactttgatgccccagtttcctcatctccaaaatgtaaataataatagtattgtccccacagcattgttgtgagaactaaatgagttataatgtagaacaatgacacatttagaactatatatatgtgtttgctatcattattattttgttcacattatttaaaaaatatattttaggaatttaaatatatgtaagtaaagtttctttaagtagatagaggatctaaaaatatcaaggttaagtatggcttatatttgtgcaagtattgttttataaaaaaactaaaatgtataaaatgttgtattcatatatttttaaattgaaaatacaaataatgctttatttccataaatgccaacttttacttcacaattatatttaaatgtaaataatcagtgtaatgcacacttgtacatatcccactctccctgcccccaaaacacggttcccattaagaccgtttttttctgcaaagtagatgagttttggtcctaaaagtactagaaactgtatgtatctgacagatggatgggtcattctgtgaacctgagccactgttcagccttcatttatcatcactaacctctctttgaatagaatcttgaatactcaccagtattccagcatagaatgcatagttttagtccctactttgccaatgatataagtaattcacttcagctacctaggtttaattggttataacctcaatccgtataattttacctgtattcaaaagtgttatatacctttgaaattgctatatgcatatctacctgtattccattaagtctgaaataagtaaacctagattaacagaaatgatttatgttgcaaaaatttactttacaaatgaatccatataaatgtcattattattctattaagaaggccttcctattattaaaaatacagttcactttataatcatacatatacatgacatccaggtatgcatttatgtactaaattgcaaattaatataaaattaaatatatgaaaaagtattctgcctgattccagattctagatttttagctaattgcagatgttcagattttccattaaattattcatttgcaaatatcagtaagttttgtaagcaaacactagctttgaaactcaatgccattatgttgatatgatttctgcaggttttctgatacacttaaccaaaaacatatgtccttcctctatgcatacttttattcaaaacattaaaatgggcttggcttactcagggaaggtataagatgcctgatgtggaggatttgttctttaggcatatgaaaattgcatttgtgttggagacaggctaggaaagtgatgaacttaatcaggcctgccacattagtatgacaagttttttggattttaaaatgaataagaacagatcagaattcaaatatatagcttgagggaaaggtgtaaaatttgttgaaatatgagtatataatttcttctatttctactcaagcagcattgtggtattctttagaaattttttattaatctctaaaaatgacaggtgtcaggggtcttttgtgatgcacattcagaaaagtttcggtcaagaggtcataccgctgtgatgtgttagtcagtagccctgtctcctgtggaatttgccttgtttgaagtgtttccaaagcaaaatattttctgtaatgcacatcagttaaataatgtgtttgggaacagctttacatggaataaaaggctatgagatttctttatgattaaaaaaacaaccactgctccttttaaatacaatgaaacccctggaagtacatagagggatgcatatttttaaaagaacaaaatattcaggaatcatattaaataattaaaatgtatatttatgactttttgaacttaatgtctctgttcatgtaaaacacaagttggtttaattttttaaagatacacgtataattttgatacaaagatatttttaaatgagaaaagcttactcttaaaacaatgttatttcttaaaatgctttgaaataatatctgataaaaggtttcaggatttgttttataatcatcaactaatgttttttatagtgtacatattgagagtaaataaaattggaaaacattaataatcaaaaattacctattttcccagcagaacctgggaagcattttaatgcttgttctttcaagtgataatgtgctgtcagggaaataacagactcttgaaatggcctcttgacacatgtatattttcatgaaattctagatctagaaaggcggtaggatagagagcaaatatattggtgttaaagtgcacatatttcggtgtgaattttgctttaaccaatcatcagctgttgaccttgggctagtaacttatcttctctaatcctcaaattattgtttgatgaaatgaggatcatactgtctactccatgtggatgggggaattaaacaactataaatgtctgatacttcctggggcacacacactcattaaatgattatagtttgttcttgtccttatcagaaagaaagtaagtccctccgtctcatccttccttcacttattcattcattttacttggccttagtatattggactatatgaaaatatagtgcttgcccgctttgagcttacaactcagtgatggagaaagtacatcatttacctagcacagtatttggcttatggtagctgcttaatacataaaagtaaatagtaagtaattacaagttgtaataagtgctctacagaaaaataataagatgttatgagaaaggactgcagagagcaccagattgagaaagtgacaattaagctaagatagaaggatgattaagagctagccaggagcaaaattgtagaagggttgtatttgagtgatgacagctgttagatgacaactaattttttatgccgaataatacattgaaatgtaattgttagtattctgttctccatatttctcttctataagctgaaattggaattccttaaactttcttatttttcttttctaaccccatagatcagacacatatttgtcttgatatttggtattctctctatatattcaaaaatcataggtaaatttgtagtaccatattctcatatttacatataggtcaaatcatttttctttcttcctggcagtgagcctttgtgatatgttcttacttttccatttttccttaaatgcgatattaacaatatctcaattgaacttattccttcagacaccaccctataaatctcttcagattgacagtgagccactggttcaagtaagtgacacactggattagccatgtgcaacttaaaaaaaaatcttattggtaatacaatcagataaagttgaatagaaggacttaagcacttctgaatatattgtgtgttctgactccaggacatttgtaaatgctatttctgtcatagacaaatgttatataggtgtgcttcaaaaaatgcatgagtacagctcatgatacctttttagaggtttgaaaattgattgattagtgtgatgtattccagacttccatataaaagctaagctgactttcacatttgcagtggtgtctctttaagatgatcagttaaatcttagataaagtctgctttctggtctttcacagtgaaggcatttcgcctctctcaaatttttgaaaagtatagttgatagcagtggtgtgcagcatcaagatgttatttaagtactcaacaatgtgtgtcatcttgctgatatgaatatactgaatatggggagcttttaagagtaatccttaatcagttcatttggcattcttgtttggttttctgctttttcatcattgatagctgtcatgttagttttcagatgatctttttgtaatgacttaagtaagagcctttgtcttctttgcaagtctaagagacttatgttaactgccttctcttagtgtttgagctaattaaactgtttaataacaaatcactttaaaaatacttgtctatttgttaaaaataaatattgagcttctgttatcttcaaaactctctgatatagttccagaaacgaaaaactgagtccatatttgtttgggaaatttgaagtctggtaaggtagaaaacacatcagggatgtgaactgtgactaatatgaaggaaacaacagttttgaatgtgaaatgatattggagaacatctattccaatttcctgtaaattgcatataaattaagagatttcctatttgtcaaaaactagtcagtaactgaagagatgggagaaccagggtcccttacgttagaaacaggaattaggtttagttgttgactgaggggttaagctgcatttaagtcctttgatttttttttgtttactatcagtacaatgtggatactatctcttctctgacacataatctggtacattgtaggtgtttccataactgttttctattccatcacattgccaccttctctccaaaagcaaaatgatataaaacagtttatttgtatattccttcagcaaacattgactgaggaccttcttggtggcaagacatgtgccagggattaaaatttaagtaagaaagattctctcagttggtcttacttcctggaccaagagatacattgacaactagtaagtctcttgtccttgctttctttggcagcatttgtccccgattctcattctgcctctctattcttggtctttttgtgggaatctgtttctatatctcccttaaataaacctgagaaaaacaagcaatggggaaaggattccctatttaataaatggtgctgggaaaactggctagccatatgtaggaaactgaaactggatcccttccttacaccttatacaaaaatcaattcaagatggattaaagatttaaacattagacctaaaaccataaaaaccctagaagaaaacctaggcattaccattcaggacataggcatgggcaaggacttcatgtccaaaacaccaaaagcaatggcaacacaagacaaaattgacaaatgggatctaattaaactaaagagcttctgcacagcaaaagaaactaccatcagagtgaacaggcaacctacaaaatgggagaaaattttcgcaacctactcatctgacaaagggctaatatccagaatctacaatgaactcaaacaaatttacaagaaaaaaacaaacaaccccatcaaaaagtgggtgaaggacatgaacagacacttctcaaaagaagacatctatgcagccaaaaaacacatgaaaaaatgctcatcatcactggccatcagagaaatgcaaatcaaaaccactgtgagataccatctcacaccagttagaatggcgatcattaaaaagtcaggaaacaacaggtgctggagaggatgtggagaaataggaacacttttacactgttgatgggtctgtaaactagttcaaccattgtggaagtcagtgtggcgattcctcagggatctagaactagaaataccatttgacccagccatcccattactgggtatatacccaaaggactataaatcatgctgttataaagacacatgcacacgtatgtttattgcggcattattcacaatagcaaagacttggaaccaacccaaatgtccaacaatgatagactggattaagaaaatgtggcacatatacaccatggaatactatgcagccataaaaaatgatgagttcatgtcctttgtagggacatggataaaattggaaatcatcattctcagtaaactatcacaagaacaaaaaaccaaacaccacatattctcactcataggtgggaattgaacaatgagatcacatggacacaggaaggggaatatcacactctggggactgtggtggggtggggggaggggggagggatagcattgggagatatacctaatgctagatgacgagttagtgggtgcagcgcaccagcatggcacatgtatacatatgtaactaacctgcacaatgtgcacatgtaccctaaaacttaaagtataataaaaaaaaaagaaaaaaaaatcttgatgtttccctaatcctacttttttgccagctattcttctcattccatagtgtatctctgacctttcccctgagtttcaaatgtatgcatattcaatgtccaataaatatttcactaagatattccttaagaattttaagctatctgaaccccaaactgcatttaaaacttttcctaacccaaattctttagctcaatttatagcaaaaacatccatttaactaatgaagccaactgttggtcatccttccactccaccctctgtatttaccagtcataaatgctaagtctttttcctaaccatatctagaaccatttgccattgtgtatattgtgtaatgcacaaagacaaccttattgcctctggaagtatagatgtggtttactaatgattttcaggcactgttggtgaagtgtgttgtacatataaaaccagtaaaaaatagagttttccagcagaagaaagtaaatcatctcatggaagcacctgttggactttggggaaagtaacagcatatggataatcttcagctctgttaccctcctctctatatcccctacaacttgctatagaagtagttaaggtacatctcttaatcaaactattagtcttatagttaactgtctccttgtccttacttttaataccagcctacttccttacacacaattaatttcccacattgcttcagagtgattcacgtaaaatgaatctgaccctaccgctttttctcttaaaagctccctaacagtttctcattgtttagtatacatccagaatccttcccacagtacagaagaccccctgtgatcttaatatctgcctgtctgtcctgcctcaccatgctaccttgaattgtgtgttttattgatacaggcaatgtatttttttgcatatagcacaccattttgtgttctgcccatgttctgctttatgcctaaaatgcccatctcactaacttagtcatccttcaatgcaccaagatccaaatcagctttccctgaaagcccctcccctcactctcatcctcattcccactagttatgtcatttattccaaggatatactaaatgtatctttgtcattgcacctaccacttttactgaaattatctcttcatccatctttaactcactagaatgtgggaggtttgaggaaggaatgggagaaggggttaggtcaaacatgataaaactggaaaagggaagataatgctagatcacagagaagtttgaatgccatgctctagagccctgcggtgtaatcaaatctgcagtttttaaaacagtaacccaggtacagtaaaacactgagaaaaccatggttactataactaggctgactcttgtttaaaactctactctgacccttagtagttttaccttgggtcttttgcaacaaaactttttcaactgtagttttcttagttttcaaatggtggtaaatagtatttgtccccaagaattatgagattaaacaagataaagtatagaaagtgggtgtcttggatttaacacattgcagattttcaatacacatttaaaaacatttctatttaaatagactacctctaagaatttcaatagggtatgatcttccatatcattgtgattgatttttaaggaaattagcatgtggaattaattattttaaaaagcataaattatatttgcactctcatggtcatggtggcattattcacaagagccatgatatggaaacaacttcagtgtccaccagcaatgaatggataaagaaattgtggtatgtatctatgcagtggaatattattcagccttaaaaagaaggaaatcctgccattttggaacagcatagatgatcctggaggatactatgctataggggaaataagccaaacagagaaaaaaaaaatactgcttgatctcacttatatatgagatcaaaaaagaaaaagagagagagagaataaaatggtgattatcaagggcaaggagtgggagatgtaagacaaagggtacaaagttgcagatatataggatgaataattctggagatctaagatacaatatgaaaactctagttataatacccaccatatactggaaatttgttaagactagattgttggtgctcttaccacacacatcaaaaaagggtaactacagaagagaatggatatgtttatttgtttgacagtagaaactgtttcactgtatataagtatatcaaaaccatattgttcatcttaagtatataccagaaaaagcatatatgtgaattaacaccattaaaaatgaatatttatttctttaaaatttaaccataatactcctttaaaggttaagttctctactttttatccccttgattaaaaaaaaatcataatttccttttatgaatatcatgtttcttctcagtctctttcagctgtctcctgtaaaaggaagtttcatttacatagaaaagtcatattgtgaaaaaaattgctgctcatatttttaaataatttatagatttttgaaaagcttagctcatagccttgttttgtatagcattagatcaaattatcatattaaaaatatattctcatggctgggcgcagtggctcacgcctgtaatcccagcactttcggaggccaaggcgggcagatcacgaggtcaggagatcgagaccatcctgcctaacatggtgaaaccccgtctctactaaaaatacaaaaaactagccaggcgtggtggcaggtgcctgtagtcccagctattggggaggctgaggcaggagactggcgtgaacccaggaggcagagctcgcagtgagccgagatcgtgccactgtactccagctgggcgacagcgagactccgtctcaaaaaaaaaaaaaaaagtcttctcatttttgtattagaaagatgacaactttacaaggcaagtgaattctgttcttcacttccaaatatattgaggaacagaaattaagattttaaaaataagtaatatatttgtaatgacaataatgacaatcatacccatataaatcaaaggaaaataaatccagaacaaaactcaagatcatctttccaagctttatttaagtggcaaaacaacttaaactaaaatatctgaacatatgggtattgcttatctgtgacatgtgacttaagtaatttgatactaagaaagggaatgtgttgttaatggtgcagttattaaataggtatagttatatgtataaaaattagaattgatatcgataaccttattacttgtatttcaatatacttttttatgattgtaagaatttgcataatcttgtgtattcccatgggagtaatgttattcttataaacatcactgataccaatttccaggcaggaaggaaaaaaaaaatctaagaaccatccttgtctaactttcaacaggaatataaactattttaagagccaattattgtagtagtattttaatcatatttcagtatttccaattgtttacagttgcttaactatgatattttcttttctttttttttttttttttttgagacaggatctccctctgtggcccaggctggagtgcagtggtgcaatgatggctcactgcaaacttgacctcctgaggtcaggtgatcctcccgcctcagccttccaagtagcatgatccaccacacgcagctggttcttgacttttgcattttcaggaaaattctgttatttttccacattgcttgactcttccaaattgcttgatgcttccaaattgcttgactcttccaaaggttcaaatacaccatctatttaatgcttcctaaaaaaccatactaaacttagtggcctgaaacaacaacaatttatttctcatgattctgtggaccgagggaggtgctggctaggtggcttctgttccacctagtaataggctgaggtcactcacgtggtggctggcttcatccggaggtggggctgggcttgaccttccaagaaggcttcaacctcctagctgacttctcagtgctcttctgtggcctctctttcccaatgttgtctctcattattcagcatagagccagttttagttcagcatggcacctggctacctagcgggaaggaaaaagcagccagtcctcttaagagctaggcccagaactggcaaagcagcacttctgcgactttctattgttcctggcaagtcaggagggtagcacagctggaaaggaggggaaatagactccatctcttgttggaggagcagtgtgggaagagccgtaggaactactgggagccatatttagtgatgagctgccatggtatcctttatgtattaaaataaaaacccctgacgatggtatgctttttcatattacaaataagaaaacagaaaggttaaatgggcaaatcattgtggagtcaaaagcaaaagttgtattttcagacttatggtccagtaaggttttcctttcagttttacttgactaacattcgtccaaatattttattctagatttccgaataaggttatcaaatcttcactcagttgtccacagtagttttgttgttgttgttattgtttctttggttttttgtttgtttttgtttgtttgtttgtttgttgagacggagtctcactcagccgcccaggctggaatgtagtggtgtgatctcggctcacagcaacctccatctcccggatttaagcaattctcctgcctcagccttccgggtagctgggattacaaacacgtgccaccacgcccggctactttttgtattattaggagagacagggattcaccatgttggtccggttgatctcgaaccgctgacctcaagtgatcctcccgcctcagcctcccaaagttctgggattacaggcctgagccaccacagccagcctacattagttatttttaaaaaccattgcttagaagccttcaattctaaaattttcagtagccattcatgcccaagttttttttagtataatgccctagtatatctaattcatgtcttcagaacgattgtgtagctagtgttttatgttttgttttggtttggtttttacctaattttaacagcttagcaatgaaaatcacatttatttatttatttatttattcactcaacaaataatttattgagctcctgcgaggtgtcatgtattatgtcaggccctgggcatccatacaggaatgagggggagtcatggctcaaagaacccacaaggttacttatcttcagtaagtaagttaggtttgtttagtcagatttatctgttgtatatcttcacaagccttcagttaaaaacttgctgttttctagagcttataaagcaatataaaatattaatcacccagtagtgagtgtaactgggttgaaattcccaaacttctgtgttttaaagtaaatgcacattttctttttctagttgtctagtgcaatgtaaatatgttatcagttgctttagtgaaactgagataccagttgggatactgtagtgcagaggtaagaatgttgacagggattcatgtcacaggtcttaacttttcatatactaattatataatcttgagaaagttacataatctttcttgttctcagtgtcctcgtatttgtgtaagtctgactagataattttcatatcctatgaataacatcagttacctacattaattgtttagcattatttccttgttctccttccttagttctctcacaaatgaaaagaaagcaaatgatttgtacacatgaaatagtttcagtggcaatcaaaaactatatcagggagataagaaaattgctttgttatagctctagaatcttcatgggaggaaagtggagggaggttgaaaggggagaacttggggattactttttaccttggagattttcatatttcttcctctatccgaatctttaccatccctccttccttgagactgtcttaataagctgcagttattgacgggaaggtgtctgctgactcaagttatgttgagtgtgcagaggaaaaacgggctagaaaatgaaaagttctcctctagtgcattttttttaagttaaaggtccaatagccctcctgctgttgcatcagttctctgcaaatgctgaagtataatcatacctatctttgagtttaaaaatgacatttatgatgatttcattagcttaaactcaccttaagcacaaagaaaacagttttatctgctttgaatatctttaaagtttggcttctctgtttcattgtacatttatatttagcttctgataataatttcattttggttgtctgatcacataagagtttgttctgtctgattttgtgaactattatacttttccttttaatgtatgggtcattacttaaccattgttagcccttatttctaggatatttattattctatttgaccatgattgtgtcctgctatatgtgttatttttctgaggtggaatttttttttttggctgaatatcaagactactgttcacctgtatgtatttatatcatgaataaaattcaaactttaggcaacttgctttattctggtttgtattatttctcatgagttgctttttgttttgtacatctaactaggtttctttctgaaatttaaagttctcctgtggcattatcttgtgactttgccatcttccatttcttctctctcctccctttctgtcttcctttcattctttcttcttcactccctcccttcccttcccttgattattcagtactttccttatctgcaaggcactaggctggatgctaccatgttttccctcaacctgcttcttagactattttaactttccccactctcttttattaatcttatttttgccaatttcttttaaattcttcacagatctattaaaaacagtttctagctcaaaaaaaaaatgtatgagctactcagatgttagctctggtttctatttgtgcatgtcagaattgtcttaaaagaacaaacaccgccgggcgcagtggctgacgcctgtaatcacagcattttgcgaggccgaggcaggctgatcacgaggtcaagagatccgagaccatcctgaccaacatggcgaagcccccgtctctactaaaaatacaaaaattagccgggcgcggtggcacgtgcctgtagtcccagctactcgggaggctgaggcaggagaatcgcttgaacccgggaggcggaggttgcagtgagccgagatttcgccgctgcactccagcctggcgacagaacgagactccgtgaaaaaaaaaaaaaaaaaaaaaaaaaggtaacaaacactaaaggacagaaataaaactcagctctgggcactcattttcagcataatatctttttttttttttttttttttttaagacagagtcttgctcagtcgcccaggctgaagtgcattggcgctatctcggctcactgcaacctccgcctcccgagttcaagcgattctcctgcctcagcctcctgagtagctgggactacaggcacgtgccaccacgccctgctaatttttgtattttgagtagagacggggtttggccatgttggccaggatggtctcagatctcttaacctggtgatccgcccgcctcagcctcccaaagtgctaggattacaggcgtgagccaccgcgcccggcttctcagcatagtatcttttcacatcatagtctttgagctgatattttccttttgagttgaaatcagacatatgcattctgagttgattatcatagctttctctgtaagagtaactggttgccaaatactactaaatgtgctacctgatgtgctaatagaaagggctttgcagtaaatgtcttgggaattaagattaactataatcattaagtcctatgatctgaagcactacaatctgtttacatgtattagttgtctttttatagcaaatgatacaagaatcaatcaatacattaattataacttgcaaatagttcatccaagatgcagtatatgcattgttgtattttttaaatttcatttattatgttttttagagacagggtcttgttctgtttcccaggctggagtgcagtggtgcaatcataactcactgcaacctggaactcctgggctcaagcagtcctccgacctcagcctctgaagtagctaggactacaggtgtgtgccaccatgtccagctcttgcatgtttatttatatatccacctgttaaatcaatgattagtatatgtagaaatttcggaggagaaaactatatttgaagaaaatcaaataggcatctcattttgaatgtttagaacagtgctttccaatagaacattctgtgatgatataaatgttccatactctttgctgttcaacacaatggccactatccacatgtggctactgagcccatgaaagtggccaatgtcactgaggaaatgaatgttttaatgtgtttaattttaaggaatttaaatataaatgggtacatgtggctaatggcaaacatgttgaacagtgaagatcaagaccatttaaaatgtatttgattataagtgatagaccactgaatatcaaaatatgtgaccttactcattttatgtgtaatcattttatgggaaattttacttaatttacatttttctaagtaaaaaaatacaattaaaatgcaaatcaacatatttaatacagtgagtggtggtgactagattagtttggtggtaggagggattagccccattccataatctctgttatggctctcccactagccacctgaatggtaagtccaggtaaaaaacagtacagtatgcatgccgtaaatattatttcaatacaagtgtcctcatattcagagtaagaagaaagaacaaggaaatgatagatctgatgcttgagatgtaatcaataactaacaaagagagagacagtgtttctagctctattttgactatatatttttttttaaatcagaaagaacaatctttggattgaaaacatgctaagcaaataccattaaaaaggaaacagaatccatagagaaaggattgtaaaaaagcagctccttgtttccaaacctagaatgaacaaattttattcccctgtaagttgataacatttgtaaatgaggtttgtgaaactgcagtctggaaatttttagtaaatgtggagagcagaaatgttgcccaaagaactgtgcaaagggtagatactaagacctccatttaaacagaaaggagagagggaggagagagagaaaaagagacagagaaaggtaggtgggtgctcaaacttacaaaattgtgaccagaacagataactaaagggatgacttatgacctttaaaaaaggaaagcagtgttcactagaagcaaccatgagttcacagagaaaaacttttgtccaactaaccacactgtctgttttgactaaggaaaaaagaaaaaaacgtgttgtaggttgtctgtatgaaagtaattgaaaaggtttcccatgatagcccaaataagataaagaaataaggaggagatgataatgtagttgggtagatcataattagttgaacatccaagcccaaagagtactgattagctgaagggtgaactctgtgtgtgtgataaagagctcagtctttagtcctgtttctgtttttgttttttcaccatttgtttcagtggcttacatttgcgatgagcaagcacctttgcataaaaagcaattcaagaaaaacaaaaatattcatggctatatggtagtccatgatggtagtattcatggctatatggttttatcacttataatcaaaagaaccaaccaaacaaacaaaaaacctgtttgtgacatactactctactcatcatgagtgttagcccatctgtgccatgttgttttagtaatcatcttccatgcccacaccttgatagagtaatacatgatttaaattttagttcttttccaaatgttccaaatttctataattatcatgtgttatttaaagaatcactcccttttccatctatttcacattatatggatactgtcttagaagatgtggacattatagttgatttaggtaagctatcaatattagattgctaagaggacaatctgattcctttttaaaaattaattacttaattatttttaaatagagatgaggtgtcgccgtgttgccaagactgttctcaaactcctggggtcaagcaatcctccctcctcagcctccctaagtgctgggattacaggcatgagccacaacgtctagcctttgttcttttttaaagctttattttcacaactattttatgaaagagaatagagacactactcccattctactttttactcactagattggaatttctcacctagttaataaattttaagaggtatcagacaatttggaaggcattcataagatagtgaataggatgcatagaaacttattagagtgattagagcccattttatttgattctaggggataaaatgagaaccaataaagtctaagccttaaagagatggagttgggctgagtttaaagaaaaatgtttcaacagagtattctaaagggttagtaaaccacctattaaaagagtgagttctctgtcacttttaggattctagcagactttgaatatccaattagcagggaggtttgttgagcaatttcagatagcccaaagataacagcctatccaatcttagattcctattttctgtagcaaagccttgaagatgattagcacactttgggaagcgagtgttaccaattgagtagccaggccatgttctaacttcatttttaccctcttctcttcccttgttcttcatggctaagtgctttaatgacaccttacctgatggcaatgttggaagaagaaagtagtcagacttctccagaaatcactcccttccctcccatctgttgaggctcactgctaagcctgaatatttgagaacccttcctgagagtcttacccacgaagcattagctttagactccctcattatacccaaatcatatttattgcaacatggactaggatttaaatttctagagtaaaccattggagcttctcttcttgttcctagatctcagcttactttctttttgagttccattctgacaaaatggcccttactgatttccaaacctcagttcctcacctatggccactttatattactggaattttgctagagacccatcactggagtaatgctggaagtttgtctattcagaaaatgcctcaactattccatgctcttgcctcttattctcattactgaaatttctaccccagagacttcagtccccaggatttcagtcaggtgagtagctccaagggaactatggcacaacagcattccaagaatccatttcttctttgttcagatgtctaagtatcaatggcctcccagatgtgttgtcactatggttacttacttttcaagaccttgtgccacctcagcttggttcttcagattttgcttgtactgtcattggagattcttgtaaatcttcaaactttggtcttttgcattcccagctaaaactttcctgatgccagtcttgatgtagtagatgaatttactgatttctccagtcttgggcccaatcttggatttgtaagctgtcaggtatcactgaaaaatcttaaacatggaacacttgtgttgaaacttttttgaaactccagtcagcttttattgctttccatattttaggaatcgttgccagataatgtcaattttgtaactaatgtaacaaaaggaagagacatgttttatatgaatgacacaaagaatcaagcttaaggttttctttttgcttttcccaaattcaaaacaccacaaacagctcactattttctaaatagagcaaggacacttttcaaaattgaaattgtaaatataacacctgtgtgctaagctctgcaatgtgtgaaatttgagattgttgttgctaatgttcaaagcatagaaagtctgagtagtgactgtctaggatcagcaagtatgctcaccacagtcagttcctggagttgaattatttgaaattggcaggtggtttctctttttccatagatttcattccccttttcctgacagtacatgtgtttggtgacctccactgtttatttagcttctgcatacattccttagaatttgtttaatgtgtggtttttttggttgcttgtgtttcttgttatgcttggctactactagaaggaggagtggtggtggtctgctttgggttggcagtcaaaacaaatgtgatactcattttaaatatctggctttgaattttcatataaaaatataagcaccatttacataagtatattgcttgaattgaatttattttccattccccacttgctcctgaaagaagtgcctgtaaactccaaaatgatttaagagagagcatcggaaggaaagaaaagtgaagagtgagagtaagccactaactgcttaatcaaacctgaatgagccaaacaaagtccaactgtgatttactttctgcatcataagttgcaactattgcaactgtgaagagttaatctctcacttatacctccttacttcttactgccttcccggttgttgtttacttgctctgtctgttacaaactggcagtcacatcgcataccctattatagttagtacactttggggtctgaggaggatgggaacaagaaaacaaagaaatagaaaattagaaaaatcaaaaggaatttatgaatagaagatactttcttattattaatattgtcatttttgaaaagcattaacagtaaggtagtagaaagcattgacatttggagtggatctgatttggaaggcccttctgggatctctctactctgggttttcagaacctttccatcacctggtattcttcacatccctttgtctgttggtttttagactctctttttttgcccattcattaaatgctactcgtccccaaaggcattgtcctcagtctactgctgttcttctgcaatgcttcagctacaatgtatacatatgttaatgacctgaacttttctatttcctagctgtgatctctacccaaagcttcatgtacatttcattataactgtttatgagaattttcccttagtgacagtaggcactttaaatggatcacttctagaactgatcattttattcttcccctaaagtagtgtttctcctctgaaatcctcttaagttctagaatcctaggagtcatttttgatttctcagttactttaaagtttgcccgtatctattcaatccaggaccaaattctggctctgcattgtgtgtgtgtgtgcatgtgtgtatgtgtatgtgtgtgtaatgcagttattgtcatgtcgctcacttgcttaagaaaattcttcaattgtttattattaccttcaggataaaatccaaactacttaaggtgacattgacatcttttaatgatttagtgccaacttacttacccatcttactgttttcacactccctggctcataacttaatctctagcaatcctgcttgtagttccctacgctacatacgggtcctcgctataatgcctgtctttgctcatgttatcccctacctggaacaactctttctcattctttttaaatctcaatcattccctctttcagaaagaatttcaaacaattcttctcaagtaagttaggcgtcattcctccttgcttccacaaaaatacgatgaatgcatgatacaatacatcaaaattatctttttatatatctatcccctgcaaactactgtgagctttttaagactcaggattattcaacttccaattctcatcagttaacacagaatctgacatgtaataagtgctcaataaatatttcatttacttaatgaaaaaaagtcattaatgaagactgaataccttgaaaattgttctttctgcttttggattttgcacataagaactcttagtaaagcacttatctattctcagtcttcacttggcctctctttttggcaactgaaatgactcattttctttacagctcctccctttggcactctcgtggcctgtgatcggctcctgttcaactcttcctcttggccttcttgaccacagactcaaatgccctgagagaatcagagctgtccccttgtctttcttcttccattgccagttttataaatggaagatctaaatatatgtgagtgtatgtgtacatatatagtatatatatatagtatctgtcaagttttcaacaggaaaaaaggtctcagtcacacaatatcctatgtcatatacaatattacaccatgatatatgagattaaaagagtaagggttaccagctgaaaacaagaaagtgcaaaacttcttttttgggtcaatgctttctggaccttctgagcagctcatccttattcttgactttgtggtcacctcaaagctgattgctagtgacattttcacagtggtaagtttttacaaagatatctaaaaaccacaataaaaagtttatattttagttggtatgcctagaattatagaaaatatccgtgactatgtttaacttctgtctttatcagacataattctcaattttataagttgatgtgttgataaaatggtgattaaaaaggaagcagatgttttgtgcattaggaagcaatgaagattatagaaagtgtgtaactggaatagctataaccttcttttatttatttattcttctgatttgccatagtatgtcaaaggaaaatattttagatctgtaataattttttgcaatataaaagatattaaaataataaaatgaaagtaattgtgatgtgaaaagtattatgatcatttgttatatttaattctttatagtactaaaccatgaagctatttaaattattttggttttgaaagcagatatttagagttttgttattatcacatcatacttacgatagtccatattgttcttaattagggagtatcaatttactgtagatagtccttaacttctaggagtttataggtaaaatggaaagaatttcaatataattttgtatacagtggccatgccatgaactgccctacaaatgaaataaattttcagtgacttttaaaaatgttagaaacaacctacaaaagcaaaattgtctcctggctccctgcatggcccctgactctactcatttcacaatattctcccattgctctcttagctgcaacttgccttacagtctctttgttataccacaggatctttgttcaaaatcttccttctacttggaatgttttcccccgaccctcccaaatgctcacaaacacataccatgctagttcctatctatcttccattttaactaaaacatcccttcttttcagagctttatcattctaaaccaaataaagtcattttataaagttattttatgatacttattactatttatgattatatgttatttgtgtggttattttattaacatttgtctttttggtagcctataagatttatgagaccagagattatttttggtggttcattgtattcccaacatctggcccagtttttggcacgtagtagatgttgggtctctatatactggatggatgagtgaatgaatttttcagactactgccagaggcttcttattaagatcagttcagttatgacacttttcttcctttgctttcctatggctttaaatactatacagaatactttttattatacccaacacattttgtcgtctttccctaacatacaaatccagttagacttccaattcatcctagatacctaccatcctccttctccatgtttttatctctccctcggggaaggggaagggggagagcatttccctttgtgttcttgttttccttaaactggacctgaagttcccattcctgtgccttttccatgctgttctataaccctggggtgtctttctgtttccttcatcattttcagggatgagcttaagtatcatttatttctgaagctttcccttgttctctgtacataccctgtctgtattaactacccattcttctgcattcctacgtttttacttgttgtagcattttctcttttgccttgtctgtattcttcagtattttttaagtatgttaaagggagggctaatttctcattcaactgtgtatttcccacaatttcaattatagtgtatagaataataacagctaataaaatttaaaaataattgaaatttattattgtcataaattatgaaaaagattgacttagttgtggactatctttaggtgatgataaattttgtattcagagaatattgcatttctatattttttctccaatcagtcccaattgatctgtcacacaactaccattcaggtatcatttcccaccataacatgacactttgcatcagctaatcaacctgacacataccttaatatctctgtgtctttgtccatctattttttaagtctagaatacactttcttaacttacactattttatttatcagaattctcactcatatataggtgaagattttggtcaatattgtcttcctcatgaaaagtttcttgattgctccttttcatttcccattagaatttattaccacttatattgtggttcttaaaaactgcttgttcctttccttaagacttattttattccagccaggtgcggtggttcacgcttgtaatcccagcactttgggaggccgaggcgggcggatcacctaaggtcaggaatttgagaccagcctggccaacatggtgaaaccccatctccactaaaaatacaaaaaaaaaaaaaaattagctgggcatggtggcgggcacctgtaatcccagctactccggaggctgaggcaggagaattgcttgaacccaggagacagagattgcagtgagctgagattgcactgctgcactccagcccgggcaactgagtgaaactccatctcaggaaaaaaaaaaaaaaaagaaagacttatttttttccaacttgtagcctaggttgttgtactccaaagctcatgtgctgcactagtgtgcaaacaggtttgctaatttgtgcactacccagtgaccactcaagggtttgggatatgactagctgaaagggttaatgagtagctgggcttacctgtaatcatggtttctttatgctacatgcatggagtttaaagatagatgaagccccttgaacttttaaagttaaaaaaaaagaacttgacaagagaattcttagcatattcagtaacatagtaaataacttcatatggggtattttatatcagctaaatttattaaaaggtgaatctttattggcctcctgttttataaattcttttttttttttttttgagacagagtctgcctctgtcttgcccaggctggagtgcagtggcacgatcttggctcactgcagcctctgccttccaggtttaagtgattctcctgtctcagcctcctgagtagctgggactacaggcctgtaccaccacacttggctaatttttttgtacttttagtagagacagggtttcatcatgttagccaggctggtcttgaactcctgacctcaggtgatccaccaacctcggcctcccagagtgctgggtttacaaacgtgagccatcgtgcccagcctagaaattctttaaaatgggagtttgaaatggtattactactccattcatagatttgtacttggtctttacaaatttactgcttttaagatttagttcagataaggttttcctttttatagtttgctaagctcaaatgttcactcttaaattaaaaatggttctgagttaccaataaaaatgtatgtgtgagtgtgtgtgtgggtaggtgtgcatgtctgtctgtctagaggaaaaaatattatgtaagtttccacagggattctctcaacacttggtagtagaacagaagaagaaaatacagtggtaaacaaataaaactcccactactgtcttcagcagacatcatgataagtacttcacatgcattatcacatttaaccttcgtattaaccacactttgtagatcaggatatctaagcttggaatgagtaaggcactagctctctaagttttatttgcaataatacatgtcaagacaggatttgacatgtattcatacctattcgatatctcttcatatctattcgataggtaatctgaagagatttccacttgggctctgactagatggacactgatttctaaaagtaaatacccaattcctgtgttaggttcagtagtctaaatatagcccccattacaagatacaagcacagaggaaagactcatccccaaaccttggtgccagaatgaaagtaaggatacagacgcaagagtcttctctgaggaagtggccccagagcaaactgtagaggccatagtaggcaggaatcaggggtctgcttattaaaagttatttcgttataatatattctacaaaacgagtgtaagtatggtagattcaaagatctagttttgagtttaattaaactttacttcttctagaacacaggcccataaccttaaaaagcttcttatgaagaaatgcctcttatctgtgcccaaaatgaattggtttatataccacaccttctaaatcgtttggcaaaaagggagccctcccttatatatattgaaatgactccaaaccacaagaaccataccatggtagcatctgggattaatgtgatagatgctgccaagttttctccccacagttctgataccatctcagccagaactgtaggtctatgtcaaaaatgtcccacagagcctactaatttacttaagctttgaatccgagtaattatccatgatcaattcatacctaatagtattttccctaacatcaagtttaaaaaaaaaagcttttcactgaatgtctgtaacaacaacaaaaatatggccattgccatctttttctttacgtcacattcacaaccctgtttgcaagcatcaccagacctgcaagagcaaccatagatgttttacattttcaccacccttctttaaagtaaactttctctctaattcatgtcttaaaaacataaggtagatataaacctcatctttctattttagagattgcatactggcatatgtgacatgtaattgtagcattgttcattgcatactgtgcatgtggtgaattccatgatatcagaacaacagaatagcaaagtccaatgtcttcgttattaaattaattaaaatgaaatctaaagagtataacaggagttttagcattacttatgaaatactcaaatcggcactattttgtctatgtcttactccagctaatcacattggcatatgtaacttatacaaatgttcactgaaaggctatacacatcaacaaaatgaatgagaaaaagaaaactttcctacagcctagtattagatccatataaattctctgtatgtgtagcataacaaacatcctgactctcatagagctattattctcgttgtaaaccagttatagctaaactcaggctctgatttatttttattgattttaaataaactgaagcttacacttttaacccaattgaatatcctagaaaatagcagatgatcagtaacaaaacataagatttagtggtgattggttagggaatttttaccaaaggacttgctataaaccattatagacctgactgctgacaaacatttacaattaaattttcaggtagttttccaatccatgcagtaattcaataccagcattccctctgctatttatatctactttttccacatgtcattgcctgagcttctttgtctgtcaatctcacatattcatattttttttcaaactggcatctcttgacttgttccagttctctgttcctacactactgtttttgaattgttttatgggcatagagataagtcattggtgaggtgatttgagttcttattcaagagaatgatgacaaacttgaaagtaaaagatgtagtaaatgagccctgcttggtactagcccaacatagtctgtatgagtctgaaatctcatacagaccataataaagcatgcatccgtctatccattcacttgtcaatttatcaaatacttacagagtactagaaatcaatgttcaagttaggtatgctacatgacatttattttcaaaacaaattgatatatagttaaaatatattttaaggtgacccctagtttcactctcaaatttttaaaaatattttaatatgtgcaaatgtgagtccgttacttgtctctctatataaatcttgcagttcagaggttacaaaaattttaccttatttgaattttaatgaagtccttcatgttctttgaacagtttttgacttatcaaaattgcaaatttgtgaatcatatattaagtcagtgttcatttgaaaacagtcaagcctgcatgaaagtaaaagagtagaaattctgtgcatatatgttatatggggcccctttgcgtgattccatgacagccagcagataggcctagtggattttatgataggagccctctgaacatgaccataggagaaactaaacgaagtagtggtcattttgaattactccaggaaatttgaagagagatgtccttggtgccttaaaagtaaaacatttttaaaatattcaaaagtcatttagagaaaaatatgactatgccaaatagtaagcatacttttccttaattaataatcaaaaacatctcttatggttttgaagaattttcagcatcatgcaaccacctaataaatgtgcacaggtaccccgtttctaatttaagcattgaaactaatatctatatctatctaaatagatatagatattgactatgactatgtcaaatagtaagcatacttttccttaattaatagtcaaaaacatctcttacgattttgaagaattttcagcatcacacaaccatgtaacaaacatatacatgtaccccctatatctaatgtaaacattcaaactaatatctatatctatatcaattcaatatcaatatagtttacaaatatcttctgtcattctataggtgtctgtttgctctgtggatacttgggttttttttttttttctgcagaagctcttaagtttaattaggtcccatttgtttatttttatttttgttgcagttgcttttggggacttagccaaaaattctttgtcaagactgatgttgagaagagtattttctatgttgtcttataggatttttgtagtttcaggtgttacatttaaatctttgattgattttgagttaatttttgtaaatagtgaaaagtaggtgtccagttttattcttctgaatatggctagccagatatcccagagctatttattgaatagggagttcttttcccatgcttatttttgttgactttgttgaagatcacatggttgtaggcgtgcagcattatttctgagttttctattctgttctattgttctacgtgtctgtttttatatcagtaccatgctgttttggttactgtagccctgtagtgtagtttgaagtttggtagtgtgatgcctctagctttgttccttttccttaggattactttggctattcaggctctttttttggttccatatgaattttagaattttttttctaattctgtcaagaatgatgttggtaattgataggaatagtgttgaatctatagattggtttgggcagtttagctatgtttaggatatcaattctcccaaactgtgagcatggaatattttcccatttgtttgtgtcatctcccgatttctttcagcggtgttttgtagttctccttgtacagatctttcaccttctgtattcataggtatttcattttctttgtagctactgtaaatggccaaataatatgcaaaagtgctcagcatcactaatcatcaaataactgaaaatcaaaaccatagtgagatgccttcttacaccaggcagaatggcaattatcaaaaagtcaaaagaacaacagatgctggcaaggttgtggagaaagagaatgcttatacattgttggtgggaatgtaaatcagtccatccactatggaaataagcctggagacttctcaaagctcttaaaacagagctaccattcaacccagcaatacaattaattactggatgtatacccaaagtaaaataaatcactcactaaagaaaaaataagcacccatatgttcattgttacgctattcacaaaagcagagacatgaaatcaacccagatgcccatcagtggtagtttggacaaagaaaatatagtaccatgtacatcatgaaatacgacacagccataaaaatgaatgaaatcatgtcttttgcaacatggatgtagctagaggccatagtcctaagcaaattaacacagcaacagaaaactaaatcccacatgtcctcacttataaataggagctaagcattgcacacacatggacatacagacactggggactactagagcgagaagggagggaagtgggtgtgggttgaaaaattacctattggatactatgctctctacctgggtggtgggatccattccccaaagctcaacatcatgtaatattcccatgtaacaaacctgccacatgtaccccctgtatctaatgtaaacattgaaagtaatctctctatatataaatataaactgatacatatatatatgaaaaagaatgtatcacgcaaattcttagaaacttggtaatagattgtagctcataagtagatttgtcacaggatttggggaaaattgaattatacaaagataatttcagataaatgtttatgagataaaaattataaatatgcttaaataggaatgggcaggtaggaagggagaattgatactgtatatatttaacttagttaagatagaagataacagcctaccagttactttcttaaagacactgccccaaagttctacaaaaacttctgggcctcatgatgaagggttctagagggaatcaagtattctgacttctaaaacctcagggtatttatagctagacacttatgaggtactatagttggctgacctcaggaagaaaatgtagtagagtgtttttaaaaacaaagagcatctaaggaaaaatagataaagttattaagaagttggagatatttttatagtaaaagagactgagtaagtatatgtcaaaataatccatcataatgaaccacatgagtatgttgaatgggaatttcaccaaactctacaataccaatgttttggggcctcttggatctcttttagggccaagcaaatcaaagtatttattgcttgtttttacgtagtacaatttgaaacttattcaggattatggcctaacttatgagtataagaaacaaaggaatttaaacaaagttatgaataaggaatgagtaattatttattatggttacaaagaaaaagttgaccttattgagaaccaaaggacttggtctggtttatcttcttttcttatacttttcctgagaaagatgagactgaaactcaggttactgatagatcagtagtccacaccacatttgtggttttaagttagcatcatcctgattcatgtgctctatgctgctagttatttctagaacattaccccttgtattcagacctgatggagtagagagttctgcttaatatggtcaaagggagtaaaatgtgaatagcagtgacaaatctggatacatcttggtttgtgtgctttttaaattgatctccctaaattccctttccttgtaaatgaactaacacaacaccacgtttagcatgatatattggtgacttggttaaattttgcccttctctccatttacaaaattaagttatattttgtgaaagtggtgcttcagtagcgtattatatggtaaaggcttgttacatctagatccttacatcactatttcaaaattatgttatagggaaagtttttctctcattagaaacaagaaataaaggccttaaaatcctttttaaacatattctgctgttttctgtgctatggtgaccaatttaaaatgtatcgcaagtaataaggcatcattttttttctgcgtaaaacataccaggaaaatggacaccaactcaaaatttttcttttgtgaatagtgacttgttccttttctagacaactttccagtagtggttttgtttttgttgtttaaatggcagactttaactctttaaaaatttaattggagtgcaggctttttgtaaaagtaggttatttataaagttctgtgagtcctgaacatcacagacacaatgtggtaacatattatttctggaaatataatagagggaatctagttcatcactagtttgcttatttgattagaaatgctgtgtgttacgcttggtcaagttactttggatcagttacttttaacatggtatttacctggcaactacacactattctgaacctgccatatgttcaaggcacaaagttgaacaaatggagaactaaatgagaaggtgttgataattttagaagagatatttgaggaaaaaaaaaaaaaactcagctctggagccaggagtattttgtaaatatatgagggagattgactgttattatttttaagtatcattctaaagagaaactctttgaaagtgtgtgtgttttaaaaatttaatttgtgaattgaaatttttgtaagtatacatttaaacacaaatctgtacacatgttattattcctgttttttcagaaataaatatatagtaaatttttattggaatatcagaagaaaaacttaacacaactgtcttaatataggttaccgcagcaatttaccaattgttaactaatgagttgcataaaagcttgttagttgttcacactagacatagtgcttatttttcagccattacatcctgatcattctgactattgctaaattctgttgtgtactcctagtaatctttatattcagattggaaccaaccgtctgcttctttctataatatttgccacagagtaatttcttgctcttaaaccttggactttagggggcagtgactaatagagacacatttgagagacacttggtcacttacaatctagcttttcatctttgttaaacttgaattcaagacagctagaaaaggttggtaaccaaaagaagattttgttattttaacttttatcattgcctgcctcaaactaataaagggccacttagacacttgccaagggtaattaatttttcttctttcccctgtccccctcttcctcctctgttgtaactggaagcttgcaatccaattacatttttaaagagttaaagtctaccatgtaaaaaacaaaaacaaaatcactactggaaagttgtctagaaaagcaacaagtccatattcacaaaagaaaaattttaagttggtgtccactttcctagtctcttttattcagaaaaaaaagatgccttattacttatgatacattttaaattgtcaccatgacgcagaaaacagaatttgattttatatatatatgcacacacacatagacacatgtatatacacatatacatgtaaatatatgtatatatacatatacattcatatatacatatataaacatatatacatgtatacatatgtatatgcatatgtgtgtatacgtatatacatgtgtgtacatatatacatatgtgtgtgcatatatacatatgtgcatgtatatacatatacacgtatatatgtgtatgtatgtatacgtatatatatacgtatacatacatacacatgcacaaccgtatattaccttttgatatcaaggatcttagacccaacagtgggaaattgctctaaagagttgacagtgatcattaaagaatcattgtagacattagtcagtctcccaataaacagtaaaatgctgatgaaacacttaactcccagtattctcgagctctggataatgaatgtggtttaaaattaatttaaccagatctaataacaaaaactaaaaccctctaaagtatttcatgtcttgtggataaatctcttatgtgttgctagaaacaaccttttattccaaatctaattttctttccaactctgaggatatgttgatatagattattttaactcaacaagtttgaggtagtacactaacctgagtcattatgtgtatataaaattgcaatcatcccatttccttttggagttttatagataagcttcttacttgccctcctgccccctcatgtacatattttaaattatctgtatacatttttaaaagttattataaataattatatagagaagtaatttggttatattttaatttattttttcaaaagctcaaagtacatttaaatctatacttaaacctagccagtcctgagaaacaactttgagtcacaataaataagagtttaagaaataataaggatcagttccgaattacagtgacaaactaataaattttccccaaagcgtaataggttaaaatagcttctgctagaggagtgggacaagtaaagactctccactcagaacaaccaatgtgttcctaaatagttcttttctcattaattgacattaatgtggacttaatcactcttctctactgactctctgcacttagagtcctgcttctctacctttattgaaggtaggatcaagtcacctctaggaaattcccccagagtatttaattccctggctcaagtaaatgttactattactgttcgttgcagatgatctcatatggaaactctatctatctatctatctatctatctatctatctatctatctatctatatatctatctatctaatctataatggttagaagttgtaacccggtaaatagaaaggcctgatttatagcactatcagcccccttatctgagacattttctgttgacattgatgtataatcaggaaaatgttccagacctgctttaaaaagaggggggcactatggcttgtgttctgattttaacttaatcaccattgattactcttctttcttagttactattcctcctagttggcagggtcccagaactacatgcagtactctagaaattaaatcctattagtactcaatataacaggaggatcgagagagagggattgtcttgtatttcctacttgttgtgctactatttatgcattgtgatatcatgcatgcttgaactagtgattgaggagaaaatagttctaataggaattggcagttagcccagaagtcctactcaatgccttgagcaatattaacatcctctcagtgttatggaggcttgtaaagactgacgttaggatttatttttcataattaaatgatatattctttgttttctttttaaatttaaagatagcagtttgctcaattgttatgccaaattggattttcaactgaaggtaatttgagattttttaaatcaatttgacccttcctgctttggcattaaaactgcatattattttatatgttttatataatacaagaatgacaacaaggtatgtctttatcacgaagaaaaaaagaatacaccaacattctcagtagtttgtttctttccctttcatttccatcttttggaaaattttactgcttttttggttctccatcacacctactcttaccttgctctatgcaatgaacacatgtagatataaatatttttatgtgtttttactgcacagcacacatgctcaatttggaacataagcttaaatgtcaatttatgaaataagagttaaaaaagaaaggtaaaacaattgaattaacttgactgttgtctaatcaatatgtactaatagacaaaacaatggagacatggttttaatgttgccactcacatttaataattgtaaactttatgatagaagagaatcctaaaatttctagactgaaattacttgtcaaatctttctcccaagtttgccctttcaccctatgattttataaaatatactgtgttatacaaggtttagctgataggcttctgaagccacagaattcttcaggatggaaattgcacttcatggagtaaatggaagctatgtaaatagtacatctccttccgcaagttagttttcatttattcctgtttcttttctttttttttccagtcagtgaggttcgttgaagtcatcctgtatttctttctcacttactccacctcattgaaaaaacatcaccagactaaaatttctgtcagaatatctactttcaccctttcttttcacttgcactgccacaccctttatttccttagatctggattattgcatggttggtctccatgcttccaaggtttttccttcctactcttcaatcttgcatatcctcctgtatcaacttatactatcataacattaaataccttgctcaaaatctcacaaccaatctcaagattttaagcttctaaatattgtgctcagttctacttggtatctctgaaataaaatttgcctcaggaagtttgaatatgctagtacccaatgtcaggtattctctctcttcattcatatatcccaactcaagatgcataattactctgaatcctgactctttcaactggatgacatctttcctttctcaagcttatctgatatttgatgttgctatcattttttggcatttaatgatactgacgttttttaaaaaggttaatattggtatctttcctctttaacaaactctataaagtctttgacatcaaagactgtgtccaaagggtgcttaacatgtgttgtacacataatagttgttcaacaaatactttgaagattaatggacaattaataattatgattataataaatgggacattttagtgtaattaaattagatgtgcattttaaagaataataaagcagtcctggaacatcctctccttattataattaaatttgaatttactggtctacttcttctagtactataagacatgagagcattttaaattcctactcaattaagctttaaatgtactttacaattgacagatataaatcacattggaataaaatatccttcttaatgttatatgaagtcagaattccctataatataaatcttgggtaacaatttcttttgcagggggaaacacccccattttcaaaacgatcttttaaagtaattttgataaaacttatcaaaggttgccgatttgtatctttttagttgcagtgacattttttaagtgtagattagaattctctgtgataatctaattgtgctctattgacctcaaaaagcaagagaatttgtgtatttgtgtatgtgtattttgacttcttgaatttttgaatatatctttcaaactcagtttctactacaaaacaggaagctaaaacaaagttatcttaaccttttagtgcatgataatattgatgttactattttggtgtacaatccagtggtcaaatggccagtatatcctgtacacaaggggctcactaacagggtttgccaagaacaaataaaaatcttcttgatttctataaactgtagtgtattcggtatgtatgagaataaaatgacttttaaacatttctttcagagcggtaataataatgacattgattgttcacatttctgcaaataaaatggtcatttattctaaaattaatatagaaggtctttttcatttattgttaatttttgcataagaaatcatatcctgaattgcacctgactaacccttaccattgtcattaaccaacagccatcatagataagaatattttgtgcctttaaaatgtacgttttagatatcacaagttgcattaacagaaaaataacaaaatcattatctattctacatcagaaggaagttagctataaagaatagctttttgggttattttttaattgtacagaattttccagtttttttaaaattggtattggttggaacattatcaatgttaaggtactatgtactctggatttaaataaatatgtagtttttatgaagagaagttggtaatttttacttgaaaaaatctgtttgaaatatatgaatttatgcatagctgagaatcctgtaataataaatatcattagtactagattttgttaacattataaaatatatatgctttgccccaaatgctgaatttataaactttgaaatgaaagcattgcttttgctaaaaatgctttacaatttggtctcaattaatctcaggtttttctgaaaataatgttttagaaaatttttttattaaaaacctcacctacgtatggtttaaagtaacccatatcaatctaattctcaacctgaataattgtaagcataaaacttcatattgctatattttcttagaacttcagtggtagaatttgaaatactaagaacattcagcatagttttagaaatactaatattatgttttattgttttagaggtcttaaattttttttaaatagtttataaaagttaatcagtgtgaacagatacttatttctgaacttttaagcaaacatataatctgaatatttaggtgaggtatttcagggtaaatgtttaaatctgtacatttaggtatccttgaaaattaagttgaaatctagacaatattacacttgctgtgtagtattgatgaaaatagtaataatcatgctaataacaataagaagaataaggaggaggataatatctaatatgtattgaacaccctacatttttgggattattctgagtactttacatgtactcacttatttaatcatctcataaccctttgagatgagtactgttcacttacaaaatttacagaaataggtaatgaactatgaagatggaaataagtaatgatgtgtataactgataaataaattttccaagttgagaaagctaataagtggtggagctgtaatacaaacatgtatggtataacttcagagccctaatcttaccctttacacggaagtcctaaagacatagtctcctcatttgcaaaatggagtcaatactactacaataactcttgctgccatggctcctgctactacttttcatgagatagcaatgtagaattaataataaaacatattttgaaccatacaaatgtaaacggaccctttatcaaacatgtctgtattagttctcacactgctaataaagacatacccgagactgggtaatttataaagtaaaggaggtttaatggactcacagttccacatggctggggaggcctcacaatcatggtggaaggcaaaggggaagcaagacatatcttacatggtggcaggcaagagcacatgtgcaggggaactcctatttataaaattatcaaatctcatgagacttattcactattacaagaagaggatgggaaaaacccacccccatgacttacttacccccaccagtttctcccacaacacttggggattatggaagctataattcaagatgagatttgggtggggacacagccaaaccatatcattccacccctggcccctcccaaatctcatgtcctcacatttcaaaaccagtcatcccttcccaactgtcccccaaagtcttaactcatttcagcattaacttaaacgtccacagtccaaagcctcatctgagacaaggcaagtcccttctacctatcagcctgtaaaatcaaaagcaagtcagttactttcttgatacaatagggatacaagcattgggtaaatatacccattccaaatgggagaaattggccaaaatgaagccatgcaggccccatgcaagtttgaaatccattaggatagccaaatcttaaagctctgaaatgatctcctttgactccatgtctcacatccaggtcatgttcctataagacgtgggttcccatagccttgggcagctccacccctgtggctttgcagggtacagcccccatcctggctgctttcacagactggcactgagtgtgcagcttttctaggtgcacagtgcaaactgtcagtgaatctaccactctggggtctggaggatggtggccctcatctcacagctctgctagacagtgtgccagtggggactctgtgtgagggctcccaccccacctttgccttttgcactgccctagcagaggttctccatgaggacctcaaccctaaagcaaacttctgcctggacatccagaaatttgcgtacatcctctgaaatctaggcagaggttcccaaatctaaattcttgacttctgtgccccaacaggatcaacacaatgtggcagctgccaaggcttggggcttgaaacctctgaagccacagcatgagctgtaccttgaccctttttatccatgactggagccgctgggatgcagggcaccaagtccctaggctgcacacagcaggggggccctggacccaacccaggaaaccattttttcctcctaagcctctgggcctgtgaaggaggggctgctgtgaaggtcctcaacatgccctggagacattttctccattgtcttggtgattaacattgggcccctcattacttatgcaaacttctgctgccagcttgaatttctccccagaaaatgggtttttcttttctatcacatcatcaggctgcaaattttccaaacttttatgctctgcttcctcttgaatactttgttgcttagaaaattcttccacgagataccccaaatcatctctcaagttcaaagttccacatattgctagggcatgggcaaaagccaccagtttccttgctaaagcacagcaagaatcacctttgctccagttcccaacaaattcctcatctccatctgagaccacatcaccctggacttttattgtttatatcaccatcagcatattggtcaaagccgttcaacaagtctctaggaggttccaaattttcccacattttcctgtcttctgagccctccaaactgttccaacccatgcctgttacccaattccaaagttgcttccacattttcaggtatatttacaacagcacccaactcttggtactaatttactgtattagtcaattctcatgctgctaatcaagacatacctgagactgggtaatttataaagaaaaacagattttatggactcacagttccacatggctggggaggactcacaattacggtggaaggcaaaggggaggcaagacatgtctgatacagtggcaggcaagagcacatgtgcaaatgaactcccctctataaaaccatcagatctagtgagatttattcacagttacaagaacagcacgagaaatacccacccccatgattcagttacctcccaccgggtttctcccaagactcacggggattatgggagctacaattcaaaatgagatttgggtggagacacagccaaaccatatcagtgtcatatgaggaggttaacacttacaaagactggctacttatactttttgttggtccactatgactggtgaaatataatacttataaaatataatgattttttatgtgtgaaaatttacctctttaaaactcaggtttttgagataatgtgatccatagagaccacaggattataacaatatacaactcatacagattttaactttcatcttcagattcaattttgctgtttttacaacatatatgtcagtgaaaaaatggccatagttatattaaaagttaaaaatgaacaaaaaactttatcccaacctattaatttaatgataaatttggagttcaaggtttaagctctacctttggcctaactaaagaaatttaggaggacacattgcaaaattcaagaattccttaaattatctaatatttaaatgctgtgtgttcattgaacctttgtaataacccccttataacttctgaaattatattactaaataacttctgggaggctcgacatatttcctgttaatgagctcacaaacaggaaacaggcttaagaaaacaatactgtgaaatatgaacacttttaaaggattgataggaaaaaataaagaataaaacccactgtgatctctaagattttattaggttactccacctgcaaaaacagatgttatatctatttcagatcattttgtcagtgatattttgggcactttctaagtgataatcgatcttgattttagaagttaaaaacctttcaagatttaaagatctctctgctatacactgctatgataacaccacatttttaagacatatatctttttgtaattaagattaatttttcatggaaaatatcacttccatttttattttaatatttttaaaaagtaattgaggaatgataatattcactaatttatataagttgggtttttttgaaactagactctagccttggatatttgttttaaatatactagaaataaaacaataaggatttggttacagcttggaaatagtcaattcatctaactacatgaatatttgagttagccagaggtagtctgaaattggttctatttacttattatttgaccctttaattacataagttgtctctagtccttttctcttttcctcctttccagtatttcccattcttctgcttgaatgaattcccaatctttctcccttcttagagcttgagagcttgaagccactagattgtattgggagtgagagtagcagaaaccagaaaggcacacataccttgttgcccgttttacctaccacaagcccagaagaatacagataataaaccttcatttccaactgtgaagttcaccaggtggaggttaaagttgaaaagtgtatttaccatctgattaaaatatggaccaaataccccaacagacactttgccaaataaaatatgtaggtggcaagtaagtatatgaaaagatgctccacattgtatgtcatcaaggaaatgcaaattaaaacaacaacgagatattgctgtgtatctactatcagaatggcccaaatccagaacactgacaaaaaatgctggcaaggatgtggagcaacagggacttctgttccttattggtgaaatgcggaaatggtacagccacttccgaagtcagttttgcagtttcttacaaaactaaacatattctatatgatcagcaattgtgctcttctgcatccccaaggattgaaaacttatatccacacaacaatctgcatatgaatgtttatagtagggttaattataaatcccaaagcatggaagcaaccaagatgtctttcaatagataaatagataaattgtggcatatatagacaatggagtgttattcagcattaaaatgaaatgagctatcaaactgtgaaaagataaggaggaaatgtaaatgcgcattactaggtgaaagaagcttatctaaaaaagctacatactatatgattccaactgtacaacatcctggaaagagcaaaactatggagatgtaaaaagatcagtgattgtcaggggctcagggaggtagggatgtataggtggagcacacagaacttttagggcagtaaaactactttctatgatattctatactctaatggtggatgcatgtcattacacttttttttttatactttaagttttagggtacatgtgcacaatgtgcaggtttgttacatatgtatacatgtgccatgttggtgtgctgcacccattaactcgtcatttaacattaggtaaatctcctaatgctatccctcccccatacccccacctcacaacaggccctggtatgtgatgttccccttcttgtgtccatgtgttctcattgttcaattcccacctatgagtgagaatatgcggtgtttggttttttgtccttgtgatagtttgctgagaatgatggtttccagcttcatccatgtccctacaaaggacatgaactcatcattttttatggctgcatagtattccatggtgtatatgtgccacattttcttaatccagtctatcattgttgaacatttgggttggttccaagtctttgctattgtgaatagtgccgtaataaacatacgtgtgcatgtgtctttatagcagcatgttttataatcctttgggtatatacccagtaatgggatggctgggccaaatggtatttctagttctagatccctgaggaatcgccacactgacttccacaatggttgaactagtttacagacccaccaacagtgtaacagtgttcctatttctccacatcctctccagcacctgttgtttcctgactttttaatgatcatcattctaactggtgtgagatggtatctcattgtggttttgatttgcatttctctgatggccagtgatgatgagcattttttcatgtgtcttttggctgcataaatgtcttctttggagaagtgtctgttcatgtccttcacccacttttcgatggggttgtttgtttttttcttgtaaatttgtttgagttcattgtagattctggatattagccctttgtcagatgagtagattgcaaaaattttctcccattctgtaggttgcctgttcactctgatggtagtttcttttgctgtgcagaagctctttagtttaattagatcccatttgtcaattttggcttttgttgccattgcttttggtgttttagacatgaagtccttgcccatgtctatgtcctgaatggtattgcctaggttttcttctagggtttttatggttttaagtctaacatttaagtctttaatccatcttgaattaatttttgtcattggttctgtttatatgctggattacgtttattgatttgcgtatgttgaaccagccttgcatcccagggatgaagcccacttgatcatggtggataagctttttgatgtgctgctggattcggtttgccagtattttattgaggatttttgcatcgatgttcatcagggatattggtctaaaagtctctttttttgttgtatctctgccaggctttggtatcaggatgatgctgccctcataaaatgagttagggaggattccctctttttctattgattggaatagtttcacaaggaatggtaccagctcctccttatacctctggtagaattcggctgtgaatccatctggtcctggactttttttggttggtaagctattaattattgcctcaatttcagagtctgttattggtctattcagagattcaacttcttcctggtttagtcttgggagggtgtatgtgtccaggaatttatccattccttctagattttctagtttatttgcgtagaggtgtttatagtattctctggtggcagtttgtatttctgtgggatcggtggtgatatcccctttatcattttttattgcgtgtatttgattcttctctctttccttctttattagtctcgctagcggtctatcaattttgttgatcttttcaaaaaaccagctcctggattcactgattttttgaaggattttttgtatctctatttccttcagttctgctctgatcttagttatttcttgccttctgctagtttttgaatgtgtttgctcttgcttctctagttcttttaattgtgatgttagggtgtcaattttatatctttcctgctttctcttgtgggcatttagttctataaatttccctccacacactgctttgaatgtgtcccagagattctggtatgttgtgtctttgttctcgttggtgtcaaagaacatctttatttttgccttcatttcgttatgtacccagtagtcattcaggagcaggttgttcagtttccatgcagttgagcagttttgagtgagtttcttaatactgagttctagtttgattgcactgtggtctgagagacagtttgttataatttctgttcttttacatttgctgaggagtgctttacttccaactatgtggtcagttttggaataggtgtggtgtggtgctgaaaagaatgtatattctgttgatttggggtggagagttctgtagatgtctattaagtccgcttggtgcagagctgagttcaattcctggatatctttgttaactttctgtcttgatctgtctaaggttgacagtgggatgttaaagtctcccattattattgtgtaggagtctaagtcactttgtaggtctctaaggacttgctttacgaatctgggtgctcctgtattgggtgcgtatacatttaggacagttagctcttcttgttgaattgatccttttaccattatgtaatggccttctttgtctcttttgatctttgttggtttaaagtctgttttatcagagactatgattacaaccgctgtctttttttgttttccatttgcttggtagatcttcctccatccctttattttgagcctatgtgtgtctctgcacgtgagatgggtttcctgaatacagcacactgatgggtcttgactctttccaaacccatagaatgtaaaacagcaagagaaaaccctaatataaactatggactttgggtgagaatgatatgtcaatatgggttcagaatttataacaaacgtatcactctgtgaagacaggaggtatatgaaaaatctgtacctttcactcaatagtgctgtgctcctaaatgtgctctaaaaagtctattagaaatgcacagagatcaatatcatttatgtacaaaacactaagtacatttatacagggagaaaatacccagaagcactttatctcacacgcacccacacacacatactctctctctctctctctcattctgtctctctcacatacacacacactcttacacacaagtggatttactaaagaaaactgaaaagttatatgcaaacaaagcatctttaatagatttgtgcccttctacgttgtggatgctttctgcatcatggagacactttgggacaatgtggccttttttttttttctttttgtgaagaaatggagattgtaaaagtagtcttttctgacaggatgttaccataagaagaaattatctaatagaattaaatattattatttgtctttaattcacagaaaacttgttctataattctaatgtcaacagttcttctttctatgctgtcagaatgagggtttttgaattaacatatttctgaataccagaggattatttttagacaattagaggtaactttttaattattacttttttttaaatgtcaagagatatacatattttaccaaattgatgttgcaataacaaagtaaagtttaagacattttgcccaaagtgataaaaagtagaactttataaaaacttaaagtattgatacttagaatattcataacataatttgaaaagaaactgccttgaaattactcctaagttgcagttcagtaccaccacaatttttgcttcaaattcagactatgatactgttgaaccagtatttctttctctgtttcttgtttgtatgtttatgagataaagccttgatgtgttgtccaggctggcctcccaaatcctaggctcaagcaatcttcctacttcagcttcccaagtagctgggattacaaatgcatgccaccatgacctgctaaaacaatatttctctttttatccttggccagcacccaagaaaactgtggaataaacattgaaaactaaagtaatctggaccaagttttctcagaccgctagtaattactagaaagaaggtgataataaaaatccatattttatagaatatcagaacatttaaaaacaatggaagatgtatttgccctcagagttatttgtataaaattaataatattgcaaattctggaataattttaaaaattgaaaccaatctaatatctctgttaacaaagactcatgccactgaaatttatatgaaaaaaacgcggtaaaattttgatgacatgttgctatatttctataaaatgattatgcctggaatgatgcttaacttctcttaaaattctgccatttaacaaaggccatatcagttttgttttactttagttttcattgcttacatttttgaaacattcaccttttcattctaataatgacaggtggcatggttttatcaacacagtaatgttcaccattcttatgaagttctttgtgagaaggtagcattacacacacaatcagttatatgtactacagagagggcatgaaagttttcatttcttactttaccccttaccacatacacaaactgtaaaatgtaacaagcctggagcttttatgaatacttgatcaaagttaaatttgatgtgcactagaatgtgttggttgtcttcaggtcagcatgtgggtccagaggcttgtatatcccctgcagtccttaaatcacagtctgttccaagatatgcttcattgcaattgcaaggttctgcagaaaacaccaaatacagtattttcattaaaatatttgttttattaatatttaagattttcaatagaattccaagtattatagtaactgctgtaatgtattttctcaaccaaaaaatttttcagactttcagcaactttggtacatttttattttctttttttctttgttttttctttctctctctcttttttttttttttttttttttttttttttgttattgttgagacagagtttcacccttgttgcccaggctggagtgcaatgttgcaatctcagctcactgcaacctctgcctcccatattcaagcaattctcttgcctcagcctcctgagcagctgggattacaggcatgtgccaccatgctccgctatttttttaaatttttagtagagacaggttttcgccatgttggccaggctgatcttgaactcctgacctcagatgatctgcccgcctcagcctcccacagtgctgggattacaggcataagcaaccatgcccggccacatttttattttctatatagtgtttgcttttacctaactgaatactcagttatgtgggacttggagataaattctgtatttactagataataaatgtttatttgttagctaaagtagaattttagttgccaaaacattttttttctttagccaaattaaattatcatagagcaggtttagagactctcctcatataaaccaatattatgtgtgactctggcaggatacacttcctttagattaaatatatgtcaatggatttgaatagcaaaagtggtgaattcattttgtaaaataaacatttacttatttactttttagaaaaaaaatctataataatcctagtatgactgtgtccttattaatagctactgttttgaaaaagaaaaatgataatatttatgcttatgaaaggataaacattaacttctgtttatcataaagaactataaatttggcagaatttactactaacataatactatgcttcattctgctatggggaaaaatattaattgtacatgcttcaattgatgtcttccaatttttaagaaattattcacaaaaaattaaaatctaattaaattagacttgtaaggatgaaatactcaaagtagaaactagaatatgccaatcagtgcattatatgttcattgagtactctggcatttctttttaatcaacagaaggtaacatatttactgataaaactagcaaaaaggcagagagagcaattaatgtttggaaaactttcagaaactgatgaacaaaagataataatcataaattactagttaattcagaagtatagaattgagttttaagatgtaagtatacaatcatacattgaactagttcctacccacaacttcagcaaatgtatagaaggttcatcttaagacctaaagttactaatgctaaaagaaatatattattgaatgatcttataaccatttctctccttcctgtactgttatttatatatcaaattgctatttcactgttcaggtatatatgtagtatttacctaaatttttaatttcttgaaggttttgtactgtttcatatttatttactgtccaataatatccagtccagctcagtggacttactaaatatttgttaatttgctgtactaagcacactcaagttacacatgttcatatgtattggaggtggtgcaaagttgtatataggtagtaatacttatttaaataccttggaatctagcagaactcaatggatcaaagaaagtcaatggcttactcagtctatgagttcaaagttgataggtaaatttcaaactgtacaaaataattatgtattgacagtgatgtaatctagactactgacacactcatagtaaaataatgttgctagtagcacagtagcagagtttaaattttgctaaattaattgtgtgcccaataacagctttcatctttcctattgtgtttacattttattctaagactggagaaagttatttttaaaattttggggtcgaatacatatatttaagaaaatacataaaggtcacttttaaaagttgcatttgggccaggcatgctggctcacgcttgtaatctgagcactttgagaggccgaggcaccaggatcacctgaggtcaggactttgagaccatactggccaacatggcaaaaacccatttctactaaaaatacaaaaaattagccaggtgtggtggtgcacatctgtaatcccagctactatggaggctgaggcaggagaatcacttgaacccaggaggcagagattgcagtgagctgagaccgtgccactgcactctatctagcctgggtgaaagagcaagcctccatctaaaaaataaataagtaaataaataaataaataaataaataaataaataaataaataaaagttgcatttagatagtttaatagtgaccaagaggtattcatatattttactgaagaccacatgctttaaagtatattttcttctaaaggtaagagaacaattttatagtgttcaaatgacatttatgttcatcatttcatattatcctaagaatgatcctttggaataagatggagacaatattatctccacttatagatgatgagattcagattaaaaaactcagcactgtacaatacaagcctacagaaactgaatgactacaggttgacttgaatttttatctatttgtccccaagtcctgtattttattctctgaaacatccactattatctatgataccgaaatgtttttgacttataaaatgattataaatcttaattaccacctctcagaatccgttgcttagttaatatagaggttttctttcttaaatttggtttcacttagctttgtttttaataatgggaggaaaagactgcaaatataacttatgtttataaatttaaaaataaccatcaggaagtgtaaacatttcatttactgtgtttagtttctatccttagatgagctccacaatggtttggacttacttaatatacttaaaaacatttgcacctcttgttcagatcattttaataaatatttattgactacctattctaggcagaggaattgcaaaggaaaatgcagtccttccttgtttagaaaggtaatcatctaagtaaaatatatatggaaaaagtaaatacaatacagcctggttaagtgcaatacaaacactggaaaataggaaactattaactttaaagagccatataaggtcagattgaggaacaacactgaagcaacatttagtaactgggaacagaatattaacagtctgtttatatgaggatggcatcatattatcacaaactataagttatcttaataggtaactatctaattaataacagaaaaatattcttataccaaaagaaagattttagttaataatccacatttttaactggttagaaattataagctattcagcagcatcttatttaattttataggcatactttgcaaataatttttttgggggtatatgtcaggatgatttcatgttgggtggttggctttgtttcattttcactgtccttgttgggcatctttctttcctcaggaggatgttgtctcttacacccaggttatttgttaaagtctttggatctatacttaactgatgacctagaagagcgattctcaaaagtgtgagtcctggaccagcagcaacagcatcaccgggaacttgttagaaatgcacattctcaggccactccctagacctactgaatcagaaaccctggggttggagctcagcaattaccctgcaggtaattctgatgcatgctcaagtttgagaaccactggcctagaagaaatgaactactgcaacttatgaggaatcatgagatccagctcacttttgctcaatacctatcaaaagcttgtcccagagtgattatggaggattgcatgcaggatacacacttcattaaatcctctaaaatctaactttgattaattaagtacagggttattgctgatagcctcataactctaccaccattataatggatctatataaaattgcttaatatcagtagcctaaatttaggagcactatagttttaaaaatacttaattggctactttatttaacaatgttttagggatgtcgttcagtatcttttccagtgtttgctacctaagatcccattggggtaaaaatataactcagatttcactgttaggaatagttctgtttgtgagcaacttacttcctttcatcttacacaatgaaccttgtaatttaattatgattttctcttcatacgagctgttagaaagctcagcattaaatttgtggctcacctctagcaaatatataacacttaatgatgtgactgtcctgattctaaaactttttctcttggttttttactttttaaataaaaacacatttaacttaccttcttatttgtgcgatgtaatggtcattttaagtttctttttaacgacaagttgaaggattgcttaattaattaacaaataacattaaaacttatgtgcctatagaagatgctgaactataccaaattttgccaggaaatagaattcaaaaggattaatatagttttgggttaataaaatggaatcaaaaatgtaatgtcagcagggcacggtggctcacgcctgtaatcccaacactttgggaggccgaggtgggtggatcacgaggtcaggagatcgagaccatcctggctaacacggtgaaaccccgtctccactaaaaatacaaaaaattagccaggcttggtggcgggcgccttgtagtcccagctacttgggaggctgaggcaggagaatggcgtgaacccaggaggcggagcttgcagtgagccgagatcacgcaactgccctccagtctgggcgacagagcaagactccatctcaaaaaaaaaaaaaaaaaaaagaatgtaacgtcaataaagatactcttgtcaaaatgagaaactgaaacttatagtggctcaaggattttttcgtcgtcagtgaggctagttaatggcagaaccaaggtgagaatttatgtcacctaacctagatgtctaagtacttattccattgtttcatggggcttgaagtctttactgctatttaaaattgtatatttacttttaaaatattatgtatacatatattaatatatacaacccattccttttagattatgtattcttcatttatattttatatgttatctacatttaaaatctacagaggaaaagtaggtctttcttttaagaaatatcattatccacagcattaagcattcagattcaaagaaccttagggatacagagatttgagggattagatgaataagttgaggcgtctaagactacttgcccaaagtaatacagctctttagtggtagaatcagggctggaacataagcctctttactccttgtagtagaggttttgctaccatagagaaagacaaacaaacaagcattttttcacataaaaagtaattatgattcgtaatagtgcaattaatgcaaagatgttttaagccaggataaagatggggaattgttgaaactaatttaattgtgcctatctgttcttgaagtagggcctgttcgtttatcacatttatcagtttttttatccattctattctataaatgcatatcacaactcactataggaggtgactgagtgagctttacagtgataaatttttcttgttaggagaaattacatccactaacttgagctacttcagcatgaaattgaaaaagaatcattttgtcttaatttacattcttgattcaatgagaaaaatgtgaattaacccaacagaactacagatgcttgcctcagagactaaaggtacactaaattggtatcattagaagttcatcaatttaaacatcatccaatttattagaatagattttaagcgaggttattaaggttagttacgccaagcctgtatattcatttttctaaatattcatttgttactttggttcaattttattatttttgatacatctttatcaatactccagtgcggttttcatttctcacctaagctgtgcacatcatttcttaatcaaaatcactgggatagtgctgcggttcactagccttaggagctgctatttgccttgaataaattacatgaaaacatgttgattgatagatgttgcatgtcagaaaagaagataactgtatcttcttcagcttactaaaaggtcatgcacacagagtgataggaacagccacagacaactttgagataaagtgtatcatgaaaaggtgtgcaaccttgaagctgtgaaacatggacttgaaagacattttcttttcaaatagaaagaaaatatatccagttacatttaagtgttaaagtaaaaataaaatatactttatcttaactatattttccaatattttagtaatacacaatctctgcttttctcatgtgattaaaacggtgaaattcataattatttttaaattgatcttttaacttaaaatcttgtaaggatatggaaaaatttgagaaaatactttaattttataatacacatttatataacagtcttagggaaatgaaagaaatctacatgtagataaatgtttaatattaataaactggaaacaagctttggtagccttttcttattttaacttaaatctccaacatatctaattgtgtctagaaataatctgtgaattgaggcttatattttgttttctgtttcatttttcaggccatcggcatatttctaaagtgtggggtttaaaactgcatttacctaaatgtttatattaagacaaaaccaaaagcacttaaaataaacctctgcatgtaaatattgtaaatttttttggtcattcagcatacttaaggcatatctgtgctaatgttaagccttatattaaaaaggaaattttgttcttttatgaatattccatatatatgcattcattaacctagctaagttaaaaaagaaactgacagtgttttggaagtatagttttaaaagtaggatggaatgtttttagtttcagaaaaaacactagccatgttcttatctatcccaaattactttgttctccaacagagctgccaaactgtcaaactgtccagcttcagtatataacaaagattctgttgtattgtaaagatattcataacaatttttatggaaggctgaaagtgtataagttcacagttgtagcgataacatggcactcttcactgtttggcttgtttatattggcacagatccccaaagtggagactgacagagtcatgcagaaatctggtatttgagttgaaatttacacactttaagtgatgaactaaacaggctggtgttttctttttcttactcaaaaagtgcaaggttacacaatacagacggttttcatcttttcccataaatgcacagattcctgggatataaatgactgaattcatcaccaattcatcagggacgctgagctatgcagttctgtttacagtggcagaaagttaatataaattttcatcagaaggggaagtttacacatatgattctatttgtccttcattattagcacctgagtctaggctagaacgcataattgtctacaggcacaggccaaatagatgcaagggaaaggcaaataagcacaccaaaaataattttatggttgtctgttacaattctctcactgggattcttaatctagaaaatcaataaacaacaggagtgaaactcttttcctctgctcaggcatggcacagccgaaagtagcacacagaaaaaccattgtgcttctttttgttttagtttattctttctgtgaagtttattggatcccctttctcctcaagcaaaatatatcctgagtcagtctgctattttttatacatttttcaatcatttttttggttcaagtgacagcaaatttgacctgacaaaatagtaccttcttttaatttttgaaaataagttaacttaggaatatatatttatcataaagattttaaaatttttttattgtattaccatctgaacatctgtggaaactcagacattagagtagaccagtgctatctaaggatgacaatggctaaaccacactaatggcaaagccaagatcaatgatcctgattttagggtaatgggcatctattctctaatatatcttgatataaaaatattaaaagtagcctatttttggatatcatgctctatcaacatcaaggatacatttattatatctaattttatatttcattcttgttttaatataatttattttgttggaattattatttaatataattatttaatataatttactttgttggaattttgtcttttaagttaaaataagccaaattaatccaccttttacctgctgttaaaagttgtttactttttcccccaatctggacctcaatatgaactttcatattattttattcttctgattgactttattaataaatagcctgtgctctttttgtccccactggttccttaatacaatattgctgcttctctgtttagtagcctaattttaatgtagttaaaattatatcaaaatttaatataaataaattttacttttattgatttgtagatgaataataatgttcattaaagaaaagaccatatatttctaacaagcattaagaaaaatgtggacttaatacaacttatggaagttcaaatgccaaatatgatggctaggtagtacataaaaataaaatattggaaaaatattttgaaagcgattgcacagtttttgcaaatatatgttttgataatttttcacacaggttaatttaaaagtaacagagttaatttttactgtaatctataaaaatcaattccatttctatgtttttctaatttacttgtcaaaatcttacagtctgtactctgcattaaatgttatattgataattttaagcagttattcagaatcttttattacttttttctctccatttgccctacttacaatgaacatttatacatttctttcacttaaatttctcaagagagtgttgagccatgatgatagtatctgcctttcacctgtacttatgtagattatactcccagcaacatttgtatagtaagtacaattccataattttactgatataccacatttagctgatgttatttctgttccttggtgtcgacattataaatagttccagtatattttactcatttttaagtaaaatttactaaagttatataaagtgtttataatacatttcatcttttatatttacggtttacctattaaaatatttgagaagaggctgtatcaatattcacccaaaaaaagaaaaagagtcatgcagagaatgtcatgtcaacatgataagcatctggatgaaaactaagcaaattatgcacatagacagatgttttctgtataaaatatgtggatagatttttcaggtttcatccagatgtttggaatcatgtggacataatcatgacctctttgtgaccctttcttttccctatcacatagagattaaatgactcagtaaaaagcattggtattagattttaaaaatgctcaggatctcactcagccttgactttagtgctttttcactagaacaacctgaactactaagtctccagacacaattagtttgactgcttttaattaaaaaattactgatttgttttaactattagttggttacttatccattcaaatcatgcatttagattttcgtatatgcatatcctcaaacatagttctgacttctcatttctccattttcctttgttgtctctgtttctttttttggaataaaacctcactaatttatatgataaatctcagattaattaaacaagttattcattaaatcaagattatagggggaaggatttaaggaatcaaattgttttcaaacacttgaactaatgtaggagcactcattctcatataaataatgtgctaattacaaattattcttttctgttaattaagacaggtcagcaggatttctacttggcaacacttagcttaagtgttcaagttgctatatgtacttgaaattaatgaaattgaattacctttaaaatgtcaggcttttcattattttctgtgattatttgcactcattccttttaattattttaatgcagaggggtttaattttatatgcatatttatctatcaattttataaattatttcagtagtatttagagatcaaataataggtgaagttaggataatcactctccaaaaaaaaaaaatcaacacaaaagtttttgtgctgtctaattgatttcagaacaaaactagaaagcccttaccaacctgttacaacttcttttgggggggtcttcatttctaccatttttaaaaccacgtcatagatggcaggtagatattctattatgtgatctatagggtttttgtgtgttcagaaggagtctacagttacacctctctttaattgataaaaatgctgttatgtaaaagtcatgtattttatgtataagggcgggggtagttgggaacatagtagtagacctgctgtatatttctatgctggttccaaactccagcaatctcattctacttacttttttgtagcgtactgtttcttgaacatgtctttatatatactgtttgccatttttagctctttgaaacaccttaaaatgaccaacctttttatcttttctacttccactcccacattaaaaaacaacaaaaaaaagctaataccagtcagatcagtacctgcaaaagtatgtagcacttggcagatgctcaataaatacttgcttagtgaatgaacaaatatattgacaagaaaacagtaatgattattgtttattcatactaaggagacatgaacaatgacactgagcaatttaccaactaatcaagatcagttacttatacattgctatatgaactttggcaagattacgtgaatctgacctaagttttctgccttttttgtttgtttgtcttatttctactttgtctttatgttagcataattcttgcaactccaaaatgtcttcatattttttcataaaactgaggtcttaatcatggtactcttgtatcaattatataagtatactttcatggaaactaagacagaaatcatatattagtactaccaaaatgttaatacaggaaaatttaatcatggattatcaaattgtattaatcactttcatgtcacctctttgaatagttatttgatatttattttcccttgagatatatatatatacatatatatctaatcaaagtatgttttcccattttttatttttataattgcatggagtcagatttcttgaagatgtttaaatgtcagcatcaaaggttataccaaatatttcacatttattgcaacctaatttaaatgtatataatattgtttcaaatattgtacaaattcaaagttcacaaggctccagaactggcctgatttcattgattaagtgctcagtggttggcactcactagggcttcctcagtgagtgatggaacattgaatgtaagaaaagctttgaggagatatataagctataagctatatttattgtgggctaatgtatgtctgcttgttgtaggagtaaatgtcttcgtagaatattgaattatttttcatatcaggcctttaaaagtaaaaagagctgttaaggataaaatataaaatgtttcctaatgctatatttaacaatctttaataaccactcatctctaaaaataaatactgttgatgtttaaaacccaaagaaacactatcagttggtgtcctggaagtaatacattcttctgaaattagattcttacccctaccccccccaaataaaccaaaaacaaactttcgtatcaataactaagaagccgtgtatctaattagacaaaatggaaaatctatctatcctacctgatttctttcttgtcaggagtctaaaattctccattgatttccttaaagccatcttagcaagtagaagtgaagctattctaaataatgttgcaatgacaactacttatttattcataattgtgatgatgtgttttccaggatgcaatttcactattgtgtacatagagtgatgatacttattttatttgaccaaaagttgaaattgaacttgatcttggatctgtaaatatattccttatctcaaggataatgagatgttaaaaactgagaaataaacaaattaggaaattaattttttcgcaaaaaaatgaaaagtctggaagtcattgcttgctggcattgactcaatgactgaaggatgtcacagctgaagttttggtaataatattggcttgtcctttatagtcacaagatagtggggactatgtcccaatacatgcagaaataaggggaaggccacagggctaaagaatgcctgcataaggaaagcaaactcccctgttgacttttgctgatgtgtcatctattaaaactatgttccatagatacccctaactttgagggagagagaaattagtttttaccagtacacattatcttctctaatatagtcatgtttgttagtaaggaagaaggaggcagtatatattgagtattcccctggaagactctgccacagagataggcataagttcatttcacctgagaacaaatgataacattcagcattcatccaaaaagacattaagtgcttgctatatgtcagaaactgtcctaggtgatagaaatgcaaaaatgaataaataggacatatttctcaccctgaaagcatacatactttttaaaaaacatattttattaaataattaaagtattaatttaaatgtgatagatagtggattggctatgaaataacaagcacaacttttttctgcttagaaaaaagtcaggagactacagtgaaagtgaaactcaagcttagtcctgaaagatgagtaggtttgagaaatacacaaaaggagaggcagagtgggaaggaaaaaagctttccctggcagagggatcatccagtccaaaagtgtagagtgattcattcatttagcaattgtttgttagcagctagttgtgaggtagagtgagccaataactggggacacatgaatgaataaaactgaaatgatctttaccttcttagagcttatgatctagtcctacaaaagcatgtggtcttagtgctttggcctagctggagcacaggatgcctgtacagaaggagagcccttatagaacccaagaaatctccttagggataccaattaaggagaagaggagcaggaaacctaaagggtggagctttaagaccccatcctcacttttgacctattaagttttagaaaggtgagccattaaagagatttttagcagagaatgatataatcaagtttgaattttataaggttagaataacagcagtaaaaaggattgatctgggctaaactttaagaattaaaattgggtattcacttagtaggttactctgataagccaacaagagataagacctgagggattgaggcagtagttacagtaatgtagagtaatgaatatagcaaatattaaggaagcagtattgacaagacatggggaaggtttggatggaggcatggccaggacaaatcaagcatatctctaaaagtttttaggcttctattatgaggaacattattaaagagatcttaaagaagtaaaagagcaagctttgtgtatttgagagattctagacagagggaacagtatgtgcaatggtgttaagatggaagtgtgctttctctatttgaggtttagcaaggataccagtgtgtctggagcagagtgagcaaagggcatgatagtatgaaagtcagagtcttcagggtagcaactggacagtttatgtacagattatattctaaacaagacaggaagtccttggaggccttagaaaaggaaatcaacatgatgtggtttaatggtttttttgtttgtttgttttttgttttgttttgttttttgagacagtttcgctcttgttgcccaggctggagtgcaataagttttaaaaggtttatttgggtggctgctctgtgcaaaatattttgaaggaaatggaaagaggaaaatggaaaaaaagagaaaggtattatgaaagtccagagaagaaacgataggagcttgaactaggctggatgcagtagaagtggtataaagttttataaattctgggtacattttgaaggttttgttaatttttgataggttgatgtgagtatgtcggagataaagaggaatcaaggatgatcgcaacatttgtggttagtccaaattagtgaattgaaaaagagcagaagggtacttgtttgttttgttttgttttctttggtggggggggggcttgttaataaggagttcagttgataaattattaaatttcataattttatttgagaaacaaatggcatgatcaaggcaaatggacaaattcagggagttcatgggagacatgggaactgtagataaaaatctatgagttgtcagcatatagatgctattcaagatcatgagtctgagtaagaccacctggtgaattaatgaagagttgagagggaaagggaaaagaagaaaaggggaaaagagagaaaaaaaaaggtgctcacaaacactttgagatcgggagaggtagaaaacatggtcagaaagctgagaaatagcagccagtgtacacagtgataagaaaaaggaaatgtagatgagaactgggaaaagactgacattaaatgatgagcaaagagaagctcacaatgcatttttgtccaaactactttcatggcttattagaagtgagattcatgttgctatatcctattttgaactttcacatttaaagtaagtgtacagaagagacctcctgaatgaggtgaatcaaaattttcatctcagtgtaattgagctgtcctgattgctggtacagacagaagattttcattttccatgactgatatgctattctctcttcatagttaaatttttgaaggccagtatttattccattcaattatctctttatggccatggcatttatttggtgtttaaggcatggtagaacaaagcagaggaaatgggttggaaaaggatggcaaataaaaataggataagaagaagaatacaagagtggagatgtaattcatacttaactgttgcttctgtgaaagggcccattttgcttatttgtaacttacaaggttgattttataatttcaaacttttccttaagatttaccatagacaaatgtggtaatcttttgaaccaaatggatataaagatagaataaagggcactttaaaaagtatgtcttttaccacaatgagataccatttcatgccagttagaatggcgatcattaaaaagtcaggaaacaacagatgctggagaggatgtggagaaataggaatgcttttacactgttggtgggagtataaattacttcaaccattgtggaagacagtgtggcgattccccaaagatctagaaacagaaataccatctgacccagcaatcccattactgggtatatacccaaaggattataaacattctactataaagacacatgcacacgtatgtttattgtagcactgttcacaatagcaaagacttggaaccaacccaaatgcccatcaatgatagactgaataaacaaaatgtggcatatacacaccatggaatactatgcagccatgaaaaaggatgcattcatgttctttgcagggacatggatgaagctggaaaccatcattctcagcaaactaacacaagaacagaaaatcaagcactgcatgttctcactcataagtgggagttgaacagtgagaacacatggacacagggaggggaacatcacccaccagggcctgtcagggggtggggggctaggggagggatagcattaggagaaatacctaatgtagatgactggttgatgggtgtagcaaaccaccatggcacatgtatacctatggaacaagcctgcacgttctgtacatgtatcccagaacttaaagtataataattaaaaaacaaaaagtatctactgacatttctcgatatctcttgatacaaactggttgtgtgctgctgcaatgaaaaaagaaaggaatcaaaaaaagactcttgcaaaggaaagcttttgcatcagtgagtgtgagcattgggggaaggaataatttcactggagagccaaagactagccagatttttagaggagaattaatcagacaacttgccttaggacatgttttggagtagaggataagttaaattttagggaggaggtaatggctagacagttatactacaaaatacaaatttgttcttattaatggtactttaaatttataattcattaataactttaatgaaggatattcattatccttgatattgtgaaggatattgtggtatttccaaccaacttttgctttcattatttttctcttgaaagctaacatattcttttctataaatttgaggttatgtttaattgctaaaattgtattctgcataatcatttactttgtccatgttttaatgttttataattattattgtaattaattttcagtaatatggcttaaaatactagtttctatccaaaaaatcttttaaaacaccaagtaaatgaatagaataaattgcaatttaaataatcatactatatgtcattattattcatttatcagctggaaatgattatattattataacttaagagtttgattttgtttttgtataatttggatattcttttttgttaatatagttagttattggctttatatttgtgaaaagtcatgttctagcgccttgcaacagccttttccaatttatgttattgcaagaccttctcaaagtgaacagaaaggcagagcttttgataagagagaagcatgtttataaatattttcattaagattcactaacaggcagtccctcatttcttaataggcattattacatacaatcatttctatgcccattgataagaaatttgtgttatcatagaaacagtgttgtgtatgatcccatacgaattctcaaaagcttatttataaccataacatgacttaagtaatactaacagagtaatgtctggatcctttgtgtctgagtctagatctcagaggcctagaacagagtgggatggaagtggctccatgtaacagttctcatggaagcacatggagcagtttctcacagcatgaatgagaggagcaggccaaaggtttgctcagtgatttctgatagggttctgtagggaggacagagtgaataaggaatattgtctttaagaagggatgtcatcttttgggaactgaatggagaataggataatctcaattttttaagccataattttgtttttatgcatacactttaaaaattagtagtatagtgaggaaaatgaaagaaaatattgggattcttggggtcaaattaattctattttaaaaactaggctgggtgtggtgactcacacctgtaatcccagcactttgggaggcagaggcaggttgatcatgaggtcaagagatcgataccatcctggcaaacatggtgaaaccccgtttctaccaaaaatacaaaaattagctgggagtggtggcacgcacctgtagtcccagctagtctggaggctgaggcaggagaatcacttgaacccgggaggcggaggctgcagtgagctgagatcgctcaccactgtactccagcctggtgacagaccaagactccatctcaaaacaaaacaaacaacaacaacaaaacactattaccttggttaaattactcaacctctctaggactactttcataatacagtttgaggagatttaatgaaatattctccagagtgcttttttagctgtaaaatgttatattctgtaattgtagtttcagtaattaatacaatatagtggaaacagaaccaacaaatggtcaggggacatctttctttccatgctttgtgtaatgcattgggatatcattatgattaataactagtaatacagatgactataattagtaagggtatcaaaatttacctagtaaagaagtagttaccagttttgacttataattagaatcatttggaaagctttttaaaaaaatagatattcttagaccccacctcaagcctaattaaatcagaatcttaaggagttgagcctaagattctgtctttataacatagccttctgtgggattctgatgatgctctgggtaaagaaccattgtagtagatgtcctcagtgaactgaaatgaaaatttagcacaatttgatattttcataaaaagttttatgtagaagagccctagcaattggtttgttttcagtatttttcatgttctttctgaagtcctatttaaacataaatctgcagagtaaactatttgttcttaaaggtaaatttcagctaaaaagtcctaatttttagaatcttgcataatataattttctaatttaagtgttctctatatttagtgttaaattatgcttttttatatatcacaaacaaaaggcatacaggaaatggcaaactgttgcctattttaaagcaaaatagggtccaaactgctaagacgtatttcaaatgtgtgaaaatatttatgacattgtggccataccccagtacaaattttatcactaacattttttaaattgataagttaacattgtacatatgtgtatatctatatatcactaacatttttaaaattgatcttaacattgtatcttatacacacacacacacacacacacacacacacacgccataaatatatacaatactatgctgtatgtatacaaagttatattgtatatatacatgctgttttcatataggtatgcattgcagaatgactaaattgaattaatatgtattttacctcacatacttatcattttgttatgatgagaatatgttaaaaatctacttccttagctattgtctattatactatactttgtcattaactatcatcactatgatgtacaacagatatcttaaatttattcctcctgtctaactgaaattttatgtcctttgaccaacatctccccaatcccttatccccctagcctctggtaaccaccatattactctatttatattagttcaacgtttttggtttcacatacaagtaacatcatggaatatttgtctttctattcctggtttattttacttaacataatgtcctctagcttcatccatgttgtcacaaatgacacaattttctttttttttagagctgaatagtttgattccatatcttagctattgtaaataatgctgcaataggagctcacatatttcttttttacattcaatttatttatttatttatttattttttatattttctttttctattttattattattgaactttaagttttagggtacatgtgcacaatgtgttatttacatatgtatacatgtgccatgctggtgtgctgcacccattaactcatcatttagcattaggtatatctcctaaagctatccctcccccttccccccaccccacaacagtcctcagagtgtgatgttccccttcctgtgtccatgtgttctcattgttcaattcccacctatgagtgagaatatgcggtgtttggttttttgttcttgcaatagtttactgagaacgatgatttccaatttcatccatgtccctacaaaggacatgaactcatcattttttatggctgcatagtattccatggtgtatatgtgccacattttcttaatccagtctatcattgttggacatttgggttggttccaagtctttgctattgtgaataatgccgcaataaacatacgtgtgcatgtgtctttatagcagcatgatttatagtcctttgggtatatacccagtaatgggatggctgggtcaaatggtatttctagttctagatccctgaggaatcgccacactgacttccacaagggttgaactagtttacagtcccaccaacagtgtaacagtgttcctatttctccacatcctctccagcacctgttgtttcctgactttttaatgattgccattctaactggtgtgagatggtatcttattgtggttttgatttgcatttctctgatggccagtgatgatgagcattttttcatgtgttttttggctgcatagatgtcttctttggagaagtgtctgttcatgtcctttgcccactttttgatggggttgtttgtttttttcttgtaaatttgtttgagttcattgtagattctggatattagccctttgtcagatgagtaggttgcgaaaattttctcccattttgtgggttgcctgttcactctgatggtagtttcttttgctgtgcagaagctctttagtttaattagatcccatttgtcaattttggcttttgttgccattgcttttggtgttttagacatgaagtccttgctcatgcctatgtcctgaattgcttcaaagagaataaaataccgaggaatccaacttacaagggatgtgaaggacctcttcaaggagaactacaaaccactgctcaatgaaataaaagaggttacaaacaaatggaagaacattccatgctcatgggtaggaagaatcaatatcatgaaaatggctatactgcccaaggtaatttatagattcaatgccatccccatcaagctaccaatgactttcttcacagaattggaaaaaactactttaaagttcatatgaaaccaaaaaagagcccgcatcgccaagtcaaccctaagccaaaagaacaaagctgaaggcatcatgctacctgacttcaaactatactacaagtctacagtaaccaaaacagcatggtgctgttatcaaaacagagatatagatcaatggaacagaacagagccctcagaaataatgccgcatatctacaaccatctgatctttgacaaacctgacaaaaacaagcaatggggaaaggatttcctatttaataaatggtgctgggaaaactggctagccatatgtagaaagctgaaactggatcccttccttacaccttatacaaaaattaattcaagatggattaaagactgaaacgttacacctaaaaccataaaaaccctagaagaaaacctaggagctcatatatttctttgacatactaatttcattccatttgaatatacacctaatagtgggattgctgcatcatatgatagttctatttttaattttttgagaaacttccatactgttttccataatagctgtgccaatttatattcccaccaagagcgtacaaaggtttcacatcctaaccaatacttggtaatttttttgttctttgttttttgttttttgttttttgttttttttttgagacagagtctcacactgttgccaaggctggaatgcagtggcgcaatctctgctcactgcaacctctgcctcccaggttcaagcaattctcttgcctctgcctcctgagtagttgggactacaggcacatgccaccatggccagctaatttttgtatttttagtagagatggggtttcaccatgttgcccagaatggtctcgatctcctgacctcatgatccacctgcctcagcctccaaaagtgctgggattacaggtgtgagccactgtgcctgacctttgtcttttaatagtaaccattctttttttttttaaataatatccattctaacagatgtgagatgatacctcattgtggttttattttgtattcatctgatgatgagtgatgttgagcattttttatatacctgttggtcatgtgtatatcttcctttgtaaaatgtctattcagtttctttcccattttttaatcaaactacttgttttcttattattgagttgtttgaagttctcatatatgttgaatatttaacttactagatgtgtgatttgcaaatatattctcccattccataggtcgtctctttattgcttgtttcctttactgggcagaagctttttagtttgatgcaatcccgtcggtctatttctacttatattgcctgtgttttggggctcatatccaaataattcttgcctacatcaatgtcttggaactttccccttgtattttcttctagtgcagttttttatctacatttacagttgtttatggtgtgagatgagggttcaatttcgttctgcatatccagaagatatctagtggatatatgcagtttccctgtaccatttatgaagacattgccttttcccttttatgtgttcttaacatctttgtaggaagtcaactgactgtaagtgcatggatttacttctaggccctcttttctgtttcactgatctttgtgtctgtttttatgccagtatcatgctgttttcattgctatagcttcatagtagattttgaaaagcagacagttgtgatgcctccagctttgtcttttttgctcaagattgttttggctattggggtcttttgtgatttcatacaaattttaagatttttttttcgatttccgtgaaaaatatcatttgaattttgatagcaattgtattgaatctgtggatcacttggagcagtatagacattttaacaatattaatcctccaatccatgaacatgggatatctgtcaatttgttggtgtcttcttcaatttctttcatcaatattttatcattttctatgtacagatcttccacctccttggttatatttattgctaagtattttttggtgactattataaacaaatatcattttaaattgattttgtaaacatcccaggtaacccatggaaaggacttctattataagtctgtacatgttttgtccacccctttttataccaatgtatatataaaatacaaaacaaattaaagtaatatatttcaaaatatacttttaagcaaaggatcaaaacaccgcacagtatttgtaagtaaaatgtgttttaataatattcaatgaaatttatgaaatttaagtgttcttattttccttgtataggaaaatacttttatgttttttccccaagtggaaattgattgactaacttaccagaaacaatgcttgacagtgaattattatacagataccacagtgtaataaaggtaatattctaagaattgcctgtaacagtaacagccctttgacccaaagattctatagaatttttttttttttgagatggagtctctgttgcccaggctggagtgcagtggcatgatctcagctcactttaacctctacctcccaggttcaagcgattctcctgcctcagtctcctgagtagctgggactacaggcatgggccaccatgcccagttaaatttttttgtatttttagtagagacaggctttcaccatgttggccaagctggtcttgaactcctgatctcaagtgatcttcccgcctcggcctcccaaagtgctgggattatgggcatgagccaccatgcttggccctagaattttaagaatgtaaaattttgcttgagttaatcactttgttagttatttgctatcattcttcaagtaagtagacatagaaatgacaggaattgaaaatatcatactgttatttgtcagtttgcatcaccgtcattattcttactttgtactatgtgtctattctactccaccattctcctctagtgcctgttatctaactcatcttcacagcaactatttatttcacagaatgttactgccctgtagatagatgtgctcaaggtgttctttatcttatcccctacatacatttcttgtctcatgtttccaagacacattaaacatggcatagctaagatctttaaaataatttttaattaaagaagagtagggaggtagcaataccatagaagctcaaatcatgagtgtctaatttggtgttggcagggtaagattttgacaagatcccttttgtaaaaatagctatttccatggctgtggaatcacaggaaggtcctgagtgtgtattttggatttgtcctcaaatttcagaatgcctcatttctgtataccagtgtcccagctgttgcagactttgtaatgcattagtattggactccgaaattgagtttctgccagaggattctggcagtaagcatcctcctataacaacagacaagtgatagcagtagaaatcccaggggacttcacaatagcatgtcttaggattcagtgtaatggcttaacttcaacataaagatgatcagagtaagtacagcttattgttccactctggtgagtagtctgttaggtgccagttatgtaaatgaagataattacaagagagtgccaaaatgtgtcctccatgagaaactatgcagttcttaaataggtcattctgcaatcataagaagcctttgaggggggcagcaaaatctcccttctatggaataggtttaatgtcttccgagacaatgggagaaaagggtcatttcaagcagtggccatttgggatttctccaagattacgaagtatttctttttcccagtgtcactctcagaaataaggttttaatttccaattttatccgtctttttataagcattctttcacatgtttcctttctgataacaaaccaaaatgattaatttaaagaagagaaattttaaactgaaatccagttttaaaaaatatgtatgataaaacagcttacattaaatagatattcctttgggcagtagctttatgtattaaaaaagtgaagtcactttttagaagaaactttagcatactctttatcacaggataagttacctaggtccttatcaatcctatgggtaatttgatttttagtagttttgtctcttaccttacataaaaattgcttctgtaaaacaatgacattatttttcatgcttcaaaatatagtcatccttttaaatcactatccacatggaatccctcccatccccttggaattttcatattttttaatgaagtgttcttcctcacagtattttgatctaccatattttgaataactagttttaaagatagagcatgaccaatgtagtttagtgatgtatgtcatatagtatgtaacataatgaaaaaacaaaaagacttaaaagcaacactatcaggtaattttttgcaaactcagacagagtagggaacattgcctctttgtctacatttttatctctagtatctaacaaaaagttacattaataaatattaatttgatagacttaataaaggagttagaaaataaatattagttaaatgaatgagtgaaatatctggtacaatggcccattcaattcactaattctaagtgaaaatacaaacataggtaggtaggtagatagatagatagacagatattagatatgttttttaaaacatcaatttctaacaaaggaggcaaagcaaggtaaaatatttttgcctgttagtttagaaaatactaaaaaaagaatgataatgtatactatggagtatggtggaatgggcattccatgcagtgagttgggattgcaaatgactataatcattctgaaaaggcattatgataatgtgtatcaaaatctaatatctgactatgatttgacccaacaattaaacttttgggagataattacaaggctgtcttataaggaagttcattgcaatattatttaaaattacaaaaatgggaaaagtctaaatatctatcagaagaggattgtattaaagaaaattgagctttctaaagactggaaataatggtgtgctttggtgaatttgaaatttgtttctatttttattgcaaaagaactgtatggtgtttgcctaaataagtgattattttacatagagtactaaagatagatgtacatggatattagtaaatatttaaaagattttaatagaattctaatttcatggataattgagtctgaaaaagcatgaacactacttggatttttatgtaaataaattcgtaaactgaataacaaaatatccattatgtgtatatcctttttctagacccatgcttaaaagggagattagagtgagaccccaactctaccaaaaacaaacaaacaaaacaactgggtatggtagcatgcctctagtcctagctattcaagagcctgaagcaggaagcttgcatgagcccaggagttctaggttacagtggactctgagtctaccactgtactccagcctgggcaaaacagcaggacactgtctcaaaaaaaaaaaaaaaaaaaaagggtaggggtgccgtttagagaatgatttctttttatacttaaacaatctgcctaaaataattgaaggattctaactggagacagtgagataacacttttgtgacaaccaatagtaattttttggatcttcacttaaaaaaataatgaaagaggtaaagaatatattgtggtaaagtcatgggtaaaaatattttaatttaacttgatgaaaagaaatattatgctaatttcagtgttattttagatcactagttggtcctctacagtcattagtgtaaaggtctgtaaaggagctactgtgtgcaaggcagtgtcatggttcctggagggaatgctactttctttacttagtgtaccatttgcatctcagatatctggaaccagctaaaatatgcatgcattttacaagtttaaaaatatctaaagaaacatacttttcaattaaatgtcttttaaatgactaggtgccagctactatgctgtgcattcttggggttataaaagatgatttagaaacatttccagcccttagaaagtttacaatagtagatgttaagggaaattgaatgtattctcctaagaagtaagttttcttaggttaggtgaggtaggttcttcaatagaaatgcttcttgtgcttcttgtgctaatgcaaaatgaaggggtttgcttcctggtttgggattttatatgtgtcttcaacaagtgggccactgacaaaagcttgttaaaaaaacagtggatgagggagaaacttgttttttctaccagcctgagtttcactgtggtaatggaagtaagtgaaaacaaaacagcagtgggatattcacctttaagaacttcgttccctagcaagtcatttcctacaagcaagaatccttccttggtcttccagctactggctcttaaactgctgatagctaccaacaggcaaccattttctaagtggatatttgtacatttttatttatatacatattttctacaatcatattgtagataattcaatataagaaataattttgaaatcagaaatatgttttaacttctttgaggttacaggacacttgacaagatttttagggcttaagtccaaaggacaccgtaaaatttacatggatgtagttacagaattattaaggaatgattattttcaccattagtttgtatcttattttattgaaaagaacactcgtatcacattataatgttcataactctaatttaccacataatatgctaaagataatctaacctaaatttcttaatggctagaatagaaaacagatttctccctcaggtgagtttgcccaagatctgtaaaatgtatctttgtgaattggcttcaaaatgttacacaggcatatatatgcttcatactttaccttacaaacataaatttcaaaaattttatatttatataaataaaaataaattatatgatctaaatataaatgcatttcttctttcatttatttcattcttttaatagtattttcaaattattataatatttcatcagtaattggttctttgtggctaacatttcttatcttcaatgagctttgtgtttcacaatttacattaccctgtaaaatttgtaaattgtaaactgggaagaaggaatttattcagagatcatcaatgatagggatgagatcatacagaaactcagaatggtgctgggattagaacttgaattttctgatgcctccttggcatctctgtcattttgtttctaagaatattctaatattagctttagggtctctcttttagatgttctgagctcactgtttatgcacctgcactaaccaaggcctctctcttctcaaatggtttttccctcaccatccctttcctcccctggttccagcctcggtgacatatgcagaggctatctgcctcaagcctctttcctgtagatctgcggtagcatttccttgccttggtaccatgcagggtcataatctttgtcttggggtttctggctactgatcctcatcatttctgaaaggaaggtgaatagagaaccgaagttttgtttttttctcttctgtgcctccaattgcagaaagctagaatgtgaactctgaaacttttcctctgcttttcttcctctgcttttcttcgactgtctttttgcaaaaacatgttcactcactcttcctttggagtggtccatctcatccttttgctatccattaataacctcgtgagcgttccttctcacagaaacgcagcctaagccctcaagtcactctcctagctaacctgtcaaatatagataagatggctttattactcttatgaggactaaaaaacatttccactgatgaaatgaatagtatatttatgtttaagtgggcaacttggtgtaaacaatcttgttaccttggttacatattctgaaatgcttactttttctaactagagttgtcaggtttagcaagtaaaaatagaagtaaattcaaatttaactgggaatcttctattttatctggcaactctacttttaacttcatcagaaaatagtacatattgtttaatgctttatttatagatgataacgttttgtatttggtatggcagaagaagattattattaaaaatttttcatctttaatattggcctttttttctgtagtgaacatcctaaatctagtgcttaataatttcagagacacttttttaaggggagtaaaattcctttacaatataagcttgtatgtcttatttatcttcataaaaaataaagaaaaagaaaaagaatttgcttggaattatttatgttgttctagttggcataactagcaatgctgttaataactcagaatttgttttgttctctttataccctgaacagctctttatttgagtcttttactgcataaaagtcccttcagttatataattttcctgactaccccttgtagaaagagaatgagcttcagagtcagccagatagttgttcagatctcttatgccactccaaggctctggtgaaaaagcttggacatataacctaactatgctgagtctcagtttatacatctgtgataaaaagagggtgttttgaggattaaatttagaattataactggcatatcatgagcacttaatcattaatttttcttttctttttactttactttgtttttactgaacccttatagtcactttttgtaataccttttaatggcttacagtacatttctacattgcgacgtaattaaattgaatatattttatctttcctaccatactaaaagattcttttgtgcaagtgaccctatacattgggtgtattgtatatgtgcatattcattcagatgtggtgatataggatgggagagtaggggttgtatctcacaatacattcatgaaattatatatcccttcccttaaagtcctagaggagattaaaatacaacttttcaaaatatcatagaacatgactctattaagatactctttctatccaatgggacattatctaacttggacagattgcagaactaaatgcatattgaggtaaactttcaggatttacactatgctagacccattagttaaaatgtacatacctttttagcaaacccactaaagctagtaatcaggagttagtttcaaatgactagtattatatatttccaagcatcatatacctagaagaactgatactcactggaggaacccgaattccaaaagtcttcgcctggaataatactactttatactttagcttttaaaatacttttaagtacattatcctctctggcttcacaaatcacttacatgtgaagttaagtattactagctgtattaaattagagcagtagaaaagtaagtagttcttcatttagtcctggaggcaatattccagaagaaaataccgatatatttatatgtcatcagtgtatgtggaagcacattttatattctaacagcagtttaaataccactttttacctatcagctatatttctcagaacagtgatagaaaagcttcccacttttgtttctcctgaaattccaatctctctctcattctcaaaagataaccacacctctttattttctgaattagttgagaccatccactgtgagctctttccatttcctacctctgcatgtcaaagtcacctgtcctcccatctcttcagcctgtctcctccccataagtctatcacctacctgctgctgctccttaatttagtactttcccatattctagggacttgctcaattttcttattaaaacttacgatttttggccaggcgtggtggcccatgcctgtaatcccagcactttgggaggctgagatgggcagatcacttgaggtcaggagtttcagaccagcctgagcaatgtgttgaaatcccatctctactaaaaatacaaaaattatctgggcgtggtagcaggcacctctaatcacagctacttgggaggctgaggcaggagaatcgcttgaacctgggaggcagaggttgcagtgagccaagatcacaccattgcactccagcctggacaacaagagtgaaatttcgtctcaaaaacaaacaaaaagtttttaaaaaatatttattaagctcattattatgtttctttattaaaaaaaaaaaaaaaaaaaaaaagggccggacgcagtggctcacgcctctaatcccagaactttgggaggccaagacggatggatcacgaggtcaggaattcgagatcagcctggccaacgtggcgaaactatgtctctactaaaaatacaaaaattagctgggcgtggaggcacgcgcctgtaatcccagatactcgggaggccgaggcaggagaatcacttgaacccaggaggcagaggttgcagtgggctgagatgcgccattgcactccagtctgggcgacagggtgagactctgtctcaaaaaaaaaaaacaaaaaaaaaaacacaacaaacaaaacccctcaaactgtaactctcttttattttagtcttcacagaagtaatcaaaacaattgtttgcactcatggctatggttttccccattaattccctagtaactctctgcaaactacttctacttcagtaccctataggaactgatatctgataggtcacatacaaatcagatcaacatgaacttttcaacaaatcttgacctttttgagtttttgatgtttgaagatagtatcattcatcctgtcctctcacaattttttaatatataacaactttattgagatataatttatgtatcacaaaacctacccttttaatgtatacaattcagggagtttttagcatattcactaggttgttcaactatcaccactgtttaattccagaacattctcatcaccctaaaaagaaaccccattctcattagcagtcactttccattctatcttacgtctgggcccttgtaaccactaatctatttttttttttttttttgtctctttaggtatatctattccaggcatattatacaaatggaatgatacagtatatgatttttgttactgactttttttatttagcataatgttttcaaggtttatccatgttgtagcaggtatcaatattccattcacttttatggacaaatattctattaaataaatataccgtattttatctattcatgagttgaagggaatttgggttgtttacatcttttgctattatgcatgatgttgatatgaacaattatgttcaactttttgtgggaatgtaagtttttttttctcttgggtatatattgtaccttgggtggaattaatggatcatgtggtaattctttgtttaaccttttccagaaatcttttggacttctattactgtgccgtcctcacaattcattgtcttccttttcactatttcctcatctcctttcaagaaagtattctgcagtttggggtcttgactcttcctctctctgtggtgtctttaccaacaatctaataaactccctttgccttaactattcacctatgtcttacttttggaaatctgttgtcagtgggatcactctgatcaccctcaacctagagctctgcattatatttactgtctactaaagaaatcttctcttgattattctgccatcaggcaaacagcataatcttctctccaaaggctgtgttttataatctaatttcctcccttttagtagtggcgcagctgttctttcagtttcttttgacttacaattttggagtcatcactgactcctcctagcctttacctcatacccatttactaagttgtgccagctcttctttgacagtacctctcatgattcttgttttgaactaacatgattcatgttattcttatctcttatttggacagttgcacgtacgtttttacaagtctttatacctctgtcttgtctcactctcttcatcccttatctatcctttacaacatgacttcagtattgtcctgggccaggcttattttattgccagatttcagcaaaatttgttcttacctgctatataacatgaagactctttgtcatttattgtttagtttatccaataattagccccaaacaatcttctcaccatgttttactagttcctagactaagcgtatgctctatccaagatcaattattttcttttcactatatataatctcacattttctttgttcctacttggcatggccttctgtcatctccacgaaatgattctcatgtaactccagcccaaatattatatcctccataaaaatttcaatgagcttctcaacaaaaaataatttcttttacttatgaactcctgaagcatttttaaaacattgtctatggccttttgacattcaactggatctttcttattaaacaataagcttttgaagatagaaacaatgtcatatcaccccttctatctccacaagtgtgcagtgctttgttctttgctaggtatatgggatatagtcacaaacatttttaaggacaaacacttttatgtattttattcttccaaatattttcccaaatgctacttctcctacagtcttacttttaagttgaagagcatatgcagaacagaggattcaaataatagtttacatttacatattgtagtataatttagtagcatgtatttttcagttaatatctgatatactggttgaaatcatagcaatacagcagtaatgagtcttcaatacatatccaaacaaaagatgatattggtctcataatgaaaaaaaaaaccctgctaaggtatgtgtaaaacaaaaagtaaatctaaaatctatttgggaaaataataaaatgttttatagcagaaagataaataatcaaaagaatgcacaaaaactattatagttactggaacatgtcccatgataaacagaagttgtaaaagcaataattttagtaaaatcagtcctaatttatctttacctttcattccttagaattttaaagttggacggaattcattaatatgtatttaaaattattgtaggtaatattaggatattacattagcaatttattaccattagcaatagaatagtgaagaacatagagctaggaatatcaggctacttgatgtactgcaccttatagaatgctgactctgaggaagagaatgtgttctcccttttaaactcattctacagctgaaaaaaacaaaatctgtcttagatgttagttactgttaactcacaggcttatttcatagaatttctgaggtcatctgggggtttggagccataatgaaatatactgaatattgggtttggacttgaaccttgaagaatttattctcacttctgaggatgacttgaggatccattccatcacacattcaataccctgattgtctctcctttggctctacatcataagatatttgattggtaaataggatgctttgtaagtaattactcttattaaagtactcttattatagtgtagtattattttataattattatacaataagtagtcttagtatctcaatttggcctccatagtgatgtacagtgtaaatatatccacacattaattttctaactttatgtaacatatactgtaaaaatatcattttaaaaatattctttaataattagcttttaaaacaaacagtctaattatgtatcaggcatgaaaaaataaaaggtcaatagctacagatgcataatttcactctgatagattgctatttgaaaataggttatgccaattagatgattaattttcttctattaaataacattatgagacttcggattacatgtatcagtatattctctttagtgggaacataatatgaagaaaggccaaacagaatacgaatttatatcctttctggaaggatctgaatatactttttatcaaccacacttgggaagataattagaaaattaaactctgtttgaaaacttattttatgtttcgtattcaaagtaacatgttgaagacacaatgtgttaaaggttaaactatatgaatatgaggaacaaaaatctgggtgtcctgctggcaatttttttttggttctggttatctgcaaagtatacaccccttgtcagtataactagggagtgactgtgtaaacattcttgtttcgcctgttccacataatcgtttcttgtatcatatttcttctgacctcactatctgaaaccaattacaggagatccatttgggctgcatctattttcataagaagcctaggcaacagtgtttttctcagagatgaatatcaacatcaaatatgcttctgagtgctttgttatttatgtttcagagagattttgtagccttcagctctcaagaatgtgtcacgctgtgctgtttatacattgaagatattaatgggcgtagatgtcatttttcattttaatgtctcaaatgcaacaggctgataggttaagcattcctatttttatggggagatatatgactgatgtctgttttttcccctaatatacatatatttactttgaaaaaaaagttatttataaaccttagcatactcaacattttttttgttttactgcgtatttctaaactattctcaagtaattttatatttggtgaaagttttcagtgttcttcagtggaaaaaagtatttatctggcaggttacaaagcattttcatccttagttaattacacaatgacctactcattagaattattctgtgaggttcataactagcataaaattactactagtgaagaagtatgcttgaactttgcttttcttacaaaaatttagttaaaattgcacagtaagagttaggaatgatgttttcagtaagtattttagagaaaaaatgcactcaattatgaaaaatgtgcattcccacatctctgcagcctaacaaaatgtttacctttaatctcttataaagattatttcttttttcaaattttttaatttaacctgatttcaaattttaagatgtcttaccttaattttctttaggtttcctaaaataggataacttcattaagtaatgtgcacccctaattaaaaacaaatgtgtttttggaaactagatatggtttggctgccttcgaaatctctttcttgtttctgatacattggaaacagactcaatcattgtgaaaggaaattttgtaaacaacttttgcttttgttcaaaattaatttttgtgggggagggtgaccatctaaatgactagatggcgcagagaactttatgaatggccctggatttaatattttgtttgtattgatagaagtggcaagtgatttttaaattaatatttgtgtcatttgacacctaacattttatattgatggaagtgttttatctaataatctttcattttttgagtggctataataataaattgcaaagatgaactaaatgtcatctaattgctcagagacctgagcaattgtttattgaaattcagtcctaaagtgttatccagaatactggaaataaaaatgtcatatgatgcatgcagtgagaatgcaaactgccagagctttctgcttatgagttttaaatcattcctgatctcctagaattgttttttgttctcccacctcttatgtttactatagcagtctctactctaccaaaagaatgttgaaataggaaagaagttattttttctaagagtttgtattaaaagatgatcctcagctattttggagataatgcatatgaaaaccaaaatactttaaagtagaagcatttttttagccagctatcttgtgatatttcattactattattactcctttactaacagtgtaggtgtatgcttttgactagtgatactttgatgagcaaaacttgtaatatatttaattttctagtgtttgtctacagacattacgtagcattgacatattccattgattacatgggttatgttgaatgaatataacgcaggttggtaaaatggatggggtcctgagcttgggagagaagagccagagtttttgaccaggttctgctactgctttgtgacttcaatcaactcatcctagtattgtttcttcagttacttgtttgagaaaggtagtctagacagtttctaaggccttgtccagctctagaattttgcagttctatagtaataattgatcagatactcaggatgttacaaaagtttagagttccatcaggcataagggaggtattaatataaaatgaaatccgagttaaggatgatgagggcagtagccattaaaacagtatatattgtgtgatagttgttctattcgtttacagattcaaaacaaaaaggttatattgttgtttcccatgtaaagtctgaaaaatgaatatgctctctttactagtgaagaataactcattataatgtcatatattctcctttactagagtcttactcattgcctactaatgattcctttgacagaacagaaacagtatggcacactggaatacactcaggcctggaagccagcagaccttgaacaaagcattcataaattgctttgtgatcttgaacaaagcaactgactattctacgtttcatttttcttatgtgctagaataaggaggtcagattagatgattttcaagatagcttttagtccttgtcattcatgattctaagatcctaaacagtgttcaataaattttttaaagaataataaatagataactacataatttaatataaattaaggcaacaggtatacaaaataaactcatcaactcgaaaagaaaaaaaaactacatgaaaacaattaggcattaactggaaaaataatcaaatgcattttttattttgagaatttcagagacacagcctgggacacctattgtaatttccatttggaaaattatcatttgtagtcactttataagttacaattaaaagaggaaagatatattatttagaatattgaataaaaagtttcaaatagtgagccttgtgataataaagtaattacagatttttttgtttgtattttcaaactgagctctataataaatagcctgagttcactggaatgtgccatccttagaaatatttacgttaatttgtaattacttgaaaagttaaaaaacaacaacaaagatgattatcgtgagtgagtgtgtctccaaatgtaaaaagtgtgctttacaaactatgccagatcctgctatcggaagacaaatcaatgttgttaggagatggtgctagtgcattcagtttgtttactttggtttctatgtctgcctgccttctcagaaaacttgcttggagccagactgtagcagccctctctgtcttttcataattgatcaggggatgactgccttccctgagggttaagacgtggcaactttttctgtttgctcagcatctcaatgcagtagttcaaacattgttcacaccagctggcagtgtaatgccatatgcacatcttgtcaaagcaactgttgtcatggatactagaaactctcttaaaaaccagatcattgattacaatcaaaattgtactttttatcagctgaaattcttaattattatataggaatattaatccaataatgttttctcattgtgaaaaatttatcttgtattcaaaaacagtaaataacatattaggccaatgatttagtttagttttatgtgttgaaaaatttcatcattagtgttcattgaactcagttgttgagagagaaaatgaattttttaagagactcttaaaagtttttgttaattagtgtatactcctacctttagaagaatttactatgcattttggtctatggcactagcttttatatgttaagtttatatagtacatttactgaactgttcctcctccagtaaatgaaatgcttacattaatttcttatgttaagttttattttcctaagtgggaaagaaaacataaaaataaatgaaaacatcttctattttgattggtattctgtcacacatagaaacaaacctgattggttgagtggtaatttctttgttctctgattttgctaagaatgcaaatgaaaaccactcaattttatttggcattaaaactgcactttattttgtcaaaggagaggtttccccttgaattgtaagtacaaaaaaagtgtttttaatctttagtgaaaatccttattaattcactttcaagataatctgatgttgatttgtcaaatttaattacagcttccggtaacacttgaagtctactgcttttgcctcaaaattgtaatatgaaaatgcaaattgaatgataatttaaaagggagtgggcattttagttaacactcaaagacaagttccttgtggaaatgaggttattgatgcagtttatgatttttatcctctttaagcactttaaacaaattaactttatttcctgtaggattgcatgtttctctgtgatttggagtaaaaagttatatatgtgtgtttggcaatttttttcatattagaagataaacaacaatgattctctctagaaaggaatatgggagttcttgtacattgaagccttttactattaaccaagagataattgataacttaactttcaacaaaatatttagataaggtttcatttttacttctaggctctccaggcagcaagacaacttcttttacagcagcaaacaagtggattgaaatctcctaagagcagtgataaacagagaccactgcaggttagtaaagcactcctgtccttggggtcttattttaaaagatgttcataatgataacagatgtgcagacccacttgtatatatacaggaaattattacatttgcatatgtaggtaattttaattcataaagagaattcattttatcttcttaaaggagaaaaaaataatattctattggcatacattttaaaagtttacaaggtaactccttgcaaaaaatacataggaaccatatttggtgtacataggcataaagaattttgcataaaacccccttgtgggattttattcatacataggttgaaatgtaaaatttttttcttaaaagcaaagttatagtaaaatggtgtggaaggaaacaaagaagtcttagacaacccttccgcagttgtatccaagtgacaaatgcagaaaacttcgaggattgccaaaaaaactgtttgagtttgccaaggagttattgctggtgctacagttataaaaacacattctggagtttgttgtccttactcactatgtttgtcctgtgaaactgatagtatgtatgtcttttaaagtaacttttgttacataatataatccttgttttatgcagtcttcttaaccaaaatatgtgggacattttcttggtggggggggaaacatttttaaggtatattttttgactgtttggaaggtaaattctggaattgcataaaacatctctcaaagtgttcaccagctctgttgaaagataagtaatgctttttcctgaatcatgtgttatatacatgtctaataaggcaaatttagttttttaacatctcttgtggctttaacaaaaaaaacaaaacaaaacaattttttatggcccagctgtctgtttttattttcaggtgtacttacagatgttagtaactaagtcttacatgatggccttctggagcttagtttgtataagagagaattataagctactgatctgtacaatccaaaaattagttattacctaaaaactggataaaatgtatggtccctaaacaaatttgagataagaaccgattttcctgggaacaaagtcatctgtggagggtggatgagaagaagagggacttggggataagggatgtttcaacagagtacagatgtctgccatgactggaaatgaaagttttatttaatcaccctgagtttaagattcttttcagtttttttttctcttttaaaagcaacttcacattcctttgttttaactaatgaataattctgtgtcactttcctcaccatcatattcttccccaatttataatgggagcgtaggtgtagtatttccatggagttatctgattgcatgagagtctcttcatgcatttacatgacagagaaaaatttacatagaagtcacagaagatagcaaatgaaatgtttatcctcactatggcttcagagtgggaattttgctttcttgttggaatagaaaaggagaattagatgctggcaacataagcaaaagataggaatgaagttactgttttgagtgtctaaaaaacactatatattttcctaattctattttatcatatttcttgacaaaatagaggaattcgcagttgcttcttcctctccaaagctaagtccagtagaagtttctatattgctggttaccagggtatctctggaaagaatggctgggctttagtttttcttttctttttttttttttttttttttgttgttgttgttgcattgaaaggcaagatttgatacttggactaaaatatacgtacttttccttacaaaatacccacaatagaatgtgggaatattaatggctctaaaataagaaattgttgtttgtttatcaatgcagaagtaaaattttgagcagaagtgtccagtcttgttgttttagcataaaagaatgattaaaactattataaataccttttaacaattgcagttgctgaccatgactacaaagactttcattgtgtcacttgtttttcattttgtgatgcatattaatctgaatttatctccattgtttagactgtaaaactagcatttcagtattagtttgtaattttaatgagatctcatgccaaaatgtcataggaacgagaagttgagttttatgaatgagaatttagggatattgaatgagacttattcaccaaaatatacactgcaattaataaaatgtgagccttcatttttagtatttcataggacttgataagcttagcaactggagacacgctgggattgtattaaaactccaataaaagcttaacagcatttgatactagcttacacatctgttgtcaaacaggaatattaggtaatgcttatccttgtatcttcaaatattatttagcatgagttgcactatttgccacttatgtttttaaaaagggtacgatgttttcattaaaattttcaataatttgatatgaaattagcattttaaaagttttcttatataattctctcaaaatttatatctttttctacttaaccaagtgtttaaccaaacattcataatagagttagtgtattatctttacatttcaaaaggataattgaaaatgtttcaaaatctgtaggtgcaaggcaagatatttggtattaatcgattatgatggttcttaatatgtcaggcataacatctttagagaaccaaaagagatactttgtattttttattttgaagtatagttttgatagagaactattttataaagtttgaaagaaaatacagatttcaaaacctgttttgatggctcaagtcacaagaacaatattcagttgcttgttaacattcacctgaatttcttttctctgttaagaagtggtggccagatttgaagtgtgacatggtacagagtctatttttagtagactagtatttatctactattattttttcatttacatttgttcaggattttgtgctacagatggcatagatattagcatgtctaatgcaaaatggaaagaagaaaacatcacatctgacaagcttgagtgccaattgtagtagtgatttcactttcatgtatagatatctgaatatatgtatttgaaacatgtttacataatctcattacatgaaacaccaattgaataggtgcaatcagcaagtacattgattaatggcatattgatgaataatatataaagattgtgatgcaaatgggagccaatattaacttaagcattaccagatgtttctgctttacattcttactgtgtataccagaaaacatatcctaataataacagccaaagacagcttctcactttaatttgttttatttatttaaatctactcctagcacttcctagacttgatatgttcatagtgatccataatgaggaaagttagccaaaaagaataatacttaatcctttcaatttcatgaaactaattttcagtttagaaagagtaagttaacacatcaccccacacacacataaatgccaacatgcacatacacacatgcacacttccataatctgtaaatttaaaaaaatttttgtaaaatacattttcaattttctagcctatgacaaatctacttaatttagtttagatatgaaaattggtcgcattattgttcttgttacatttacagccatgagacagacttgtggttcttcaacagctgtgcagagcagagacgtaaatttaaggtaaatgccattgctttgctatcttagatgaatattagtttttagcattgtaatcacttccttcattgtaggttaatatataatataaagttctcctttctaataggtaattgaattataaaatttttataattctgtcttaagaagttcaagaatggaatagtaaaaagcaaaagagaattaaacttgggggtcttattctgactctgccacaaatagttatataagtctgaggaagttacttgacatctttaaaataaagatgttgggctaactgatgaacaagatcttaaattccatggttaaatatttattttgttttcattttgaagtaagtgaggtatcttaatttttcaggattgggtatatcttgttggatattagcttatgaagtaaggattatgtttttgtttgcttggtttttgttacaaaacatttgtttattaacatgccccatccagaaaccgtgccttggaaacaaagaattaaagcccatatgtatggaaaaccattgcgcaaatataattctgtatatctgaggaagctttgttttgtttcatgccttgaaatattataaattgaatttatgagaaatgtaagctacataaaaattatttaaccaatgtatttactttaataatctacattgaaatgttttgatataatgcaacttaacattatttttactttactatttcattccaaattgttaaaaataacaaaagctgacaaaaccagaaattctatagtaaactaattatcaatactgaaattccatatcatatgtaaaaaatcaagaataagttataaaagtttataatttgtaaaatattctggaggaaaatactaacttatatgggatatagaatcactcattacaaaatgtattttaggaaatgataacattttatttatgtagcgataaagaatgaaactgaatatttctttttcaatattacttttaaatataatgtagaagaagtggagagaagctaaaccacattttattccaaataaaagcagcattgccagaaaaattgagatacgggtttaaatagagcgatctattatgatgccagaaatttagaaataagtttttaaagatattctttactatttatattttctgtgtattgaaagatgaacaggaggtctaatacattatttcttgatcataaaaaaaaagaatttaattcaaatactgtttacaagaacagactatgtcttgtgtggctcttattttcccattccagtgaagaacatttcttaaaagcagcatgaaataggcaaatttaaaagaatgttatagaatacaatttttgttaaactatgatgatgtttctatccatgaccagctgctcaaggctgacaccctgtggaggagggtctgggacattgagctcattactactaagcatgcttgtgtggaaaacttaccatatgatctactcatggcatatagaagaaagaaccatgtgtttcagatctgaagcctgcccttgccattaatagggctttggatagaataacctgatctctttggagcctgtttcctcatccctaaaataggaataaagatacccaacctttcaagattgctgtgaagaccaaaaattatattatataaagtacttaccgtaaatccttggataaggagttaataaataattaccattatgctagcctccaggaaattggtatttaattcaaggttatgatatatattctatatcatagcatttggccgtaatttgttactattaccagctatttcaatgatttgaagtatgcccttgttgtaattgtgtgttttaaggaatactagagtgttttcccctgctataggctcccaccacatatacacttcacactctccaaacataaacttatattttaatgtttatcttctctattaaactgtaagcgatatgacagcaggaatcatgaattccctgctcttctctagtgcctagcacagtgcttggtgaataatagagctcaataaataaacttgttgaaacaatgaataaggaggagattgtgctgagtggagcaaaggagatttggaggaggaaaaaaaacatccattcattcagtataaattgagtgattatttatcatgtgtaatgtcctcagggacttatgacttagttgaggagatattcagagttgcacacaaataaaaacattattggccacagtggatgtgctgtagaaatgggaaaaacagaaagaacaataagcatttaaaaaagaaaattaacaattcaggctacaagtggtcagggaagactttgtagagaaggtagcatgtatgttgggcttaaagaaaaggaatttggtagataaaggaatttttgatggagaatttttttaaagatgtcatcaaagatgtagacataggaaattctgagatctggttgggcaagtgtaagtagtttgttttgatcagcctagagatatcatactaatgtaataatgaatgggttgggggttagaaatacacatcgctgattgtatagaacttaaaatgtcaagccaagaagtcagtattttatagattgggtagaaggggctattaaagttttttgatgaggaagtgaaattatcaaagcagtggtctgtgaagatagcaatggaacatttggaggatattttcagggaagggaattctgagaaacctggaataaggataatcctttaaaaactatttaggtaggcatgatgatgacttaagctttagacatgttgagtttgaggagatggcaagacaagaaagtagaaatagctagcttttagcaaatttgaaagaaagcttgacagaggggtcaagactaaagagaaacatttggggaacaacacagaagtgatagatgaatccatggtagtagaggagatttctgaaaaaggtaatttgaaaagaggagctcagtaacattcacatttttttagagcaagaagaaaaagcagaagagacaagaggggactagagcaatcaaattagcattggatttattaaggaaggtcaacaaaagggggagtttataaaatttaaggcttttcagccacatcaagcaatgtccacaggaaggagaacatttttgcataaacactgtttagttgattgcaacacagatacttttaaatccccctcattcttcatcattcatcagtaactcttaggatctgcagtagtaatgtgatcaataatgaatagagaaaaatcatactttaacatggatgataaatagaaaatagggaattaataaatgtacttttgcttctcagattgcagtgaaagaaaatgtatgttgcatatataatactgaacttccataaattaaatatggaagaactttgctatctaggaggccaaattttcgcttttcttagctacccaagcacttgcttctttctgtaatccattaggattttaagtatatattcccttgacttctctaccttaactaaagtgagagcaagaaagaaatagaacattaatattgttatgggagctctctataggatttaagacttaggctgtgaattttatcttaagatgtttggaagtgttatacaagctaaattgaatgacaaagtgaagaacagtgtcagcacatgattaaagacaaaaagtaaaattattttgaaatttacttacatttaagattatttgtgtttcctctgtggattctatttatatatttctatcaacccaccttccacatttctattggttttcttaaaatgtttctgctaccttcaccttttctactgctttcttttctcctttaccattttattttcttctctcatcatgtaatcttgtctattttactttattgctgtctctttggtcccataagaacacagaaactttaaatatctgtaactatctaattctatcatgaacctaaaagatattctaacatatcattttactaacaaaataaaaccgtgcccattcttacaatccagtatctaatatttaattcttgtcttacaccaagaatagtttgtgctatttaagttggagtttcaataggaaagcacttctccacttgagtatctcagctacttagaagaacctggaaccctaaggctttttccttttctttttagtgctagaaatctcctgttatgacatttatagtggctttttaaaaaattgtcttcagagtggcttcattgttccttaatcttttggtctgctttttcagaaaatctattacaaaatgaatatttaaactagaattgcttgggatagttgtattaaatggtcagttcttctaagaacatctaagattgtgctgattgtgattattttagtaatgctgaatttggaagcacagcatttggaaagatgaaacaaatgtggaataaatttttttgtttttgtttttgttttttgttttttttttttttgaggcagaatctcactctgtcacccaggttgtagtgcagtggcaagatttcctctcactgcaacatctgcctcctgggttcaagaaattctcctgcctcagcctcctgagtagctggggtcacgggcatgcaccaccatgcccaactaatttttgtatatttagtagagacggggtttcaccctgttggcctcgaactcctgaccttaagtgatccacccaccttggcctccaaaagcgctgggattatagcatgagccaccatacccggcctgttctttcaaaatatttaagtcaccctatcttaatatgaaaatcatataagctcattacaagaatctaacatgattttaagttaaattatgtttggtcactaagttccttttcccaaaggcacttgataaatgctaataaattactatagtatagtggatttttcaaaatgaaaattgtattctctgtttagatgaggtagaaaccactcataagaactaattatagtagaattatagtagaccctcaacattcacaatgtgtatacccatagatcttcatgaatcctcaaattaaaaaaaatttccatttcaaaaatttggtgaggtttgtgcagactttcttgtatactgtccttggtttgaaccaagctaccattgccttatacaaagtctaatacaagttaacaactttaaaaatttcaataaaatttgccctgtgattaattttaaattttcaaaaaagaaacttttttacattataggttctcctacattagtgaggtagataacagcaaagctacattagtgatagaaaaatccatatgagtttaggaattaggttatgtgtgtgagaagtcagcaatctgaagaccagatcaactttttagctacttgattgtttcatggcttgaagaactgcacaggtgcaatgtctagagacttagagatcacatgtaaataaccacacaaatccataacatctgtgaattttaaagctctattctaccaaatcttgccatctctcatctatgttcacccaaagtgaacaaacaagtatcccctactttccttgttattttattgaagacttgagagaagttccctgtcttaaaatttttatcctccttaattgtaaatctccactggcatcaattaattaccgggaaatgttccttcagagtaaaagttgcctagcatgtttttttctttttcttcttcattttctttttcttaatagaggcaaagtctcactatgttgcccaggctgggctctaaatcctgggctcaagtgattcttctgcctcagcctcccaagtgtcagggttatagtcatgagccacagcacccagcctctagcatgctttttaagggaatagggaaaggccgtgtcttgagtgtgaagagacctctcacaaatattgagtaaatcagtagagactttaatggccattcattgtttcctagtttggttcagggattctgaactaaggattttatgcgcccccaccctgcccccacccagttagagagcctctgtctaaatccagttaaaggtcttgaatcacatcaagctgtggctaaagtttgttatttgattgtgcctatttttacagttttaaattttatcagtatagacacagaacctttaatacctaatagttaagcattttgataacacaaaatacacagaaaattcctttgcatagtatataaatccttacacttttaatgctaacacttgcattgctgtaaaagctgccaccttgacatcttctactgctagtgctactgcacgttctcaagtgacaaaccaaggaacacattgaacattatgatgtgctgacatgaagaaatctgttgtctcacagaaagactgtagcaaattcttggatagaatatctctgtaaaagcaaacaaacataaaagacaaaattattctctctacattctaccttcttcccatagtatccagcatttaaaacatattaactggtgattaaactatgactaaaattatagtttctcaaaatcatggccatcatttgaagttttccttcatttcaggagaatttattttccgtaatcaataacaatcaccactgccatacacagtcttagaggatgtttatacaattcattttcaagtctaaaaaattacttgcaagtcttataaaaatggagatcttgggtctatcccagactactgaattaaaattgaggaacgagggatggaatctttggaaataaagatatttaaaactctccaggagatacaatacagttggcttacaatacagctggcctacagagtagtatataagaaccctgaactgttctcttttggaacaagaaaacagtgaaccccaaattctattaatatactatatgattttccctctctgggattcagttttccaacccttgcaatgaaaaggctgaactagtgatacttcaggttacttccagatgcaacagttattgaatctcatcccagcactaacttttgtttgtttatatatttattgtctaaatctgaactaattaaaaaggattctgagcaggtttcttgagatgtctctctctgatcagccttttcaggatcatttccaacaatattgtcagaattttatttttactattatgatttcttatgtaaaatcttcagtaatgaactcattcccttcttatctttaattttttaaaataatatctgcatcctaaactcctcagtatatatctgctacgctcaatgggcttccctttggtattagaaaccttaagaaaagatggattgtatagtatcaatcaatagattgatagttgtctgctgcaatggctctggcactctgatctcattgtccattacctggtttttacatgttatatgagctgcaatatcaaactacttatagttactcaaacatcggttcatgtctccatgcagctggccttcccttcttttttctcaatagggctttcttcaccttttcatctagaaatcttttttgcatcttttaaatttcaattgtgtgtcaactacttggtgaagtctcctttaatatcctccccatcctcaccagcacacacacactatgtggaattaattattcccttccatatattacttatatgtcttgttaatgcatctattcttatatgtatcacaccgaatcataattactataagcttcttgagggccaagactgtgccttattcatcttcacatttccactatctggcccatcataggttttccataaatttgttgagtctatagaccagtgtttgtatataactctatcaagcccctcttttctcctaggctccagatgtgttttttcacttgcttactggggcatctcaacctagatcaacatgttacaaagtgaagttatttgtttttccttacccagtccaatgttttcatcagcttttcctgacgccattgtgtagactccagccattaagcccttctgatctgttagtgtcgtattcctctgccctaatcatgtaaaaattgccctgttcttcaaccccagcatgtcacaaaaggcttttcgtagactggtgctatgcaccatcatacacctgatacattacaaccatgacttaatccttactgctctccaaacatactatttatttgttcatttattcattccttcattcattcacaaatgaataggttgttttacatatgccaggcattttgcagggcatcattaactatgcaaactttgataccaacttgtttttgttcctgcttctcatctgtttagaaggtcattccaacaccccttatctgattggcaaactcctatacatactttgaggcccctaatccctggagtcttctctgatgcatcaaaaagaaaaaaaaaatccctcctcacacaagtgaatatccagaattgcatttttattttattatcccaaatatactagttacaaagatagattataccatattttattacagttatctgattacttctctagctcaatcgcagactctaaatcttttcatgtcagagattgcattttattcatgtttataacttcacttcttaccacaatgcttgacacatggtagatgctaaataaaattcttgccaaattgaattggattttgtaaactgtattgctccttctccgtcctataagactacagtatcacttccattcaactgatgaaacaagtattaaagtatgataatttatcaggttgtctgtttttgtagaaaaaaacttagaagcagatatgaatatcattagaagacatgattcaggtcacattacccaactctctctcatattactactttgtctgcttccgttaggcaggctctttatgaaatactatttctaatgattccttttcttggctagatagcatataacatactttttttagcaataacaattgattcttttttatctttatctaaatattttcttctttgattatatcttctgttttatgaattttatacacacattgtttgcttacaataaatcccttacttcctacctattttctgaacaaaatacactcctcattgctctatattaatcatgtttctaatgccactacgtatcaatgatactttcactagggaaccggaagtgagatacctgcttgagaaaatggtattagaaatatatgtgattggcccagcgctgtggctcacgcctgtaatcacaacagtttgggaggccgaggcgggcgggtcacgaggtcaggagatcaagaccatcctgaataacacggtgaaaccccgtctctactaaaaaattacaaaaaattatctgggcgtggtggcacgtgcctgtaatcccagcaacttgggaggctgagacaggagaatttctcaaacccgggaggcggaggttgcagtgagttgagatcatgccactgcactccagcctgggcaacagagcgagacgtcgtctcaaaaaaaaagaaatgtatgtgattacaagtaagatgatgtgagggtttctcaaatatcaagaaactatacttcctacatgatttattggtatataatttttatgatttaattatatctattgcctcagccacatgaattatatatgtttccactaaaatattatctaaatttactatgaagtttattagctactctgtttaggattatagattttatctttgtcacgaaacatgaactccacagacaagtagtccagagctgactggtacaactgctcaaggatgagacagaggaccaaggttccttttagctagcttcctgctctgcccctatgtagcttttgtcttcatggccacaagataactctccttcctccaagcattatgcccatgttttaggcaaaaagaagggaatgacaaaacagaaagggcaaaataaaaataggtgcagaccagctgcagctgtcccctttaataaccagctttctcagaagcctcaaacagtgacttctgtttctctttcaccatcctgaacaatatcatatgaatacccatcacttcagctgagtctatgaagctgatcatgtttagtcaggcactttgtcacattgaacaaagttggggctcttttggtagagaaaaatagaaggatgaattttgggccaacatctagcaacttctgcaaccaatagtttaccaaatatattagtactgactcatgggaccctagggatatgtgaaaagagatgttatgacttgaaatactttagtgaaataaattttgtttgtttgttttctaggaaaatttagaaatatgcaaacctgtgtattaatgtgactaaagttcagtgggttatagggttttaagttttaatatttttatttatacttaaaatgttattaaagactcagtatatactcagcactaggtaaagaacaatacaaagtaaactatgcaaagatgattctttccctccaggaagttactgctgaatgttggcagtgctgatgagggattctgatggaacatatgtaagcagtggaacaaacactggatagtttaactaaatgaaacgtttgcatattttgcaagtagagaagcaaatgttttttgaagggtggggaaaaaatcatagaagatgaattttaaacaagttcagagcagaaaaatgtttcatttagctattacatattagattctggtatcactggaacacttcagaaagtcttaacattcctcccagctttagtgaagtaagacatgttccataaattctctgtatttcctgcagcaattactataaccacctatactgcaaaaattgatacttgtggggtacctagaagaaggttcaaggaaaccttgctttctcttaccccaaacttccattgacacatactgctgtgttctacgatccagatactttgccccagtgatcattcattttgttcccacataaaccccagaattgattggtactgtccatatattccttttgaagaatctgagagccagaaagttttagtacatttcccaagtacctagggtcaaacttcagttctgtccgattacaaggtctcttttttcccccactcttttcagggtatatttgaaataaatgaatcatatccttgggaattcttggtttgggggaatagaaggaggacaggaagggcagaaatattttattctgtaaaggctgacatcttattcaaggctgccccattctggaggatggaggaagaatgtgaggtctctatgctatatgtgactctgcccagccaaaccaggattattcttggacatgtttgtcatcacttcatccagggcaccatgagaataagaattagtcattggtttggccctgagatgctgtaaatgcataatgatttttaataatcagacctctcataataggtcttgcatgctttccagtaattatccaggatgaaattaaaatgaacttagtccatcttttttaaacttgtggtttgtggaatttgtgccattggattttgtctttcaaattaaaaaaaaatagtataataaacctttctttaaagaaatacattataaatgtggaaaacaaataaaataggccaataacgacatcatttttattctctgtagcaaaactgttatgaaaggtttgtctacctgtttttatcagtttttcttttatgtggtttcttctaactttaattttaatatttatttgcagtaaggcattttgtgtctcataggccagtaagttaaactcttaataccaaggcaggtatatggtagactgggtgcaaggtaatgaggagagggaaatatgttgtacattgagagctcatcttcctttcttttctttttttttttttttttttttttgagatggagtctcgctctgtcgcccaggctggagtgcagtggtgcgatctcggctcactgcaagctccgcctcctgggttgacgccattctcctgcctcagcctcctgagtagctgggactacaggcgccctccaccacgcccggctaatttttgtatttttagtagagacggggtttcaccgtgttagccaggatggtctcgatcccttgacctcgtgatccacctgcctcggcctcccaaagtgctgggattacaggcgtgagccaccgcacccggcctcatctttcttttgaagaaggtatttctttcttccaacctccctctctcccaccttttctctctctctttctttctttcattgcacaagtcaaaaacaaacaaaaaatcagcccactgtttaatctggtcttcaagctccatattgcaacccctttgttaaatgctagaacattgaagttattcaatgaagtacagaaatacatatacatatgcatgagactctgatacactctagtgatttctggttccttgagggcatcagaacttgttggaagcaatgtgcattatggtaaagttcttgatggagcactgataaatgggttactcagtgctaagatgtatgttaccatcactaaatagtcaaaatagccttaatttacagtatcatgctaatgcaaattactttaaaagaaacatctgttctaagaataattgatagtaagcaatattttttgagaaccaaataagaaataattatgttagtgtttgatatggttatgttatttggcgtttccatacaataatttccaatactgctagactaaagcagaagtggctaataaatctttttttctacctttttgcttgatattccatacaagtagatatttatgtactagaaagagagttcaggagaattgtagaaataaattgaaaagctaatccacagaatttcatgtgaaacaaatgattatcatgctccagaggaaaaaccacaaactacacacaaaacagcagcatgatttgcaaaactgttttacagtatttccatgtattagaatttatattctcaatcaatccatcaaagaaacatgactaaatgccctcggtatgttagacactttgccaggcagacaccaggtaccataggggattcaaagatgttcctatcctcagggagttgacaatctggctagataaaaattcagaataataaccattcttctaagccattttaaacaaaactcgtggtttgaggttaaaaaaaaactgcttgacagttatggctttttagagtgttctatggaataaattgggtttctcttttttgtgtttagtgaattacccatcacttacagatgagcagaataagctagtcttcatttttctgcctccagaggctttgtttgtggttctttaaaacttatttaggtctgagttccagaatttaaaagggatatttaagtatgaaaacaagtctgaggtaagtaatatgattagaggtgattaaaaggaaaatgggagagtatcacaaaagggttaccagatattttttaaaaaaagagagagaaaaatgtagaaaagtattttaataatttttttacattaaaaagattatcatgctaagagtggcaggtatttatttttcttcattactgagactaacactagagtaataggtgctgttttaacatgagaaaaatcagaatacttgtaaggtggaccctcatactagcaaaggtatctaagagtacgttttatgacaaatatttataactactgttctttgttctatttgaaaatagccttatttctagtgattaacaaaacaatatgtgctttcaaaaataatccaggcaatacagaaaactatgaagaattgaaattcacttgaacttcaacctcccactgatactgttaacacatattttacattgatgttatcagaatttgtgcgtacattcatgagggatcatcaagccaggctagaaatgtatgtagtttcatattttgtagctatttttaaatgtgagattgatgtttagggtatagcttactttaggaccatggaaaagcaagaacatcttttaaatgacctgctggtattcccttcatgctttataataaaggtaagagataagcctctgtgctatttgggagaggcactgggttcaaggtttgactccaccatttactgactgtatgaccttcaggagggcaattcggttctatcgcctcattgcctttttctgtcaactgggaagacgtgactcaagctctaaaattttgttatctcaatgctttattatagtaaaagagtataccttctcagtaacatatatttacaaaaaattatccttttttatgtgttatatgccaaaagttttaactttgataatgagttagttttccccttatcagcaaaaacaagaagaagcaagattattttaaaagttggaagtttattactcatttattcaggttaaaatattctgatactcgaagaaaaagtgaaatttgttgttggatataatacaatgtccatattcattacatacgttattccattatgttccacatacccctatgacatgccagcttatctgtttagagtacttttctgtgtgttcaattatatatgtaaggtttttaatgaagaaaaataaaataaggaatatttagaaatataattatatagaggtcaggactgtgaaaattgcttttctcacaatcactataacttagctttatcgcctaaattatatagtaataaaaattgtttttatactcctagagtaaccagtctgttcttggtaagagattgcaatacatttatctaaatagaaaatagttctctctcccagtcacttttgaaacagtatttccctagtatgtttgaagccatgtgtggaagcattttagatactgtgaaattaaaaaaaaactgacaaagtattttaggaaaaacccttgatcctatttatttattgtaatagctgctcttcattcaaacaaacatcagtgagttatggaaacgaatatagagctggagttaggatacccaagtcttaacctgttacaaaatgcgagcacttggcaagttctttaactctcctgagcctcagttttctcttacgtgatgaggattgctgcataatatacatgaaaatgtcttagaaatgtaaacttcaattattttgtttagtattttatttttcacatactatctgagctcttgtaaaaataaaattctgaatgaaatactgaactaagaattcagccatttaatgtttacctcttcagacttcaacatcacttaaacacattgacttcctttgttgctccagttcattaatgtacatgctctcattaatttttcaagaaataaataacatctttagaaatctataacttcattttcttttcacaatagaagattaaagagagacagttttgttgacagattacatcttatattccaccagctagaatctagctgaataatttaagtatatgaactagaagaatatgtaacaataataaactgacttccttttgatagctctgagtatgtgtttgaatacacataaaaacaagctggatcctttaaaagatgaaagtaaagttctgtttattaaattttgaaatatttgctaaacataaatggaatccagttttgaaaaaggaaatatcaaaaagataccacaatgatctatgctgcaaacacactttcagataatctatagaattttgctctgttcttcattaatggatttgctattgatcatctgattgcattccttattgatgttttctaggtatatttttattgtgcttttaaatctttttcatataaacaaaatacatattacttaaaaacctctagaaataaaaatctctattattatgtcaataacagaggataccaaaaatgtaagggatagcattatttggtattatgcttattcctctttaatattaaccaaattttaaatatggcttctaaaaatttttaccatttttagggtcaggaaacatttaaaaaataaaatatcttcaaaacattagatgtatggattactgtttggaacatccaaatgccctttcagattagtgtgatatgatttgtcttgcagctgcttcaaattaagaacaggtgtagaaaacagattatgcaaatactcatattaatgtaattgtcaaattggtgtgtttaccattgtgcttttaaagagtagactgtacttctattttaaatgtaaattagctttattcttcagagcagctattggctaatgaaacttggttctgccttagaaatgagtaatttctaatagtaacacagttctaaaatttgacattttctttcccagtaaacttatttatatgtaaaaatgacaattctgtattatttttagtatgtgtataatcattatgattttttgaaaatttaagttgattctggactactattttcttttattgtggtaggtagtgattacaatgatagcttttgatctcctaacatattttctaatttagaactcatattagcttttcatctttatagatctaaactgtcaataaattaagttctttccagaaattgcattacatttattttaatactaattttttttcatagcatctagttactttcaatagtattttaatgttaaaaatgcttaaattctaattttggtgttttgcgttttactactagttcatccacgtatctgtatgatgattttgtgattgcctaatgtcaggattcagacacgtgtatctaaaaaagggcttcctcttagtccctggctagtcttctaggctctttaatgacaagtagtatgagtcatcatatctacatgcgtgactagggccagcgaaaaagaagaccaaggaatcaaccagagaggtttcacaagtcagatgttcccattacttttttaatacttttttctctaataaaacagagatcctgaagcatttataattttaatttgctttggtagcatatatttagtgcttactttgtgtacctggtaatgtatttatttatgctctttcatttcacagaataggcacctgaggcttagaaaggttaaattacattatctacagtcaagtagctggtagtttgagctggaagtgaattcaagtcagtctgattcgaaaccatgtgccttttactcctttgttacagataatatagtgcctcttagttgctgttcaggtttttcattttcatttttagaaaatgagatcaaagtaatgacaaggggatgacaaggtattacatgtgaatattatcttttcttgaatgtcttcattttatgaaacccaggtatattgttttgtggttaagatagagcaaaggggctgggcacagtggctcatgcctgtaatctcagcactttgggagactgaggcgggcagatcacctgaggtcaggagttcgagaccagcctgaccaacatggtgaaaccccgtctctactaaaaatacaaaaattagccaggtgtggtgttacatgcctgtcatcccagctacacgggaggctgaggcaggagagtcacttgaacccaggaggtggaggttacagtgagccaagaatacaccactgcactccagcctgggcaacagagcaagactgtctcaaaaacaaacaaacaaaaaaacaaacaaaaagatagagaaaaggaagggatgaaagttacatggctccaccatggaggggatctccatcatcactacgaatagaagattctacaggaatgactctgaatctctttattcccttgtcttctatacctccctcatctctacctgtcaggaaccactgctgacatggcagtggacttggtaatgaaattagatgtttactcagcaatatatgagataacttttaatatgaaagaaaaagcaaatggcagtgatggactactactactggagttttcttggcatcatgagaattttcatgtcaaatggtaagaagttttttgctggagttttagtttttaaatcactagtcctcttcctccaggtttcttccccctacatcacatcaaaatgaggaggggtaagaggcttatagggaccaaatcataatcagagatgccaacaagataagcttctgtatatgctacatgcagtctctattcctcatttatccttattggcaagaaaaggagctgccactgagaccaacaccagataagcagaagctgatccaagggctgctgctgcttcccctgcagggatcagaaaaggaactaccacatcctccagtccctgcatacattcctaggggtgatcataatctgaattgctcccccctaaggagtaaagcatacagtaaaagagatgctggtactttgtgaatttggaaaaaaattataatatgatagtgtttttctttacatataatagatattgcagtgaatcttcatgcttcattcattgaatcaagttctaatccaaaaatacattccaatggaaaagaccaccaaaaagtaattaaagggtttgcctcatccacaaactctgaggacatccatggtcagttcttctatgaaagcataataccctatataagcacagcatttattgatgctgataagctttctgattaagttcacaacaaatgcacttcagaattattttgaatagttttttctagtttattgcttaaaagtatatctctataagaatatcagatacagtgagatttctattaagaaaaaattagtaactttaaagatcttttgtattgttttttgaaaaaactgtaaaacaagctaaatgctatataatacatctactctgttactatgctgttttattctgcctactatattattttcctttttttagtaattttatttttcatttcttgaaattaatatgtaaaatacagtttttacattaagcaagaaagacttttgcttgaaaattaataaatctctacattaaattcttgggctcaaaccctgttttcatacattgttggacaatgagtcaaggtggaaaatctaaggttgttctgtgctcagtgtcttgatttgaaatattcagaatatagacttactcagagattggaagctggtttaatatgttgtgccagccatcctgaaagccatttcagtagattctgtaccctggcatttgttttagaaataattgcatgcaaatgtagttttacatctgtactatgtctcttaatcctaaccaggcattttccattagatgtagtctgaaaatcgcattaagacaaatatatatcatctgtgaaattatgctctgaatgctctaaataggaaagaaaggggcaatattcatgttaaatataataagaagtatttttttctgactcccaaattttaaaatgtcgatttcacttcctaattagtgctgcattataaaattgagttaagattgttttcaaaattttcttattcagtttatcacaaaactttcctacaaatgaaaatgtactaccatgtcaaattattttcttactttatgcttggtactacaaaatttatttttgtatctctaatggcaatgcaattcagataagttatattgtagtactcttctgttctcaaaagcagaattttgcaacaagaattttgacactgaactgactttatttatttatttatttatttatttatttatttatttagagatggagtcctgctctgtcgcccaggctggtgtgcagtggcccgatctcagctcactgtaacctctgcctcccgggttcaagcagttctctgtgtcagcctcccaagtagctgggattacaggtgcccaccaccacacctggctaatttttgtatttttagtagagatggggtttcaccatcttggccaggctggtcttgaactcctgacctcgtgatccacccaccttggcctcccaaagtgctgggattacaggcgtgaggcactgcacccagccctgacttttaatatactagagattactagtgtatttgaggaatctaagaaagaatattaagtaataaaacacgtatgaagtcatggggtatcaactggaagaaagaggaggtagagtttatagaaacaaaaagttattgcttgccatcaagaaattaccattaaatctttatttaaaacagtatcttgtctcattctgcattcttcatagttacttcttgaggataacactaataggactccagatatgtcagaaatctttttatgtatttgaactttcatttatcctccaagagaaaagacaggtggtgtacacaaaaatgagaaatctgaagttcacaaaagccactttgctttatatggggctcttgaagagctggtttttgcttctctttctgtccccaaccctaccctaccacccttcttccctaaaattaatttgtttgactttatgggactgtctctgcttctcagaatacatcagggggtcagacaccttcaagcctttatgatgtctacttctttagtcttaaatgttcttcatctttaccccaccccgtctccatttgtatactaatgtccatttattcttacctagagtcctttattatctcatctgccctgtaagaaccctttctctatgcttccttttcaccagtgcatacgtctgtcatagtataatacttaatatgaaactcatcactaatagtttgccttcctgattcgtctgtgcattcttgaggttagtggctcctacttgactttgcagctgattcttaacctagtggttggtgttctgtagtgtcccacagaactgagatggcaatgtatacattaaatctgtttgttttgtctattaaataattattatatatgttaaatttttttctttcactctccaaatgttcataagaaatccagctacctattttttttcaaaaacgcttaaactcttgaataaagcaaacatgcatacaagtaaacaaaaatgcagtagtccataagtactctgcagggatatctcatgtcagaagcacctgttgactgctgccctcatgctctctcaaaatataataactatatcacttacaggagcactatgcacatcttttgtttttcagtgattttagacacatgaaagatacttgattagcattcctgcaaattcaagttgtatatgtacacagtgaacaggtgtagatcttagcctgctcacagacaacagccatgtcatctgccctttccttgatttcagccttacctatctgtggagacagcctggattctggataagaggtgtccatatcctctttctgttattggcttctctactgcgatttctaacaagggtgccaattagtgccagctatgatagcagctttttgaagtgaaaaagaaggggattgactaaaaacagggtgaaaaatctgaacttgacactgttatattctgtaactcttgttaattcctttttaatcttaaactatcttattttggctgggcgcggtggctcacgcctgtaatcccagcactttgggaggccaaggcgggcagatcacgaggtcaggagatcgagaccatactggctaacacggtgaaaccccgtctctactaaaaaaaacaccacaaaaaaattagccaggcgtggtggccggcgcctgtagtcccagctactcaggaggctgagtcaggagaatggcgtgaacctggcaggcagagcttgcagtgagccaatatcgtgccactgcactccagcctgggcgacagagcaagattccatcttaaaaaaaaaaaaaaatctcattttaaaattaatttaaaatcttactttaggaacgtcaatggatttaataggttaatatgaagtactttaaaatctcaaatccttcaaatacctatggtatctgcattgggacagtggctgccactaaactttctttgttaagctagcctttcttttaataaaaagaaaagtgttattttaaatttagaatccattaaagaagaaaagtggaatgagtatatagtgttaaattatgataaaaataaaatatgcaaacacccattaataaactgtgctgtgttgctatcttgtatttaaccccaagttttattttgccgtttttacatattaagttgtgctttgtacaatgtaaagtctgattatacaagctgatattttagggatggaagaatatatttataaaagtgaaattatggccaggtgcagtgtctcacacctgtaatcccagcactttgggatgcagaggcgggtggatcccctgaggccaggagtttgagaccagcctggccaacatagggaaacctaaaaatgttaaaaatactaaaaatactaaaaaattagccaggcatggtggcacacacctataatctcaactacttgggaggcagaggcacaagaattgcttgagctcaggaggcagcagttgcagtgagttgagattgtgccactgcacaccagcctgggtgacagagcaagaccctgttcccccgcctccgccaaaaaaaagaactgaaattataatagcaatttttaattacttcacttaagtttgcaaacaatgaaaaatgagggcaagatcatgacttttagtcttatctttggtctgttccatataaatggattgtaacccaaaattcgtccaaaaagctattaacatttaatcctaattttgacagtatgattattgactgaaatatgtatattaaatttatcttgttaattttaactttgatttcaagtcacagagatctttaaaaaataatagtagactttattcaaaggatatagagttgcttacaaaatacaaggcaaagctaagcaaaaatctggtctcagaaggagcaaaaagcagagcagaatctaataggctgcttgcatacctgtcactggattcgatgaaatcctgttgtttattatgcacatgtcacatacctcaaaaatcaagttcttgggagagggaatttaattggcctaacttgtatcacccgcccttgggccggtcccaccaatttgcatgtaattgggtacagaattaaggtagtccccaaagaaaaaaatggtgctattaacaaatagagaagtatatgctgggtaggcaatatcaacagatttcgtctatgttaactttgagttggtttgctactagactgcagccttgttttaatatcaatttttttttctcgaagaattttactctttcaacaccaaggaaactcaagacaatagaaaactactggacaatagcaccaagtaatgtatttgtgtgtgaagactcactaatttatgcaattatacctcaatctatgcagtatcttcctaaagattattaaaaaatcaaatgtattttaaagaagtttgccttttatttaaataaatactgtctaaactactcttttaatttgaataatttcttctcttaaggaaatagtaatctcttatttaattttgtaaatttggatcttcatgttgagtttgttagcaatgaatggcacctgtcacgcgtgtacttaacaggtaaaagaaaatatactgcttttaaaactttacactaatccatgaagaaaacaaacataaaaattataaacattcatgaatcgattcctttaaaaatgttctccatgaccttggacctatcttttttgttgtttttggtgttgggttttgtttgtttgttttttgagacatggtctttctctatagcccatgctggagtgcagtagcatgatcatagtttacaacagcttctaactcctgggctcaagtcattctcctacctcagcttcccagagtgctgggattacagacatgggccaccatgtctaacttggaccggttctttaaaatcactttgaattggtggacctaaggaattcccattctgcgcactatgttctcttgcaccatactctcttgttattcccacatatagcccaccaccacctccacccacctggacttcttcctgatcatgttgtatcccatgattaaggatgaccttaaacactttcactattctgaatcatcctttctgaccctgctgcagcatttgttcttccatttcccaagctgtggcttttcttattcctactcctaagcaagctactgagtattcctatagagaatcacaatatcaggcagtattggttctctgactttaggctagtgttcactgatgtttagcaattgtttcacaagtctctagacaccttactctgcctctctccctgcatcagtttctagccttcactgtacccttaaagcacttaagtcacacctgtttccctgttgtcagtgacttcaccttctacttcagagaaaaaatttagaggccaccaggcaagaactccctctgcctctctcatctctactcccaaaacaaacttatattagcatctctgattatatccctttgtatcataatgagatttctctccaaggccaatttctcttcctgtgcatgatacctacccctttccacctcttccagaaccttgtcttgtcagctgcacgtcttcattcctatgtcttcagtctcccatttctctgtgtgccttcccttcccacctttaaaaacaaaaaccaattaaacaaaatcacattcatatgtccatattagttttatatcagacaattatcttaaccctgttttttttttcttcatcattaggccattagaaagaattgtttacacttatcttccattccttttttgacacattataccttgttttctttccttactacctgtattactctgtcctcacgctgctagtaaagacatacctgagactgggtaacttacaaagcaaaagagatttaatggactcacagttccacatggctggggaggcctcacaatcatggtggaaggaagaggaggagtaaaggcacatcttacatagcagcagggaagagagcatgtgcagggacactcccttttataaaaccatcagatctcttgagacttattcactgtcatgagaacagtatgggaaatacctgcccccatgattcagttacctcccaccaggtccctcccatgacacatggggattaagggaactacaattcaagatgagatttgggtagggacatagccaaaccacatcactacctttctgaaatagttctattgaatatgactgaccacttcagagagttgaattcaattcttaccttagtagatcctgtcaaaacatttgagacttttgatacaacttctcttaacattcctcttctttgtgttttgttgtcttttccccatctcctgcctacaactttttttgttaacttttatatcagtggtccaaaaactctggctgcatattgtcattacttgggatgtttatttgttaaaatgcctggtctccaccaagcccagttaaatcaggaagttgaatgccaccatcattatatatacagagctctgcgtgtgatgctgacccacaaggtgggctgagaactactcctttagatgacctcactgccagaacttatccactccctgtggtattaccactacataagttgctgattctcagggctttatatttcttgtcttgtcacctgaacttaagtcttgtatagccagaagtcttccagaaacacctacctagatgttcagtagtcaacttctcatttactatttccaaatgaaccaaattctctttcattcttgaaaaatcgtctttgtttctactgtctttctagtggcatcacagtctgcttccaacgtcccttatcctttactctctcactcaggtaatcgtcaaatccttagaagttagctggtcctcaaattgggtatccttttttcatgtaactgctactgctttggcacttaccagaagtaactgtctaatgaatctttgggaatcttaggctgccagccaattccagtctgaacccacaccctcatgctttctacttgtcaaaaaacaaacatacaaacaaacaaacaaaaaacactactaattcctgatctgcaaaactcttcgtagtcctaatctttctagccttgtctctcagcagctggccccatttatagcatactcctaccaaaactatcacatattgtcggcataatcctaaaccttttgcattctccctttctgtttttttttacttctttttcccaagcaatgaattcctactcatttcttgggagccagctagaaaatgccttctttcaatatggtcaaactgtactttttacgtcttcagcaatgcatttctctctaatatgggggctttaaaacttaggatccatggatagaattcaggaggtttatgaactttaatggaagaaaaagtattcttatttttattcccaattgaaactcaacatattcataggaatgtaggcaacacaccacagtattgttaacagtatctgtgacattgtcagtgatagaaattataaaaatattcctatctcatcacagttgataaaaaatatctagaaatatgaccaggtgtagtggctcatacctataatctcagcactttgggaggccgaggtgggaggatcacttgagcccaggagttcaagaccagcctggagtatatagtgagaccctgtctctacaaaaatcaaagaaaattaggtgagcatggtggtatgcacttgtagtcccagctactcaggagactgaggtgagaggattacttgatcctaagtggtggatgctgcagtgagccaagattgctccactgcactccagccttggcaacagagcaaggccctgtcaaaaaaaaaaaaaaaaaatctagaaatgttatttgtgcccattactcaaaattgtggtggttgttatatcttattaattcattaaatatactattatatcttatttaattcattaataaatagcattaatatttcacaaatgtagctttttaaatattttgagaactgaatctcaatataattctcttttttctaatgccttattttattgtatgcaaccaattttttttttttctgagaaggctattctagattttatcagattgccaaagaggttcagatacaaaaaagaaaaacaaaaaaaaaatcaggagtccatgcaaatggcacttatgttgttgtatcctaatttacaagactttttttattttccaatctcctttggatggagggaccattttcattcacctttttatatccacatggctaatatagtttatagctcatagtaggtactgaaaagtgttgactaagtgaataatggactaaaagaaggaaaaaatacctgtcatgcaaatatcctgaacagagggcaatatttctcaaagtatgatcatagaccttctaaaccacaataatcaagatgcttgttaaaatgcatatccctggacgctataccagatcttatgaatctgaatctctaggaaagtagcctggaatacacaatcataacaagcttctccaaagattattttacatgcttaagtttgggaacctgtatagtcagatatgtgatacacagtagcctcaaaataagtaaataaaagcataactttttacaaataattttatctccctggacttcctttccttcctcagtccacaccaccatcttctcaatgttgtacaggtcttggagtctgtgtcacaaaagaaagtaagacaaggctaactatatcagaataaagaatgaaaattttttaggcatgcacttacttagatttggtcctctgagatacgtaatctctggttcttgttgactgactccaaacaagagaacaacttctgtgatccacttgtcttgtggaatactcaagacagttttcaagtatgatttctcttttcttcatcttctctgtctgacctaggcaatttaaaatttgtttttcagaaatggactggagaaggatgaagttgtatgaatttgcaaagctaaaatgtgttatcctggcacaacccaatagctacattgtccctgggacagtttccatcagatatgatggatgtcctaaaaacaagccatcaaatcagcaaacacaacaaaacagttggaggggcagaaaggcaaatcaaccatttgtttttgttattgttttaaagtttgatttgttttataacgtgaagaatgttcatacttcttgactagctgaacattgatgttttcattttttcacttttaaaatgtagaccactcctatagcttggatatttttcctccaaatctcatgttgaaatttgacccccagtgttggaggtggggcctcacgggaggtgtttgggtcatgagagtggattcctcatgaatgtcttggtgccatccttgtgatggtgagtgatctctcactctactagttcccacagagctggttgtaaaaaagagtctggcacctccttcactcttgtttcctctctcaccacgtgatttctacacacgtgtttccccttccccttctgctatgagtggaagccacttgaggccctcaccagatgcagatgccagcaccatgcttcttgtacagcgtgcaaaactatgagccaaataaacctcttttccttataaattactcagcctcatgtattcctttacagcaacataaatgtactaagacaaccactattccattcagatattattttatcctaaatattataagacactaaagttttaaacatgttaaagaaaaatttgaggatcctttcaaccaacctcaagaaacaagttatattaaataatttaaaatattttcatttgttaggaaagatcaatatataatattaataccatgtatatattatatatttttgtatgaaaattacacttcatcaaccttgcaaaaaatattatttttcttacaagtcatttcaccttatcactaaatatgaacacacctatattaacgtcttgctttgctgcaccaggtttctaaaaggaaaaaaatatttctttactcatataggagagtctttagtatgtatcatgaagtctactgataagttgaagacagatacaatgtcttgggaatttttgtaacctttataatatgtaatagcattttatacatagtagaaacaataattctttttatctgaaaattgagtactattttgaagcaataatgacagctaacaaataagtatatagctaataaatgttaagtatatgggtatcagcactgttctaattactttacaattgaacatatttaataaccatatgatgatgttacttattatcctctgtctagagagagaggacaggagaagctaagtattttgctcaaggtcacaccactattaagtgtaagatctgaagctcagcatactatctccagagccagcattcttatgttcagcactaaatgacttactctttattgttttaaaagaaaaattacaatgattcacatttaattgaagaatagattccttctgaagttaatgttgaaagtccattccagtacattgtgtggccttttcactcttaaaagattctttggtgcttattagaaacagatgcaaacacttaaaatatccattcacattgtgtttccttgtgatttgctggagttatgtcaggttgcctcccggagatccatttctaaatgactgtctgattttattcatcttgctacttctttacaaacctgcggcttcatggttctgttaaagcatggttgatcagagttttattgcatctcttaccaacattctgtctctgcctgtcatagaaagctttagggaagccatgagaagaaactgggtggggttttcaaagcacaatttcttcaaccaaacctgttggcaagatatgatgatgggctaatgatagtgcaaagtttattctcacatactttcagttgcagaaactattgagagttacttaaaagcatttgcctttcaatagattattttacttctaagttattgttcttcagtaaagtttagattaatttgactttctcttttcacaaattttgccttttaactctttatttttattgattgtttaactgttatgtttcataattttgcatgcagagtgacagggacagagaaatgtcactatgagccctcagtgtcggtggcagttctgtgtctcttgttcctatggatagattaatgacagtatcattttaatatgctttatttctctctatgttagcaggaataatttgataggagtgttttggttagtcagtgtttctggttgataaaaataagatatatcatagattttagggaatcgaaaaataatgagtatatactgtgtaccaatccaaatatatcattgtggatgaatgaaaaggttcttcatggtcttgtgggtttttttttgggggggggttattttgttttgtttttgtttgtttgttttttgtcaaggttatcgtttcgttaaaatttgggaaatatttgagatagtaactaaagaagatatgtttgaaatatggttaggtttatgaaattatgtttctgagtcagttgagctaaaaatggggaacattctcacggtgaagcctcaggcagcttccatcccaaagctacaaaactagacgaattctaatgaaaacctcttttttaatgaaatccttctaatattttggcacctgtatcccttaaattaccatttaagggatgtttatgtttaagggatcagtagcattaactgtatgcttttattccatagtatttttatttatttttattactgtacttgtacattatctcaatatttcctgtttatgtactcctccaggagtgagacttattcttgtatcaccttctgacctgactgggatgacaaaactccaggatcccaacatgttttgttaatgctggagacacaaattatgatattttatttaacataggtgcttgtgaaataatttaaggtggtgttgcttatttatagagtttaattttcagtattaaatataaacagtgtcacggcagatttggagttattgtgacaatcacttattttttatgtaaatgagcgtctatctgtgggctttattagtgttagtatggtttattgttatggtatggaatagcatttcattttttaaggatgcttatgaccttgttaaatagcagtccaaaaatcgtgtggcttgttttgattttaacgaaagttacagctgcctagctgtaaagcagtcagtttctcttattatgactagttcttcaacttgtactgttttaaattgcctgcaattagcatatttcaaccttatttttaggtttaaattgcagtgtgagagtgtactctcctcttagctaattagtgctatcaaggaacagtaggttacctcattagtaccatgtgcatttaattaaagaagagaaaattggagttaacctcatagttaatattttcacagagtgctacaatgccaacaccttcaattggagtcacacccctgtgtatctaattatgcactggtgccaaataaggttagcaagagtagcacaaggctgttgacaaaataatttagatgaaaatgtatttgttgaattctggcagcaaaatacaacttgaacaatttccccccagtcttcatctgctagcatcaggtgacatacttcttcctaagaaacagagggaaaaggatattttggaagccatttttcctgtttgaattaatgttgctcttatcttcaagaaaatggcatgtagtcatgtaaagcatatacaatccatttcttaaacataacctctgaacttcatagatgtatttgaaaagaattatttataagatgtaatatttacatgcatcaaaccactacaaatactccctgacacccttaacacatccctccttgcttttcttcttttctggtatagttataatttcccttgtgtatcttatgaatggggatcatcctttcatatcatacatgaagtctaaggatgtgttgtcacatggtctattcaaatgcagtgtaatccttttgcccttcagacaaaatctttgaaatgtctaatgctacttaggtaaaaaatgacattccatgtcatttatcaataaataatcactttgcttttacagataaaaatgcaaactatacatttgagtatatagagtagttttcaatgcaaatggtacaattcaacaggttttaacagatattatggaggttggttgcatagtcttagatacattttagtggcaataatttttatataataatgcataaccacagcagtttatttaaataaattattgtgattgttctcacatgacatggtaggacaggatttatgttcatttgtactggttacaattgattacaagtcagaggtttgctttttcaaaattattgatgattacattttaagaacaaataacatcccatacaccaaaattgcagtgtcatttaaatatataggagtatttatgattcttagcttacaagtgggaagtaaggttcctacagcagtatcatggtacataatcagtggattttttggttatgggttaatttattatggtccaaatattcaacttcccatctgccaaattatttctatttttaaaatctatactggttaactgtgagacaatgatggaatattcttccctattttgacctaccaagataataattccaaaaaatgatttttaaaaatgataatgtacgttattagcacaagccttaagattgaaaaaaaagccaactcctgattctctttgtttaacctaggaattgcttccagaaacaaaattatgtatctgtggccactcttctggtgatgggcatcctcacaacacatttgcagtaagttacactgggcaattagaaattcagctactaggcacaaggccatggacccagtcccactaaatagattatatgtgattttctatagccaatcccttccattggtgcagataatcaagatacaaataatgttaagatacagatgtaattagttgattttctcttgcttcttttgttctcaattcagttatgtttcactagatatacatttcacagtttctgtgagttgactaccttaaatatataaaggctttaacaccactatacttttcttgaacttttaggaataatttgatgagggagtgttgatatgcagaaaataaatgggcatagataatgagttgattcaacacatattttagaacaaaattgctgttccatgttcttcaagttgtcatataaatataccaaggccttttaaacattctgccactagagtgtaacttgtcactttggactgtaatatttggtgaaaattgcttatgtggcccaattcacatagcagttatccttggaaatggatagtaatcctcaagtctccattcaagaatttttaaataaagcaacttcatgtttctattgagcatagattctacctgcattcttaagaaatcttgaaacatataagtcatgtattttcacatatagtcagcgcttcacagtcatgggttctgtatctgtggattcaaccaaccacagatccaaaatagttggggaaaaaatggatggttgcatctgtaccgaaaattttcatacatttttcttccttatcattattccccaaagaaaacattgtaacaactgtttatacagcatttattttacattaagtattataagtgagctagagattatttaaagtatacaggaggatgtgcataggttatatgtaaatagtacatcattttatataagggacttgggcaattatggattttggtatctatgtgtgtccaatcccctacacataccgaaggactactatacacaacttttgcttgtttccttaagaactgatgtatttatttaggctcaatgggctagagtatttctaaattgactcagccaaaatttatttactttttactactctatacataatatttcctaattataattggcgaacaacaacaaaattaatactcatatacaatcaatgtcttcctgaatgaagaagaaatatctaatttcatactttggttaaaaagacagaaaatgttttcaataaaaatagtgtcaaattaatgaaaggatatttatggaaaatatgaaacttttaaaaatgttagttgcaatacaaaataaactcaaaagtggaagcagcaataacatatcctattctatagaaatggtaaatgtcaaaaaaaaaagaaataatgaatgtcaaggatgggtaataaatgttatatatgctggcttcagagagacctgaaattacctggtttgtggcctcattcactttaatcctatagtatttggtccccctaagcttttttgtaacaacatttgaaagaaaaatagcaaaaatttaaaaattttggtaagttttgctgatatgatgtaattaagtttatgtgaaagattaaaataatcttaatttctaagtattctctgtatattttaaaggaagataaattttaggtaaacatcaagtgcaattatattctttaacttaataaagaaaattaacttaaaattacttttaaaaatagtgcattaaaaatgggacagttttcctaattagtaacactttttcctaaaataatcccaatggaaaaattgttttcgttaaactgaagagtgaggtggcttaactgctaatagtgtttgctctttatctaaaactacttttaccatttaaaacaaatctctgttctactgatgagagaaaagaaatctatagcatcaaaagtttatattttcactgacatattttcagaatgaaaatgagacatgaagaagtgaaatgattcaccagaggccatgtaatttctcaatggtaaagctgaatttgttgcccaagtttgtgatgaccagtgtattggtctttccaaaggtatcccaaattgttcttggctgctttgaatgcagtgttaagtagaagtctgaggttcttccatgtgtctgagactgcatctcatctctcagatagaaagagcaccagtatgagttagtgaggtctggtatggctggtgggaaaggaggagcatagcagtgcatttgccatcagatctttgcaatctctgtagtgtgccaatttcatacttcatcttggtcattgagtgactttacaaatggggcttcttgacttcacaacacaagggaaatcttatttaagtaatattaactgcttattttcctttattgattaccttatatataaacttcagcatgctaaaatggctacataatgtgatttaaaatcaatatactaaccagaaaaaatactatattattcataattgacaattgctatgtgatggacatggttctgttttagtattcattgcatttataagtaccaatatgccagagtggtagtctggaacaccgtaagagtactggtgggctaaaaggaagaaagaggtcaaaaagatagatggggtacctaaagcctgccatatgttttcatcactatgtaactaacatttataataaattgtgaattattacaatagttttcaaatgttaagagaccctacataatgcctatcaaagtgttaatttaatgtcacacatattatttctgtggttgatcagccttcacacacacacatacaagccaaaattttcaggcacatgttccaaattggtggattaaattaatttttactcttcctatcacacctaattcacttaaaaaataaaaatcccttaattttattaacaagcctaaagtaaagtgccaaaaattgcagggagagaaaacaacttacattaccaaatgaaggttaaagcccaaattatctcaaggaaataatattacatatttgatattaaacaggggtcaaaagctcttcattataccatcctttttcagatataattgtatcaatttactagcaaaacgtgcaataaaaatattttttcatagggttttttctggtgtcttggcttattgataaccatcaaacattggccatcccctgagtattgtacctctctattacattgttgaacagtagtgatattttaatgtactcactaataaattatatttaatgtgcctctatcaattcattgcaatactgggcagtaattgtatgcacttatctttatgtgaaaattgtgattctaaattttatctacttgttgtatttgaagaaatatttgggctccttcgattgtatattcactattctaagagtaacaatataatgtccttattataactttgaatttaatagataaaaaagcattaatttctggctttaaaaaacgagggtataagaggaagagagcttcattgttagagaatacttttcaatgcttgcttttcacattcagttttttcaaatagtgtctagtttaatctgaaaaaaaaaatcttcacttattttacttttctttcaaacttcggtactaggatacatgcctttctttttttcctgatttatcaaacagctttacaaatgagtaactctgccaagattctaaatttcacaatctttggcttccttacaagatagtaacccaagttcattcactactctctaactataccctgttggcacctcatctgcctgaatgacctcattaacacctatctgcaagtaatcttattgttagtgttgcctacaggtttttacacctaagtgaatactgttttacataggattctgccagttagttctaactgcgaaggcaattatgtggaatgcttagggtctttgtctttggctcttatttttaaaacctttagcattgagccagatgaatcttttgatgaacaaaatgtcttgtacctataatctgaaaatgaaacttgcctagccttggtataactgctttattttccattcaaattaaattacactggccacatttgatctttctccctacaggtctttactatgtttatctcctgaatggttcatttttaatggggatgctgggttggaagaaataagaaaacagggatagtgatgctttttaaaatatctttttatttgtgttccaaattcatctctctcttgaggctttacataaatcctacacaacaccaaataatccaccctgtgggctctacaatcagctttctggtttgataactgggccagtttcttaactttgctttgcttcacgttctcagtctgtaaaattataacaataatactacctacctcataaggtggtaacgggaattaaatatgataaccctaaaaagtacttaccataatgactaacacatagtaaacatgcaatagatgttgagtcagtatctgtaatagcatcaatatactcttgcttggatgaaagagtcaattctgttggatgtaaatttaagtgtatttttggtattaacactaaaaggggcattggacattgtaccttgggtcacggccaagacagatggcagtgttatccactgagaggagtctttatattaatcgatttaagttgaatactgacttgttcactcacacaaaaaatagtgttctccaaatgctttctgacttgttaagtggaagacagctaagtggaagagacagaactctgcctcaccagacctaaatgatgcctggcagccaagaagaatcatagctaaaatacgtaaggaaaagtgattgtcccccaattgtctgtatccagctaaagcacatgaagatacaaagttaggaaatacatgcagcaatgttgaattgcctctacttttttcttccttcctatattataaacatattagcagagagtaacctgaagttaaagataaattactacttatcagtgttgacagttttaactaacatgctgaaaacaattaaccaaggataatgttgcagtaatttttttcacagtaccctttttataaaagctgtttacagtattttataaaatactgtatacatttgtaaaaagatgtgtggtgtacacttgaattcattgttttactgtgtttatttttgagtaggttttattgattattatgatggtattcatactatggcccttcagtaagaaataaacttaagataatgaaatgtttcaaacacttatgttatagattgttttgttaattccataaagatataatatagtgaccatgtttagggttattgttaaaaacattccagatgctgtatccatctgaaacagaatacaggtttttgtttttaaagccttggctgacatttgtatgcaatttcttgattaatctttttttctactgtaattcaacatattagtaattatctttacacgcccctctcagctggaatgtaagctccctgatctcagtttgttctagaaacttctcctgagtggttgcccctcaccttctatttcattgtaatatttgagattcccaagaatattcttgatgttggttccaagaaggaccagtagcagacatttttcaatggaggatcgtctttgaaatttataatgaataatttactccccttgatgattcaccaaagcattagtgtggcagaaattgggggctgatgaaaggattatttatttagtcagtggttttgttgttgttaatatatttttcatcaaataaattgatacacacactagaatatgtatgtggcaataatattcatttatggtgcttattaggacagactgtattttgcattttaaagttatttgtaatgagctattagtgatagtctctacaaatatgacatataattggcattaacttctaaccgtttgctctatagtttttgcgtatttctaaacatcgcttaaggaaatgtgtattctgtatttactatttgtatttctgttccctgtctatttccacatcatcttatttagcataattgaaagttgtagaaaatgctttgtcattttagtgtataaattttcagaattgtgctcatacacaaacaaacatacatcatacatacacatatctaagcaattatataaaagagtgtgcatgtgtctttattcaattctatgtgcagttcatttttctttcttattgaagttacatgttttagatttgtaattttcattaggtgagtgaattgtttgttatgcataggattaaaaaattatggaaccaaatgtttataactggcatagtttatgcagtactgtatttacttaaaaatatttctaaataacaggatttttaaaatataaatttcaatacataggatgtcaggttaagcagcacgggacttccactgacattaaaacttaaacctgaaaaacaacaacaacaacaacaaaacttaaacctgaagctacatgctgtactttaacaatggcgttcagaaagcctactatcaccggagttttgttatgctataataactttatgcatcagacaatttagatgaaattgcacttatttactttcaaataatcctactgtttatcataacaataagttttatgcaaatagattcatactgttaatcagataaagtcttataccaagctagagaattcaaaattgaacttttaaaatacttagcataaagaacagtttgtaatgctcaggctctcacaatcatgctccttagctcctttccatgaagtcatgatttctttcccaccagacttcctcctgatctctcttaagagagagattgtgagatattttacaatattggaagaaaccatctcctcttcagatacagaattctagttataacgttatacttcagactacatatgttatatatttaatattttatgagtaatgctgtaatttagaattttatatgtagaatatttagtctggcatacaacagccagtatagaatgagagtaagaaacgtcatcataagagattataataaaggggaggcatcaagcaaacatctcagagataaattctcattatattaagcataattcataaattcacagagagttgtgcaattgtagagagaaagtgttaccaaaatgatgtcctcagaattgaataaacaagcttaaaagtttaggaattcaatatttctagtgtatcatatacatatacctgaagaacattttctggtgcatagatcattaatacaaaattcaattaatatgaataacaaatgtactgttgttttatctttatttccttccctatccttgttgcctgtaagttgtaggtataagagattatatctcttaaactcttttatattgaggctggaaaacatactttttgcgtatctactcttttctccttttatattacctgagtagatactgctattttggaagggttgcgccctgttactacattgccagattgccaaaattcaaaggaagggatgtctgcttaacatctattttcatcctatatggactgatagtgtattagagctaaaaggggccttatagttcatctaattcaactcctttattttacaattgaaaaaaagtgttaaagaataaaagtgacttccttaatatctccccattattaattgaattcaagagggagctactgatgatctgacagttctttagtcttgactcatggttcatatgtattaattttgccaggccatttgttttagttttaattctatgtgtctgtcaaataaggaaatcactctgtttttccaaatgttgctgatcacatttacctatgaaaaagctttaaaaaccagatggatatcatgattacttttaaaattaccattcatattacttgactacatgtggtttgtttaggacacttttaaggtctcaatcatatctatgacattgttttatttataggtcaaggttttagaaaaggaaaatttaaggttcctcagtgatttcctcagaaatgttttaaagtgaattgtgaaattccagactgtagatttattgcttatgttcttttattaggacttcagctaggagaaaaaggacacttaaaaaacatatataaggcaatagaaatttagtttctaattgttaaaagaataagcctaatggttaccaagccttaatcctttacattttctctttatttagactatacattcaagaaggatcaattgctttatagaaaggaccaaaatctgagggtttcccaggcttttccagttgtttgtttactatgtgtcagataaatatgtattaacaattaaaaataagtggttagtggatctgttgtgccatcctgatcttgaaattctaagaaaaggagataagacaatacttttaagtctgatatttaaatattggacactcaatgtataaagcccaggttagatgcttggattgatcagataaagatactaagttcataacataaaatttttatcccataaaatatattcatagcactagtgatagaaatgggtacagaggttacagaattctttcgagaaattgtgattctagatcagtcctagataagtatctgggacctatcaattctactattgttaattagcacatacagttttgttgctgtgactgtcgcccaccccaaactaggctcttttctttccctaccttctgcttctgttctcagacataccatattccatttttttttctgtccccccatagcctgtttccttttcttccctgtgtatcaacttccaccggttatgcaagcccgaactcagttactgttgcacatgaatgctttttcaccaacacagttcacatggattaattccttttttaatattgtgtatcatacttgtctaactcacacttttggaattattcttacatagctccattgataattccattcaacacatattcactgagcatatactatggataactcatgttgctaagcagcattttcagtaaaatgatgagttatcacctatatttgagaaatttagattatgaaaatgggaaatacgtaatttaggattcaaaaatctatgcactctaaggagagattcaatcattgtactgaggaattaaaaacaagtagttaggagaggtttatttgaggtaaagtttaaaaaaaatcttattggcatatgtagcctcttattgtataggctaatgccagaatttgaagttttaagtgcctttttaaaaggaccttaaatattctgtaagattttgagtaaggaagtggatgtaacataactagaccatcctacttaagatatccttagagcaatagaaagcagtcactggaatgacaaaaaatattggagtctatgtgtccaattagagtgtgttatcagaaaaaccaatcatcgtcatcttaactaggggaaggataggagaaggaaataagacagatttaagaaagaaggacgggcacggtggctcacgcctgtaatcccagcactttgggaggctgaggcgggtggatcacctgaggtcaggagttcgagaccagcctgaccaacatggtgaaaccccatctctactaaaaatacaaaaattagctgggtgtgatggcatattcctgtagtcccagctacttgagaggctgagacaggagaattgtttgaacccgggaggcagaggttgcagtgagctgagatcacaccaccactgtgctccagcctgggcaacagagtgcgactctgtctcaaaacaaaaacaaaaacaaaacaaaacagaaaacaagaaagaaaagaaagaatacaacgacttgatttttaaaagcatgttgacaattagaaaaacaagaaattgagatttctgtaattttatacctggttatctgtatttggcaggttgtgaccctggtgatacctaaaacacaaaaatagcaagtttgaccataggcagtaggaagttgaaagtgacaatcggttttgtttgagaagtgggatatctgcgtgttcagtaggcagttagcactgagttggagatgatgcatttttggaagtcatataatatacaggagactagagtcccataatttttaaggagatcctgaagagtaagatagcacagagaagttgcaacacaagacactgggtaaaggaagaatgagccagcaaagaatgtgaatctatagcagtagagaaccaggagccagcagtcaaaggccaatagaagatacactcacataaacacatgcacacacacacacacacacacacacacacacaagcacacggacagattggatttgttgcttctatcttcttccgtttatttatttatttatttatttatttatttatttatttatttatttattttgagatgcagtctggctctgtcacccaggctggagtgcagtggcacagtctctgctcactgcaacctctgcctcctaggcttaagcgatcctccctcctcagcctccccagtagttggcaccacaggcacaagccaccatatctgactaattattgtattttttgtagagatggggtttcaccattgcccaggctggtcttgaactcctggccttacagacgtgagccactgtgcctggccatcttctttataattaacacttttttgtgtgtcagattagttttaggttcacagcaaaactgagagaaagttaggagtttctcatatacctcctacacacatagcctctcctattataaacatcccaaccagagtggtatatttttaccactgttcaacctatactgacacatcattatctccctgagtccatagattacattagggttcactcttggtgttgtacattctatgggtttggacaaatacatgatgacatgtatccaccattgtagtatcatacacagttttcactgcgctacaaatcctttgtgttttccctatgcattccttcttccccctagtccctggaaaccactggaagagatttgttttaacctaagggtggttagcattattaaagactgaaatgtgggtttaagactgtggataagacacgaagaaagtcattacatttggcaagagtgttttcaatagaatataccatagagcagtgatttgaatggtcaccaagaaagaagtacatagagattacaaatagtatacattagtctttcaggaaaaatgctgctaaaggggaaaatgatagaatggtggcttgagacgaaagcaaattgagaaagatcttggtgcaggggagatgttgacagtggcaatgaaaagactaaaaatgcaagagggaaaacaaagtattgaaacaaggtctgaaacatgatgggaggagggtgatgccattaagggcacagagagaggtagttaatctttaaaaagaatagaaacccactttcatctgaattgggtagaaatttaaagatgagcaaactcatagttagactgttaactcttatattatgtgttgtctgtttatagtttgtgactaccattatatccttctcccgcagtgtcacaacacctgaattcaagctttgattatcaccttttagggattcctgcttctaggacaaatcatgtaaaataaggaaagggtgtacttattgctgctaaaattttctagatgtgacagctttactctcaccatatttgttatatttgatgatattattagctaagagaataactgctcattagagttaaaacttagcaactattttctcataaaagattgtaataatacagtcacaatgtaagtgttctttccttttcctcagagttgatggattagcataaatctaaagaacataaattattttatgaaacatttagataatattaagattctgaactttttaaataattggttaatttttggtaattaataaaataaagcttttcaaattttagaaaaaccttttagtgaaaacagatcatgtaacaggagagaattatatgcagaaatattatgatagtttcttttttactgtattgtcagttttaaactttaagagaaacccaataaaccaaccactgaatctgcccttagacatatttaactacctaaatctgcctttagtactgtggatataaagatatgccaagataacatctttatatacaaaaacctattttcttgctgtttatgaaattggaactaaagtgtaaggaaaacaaaaataaataatgaagaaataaataaccacaaaagacacaaataggcaccttgctcttagagtccacagttgaaaaactttgctttgcccaggcgtggtggctcacacctgtaatcccagcactttgggaggccaaggcaggcagatcatttgaggtcaggagttcgagcaagaccagcctagccaacatggtgaaacccgcctctactaaaacccaaaattagctggtcatggtggtgcacacctgtaatcccagctacttgggtggccgaggcaggagaatccctttaacccggaggtgggggttgcagtgagcagagatcactccactgcactccagcctgggcaacagagtaagcctgtctcaaaaaaaaaagcaaaaaacaagaaacttttctttatcactccactttcactgcatatttgtcatttctgagcctcacgactttgtttctaaccagctccacccaagtttttttttgtactgtgtccagataattgtccaaaagctaacccctaggccttactatcaacatttttccctgtaagcttgtactgatctatctttcttagagaatgacctgaaaaatatgttcattctaagttgatgactgataagaataaattattcctttcaggaaaatagttgtctgttcaccgatgatatttctgcttcacagagtagaccttaaacagcagaatttcaggaattggtgacttttgattgagagtatattccgtccaaatatttctgtgacttctgagcaaatagttcctacatcatttttaccaaaaattattcattttaacatgataatcagaatcctttccccacattttttaagttaccagccatgcaaatttgttgtagctcttcaaactaaattaattttttttctcttttgcccttaactaaagttttggtcaaagtatcgagtatatcaaattttatgaggcaaaaatcacagcatgtttactttggcagaagcccacacaccctttgaggtattacaactctttgcaataatttttattatacacatttacactattttttttattgaggaaagtgagcataactaaaaaataaaataattcatttggggtcacattttagtaaatgattgagtcaagatccaaactcagggctctctgactctaagatctgacttttacatcacaccatctatggcatttttctgatttttttttattactccactacaaacatcatttaaaggaaatcttttcccccacattagaacagtgcctatttgaatgaacttatctgccttgaacactttggctttgcttttctccacctcccataaaccactgattttcagctccatccactgcaaacaagcacacaaaaaggatttgacagttaataggcaaagttccaacacatgctctcaaaaacttgacaattttttaacattctgatcacaacttcctgtatttctcctattagtttacatttccttatctttcttgaactaacaccttactttcattaaattttttcttctgtttgtcagtctaaacacttacttgacattctttagtctatgctacttctctaaaatctaccctctttctgtcaactatcttgctaacagactctcaaaaataattaaatattcactctgccctgaatatgttcacttcttgcccaccaccatggctttgcccactcaagtctctccttcctctctgctctgaaagtggtagagtttgtggagtccctatcatgtcctggctaaggtatttttatcttgctacagtaacatccgtatggattggaaagttcctccttttttacttttctttgctagattattacattgggggcatattacttcattttcccatgctacataatgaaaaatacacacaagtgtttctagcacaatgtctggcacatagtgggtactttaaaaagcaatctcttttttatttatttccatggaattttgtttgcttttgttattcataatgcttaatctaaacacctcttcatagaatcaaaaacgaagtatagttttgagtgaaactgtaagcaatattcaattttatttttcttccatgtctcatcaaatggtagtttatccttactaacgaagaaaagtttctgaagtatgtaaagatttttggaaatagtgagacattaaggaatggatagagtttttgttaagtttactagtctttatgtcagcatgctccaaacttgcgtagccatcagggtgacatttttctgagtctcaccccaacttacaaatttatgatttttggtgaaagggtcagaaaatctgtatttctaccaagagctcctggtgattctgattattagtaaaaattagaaaccacttattccaaagcctctttaaacaaagtgtgttctatgggccaaccaaatctcctgggaatatcttagaaatgtagaatctcagtggggccccaccacacacctactgaattacagtctgcattttaacacggtcttcagttaatttatgctcattaaaatttaagaagcactgtagtaaacgctactattaaaaaaaaaaaaaaagccaggcatggtggctcacgcctgaatcccagcactttgggaagctgaagtgggcagatcactgatggccaggagttaaagaccagcctggccaacatgatgaaaccccgtctctaccaaaaatacaaaagaattaactgggtgtggtgacatgcacctgtggtcccagctacttgggaggctgaagtgggagaatctctttaactcaggaggcacaggttgcagggagctgaaattttttttaacatgctacaagatagtgtgcaaatgcatttataatttaaataaatatttaggatctttataatttgctgtaatgtttaatgtaattattaaaagtctgaggagaacacgattttatgattgtgtgttttcatggtctatgaatatatttagcataattttcagctatgtgtttggttggggtgtgtgtgtgtgtagtgtgtagacaaaactgtctgcacaaaccaattgttttgggtaaattcagtttaaaagcaaaataattccagtaatataagatttttggagttttgcctacaaaatacaactaacttgcttgaccaaatttttcagctggttaactcctgaatatcagatagacacaatgattgaactaattaagaactatactattaagaacttaatagtgtgaagtttaaaaagagttttatacatattggagattaagttttatacttagtctctgatgggtcttgtcaaatgtctctgatactcctttttatttcaggaataaagagattatttgtgggatgtaggaaggataaaatatttactgtaatgtgatttattggaagtgttttagtaattttactcttctattaaggttccatttataaataccttattataatttgttaaatgacactttatagaatgcttgatggctttaatttatgcattgaatatgcaaatagattttaacaatatttgcatcttatatataataatcatttgtagtgttgttgctgaactatttgcttttatttaatttttacaaattaaaaaatttatttttaaaataaataaggtgctttcagatattatatgaatttaagttatttctgtttctttattctgaaatggtgatatatatcagtatttcactgggtatatttccatcttaactgagatttttatagggaggtttccctcaaaacagtcatttgaaatatatcaagttgtcaagattccaaacagtaggtttttttcctaattaatgcttaaatatattttaaacaacattgcttactgtaaataatctattttgatctagtagaaaatttcacgctattgcaatgcacttataaaaatgtactttgtactagtaaaggtagttttactaacaaagtactactagcactagcgttaataaagccaaattaaccaatactatttaaagctgagtttagtaggtattcattatttatttatttatttttatgtgctatttcttttttttttttaattttactttaagttctgggatacgtgtgcagaacgtgcaggtttgttacacaggtgtacatgtgccatgggggtttgctgcacctattaacctgtcatctaggttttaagacctgcatgcattaggtatttgtcctgatgttctcccttctcttgctccccaccccctaacaggccctggtgtgtgatgtttccctccctgtgtccctgtgttctcattcttcaactcccacttatgagtgagaacatgcagtgtttggttttctgatcctgtgttcatgcgatgtttggttttctgttcctatgttagtttgctgagaataatggcgtccagcttaatccatgtccctgcacaggacatgaactcattcttttttatggctgcatagtaacccattgtgtgtatgtgccacattttctttatcccgtctatcattgatgggcatttgggatggttccaagtcttaaatcattatttaactcattttaaaattctggatgtttcattattcgtattaagtttcagctttaattctttgaataaagcaaatccaaacacataaaaatgtagtaattgttaagactggaaaactgtgactaattcattttaggttattccaattcatattgtcagtattttcaaaaatatgtgaataaaatacactaccctaaggaaaggcactgatgggtaaaaatacagaattatttcctaaacctgtataggaagaggagaccattctcacactagttctgcaatagtttctcaaagttcaaacaactgctaattgggtgaataagagataattaaatccacctttcttagtgccttattaatgattctgaaattctcatccttattttaagtgcctttaatatttattttaagctcattcagttttctaaccacctagtctcagccctattgttgaattagatgttttaaaaaatactttaaggccgggcatggtggctcaggcctgtgatcccagcactttgggaggccgaggtgagcggatcacgaggtcaggagatcgagaccatcctggctaacatggtgagaccccatctctactaaaaatacaaaaaaattagccaaggatgatggtgggtgcctgtagtcccaacttgggaagctgaggcaggagaatggcgtgaacctgagaggcggaggttgccgtgagccaagatcgcaccactgcaatccagcctgggcaacagatgagactctgtctcaaaaaacaaaaacaaaaacaaaaacaaaaaacaaacaaacaaaaaaactttaaaggaaccaaataatgcctgcatgtttaaaagtatatataaaatgctccgaaatgataagaagtaggtcttctccctcaaccccagaagaaaacaccttttataatttttatttttttaggtctcttgaaaatggaaaatgaaatactctctgcccacaagaaactgacaatacatatcttctttatgatactgctttttgtggtcggaagggaattgcttttctgggagcattcaacaataaacaagtgtatggtcactggttggatttgtctttagagagatatccaagcatattgaataaatccaagcatattgaatatatactggttcatgtgccaattcatgaactgaaaggagtcctgcagatgtttcttaaacaggacattttggctgtctccatacacaaacccttaaaaataaactaaaccagagcaatgtcttcttcctgccaactaactgattgcttccactgatttcctgcagcatttattatgtgaaaaatactgcttctatttcttgatttatcaactatagatagaatatggtaactccctgctataaaaagagtaggaatttagtgatttttcgtcaccatcaatttttgctaattctattaagttcttctggtggttatctttattactcaaaaaataaactcgagtatatattttttctatctattgattctctgctctaaatcatagctttttaagaaaaaataatcgtcatcagactgcttttgttttataaagccactaaaatctttctgaagatacaaattgcaagattcttaaagctttcttctgttccccggattgtctgcttcatctctcgccacttgttaacattttttaacttttcattattttttaggctattggctttttttcaaatacctaatgttttttggttgttggttcaatatatggagtagccttattaatatgattagtatgtgaagtaaatattctgcatgattgtttagatttctttaaaaaaaaagcttctcccttaaaaaggggatgtgttggtagacaagtagacaagtttcaccttaggatcttcacttcaggactgagcacaccattgcccatacaatgttactctggcatggctgacttaattagtacatttgtaattgaggctgagagttaaggctaaactgctctgcttatctttctcattcctccacccatattgggtgctttctccaggcagatggtttgttttcttagactaataatgcacttctcagactttatcctgggggcaaaagccatttctgtgagaaagcttgctctattaagtggtgagggtgtgggaggagtaggtgatttaccagcctgcccattctcaatggtggattttaattaattcccctgatgattgtaccatggcccaccctttgccctttatgttccctgggaagtaagagcttactactggtattttaggctgcatttttccatactcttttgtttgtcaacttaggcccttccatctgctcactttctctctggtcctctgctggtgccttttctgtttttgtttttgtttttttccagcattagtatagttttattgttttaaaaacagagagaagggaggtggacttgggtgcttattctgttatactaaaccagaactcaatagttctgtaatattttatttaaattacagtaatgtaagcaacaaatattcatgatcatctaccatgtacatgttacaaaatgcatacactgatgccacattcccctacctaacgagcctagtgtactgtggcttacagagttgcaaaaaaagtttagagcacaaattttgcttattaatataattcacttggttctggtagttttgattcctatcaaagacagtttgagttactttttccttctctagtattaaaaatataacccaaagaaattctgcctacaagtgaggctgtaataaaaatttaaagccatgatcttggtcaaatattacagggaaaagtcttgctgttttaacgtgcactgcagcaacttcatagcctgctatttcttgaggctgactctccaaactagggatcaagaaccagaaagcccctcacagggttatagtgatacaagcttttctcttgttgcctgcaggctgcctaggggttcctgtattaacttcccaagtctgctcctaggggaaggcagatgtaactaagacaggccatactgtggtgtttcttatatgcagatatatgcagagaagtgttagagccatttatgaacttttaggtccgtcttgctctgaaattttgtggcaatagaacataccacatttcacaataaaattctgtttgacaataatatattgtaagggctagtatttgagttgggcagcggggaagatgatgaatgattatctagggagaagtggatgaagggtaggtatgtgtgttctcttcttggcttgcaggatatgatcatctggacaaagaggggaaaaatcagacaatctgtttcaactcaggtacaattaatttgtttttaataaaggataatgtgattatactacgtactttttctggggaaggcagactcaggaatctcattgacaaaagcaactttaaagtaaattcaaaactcttattcttccaaaattgtttacaattaacagagtgccatttctcaaagtgctttttatgctcaaaccagtccaataatcattttctttccatatgtgccaaagatatcctaaaaatattgctttatgtctactattttcctgattacagtagagtaagttaaagtccattaaactctgaataaatgtttactgtgtatatgctggacattggacaccgattaccttgaccattgtaacacatgcacccaaattatctaactcattaagtaaactttttaatgattggtttactgaatttttactcttttactgtaacacagtaactgtattgatcaatctagttatccttgatcatctgtctaaaattcagtacaaaatatttttggagattgtttatatccagtttaaataagattaagttagtggcaggctatagtttttagtgacataattacagaaagaaaggttgagaaaaattataatactttttatattgaactaacttatagtgaattatgttcagttatatgtcagatggaagctatatagcttccatttttttccaagagtgtgatctctgactaccagagatctcagactcagattctgctcatgggtctgtcatgtgctatgtgtatgacctcagggaaggcattttctttctctgaacataaatcttagtctataaaatgggggtaaccatttccagtctgatttaccacactgtagaccataaccatagaagctatgtacagggttacaccttgtttgctgataaggatttttttccacagcctacaagctgcataggaggtcgagtgttctctcccaagattctgtgtagccttgtccagagaatgttctacaatgatgagtatattctatatctgtggtgtccaacagaggaaccactagccatatgtggctattgggcacttaaatatggctagtgtgactgaggaacttaatgttcactttcatttcatttgaagttatttaaacttaaaaagccatgtgtggctagtggctactgtattggatcacatagtgagaagatagaaatacctgacgcactatttcttaaatacagatatacacatatagagagtttctgtgagatttggatcagataataatctaggtaataaaacatttctctacattaaaaatgcttttcaaatttaaattatcattacatgaaatagcacttctatttttgtggcactattggtggataaattatttcaagacattccctatttacgtaattctaaaaataatattcatatgtagtattcttatcagtcttatatttttactttattattttgaattctccccatatgctattaggtcactagagtagtatgttgtgtcccttaagttggttttaaatgaaaatgcttaccataacaagagttccaatgagcatatattttttttatttaggtagacttttaaaagcagatacaaatggctactcttaatttatatctcctctttgcctttcatgggatagcaacactgcccatttctattgaacctatcatgttctattttctttacagatctattttaatccaaatctgtaaatattgcccccctttcttgattagcctttcttctctagatttactttcaaatagtggtaatagtgtgtaactatttttaactgtacctttggactaataaggattctacatttaaggatatattgttttagaacaatgaatattacaataataaaattcaagtaatatatctaagaagatgaaataattcagcattcaattatgtttaaaattggcatctttttacatcagcttgatgtttctgtgaatgtccatttacctcaagtggtttcttcaaataatgagttagcactctccttcattgcaacaaattgaagcaccaaatttggctttgtgagtcgaatgataaaacatttgagagtcgtctgaactattctttgtttatattgtctttagttttgttcacttggttttcagtacccgtattcacacagttccttggcttctatatcaataaattttatgaggttcaaggtattgactgctaaaaaatcataggattgaatagtttctgttttggtttctttcagttgttttttaaaaaaagaaggatttgaaagtttactctgtggttttagattaagtaacttcagactttttttcaagaaattttaatgtctctttttaaaacttcagttttgaataagtagaaagtatagataagcaaaaagaaaaaaaaatcactctattccaccacatagttttaatatagcccagaaaaaaatttcaaacttagagagagagggagtctgtgtgtgtataaatacttgctggttcaaatgtgaacatatttttgacgtatcctttcagccagaaagttgtaccaattgacaaaacaaccagccacgcttgtccattctatgtgtgggcattttcatttccctactaccttgtcaacactgaagattatcttctttttctatcaacgcttatatgatcaatgataaaaatcattattttaatttgcattttcttgattattaaagaagataaatagtttttcctgtttttacactatttgtatgtctctatgcaagctaatggtgatatgtattttaaatcttcacatacaatgtatttcccaaatttacctggccccagatctcctttttgcagagtgtctggttttctataacacatctctgtgaaacactattacatactatagcaccatatttaattaccagaaattttataagaacttaactgtcaaatgtactaggttgaataaagcagtttatattgcaaataagttctatgcttcattttatatatatgatgctaaaaatgtatagacgctaaataaatatttattagttgattgaaccattaaatattggcaagtcaagtcttcaaatattagaaagatctttacctaagtttacctaaagaagtttccgtgtctatattggaagcattgctattgagtgaagttacaaggacctattttataaaatagatttctttcaaaaagccaaaaaagtataaatatgcatacatttcaaaaaacaaaaaataacagttggtttattgttacatatcaaaatgtggcctgttttaaccatatatattaactcatcctctaataatccatttgattatgatacttaaataatttctacataattccttcatacaatgaagtatagagaatttaaactctttttaaggaaaaaaatcccaagttcacctattaaatttgctcaatgtcctgtggggctaatgagaggttctatttaggtgaataatctgcattgggctacccacctcaccctcacccacatcttccacttaattttgctgtcaatcacaattagtggcattatacattttcacagctgcccaatcagtgctttgtttcagccatgcttagcaaatagagagccgactgattatattgttgagcagggaagtatactgactgtgtggagttgtctcactttggagctttttcactgttacaagatatgtacataagctaaataaaaatgaagtttgcaaaaacaacaaaaaaatagtatgtgagaaccaattctattcatttgactaaaacacaagcatttgggagcaaaacttggttttcagtgtttcattggaagcttctgaaaggtggcagatatttaagcaaagcctatgttcataagaatatgattatgttgaaatacactatattgctgacacctcctatttgggcacaactgctattaaaacatcaaagattgaactgagattttacttagtttttaaatgccaggagatagaattaaatcttcaatgtaggtacctgaatatatatttttttatattcactagatgaaggtatgttctcagggttgtaaactgcttgtggaaaaagaaagagtcagaaccaagtagaggtagacactgtaagtcatatacctttaattaacaacagtgatttcaaacctcgtacttcaccctttaagactgtttatatgtgcaagaagcaagtgcatgatatttataacataatgaacttatactttgaggaaacattcttaataaggaaaaggcacccttatgatttatcaaattgattatatcgtacatgacctaattggtcattcctgcctagtaatgaagagaaaggtagaaaatcctatgctgttttaatttcctatcttaattataatactcttttgctacaggctttcttgtattaacttcccatgttatataattaaccgctacatatctacattaattttcctattttctagaaccgatgaagcatttttattaccagaaacagaagggtattaaacactcactttaacgcagatctaaggctccaataaaatagtatctgtctcagaactttcttcctaaaattcctgaagattatatgaaattgggtataaaaagtaactactatttttttgcagtctgatttatttccttttctgaaagatgtttatgataaaagcaaaagcagaggttaatagatacccaattctgcaatgtaaacagtatagatactaattttggtatataatagaggtaagttgtttgtaaggattttatcgttttattcaccattagtttaatgtcaaatgaggggcaagcattctctttagcattgtaggaaaagaatccacagtgctaattccccagagattacaacatggggagtcaaaaaaggtgtaaggatcaacaacctaatcattatgatagagcgtaatcaatgatacagaagggtacaggctgataggagaagctgtaggagaatcacatatccagttcagggtgaggatcagtcgggggaaatgtccccaaaatgtgacaattgaagtcagtttgaaggagtttaccataagagttgggaatcttattgaggcaagacatgaagtaaaaaaataataataatatgagtagtttcagtgtaattcagctacctaataataaggaaatatttcaacttctttttccttaatactgaaggcatgtcccacatctgaacaggtcacgctgagtctcaggtagaggcagtcatctcattatcaaaacacagtgattaaaaatgctagtacagttagcaattctttttcctcactaaaatatcacatttttctctttttattaaagagagaagtcatggttcccttgatgttatctccttatgaatagctccatttccttggagtggtttttcattgaccacttggttgtgcttcttggaaatctggagtttatggcaagcaattgtcaaacttgtgcaggcatcccagcttggagtagcagtgcccagattgcaaattgaaagctgggattaagctgtttggggtgcccaaatcagcaccatttcactgtttaaaggcaaatggaaaatatctagaaagtcaggtgcccctgagagagctttctgaagcagatccctcttgggctgattgtgacactaatcagccatttaacaagcaagccattatgtatctgaccatatgaataatcataattcatgcaaatataatttatgttaaatacattctcaagttattatgcacaatattttcttcatcagaaagcagtgaccaaaggtgcacatgtatttataataattcttttcggcatcttgaatgtcccattttcttcttgccattaataaaagacttgtctgataaagctctcccctccatacgtgtcccaggtatttggaagagtgttacttatttcagaaactatacattaggatgatattcttagtctggtatttaaggcagtacttgaaattttgttgtgataacaaagcttgatatcctaggagcacagtcttgtcctggcctcattagtaaacaaatttgaatgctatagtctcagccctgacctcagaggtactcattagtgacttttctttattgtactgtaatgtaatcacagacactaaagtagcagaccattttctttgttaatcaaaaataattcagaattattataattcaaaaaaactttcttttggtagtgtgtaagaaaaaattgggggctcttctcaatgctcattttttatgatgtaaatcatgtattttaaatcattgctgcaaacttaagatgacaatctgtgtgcaaccttttaaaaaacagtcagcttattatatgtaaatataaaaggttagagttactaactctagaaatgaaaaagtaccataatttattaatgtgcctttctggcaggagtatatatctgtgcctgaaaccctcagtctcttcatacttatacagttctttgaaggcaaactatgcaagtgtgccaaatataatttcactgaaagtatttaaaaatctctttaaaagcaaagtattggtggtctatgaaggagaggtaatattccaatgttatatacattatagtccattctgaaacctctacttctttgccaattcattaggctgtatcattacaaacagataatcttatgcaaaaggatatgacattcaggcatcaccataaatgccagcaaaatgacaactacatttcatcagtaagccaatgtgcatccaggctgggtgactagacagagttgtgtaagttgtgtccaacacaggggcaccaggttacagggtaattggggccataacccaacctacatccattccccaaaccatgtacctaaggggttaaatctgtccagtgtacaaaagcaccttgtaggttactggaggctctttggcaagatatattgcttcccatcaagtatgacaaaacttgtagagctatttcaaccagcaagcatgcaacagtcaactccactgaaggagcatagtcagtttttctttaagaaaacgtgcacttgtgcacctactcaggaattatctcatctagtcaagccaatatggacagtgaaagtttctatctcagatacaatgcaaaaatgttcttgtaaaccttgacttttccaggaactaacactacttcatattcttatatccctgatactttgcctactaaatagttatgttcatgttatttaaacaatatataagtaaatatatgccagaataaatttttgccttctataatgttgactttttttgtattctgcttaattgtgagtgctactgtggccaagatccctaagaaaaagggattgttagttctactttgagtcattggttttacattccaaggacaggcaaaccaatccatcttgctttattctcatatgcatcccacattttctgtactggtattctcatacactttcatatttagatatgtctataggtgaggataaatgtggtgcttttttccctacacaaaagctattattaaattagtgatatatcttttgatggatactgccttacaacagaagaattggatttctaagaatggtgttccatctgtcatcaatgttctgataattttgctcagcgtagagtggagggagttgaagtttctctaatggtgggctgcaggtttctcactttgatttttttttctatgttatttagattcacctatctttaatattcattacttaatgacatttatcttttctaaagtcacctttaatttttaaaaaatttaattgtggtaaaatgcataacagatttctaagtgtacagttcaatagtgttaagtatattcacattgttgtgcaaccagtctccagaactttttcatcttgcaaaactgaaatgctatacccattaacaacaaaaagtaagtttttgtgaagtataacatttctataaaaagtacattaaccagaaatacatagctatggattttcacaaagtgaatacacccatggaaccaccattcagattgtaaaatagaacatttaccagcatctctctctctcacacagttactactctgcacaattgttttttatatgttgactttggattaagcaacactgcaaaattcccatattcattcttatagattgtagattcttaggattttctacatacacagccacgtttattcatgaataaagatacttttatttcttttccaatccctatacttttttatcctcgactttattgtactgcctgggacttccagtaagagtggtgaatggatgtggggacaatggccatctttgtctcatctccgaccttgtgggggagctttctatatttctttattcagtatgatgtttgctataggtttttgtagctactctatcagattaaggaagttcccatctgtttctaatttgccaagggttttcgtctaaacaaatattgcattgtttcaattgcttttttctgcatgtattgagataacctcattgtcttttcctttattattacattgacttatattgatttattttaaatgttaaattaactgcaagagtttgtggaagaatagtattgcttctttcttaaactttggaagaattcactggtaaagtcaactgggctgtaaatttctttgtggggacgtttctaaatcaatttatttaatagaattgggattatatggattttctactatttttggtaaggaatctctaggaacttgttcagttcacctaaaatattaaattaagtagtataacttttttttataatgttcttgtattttaatgcctatacaattggttttacagtccaagaacaggcaaacaagtccatcttgcgtgtatctgtagtgacagtccttttctcagttccagtactggttatttctgtctttttttaaaaataagataagtattgatgaaattatgtcaattctttttcttaaagaaacattcattttatttttccctattgcctttaaaagatatggctaaacttttaaaaaaattaatagactttagtttttagaccagtttttggtttacagaagaattaggcataaaatacagagagttcccttatactccctcttccactccccaccctcagtttcccctgttactggcatcttgcactggtatggtgcattcattataaggatgaagtaatatgaatatgttattattaaccaaagactgtggtttacattggattcactcttggtctttcacagttctatagtttttgacaaaagcataaattcatatatctaccattacaggtagttagtttcactgccctaaaaatcccctgtattccaccttattgttccatctcttctgctcaaccttaggaaaccactgatatttttaatattgcaatagttttgccttttccagaatgtcttatagttggaatcatacaatatgcagccttttcaaatttacttcttttacttggcaatgtgcatttaaggttcctccatgtcttttcatggcatgatagatcagttttttttcactgaatgataatcccattatgtggatgtatcagtttgcttatccattcacctattgaaagacatcttgattgcttccaaatattggcagtttcaaagctgctataaacatttgtgtgcagagttttgtgtagatatgtctttaactcacctggataaatatgtgagagcacattgctggatcatatggtaagactatgtttagctttgtaagaaattgccaaattatcttctatagtagatgtattgtgttacattcccaccagcaatgaatgagagttccacctccttgtcagcatttgatgttagtgttctggattttagccattataataggtgtgaagtgctagctcattgctaatttgcagttccttattgacttaagatattgaacataatttcatatgcttatttttcatctgtgtatcttctttggtgaggtgtctcttcagatcttttgcccagttttaattgggttgttattttcttattgttgaattataagagttctttgtatattgtagatataagtctttatcagatatgtgttttgaaaatgttttctcccagtcttggcttgtcttgtcagtatctttttttttttttttaattttttgaaacagagtctcattctgtcacccaggctggagtgcagtggcatgatctcagctcacttcagcctctgcctcccaggttcaaatgattctagtgccttagtctcccaagtagctaggattacaaaaatgtgcaccagcacaaccagctaatttttgtatttttagtagagacagggtttcgccatgttggctaggctggtcttgaactcctggcctcaggtgatcctcccacctcggcctcccaaagtgctgggattacaggtgtgagccaccacacttggcctcttctcattgtcttaactgtgttttgcagaatagaaatttcatcttactgaataaaaattaattttttaaatggatcagcctttttgatgtagtatctaaaaattcattgtcaaactcaaggtcacctagattttctcgtatgttaattttagaagttttatagttttatgctttacattatgtctgtgatccagattgcaagtttttaaatttcattaatttctattcttaacgttattatttcttttcttctagttctttggttttcatttactgttcctttttaaacctattaaggtagattcagatgatcaatttttcagcctttctttatgtctaatgtgtgcatttaagactataaatttccctttaacacgcattagctttatcctacaacttgatatattgtagttttataattgctcagtttaaaatatattttaatttcaattatgacttttaacacatagattacttagaagttcatcattttatttcaaatattttaagatctcttaagttataacttcataggggtcaaaaaacatcagtgatttcaagcctttgaaattcgttgaaatttgttttatagccaagaatatggtttattttgatacagttccatatatactagaaaaaaagtatatatttgccatcattttatacagcactttatatatataatgtaaagatctatatatgcatagagatatatgtgtgtgtaacaattagatcatgtattaattgtgttgttcacaacttctgtaatcttatggaatgtatatctgcttttcttgagttactgagagtgaggttttaaaatcccctactttgaatgtatatttgtttttcctttcagttgtgtaattttttcctttatgtgttttaaagtcattttattaagagaaagcatatttagaattgttatatttgtctggtaatatgcaatttttaccattctgaaatatccttaattctaataatttttatcttaatatctttctgttttgctgttaggatagctacaccagctgtcttttgcttactgcttccaagttatatttattactacctttttactcttagcctttctgtgtctttatatttaacttgtatctcttggaagcagcttataaatgggttatatttaagtattgatatatagctataagtatcttttaagtaacatttttactcatgacttgtaagagcacctaaaagattcctatgattactttgaatttagtcacaaggaaggaatatttgctacagttttctgtggtttattatacaaataataatatatcaatataatattttatctgtattccagtgaaaaacctacatttcacccccaaagcctccttaaatcctgtcaatcaggttgctattttcaatatggagacaacaaaatcttaataatatttcagtatattttccatgcttatgtgaatccttcatgagctacgcatatcaataatgggaaagaaaaatacttatttgcatttattttacctattttaatcatcctgtattaaagaccattgtattaaaatccaaggaatctaggaattggtctaaaatatgggtcagtaaaatactaatattctttagcaaaaatagaaaactatgcaattgtaatactgtagacattatgaaattatttcagggttacttttacagccttgaaatagccctaaaaataacacaaaaacgttatgttgacaacttgagtacaagacaatcatattctggtgtcactgtggcgttaattagtgggccagtgtgtgcagcaaaatgatgactatttggaagttttttcttatggaacatcatattccaccatgatgccagtttttccagtggtgcctagtcaatgccaaagagcaactggtataatatgaggtcatcaaagttgaggctcaggtgcaaagacatcagagttgtattaattaatatcaaaactttacccactatcataaaaccaaagaaagataccctttcaatgttgagcattacaaactgttctcagagtactttacttacattaaaatcttacagccatgcttatttgggtattaaattactttcccaaactggaattaagggaattcatgaaaacctgatttgcctcctcaggatgaaactgttcctgaccttttatgcatggcatttctgccatcagtaaatgtttgttggcctaatgaccaattaattggtagcagagttttgaactacctgatttctcattggaagaatgttttacatattgactgagctggaggtgttgcaaagttccttgaagatgttttatcattttgaattaacagataaatgtcagtgtgttacataaactaacaagttagaatttttcttaaataattagaatttcagtgatttttcaataagaatgattgccctgtggctagcatattttataatagaagactaattttgcttacaaaaattttgattattacgagtgattaaattttaccatggaaaagtaaggctgaacttcaagttttgaaagcaaactcccttcaaatagccttaaaaaacgaaattatttattgatatttgttgtaaatcaggctatagcctttagcttcacagcatggtattttaaggttcttaattagaaaaaataattaaatgaatctgtaaaacatggagatagaaactttagatatgccagagattcaaggatactttcaatatgttgcctgatttcaactatatgactatttacaatgttaagtacaggattgaatatgcttgtctgggctgccttaacaaattaccataacgtgagtggcttaaacaacagacatttctcagttctggaggctgaggtccaggatcaaggtgctggcagatccagtggctggtgaggatctacttcctggtttgcagatggtaagcttagtgaatccgcacctggcaaaaagagaactggagaactctatggagccttttttatgaggacacgaatcccattcataaaccctccaccctcataacctaattacctcctacaggccccacgtgttatatcatcacattggaggttaagatttcaaaatacaaatttttgtgtggatgtaaacattcagtgtataacaatgcctaagaaagatatttgttgttctcagaatagtctctcatatgttatgtcaatctatggttgttttggatggagggacaggttttctactgaaagcttgcattcaaatatttttacagtttttctaatagaaactaataaatatttcaaaatagaaaattcataaataactttctatacgttggttttgcagaggaataaatatggttttcttcatttttaaggctggtagagatatcaaacattctggtttgcctaggtcagtcctgatttacatctgcctcatctggtaattatttataatgcctctatttgctctcaaatgagtccgggtaaattctgtggtcaccccattaagtatccaagcttgcttaggcctctactaaaggcttccataaaccgtgatatgcagagaatggggcttgtagacataatgaggattcagcaaaatgaaactgtagtaagcgtagtgacccagaccttggatttggaataataataatttaaattcagttccagtcccaattctacctcttactattctttcacatatcttgatctttctgagcccataagaaatgggcagtaaaaagtatactgtctcacaaatgacataatgccattagaaatgtgaaaacattttgtaaatttctaaatgctatacaaatagtattaacaaacccacagtatggtttaactcagagttgtacttgtcaagaactcagagtcaaattaagtgtaattacaatgcaggatggaagtctgtgccctggaaactcacatttacatgtttttttcttgccattacatcttcacagcagattaaatgaccccaaaatactggagcatgccaaaagtagaaattataatagaatcttactcattttcatttctatatgaaatatgagcatttgtgtgcatattttaataataatgattatcataaaacagtatttacatattatagttgttaaccctaataacctcaaggaatattttttgaacccctactatgtgcaaaagcattttgccagattgtagatcagactaaataaagcacatcatttctccagtctggtttatgtgtaaacaataataataaaagacaaaatgagataaatgtaatagaagggaaaaattaaagtaaggggatagtagagaaataatttattgctaatccaggtgccctgtggatcactgcatagaggtgggcgctgtgctgagcctcggacgataaggtttgggttgatcgaggtgtttgccaacatattgtgctccaatagaaagagattaaaaaactaacctggtggaaagcatgtgttctaaatgtgctctgagggtggcactggcactgaagcatgttgtggggacatggacctggaaaggagttagtgttaggtggtgacagactttaaatttcaaataaggaaaaataaactatcttctttaggattgagagagctttttagtatttgacaagggtagtggcctaaagagatttgtattttgggaagataattctggcaacaacgagtagtattgattggagatgcaagacattagaagcctggagacctcttcagagactgtaacaatagtccaaatgaaaaatggccaggtatggaattagaacttgagggagtataaatgaccaagaaggcgttggactgaagaggcattatagaaatgattgggcagtagttactggctgattgaaaatagaattgaggtagaagaaagaataaaaattaccttggggtattatgtttggctgactggtaggataatggtgcattcaaggagacaggatctacagtaaaagaaaccgtttcacagattcagaatcaagcactgagtttcagggatgtgtagttatttgtatgtctataactggcagttttgttttcaaatctgaaactcagagaaggtaggtacatgtgcttcaagggccggtagataagttagccaaagccaactcacagtcaaactagacctagaatttttaagaaatttgcactgtcctcaaaacctacaaaaactctaaacatttagtgaaattggaggaccacctgagctactgtgatgccactctcatcttcttctgcaacatgaggtatgtcacaatgagaaaattattttattaactttatataactagttttttaagccacttgagtttctattccttttgccctctcctcattctttctttatgcataatgtcagaaacagcaacctgattggggaaatgagcatgatattttagagaggaagcctatggtaacataatgttttgtattctggaaagaacattcttacaaataaagccatagaggtggtagtgaacagaattttgggaacagtatacctagaggggtttggttttctgtattatattaacaatcaaattagaggaaggctaatatgtgagtgcttacaaagttaactgctgtgctaagcctttcctgtgcattatctcatttaatgctcgcaattaatgggattttttgtactgttgccccattttatttacaagaaatctgaagtggagagttgaatgacttggccactttagcattactagtgacacttggtatattcatacagctccactgattctagagcctacccttctaacatctgtgccatattgttatgtgaatatgtcagaaaggaggagaatagggattctccttgatccagcagagctacactagatgaaagccatcttataactgggctagtatctgtttattttataaggccacagatgcagaactgagaaaattcagtcttatccatgtgactagaggagactttagtaaatgtaatccaccaaacctgtctgttgggaagtgcaaggctagatatatcactacttaagaagaagatgaaaaatgtagaacaaaaggaaaaaagtcctttctatcaactgaggggaagaatacattcaaagagcactgtactatatcatccaaaagagaagaaatagaaaattgcttgacatattcaataggattcaagaaaacacaagatttgtaaaacagaaagagacctataagatcagaataggctgagatgagaagaaaatggtgtaagtttgaagagtgagataaaaatgtgataaaataaggattgcagagacattaaaatataatgttagaattaaattttgctttggagagataatagttaaaagctaagtcaactccctaatagaggggagaaatttgtattctcccacaatccaaagaaaatgcacagaggcttcaaaatatgaaataataatagggagagaatggagttcaaacctaaacatagtgggagttcctgaatacagacatttcttgatcaaatgtttgggaagaaatcaaaacacttcatgtagaaatggtaacctaacataaactctaagaaatatttttgaaaactaatgaataaaagaaaataaagatatgtagaaagggaaagagaaggagattatatcttaataattcctggctaatttcttataattttacaattttttatcaattatacaggtgggggaaggaggtttcctataaatcaggtgggcaaaattcactgatttcagattttgtttccctttttaatgccaaatatttagtattacagttgttaaccctaatacttcatgcattatggcttgggccatatctacacagttttgaaggaacaagtctctgaactaaaaattttatgcctggtgtatttgttttccagtgctatattgccaggtcccacaaatttagcaacttaaaacaatacatatttattattttgcagtttctgtgggttaggaacctggtgtggatcatctaggtccttttctcaggatctcacaaggctgcaattggtgttggctgtgttgtggtctcacctggaagtttttcagaagaatctgcttccaagctcattcaagttgtctgcaaacttttttttatagctaatatgattgaggtcttggtttctaaaggctaccgtcttcataggcagttcacagcatgacagcttacttcttcaaggccagcagaagaaggataaagtctgctgaaatggagtcttgtataataaaaagtgatcacagtagtcacatctcatcacctgagctatataacaaaacctaattaaggaagtaacatctcaccacctttgctatattctattagctagaaacaagtcacaggttctacttactctcagggagagtattatataaagacttggatcattgagaaacaccattctaggggacacttcacctgagtgagccactcaccactgaccgaagtaataaacttgcaaaaatgtggaagaatttgtgtctccttttgcaagagcatctgtatgaaacataaaaacaacaacaaaaggtgttttattacattaataaccctgtacctggataaaatagctttgaaaaagagtcattctggcaaaattcactaccagatgcagaagttcaatttaatcacaaccatatttttaaaaagaaaccttcagctgggtgcggtggctcacacctgtaatcccagcactttggaaggccgaggcgggcggatcacctgaggtcagggtttcgagaccagcctgagcaacatgacgaaaccccgtctctactaaaaaaacaaaacaaaacaaaacaaaattagctgggtgtggtggcgggcatctgtaatcccagctgcctgagaggttgaagcaagagaatcgcttgaacccaggagtcggaggttacagtgagctgagattgcgccactgcactccagcctggatgacagagtgggactctgtctcaaaaaaaaaaaaaagaaagaaagaaacctttatatatataggcagtcactgtttttcacagtagtatgacactataaaaagaactgtgcaagttaaaattgtgcaaagaatcttaatgagcaatgggaaaaataatgatcgttccatgcttaatatatttgtgaaaacatttaaaaaactcctactgtcaattataagtgtataaagaaatgagaaaatagtaaaaccaacatgaatatagtgtactatattgtaaagcattcgatattctaagaagtgaaagttttatttctttgtaaaattcttttcgagcatagcttgaacagtgcttgtcatcttcgatatagaatgtcttttctatgccttggtgacttatcacactcctttctaactttggatcagcttccaacattttctcttttgagccttcaattttatgaaatgtctctgaaaatttctttaatgtgaacctttttgccagtgtcagttccactggtgcatctttgtctttttgtcacaatcactttcctcatttatgtcattaagttcaccttcactaaattcctctggttgtgtacgtagtcttcaacggtggcagtgtcaacattcccattgtcagctctttattctatagcttgatttaccttctatttgaatttcacattcagcattatcactttttgtttctttgctgcattttcatcgtggttggccaatttcctcttttgattctccgttttgtaaaatgtcacataggtttatcacttggagacaaggagacaacacaactatattacttgctgtctgtgcatgaactaacagttgctcagtgatcagtcaccaaaaactttgaaagaagtgacatgattggttattgatcatgatgcatatctgttgtgtatgtagtgatttatggactaaagatctagcagtgaagtttatactttattcaattattcatagttaacatgctatggtaactaaaatttgaactgtgttgttgggagacttagattatttagctacaccattgcaactgaaattcttgtatattactgtctatatctacaaaatgattgagctttgaatggtaaaactgatttcaaaaaattccatgttttggtatttttcatattaactttaataaatttacattactttttaaacaaataaattctgttaaaaattggtatttaaaatgcagaaaatgcatcagttcttgatgggagtgaaggtaacagaatcgtaagtgaattttaaaatatattttaatattttaatgttaacattgtacatacatgtatatatatccttaattgaaaccatttgtaaaggaagattacttttgtaaactttttctacaattaactatgttatatatgtagtcagtaaaccttattgaacaccaggttcattgaagatgaaagacgcccagtaactgctaagaggttttttcaatttgtgttttttttttgtttttgaggtagagtcttgttctgtgactcaggctggagtgcagtggcacagccctcttacctcagcctcccaaatatctgggatcacagacatatgccaccatgtctgactatttttttttttttggtagagatgggggtttccccatattgcccaggctggtctcaaactcctgggctcaagcgatcctcctgcctcagccgtccaagttgctgggattataagcatgagccatcagcctggccctaagagttcttttttcattcttatactattatccctatttcagtgttgaggtttctgtgaagggaaataatttgagataaaacgtgtgaaattctgtgtgtaagacataagaaatatttcatccgtatctttgtgtatctatagtaagttattcaggagtaatcaaaaagctagtggtggtatcttgttctataatgtttctttatgaatgtgccaaaagaaatgcatatgtaaactcatttgccatattgggaatatgtgaaatttgttctgatttttcttctgatttgtaaatacggatttctactgagccacttatgtgaacacatttaaaaattttactctaatttcaactctattcattttcactttagtatttaaagggctggtatgttgaaaaagctgtagggaaagcattataattttattgccctcagaagcatctcaacagtattttcttattttctaatcaccacattccacataacatttcttcattaatttatttttcaaaaaaattttttgagtatgtattttgtgtcaaatgccacactaagcacaggttacgcagcagtgacatgatgatctagcttcctcatgaagcttagggaaaaacaactatagaagaatatagttatagctaaattcataggacctagctctactttgaatacattttcatagcttaaaataactacaggaagttttcttttttttctgtgcatctccatcccatccaattttgactggcatttcgctataatcgcaattccttttttcttcttcccaaaagaaaacatatatactgacagcaccttggtagtaacaacatcatgaaacttgcatccttagaggttttagtctaacataatgataacaatggctaccatatggtcttaatgtgaacaaataatgctgccttcatgggtaaagacatgtagataaaagttaagagagtgggagagggattaagtggggtatggtggtaggtgaggaggtggagagtaaaaaagaaatgacaaatggaataaaaatgaaaaataatttaggttcattatggaaaacttttaagtatactttttaaggactctataaaatttaattatatgaaaaaccccaatagcagagagtttaaaagtgtctagagatgagactatatatagtatatacccgcacattttgtttctcttcatttcacatgacgaaaatggaagagagacactggccccttaggaactgagtgtttgatatcctggatttgatctggaagatattgagactggaggggaagagatttgggaagcagctcctatattacgcccagctgccttgatggaagagcaacacagggcatccttgctcatccctcatgtgggaagaataactagtaatgtcacccttcatatattccaactacatatatatgtatatatatactatatacacacacacatatatgtatatatgtgtgtatatatgggatttttaatacacacacacacacatatatatatagtcctccctaagggaatgaataaatgaataaatgaggctcagtaagagttcagataaattttgattaatagattaagaaatgacattcaagagaaggttaagaatgttgaaggtacagtcctactcttttgagggaaatgtgatgaactgtcaggaagcctgactctaaattagtggcactaagtgacagaatacagggcttgatacacagtacattttttatgttcctataaccaaaggattactttctgcttcctcttccaggttttgttagttgtataggatagtagcattgatcaagtttttgatatctgtagatgggaaatatctcattcatggatataaattattatatttccatagtgttcttgaagcgatctaatacagtttatctaattttcctctcacctgtttatttcgaatcagaattcttgcttactaattctctcttcctatatataataccagaccctgtcaccaacctgacagtgtctgttagacattaccatggcactagggtcaaagttaagttacttttgtgttactttgtcagtggttttactcagggaatgttttataataacttaaatctggtgagaagtatttaccagcatatattttgcatgtgattgactctttgaagcattattcttggaaaatatttctttttaaaagctgtgaattatttataatcttaatgtgcatcataactgcatttagaatttttttaattaacaatatatttatgttttaaaattcaaaaggtacaaaaaagcagaaagtgtttttatacccttgtcccatagacagttcccttccctagagacaaccaatattatcagtttcttgtatatccttctagagatattttgtgcatattcaagtgatatgtatattttacacataatggtacttgctttttgcatcaagaaatatattttggaggccgggcttggtggctctcgcctgtaatcccagtactttaggaggccaaggcaggcggatcatgaggtcaggagattgaaaccatcctggctaacaaagtgaaaccccgtctctactaaaaatacaaaaaattagccaggcgtggtggcaggtgcctgtagtcccagctactcgggaggctgaggcaggagaatggcatgaacccgggaggcagaggttgcagtgagcctagatcgcaccactgcactccagcctgggcgacagagcaagattccgtctcaaaaaaaaaaaaaatgtatatatatatatatatgctcttatatatcaatgcttagcaagacataaagagtctccttgtttctttcttttctttttttttaaaatgtttttaagacctacagcacccactaccaccaggtggtctcccgtaccaagtagttaccaggcctgaccctgcttagcttccgagatcagataagattgagtgcattcagagtggtatggccatagattcctcatttatttctgtgactgaatagtattcattccactaatggctgtcatttaaccagtctcctattgattgtcatctgggatgtttctaatgtattgttgttaaaaatgccttaattaatatgtctgaacatatgtcatttcacatagatggaatatgtataactgaggataagtttttagaagtagaattcctaggtcaaagaatacgttccgttttaattttgatagatactacccaaatactgtctatagattcattgccactttatacttccataagctagcatctactttctacagcctcaccaacacaatattattacacagtctgacccttcccaatctgataggtttttcaaaatgtctttaatttgctgtctttataaattaggttgaataatgttcatatgtaaaagagatattttctaccaactgtagtacgttgccccttttttctatttaattgttggtctttttcatattgacctataggagtgtttcacatattagggaaattatgagttgaaaatatcattcctactttgccatttacttttaactttgttatggtttgtactatgaagattttttttaacatagttaaattattggtattttcttttatggactctgggttttgtctcattgttaggtggaccgcacctccttcacaattattccacgtgtgttctctagaatttttatggttttattttttacatttatttattatatctaatggcattgatgctaacatacggagcaaagtaacaagagactttatagattcacataacaattcaatcagaagacataacggaagaaaagatcatcatcatcaacaacaacaaaccaaagtaaaatatgtaggaataatctgtccatctgatgctttgaatgtaatctccttctgcctagtaaaggactttgtgtttttcccctcagatttcggcagcacccctctaactggggaataattaacagataaatatgcccccagatcttctcttttaaaaaaataaaactttggctgggcgcagtggctcacacctgtaatcccagtactttaggaggccgaggcgggcggatcacaaggtcaggagttcaagaccatcctggccaacatggtgaaaccccgtctctaccaaaaatgcaaaaattagccaggcgtggtggtgggtgcctgtaatcccagctactcgtgaggctgagtcaggaaaatcatttcaacctgggaggcggaggttgcagtgagccgagatcgtgccactgcactcaaacttaagcaacagagcaagattctgtctcagaaaaagataataataataatacaatttcttctaatccacatacccttccagttaagcaattatattgctcactgtcatcataatctagttaactaaaaggatgatctgctctctactactattactgccctcttcctcctcataaatctacaatatgacttctgacccaaccctcccttaaggtgattttagcttagttgaataacctccatattgaccaagacatagaacattgtctcatgattatcttcaactctttgcttagactcatggttttcttcttaaaacattcttttggcttggatattataataccattctcccagtttccaactcgacttctctagctgggctgtctcaacttccttcccagactcagcctcctcactgtgccttcaaattacagtgtttctctggtgtcagctttcccctttagagcactttctgctactgctattaattaattagtagcataatgagtggtttactgtgggtctcttttgttagaatataaagagagactgtacgggtcccttgctgaggtctgcagcttcatctccacctaattccccttttaccaggccaagttcgggaactttgtgtgagcttgttctcccttagtttggatctcctttgatcctatcagttcatggtcctttttattcctcaggttttagcttaaatgccacctcttgaggagaacttccctgactttatgatctaagatgatcctctttgtttaacaccttctttatttctttcctagaatatatcaatgtttgtaattattttatttcttctccagtttattgaattatttctcttacattagaatataccctttgcaaggacaggaaccttttatgtcttgttcttgtagaattcctagcatctcatatgtatcttgaacacaacaggtgagttgttttatgttttttgttgtcgtttttgtttttttttgagatggagcctagctcttttgcccaggctggagtgcagtggcgcagtcttggctcactgcaacctccacctccagggttcaagcgattctcctgcctcagcctctggtgtagctgggactacaggcgtgtgccaccacacctggctaatttttgtatttttagtagagaatgtgtttcaccacgttggccaagctggtctcgaactcctgacctcaggtgttctggctgcctctgcctcccaaagtgctgggattacagatgtgagccaccatgcctggcctacaataggtgattaataaatatttttcaatggaagggacagaagaaggcaagggacggaggttggttttactgatttaccattatcaccttaattcttagcacagtgaatttcaggggtattcatactgttatggggattcaattaaatatttcctgaagatattaactccttagaattctattcccagttctctgctcttattctgcaggcctcatatagattaataggtaactccacctacactatgactttaactatcattgatatgctaatgactccccaaatctaaatctagagccattactctttggttcaagaaccacatatgggctggtatagtggttcattcccataatcccagtggtttggcagctcgaggctggaagattgcttgattctaggagttcaagactagcctgggcaacatagtgaggccctcccatctctaaaaataaaaataaacataaaaaattagccaggcatggcagtgcacacctgtagtcctgcctacttgggaggctgatgcaggaggatctcctgagcccaaaagtgcagctgcaagtgagctatgattgtgccactgcactccagcattagcaacagagcgtgcgactctctttaaaaataaaaattaaatttaaaagaaccccatatataacagcctcgtggacattctacttggatattctacaggacccttaaatttaaatttagcataccaagcatgctgagaaccaatccacttcctgtattctttaccatcataaatgagactgtcgtcttttcagatgcctttgaatctcctagatttctccttttcttaaacagtttctacatataataaagcacatcctatatattactgttaaacatctctcaaatgctcccttcctctctatcccattcaagtaatctaaaaagtccacaccaatctcctaaccagcattgttgccacgaatatttctaaaattcaaatctgatcatatcaatgttcttaaatttcttaggatctccatgtttccctactctctaatccagaaatgcttggctgggcgaggcggctcacgcgtgtaatcccaacactttgggaggcggaggcgggtggatcacctgaggtcaggagttttagccaggtctggccaatgtggtgaaaccccgtctctacaaaaatacaaaaattagccaggcatgatggcgggtgcctgtaatcccagctattccggaggctaaggctggaaaattgcttgaacctgggaggtggaggttgcagtgagctgagatcgtgcccttgcactccagcctgggcaacagagccagacccttctcaaaaagggaaaagtaaaaacaagaaatgcttattgaaaaaatgtttaacttctaattctgatagccatggccctttcctgatctggctcctacctacctgtgctatatgtattccatacccctagtcataaagaataacttgccattgcacaaaaccatcctgctttcacctagaaaaggcctgcttttcacacttttgcctagggaagacgtttaacattcttaattcctactcattcattgaaactcaacttaagcattacttcctctcaaaatccttttctgattctgccatttcttcctgtgcattctctattcttcctgtgcattctcctatcatgacccatgccacactgcagtctaatcatgcatttactcaccttcccctctaccaccagaagttagcaaacttttgctggatggatgaatgactatggagggtttattcttcaagaatttataaaacaagttgaaagtatggttataacatagagccagtgttggaggaatacaaaccctagatataaaattaaaataaaataaaacaaaacaaaattactagtacagactaaatcagaaacagagaatggagactaagacacaaagaatcttatgagcaagagttggaatcctaacacttgaaacctaataattagtactagtagagactatggtgatttatcccttattgaactgaacttaggctacctttgaagctgggctgggatttaacaaggacatacttcctcagacaaggtttgcctgctatacaacagacaaaaccaaaatatttagcttgacctatattttgtaccagaatattccttttatataaatggtagaatgcataaatgtgctcatagaacactatgaacaggtattatgtatcactttgttctatttacttatttttgatgtaaatccctcaggagtctatgtaggtgaagatcctcaatctttttaatgtttttagatattaagtttgagtagagactgcagataggataatatgtcttagtattctacttgtataaactgaggcaatttattacatagaattaagtctcttgaaggggaagttcatctgggtaaagaggcaagaaaggaaaagagataatttgagtggttactgtgtgttatgtatttacatgctttctcacatttaatcctgaggataactgagtgacatggatatacttacccttatttcactggtaaaggaatagaggctcagagaaattaaacagcttgaacaaagtgacaaagcaagcaagactcgatattcaacttgaatttttctagtttcaaagtctaacactgcaaccaagctttttgttgctgtctgcatcttcattaataccttttaaaatcatcattcataaagaataacattatctgtttgatcctctggtgtggtataattgtttgaaaaaagaaatggccactttgtgaaaagaaactgtaaatactagttatattatcttcactaatcacaaaatagctgtctttgtaaaaccaaaacgggcatattttcaatccgtaagtccagtgttatcactcagatctcatctataaagagcaattatttttaatacatcatatgaggagaatgtttaactgtgttttcatagtactgtgacagtttatattggatttgaaatatgtgttctgaatctagaaacttaaatctgcattaggtacaatataaaagagaataaagaaatagtaatttataacatatatgtttcattgtgaattggatatggtgctatttcataaaatgttgctaggtttttttaatttatgaaatgtattaggccattatggtttaaaatttgcagtagtggaatttgtatatccaattttttaaaagaaactgtatcctcctttgggtcaaattattcttttccgatttctgttcagttagctgtaatgtttgaaagtatgaataaactggctgtgaaagtattgtccaaaagtaccaaatatgagttatattattatctcattagtcttcccctgaaggacctcttctaaataacttaccatggctagaaaaatgatatcaccaaatcagaatgtttttcattcagtcatagtattatattcagtgtagcaggaaatagttttgcatgacagtacacatgtaaatgtcattgcatatgtcatttatggtgtatagggttgttatgcaagattcacagtgatcatgttggagaaaactgtctctgatacactaaataaaacgtgcttagtaaaaattgctagcaattttttctagacatataatgacatggtaagagcacaaaacaaaactaccagaaacaaaccatttctcaagctgtaatatctagaggacaagtgatttaaattgctgaaacaaggggagttcatcgctacactacaaaagaacttgatatataacctaacggcaagtgaaatactgattccaagcttagaaataacagcaacaaatttacctacagacagaagatgcatggaagcaataaacgaattatacaagtcttacacatgcaatctgattattctgaccaaagtgttaattggatcctaactgccagatctgatatctgtggtcttgtagtgggaaacctttcaaagtgatgaactgtaattcactgagctgcttgaagcacttgtaatttactgcatgtcatgctatgaagagcctgacctaatggactttacattacctataatgctagaaacctttcagcaagactagttggtaaaaagtaatcgtagaagtcttttcagacaggatgagcaaaattatcttaaatacagctttataatcttatggaaagtcagttattaaacaaaatattgcaactgtgggaaaatagtctagccaacataatttacaacagtatgatatctcataatttgaaattaaatagaggtagaagatataaaaaggactctgcatagttttataatctaacctaaatgccatagaatatgttgggtttaagctattcatggatgaaattcctttgctttcataaaaaaattgtatttcataataatcagatttggtatctactatgtactttcatatgtattaaaagttatgggaaatgttataggtatgacagacctaggggtatactacattaaacaaatagctacattattttgaggataaattctgggcaaaactctctctcctaccatttgaaattatgagtttaaaatgtgaaattttgctttgacagcccataagtgtttattaactacagctaaactattgtctcttttatatattatgctgaaatttggggtgttaatttttgcacatgaagcccttatactataactaatgggttacaactgttctcgatgatgcagtaatagaatttgctttggttacacattttggaggagctagtttgtttttaatttatgttagaaatatgattatcagcatctttttgatatctataaagaatctttatgggataggtaacttaagaatatttttctaattctgagggtagacagatatgatttatgtacaatataaggcaaaaggtatagtttgggatattatggttctttcaccaagatgcatacattactattgttaattgtagtagcaacggaatcttaagcaactcacagcctggcagacatgaactaatcttggatctgtacaaattaggacattcacgaaaaagtaattgctaagtcgttggtacaatactctcagtaaatctacctcagttttccctttttttcctgccttcttccattgaagttaggctgtgtcagttagacaggctcatcagcaactccaagtgcagtgagagcatcctaatgatctgaaattcttcttcgaagttgatgttttctcatcaggatccaatttagagggctttgttccattcttgaactttggaaccagagaaaagcaaggcagtttatagccttctccatggtgagctagaaagacaaatcagtgtacccagcggcaggagggttttgaagagtttcagtctttatcaataaacacttaaaaataatcagtaactacttctccctatagcaaaatatgtccccttggcaagacctaaacataatgtacatttcattgaaaaatatttaaatatttaaattatcttgaaatataattaaatacttcaaacaggctaaatattttattgctttgcaatggcatgactgactgaagaatcacaaccaaagggtatagtaaatgctgggggtagactctgtttcatacatttgtgtttcagtagatctgctgttagtaattttactatttcagtagtagagttcagtgtaaagcgcaaaaagctgagagactgtaatatctgaggttctactgttcattttatgtgcagatgtttatggtgagactgtttcataaatgcttattaccgttagcccttctaagtgtaattttcaggagaaaaaaaattctgcaattcaaccaccattcctctctattcaccacacttgtcaatcagtgagttgctgtatctacaggaatggaggtgttatattctggcaattatcatgtcatatacttgaaaatgtaaatgaatgtgtgtgtaaaattaaatttatgggttgtcagcgaagaataagaaccaggagagcaattacttctctgctttcatcttttgagctctgatacatcaagcagaaaaccatgcagctaaatgttggaattagcattgtgcaggtcctggaatgaatgggctagtgtcttttgctgccttttctgactctttgttttggagagcagtgaaattaagatgattatcatccatcaatctatgagagtgcctatgaaagcatgaaaggatcttccttctcatatttatttttgctttcttagagagttgtcttttatcaggacagcttgctttttgttgaaattaatattatttgggctaattttgtgagttccatcaaaatcacaattgttttgttttttgtatctggtgtattaaaattcattttaaaatttaaaaggaaatatttatgaatgtagacaaataaaagattatttttagcacagttacagaaaagtagtctcccagtaattctgagaaagatcttaattctagggaatggatctagctcactagccatataaacatttttagttactaaatgtaaataaatgatggtgttttcataaatgactatacctctttttatggtgcacctgtctcccaaatgagcactcaagtattctaattctatgtaattattgattatattctagattttacaacttaaaataaaaccaggcacttcagttgcaaagtattattgattttgtttgtattctgcttttatctgttaagcattaatgttctataggttaagttttggaaagaataaaaaataagtgtaagatatgcagtaacttctttccaatgattttaggagcctaatgaacaatggtgcagatttgtttgaatatgaatgtgcagtgggatggagtttgaccttcagctgtgatttcagttcgtcaggattagtcaagctaggcgatcaagactgctaatgcataatgatttataaaatgaaaaatgtgtagtgaccatgcaaatgtttcagctaatcaaagaggggaactttagctttgagaagaaagaaaaacaagggcatgtcaaggcctaaaccaaagatgtatgtaaaataatctgagcagcttgattagacagagtgagattagaagttgcatttttggcctttaagcatatggtgtagcatattatatatcacttctccataatctctacaatgacacgcctatatgagcagtctggttttcaccagcagtacaaagaaaagaatataatctcttgaaatatttgtctgtaactaagagaaggacaaagttggacaaacactacttcccaaaacaggaaacactttgctttatcaaggtaactaaatgaatactaaaacttgtgtcttaaagtaatagtagaaattaagtgatgaatcatgattaaagggaatctctgtgtctgttattgcactgtgctatattatttctgttgttcctgtcacactatttccctgctgctaaaagattgcgttaaatagcaatatgcactgtccatcccagatctctttaaatactcctgttcagtacttgatgaaaagcttacaaataccactcaattcattgtcagttctttttaataccataaaaccaggaagcttttgaaaggctattattatgatagacattacacatgtaaccaaggcttgaatttttaccaaagctggcattccaatgcaataaatagtttcttatgttaaaataaactgtaaataaagatatttcagaggatagaagcaaacagaagaaatgtatcaagaatgtacaataaaatattttctattaacagtcaaaatcagagcagatactgtaacataagaacaaattttgcttttattacacaagtagttaattgtcatagttacatttttcaagtaccaagaaggcaaaagagtcaggacttcctttgtcttcaaattttaaacaaagaataaaaagatctgtgaaccactagttggcagaccgtgttaacaagaaaataatgttttatactgtaaatgtcatctatagattgtgactgctatttccaattcacatttagtgaagaaagaattggactgacactatactaagaacctttccaatctcccacacataattgggatgtttaattaagggacgcacttaatatgcaagcccagcacattcagaaatgctccaactgaactggactgtgaaaagactgtgcttatgtaaaacaaaaattttggtttgttttcataactcttacaataactctttagatgcatttgaacagagatatgatctttgcattgtctcgtaataacttttttgacctcctcaatgacatatactttaaattttaatataaacacatttataatatatatgtttttatgatttacatatgattttaggatgacaatgatgtgatggttatatttatgtcataactaaatataattatatttaatatcacttattaataataggtgatattaattgtaatacacttacctattctaggataatacctaaatttttttttttacttttaccactgactctttgcaagacagtttcagaattttccctctctcattttagaatgcataaacctatctttatattcagttaagtaatacttcttggtgcacagagcagcatgtcgggtttgtgcttaattcatgtaatgagataataaataagggtaaatggagacccttgtttggtgcatcactaaggagaattttaaatctgtcagcaaagtaaataccaagagagaaaaaaatctcccaaccaaagatgactctatttccttccttcacacttgaaaattttaactgataaatacccattcaaaacgagctatttcttctgatttcatttggtggtagagccaccagacttttatttgccttctcaccagaagtaagttctccctcctcttctatgtcacacgtctaaattgtgatcccttattagaatatttgcatgaatcaaattacatatctagcgaaaaatagttccgttagtctaacaccgaacttgctaaggtatatgtatttcaaaaaaattgtccataaatttttaaaaggctgatactgatttcgtctatttaaatattggtaagtagtataatctctacatttttatatgtatacaattaatctttattcatttttatcaatgaatacctagagtagcttgtggtagcaagtaaatgaaatgggacagtaacaagaaacgatgagggaggagaagaggaaatgtgtcgcttaaaatgagtcacttaaaaatgaaggctatgcatttctcaaattgcaagtggacgggtctgcaatattactgacctttgttgtagaaaaaactagttttttgtttattaaataacttagatttttagtaatctccttagtttgggtgagcttatatatgtggatttagagatgatagtattactttatttgtagtgttagaaaaccctcagaagtgtacctgtgtttttctacctatataattaattttaatttatgtcttttgtacacatagaagtagagtgttcaatcatgtatgtgttaattggctcatctttctatttatttttatatctgtatattgtcagttttagatgacagtttcctagaaggtaggccagaatctgattaaaattttaaaaaatcacatacatgtgaccatttaaaacatatgatcttaagaagagaatgatttcatggttatatttatgtcataactattaattgaaaggcactcaaaccattttaggagtaaaaataatcatctaacttgtgtagtgtaaggactagtataccctaaagaatatgaagatagaaaacttgcttaaatttgctcatttatcaaatcgatcacaatagcaacaaactctacagtcatattatatgtataagctgaaatctgactgtaaactcgtagtggtttacggcatacctccattcatagaactttccccataaagttcacttatcattatacataaagtatttgtacttgtaggtataggaataattaagggataattaaaacgctatatactgaaaaaataaggagcggttatgtaagaaaagtgcgacaagcttcttatctatgattaactttatagaaggcatatctctctctctctctctctctgtctctctctctatatatatccatgtacatgtctataaactctatatcatctatctatctatctagattgacagcctttttagtttaatatgtctatctttctctgtccatttcagtttgattcctatttggaaatagtatgaggtaaatgattgttcgaagaaaagacatttttgtagattgatacattttttttctcactacttgttctaaccattctatttgtttatcagagaacaaggagttaagtaaattgacagtcaattttataaaacaactaaacacttgaaaaacccattggtgaattaattttacaatatacatttcagaatgctttttattataataaattcaaattcagtaatttataggtatgccagtatattccttaaaaatacagaaatattttttaaaatcactgaatttctgtcataatgtttcaagttactcaatttgatctaattaatctgaatatgttaacatggagaaaagaaaagacattgctatttttcagtattaaatatgaaaggttttgatttatttagttctttatttctttttggaggaagaattaatccaccctggatttcttttttttttttcttctgcttcttctaacctcttttcattattaatttctcctttctccataacataaagtgccacactggaaaagtttgaactgcattaaaaataacaattaaaataagttgtgaaccgtatcttttaatcttttttggttttgaaagacatgttgcccaataaatttgttctaaaacagtatttatataaatacttgcttttatttacagtaaaagtgaattgttttttctaggaaagtgattactatttcaaaggttgcaaacttggaaagttgacaagttgtttgattgcatactggttagtacaagtaatgcctcaaacagtgttacttgccttctatctgagcatatactttaggtttctataccagtttttctgatcaccactcttaccatcctcttcctatttcttcacatccgttctgtctttctctagggctttcccactagcttgtcctttttctacgttttccttacttccttccttctttctcctttatatctttttctccttccctatcttctttttgcctctcatcctcagaaccctgaagaaaaaggaagtagtgggaaaaaagttttaagtaaattacttttattattttcagtttaatgaataacctttgagtaattgctaaatttttgtacttcctattataagctcagttgcttgaaataagaaaaggtttaaagaagaagctagaatactagttatatgagttcttctttcagaagacctatgtatgtcattatttttctggatatacacacagacacacacacacatatatatatatatctcctatatgaattatcacatgttgtcatatgctaaattttattactatatttattttctagtactttatcatgtgtatataaaattttgaagatgatgattatatttttaagtaagtatactgtatacatgctttttactagtttagataacatttatatgcatatttccaattttttaatctttaatatttattttaattacacttaagacatttcaaaaacctttgtatttatagcacagtgacattgatgagatgatcaattatgaaatattgagaacaccaccatgtggttgtttatttttaactatagatattatagccgaaattcaaatatctaagcatgttaaataagagctatggaacataaagagtttgatgaaatctgatgcctctggaaagctagtgtctatgttttgtgatactttagtactattgtaaagaagttatagtaaaatgtgacgtaaaaattattgatcatcaatgctaaagaatttataaaagataacatactatttgtgaagttgattaacagatatttggttatgaccacgaattttctttctctttctctttctgtgcaaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcaggtaatgtgggttacctgctttggtgttctagcatgacttagaaggtgtgcacttattttgaaagtgcagtgggcatatttgacaattcagtctccatttgaaaggacaacctattagcgtgctagaacataaatgtaacattttaattatagaactcgtaagaaaatctagattgcttatttttttaaaaaaattattaaagtctagaataagagcagagagaggtgtaatttgaaaatggaatgagccaatatttaaatattcaaatgcttaggtacattcaatttgttgacttgccaaaaaaatgcaaaaatacttaccagataatagttgcttttaaaactcctaacaccttagcattccttttagttcacaaaagtatgaaatttgaatagaccactttgttaaaatggagaaagtaacatcccagattttccttttcatagtatactcatcgtttagaaagagaagaatctatttattataatgaaattaggcagctgagccacaaatttatgaaatttatctttttaaaataacaaacatagtgttcctaataaataccatatcggaaccctgagtgtatatagactttatattatacacttacttattagagagaaaataatgtgctttacctgaaattttttatatataattactttgctaacttctgtgctgatgactacagaccaattacttgtaaatctgacattgttctgaacatctgatggattaagttttttccaataagaattgagcaacctgattttatgtagctaaatttttattcttacaactggtagagtatagcctagtttttatgtgtcagtaggacttcagatgtaataatttaaagtattaatttggctctgaccctttagaatacagactttctatacccttttgtttaggattttttggattctggattggaaaatttcagagctgccttggaaaaaaatgtatgtagagctgtctctttgaatccaatgtatattttttgttgtttttatgggatgaatcttaatggatactctgccatatgccagtctagaagagtttaggagatttataatacgtgaaacttttgcctttatttattaaagtcaaaatggtttattcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagaggtaggatccggttatctcattgatacataacagtttgcagttaagaaggggctttgagcggcaagaatagtcttagatcttcctataacaaggctcttgctgtcaaagttgcagtattatatatttttactgagcttagtaacagtgataggctctcaggctctccttttttgtagcatgggacttgagagttacaatctaatgctgctgtcgtctaatgttcactaatgaatcacagtacaaagagcaagctgtgtggtggacaaagtactgattatcttgtattcatggagaatctaagttggtgagggaaccttatttttctcagtggtgcagctcagagaacatgcatgcaaatttggcctattgttggggatagggagactaagggagaaactggttgagcacagatttcaaagtcacaggcccctgattaatgaactgctactgtaggttgagagtggcgagtagaaagttacagtttgatatgctcataaatcaaactgacaagcgggcttctcaggcctagcttgcacttgtcagcaaactgcatcaaatataaacataatacaaacaaaacatgttgaagtagttaaattgatcagcaggtatcagagccgttgtcttcaagctgcccattatgcaaacgtacaggaaacagttttgacctcctgaggctttgtttctgtttggatttgtgaatagaatttcagacagccatcaactacagaatagaaattgctcccttatttctcccgaggctttgggcaatccacatgacttgctccattatgcagtctgttttccagtgagcgcatcagaagtttctcttttccccaaactaaaaaggaaaggtcttccacagcagcttgtctttctctgaagtgtgactgccatctcataccctctggagtccaaagacctctggtttgtcttgtaatgcctcacaaaattggaagttatactttttcttataataagggcacttttttttaattttgccttgaacgtaattctgcctttaatatattccaacaagagaggagaagagagagagagctaacaggcctatcctatgaaggctacatcccagtttactaatagatcaccagagaccacattcatggacctctgcagatcaaacttattcaattggaaaaaaaaaaaaagtgtgtcgtagaagttgactatttctgtaaagtagtgttcttttaatctagagaaatgtattattatctacaataagtagattttaaaaagctgaacctttattacatactatctctagaccttgctccatacttttttgatgcaagttccttggttctctaacacgatttaataattgaaagaaaggcaactgaaaatctagaacttactcacattctgcccctgaactttgactatgggatgaccaaaaaacccactcaggcaatgaaaggagtgtgcatttccctgtaagagagctgtttgtacagaccatgttctctgctgtttactggtttgggttttctgataccagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaaggtacatacaaaatgttgtgcactcttcatttcaaatcttgtactttctaccatttcatggcctttctgccttataccttgagaaattttgtttttatgactttcaaatttattattaaaacgtgattgttaacatggtggcatgacctattttctagaggctcagaaattttaccatatactgctttcaagtgtgaagtaaaactttcaagtttttgttagatattaaatgtgtatttattagtcttatgggcacaaaatgatttataagggcaacacaattcaagcactattgaatatacaggaggatctgcagggaattggattaccgggaaggctgacagcagtccatttaactaagtgagctgataggagtttggtggacaactgataaaagaaagataaaagttcctaatcttcacactgaaatcaccacatgttaaagagaggcttcatgaatttctatagagcacattccaagttaagctttgtagaaccccatcctatgtcacaggacatatccattcttttttctctcaaataacattgcctatgcatgtattgtaaactatagttttaaaaaatctgtcaactgtattcttatttttctgtaagaaaaatttggaggtactctgataatacaattgtgtgtcatattcataacccttcacttttgggaaatggaaagttttaaattataaaagacaaataatggtaaacaaatgactgtgtgctaaaattctcaattcaggtaagttatatatcattacacttcataaagggatattctaacttgcatttggattttgttgggacaaacattaatccaaatgtacattaattcaaattaattatatttgtcacagaggagttcatattgtagtagcattggctgcacattttgctgattctaaaggacatactaaagaaaatcaggagtatcgtggtaatgttctctctttttatttatcaaattaatgtctctttggcttttttccccctggttttcctctctcacttgacttcaaatcttctaattcacctgattgaagcactcctaggaaatcaaaaaagataaagtttaaatagcaaaagaaaaacaaggcaataagcctgtttttctgagttattctaaagaatattggaataaatattctaaagaaaattggaataaatcatagcgtcattatatcaatgaatgttctattcacaattattcttctttagaaaaataaatacaatttaaagacatgaaaaagtaaaaaagaaaagagtttataaaattatttcagtgtaaacatggatttcttcaatagaaactatactaaaacagaaaaaatattgtttttgttttcagtcaagttcaaatgaaatctgtatttaggatataattatatgagaatttcctcaacagaaaagcatgatctaccacaatcttattttattaattcaagtaaacattttaagaaagtttaaaatatgtgctaatagaaacacagtgtaacttgtttaaaacatattgtgaaaccacattctttcaaaacaagcaaaaaacatctctctgaacaatgaaatttataatgatataatcattctaatagttttcaaatagaaattacctgaattcaaattaaaatttattttcatagaatccttgtttctctcttttcttctgtttttatccttgataagatatatgagagataaaatgagaaaatggcgaacatttagggaaaggaaagtaaatctgaaaacatctgtctttgagcagaaaatttatattttaaaaaggaaaaaagatgaaatcactcaactatgttttcattaatataggttaaaaaaatccatctgaataattgtagggccacaaatgtagttggttcaccgatcaaagtttgtgttgttgccatgtaagccacacaaaccggcatcattgtcttgttttttacgatttagatgattaagtgtgaaaaatatcattgtggaggctcattgtgtgttgaatacaccagctacagtaaaggaacctagcgttacggtactatgctggaatcccaaaactagagtggggaaaaagtaaagaaatatttgttgtacagtgtaagccctgaatcatttcttccttagttagaggttaaggtaaaagtattttgaatatttttattatcagtttccagttcactttttaccaggtttttgattggttgattagccttgaaaaatagccatataaaaaccaagtaggattaatgtgcaatgatggatgatgttctcattgtgtctttaaaactgtatcttttcttttttttttgaggagtctcactctgtcacccacactagagtgcagtgacgctaacttagttcactgcaacctccgccttcccgggttcaagcaattctcctccctcagctttccgagtagctgcaattacaggtgcccaccaccacgcctggctaattttcatattattagtagagacagggtttcaccatgttgaccaggctggtctctaacccctgacctcaagtgatccgcctgcctcggcctcccaaagtgctaggattacaggcattagccactgtgctcgacctaaaactatgtcatattttatttgtccctataatttaacacaacttcttcatgctacttttagagttacagcttcttaaattatattaaaatatatgtatataacaatgagaaataataaaattataagaattttggtagaatagcagatttatctacattcctctctaaagttttatatttatcatcttagtgtaaattattttaaatattaatcatttaaggtcatcattaatattactataatattggattttagttaggcaagtaaacttccatctcctaagtatatgtttggtttgtatacagacagatgatagatatatacataagtataggtatataggtatatacatacatatgtcaaatagaaaggatggatttatagttccatcttgaataatatactgattataaatctatgtaatatatgaataacatagtttatatattatctagtaatgaaatttagtgaaacattttaatgaaccaggataattgtctattggttaggaaagatgaagttaactttaaacattgatatgtttgtgacatccgataaacgtttgtgtactctaatccacaatatattcacacatttactatgactatatggataggaatacatgtggtgatgattttctgtcactccaagtttttacatattttttaatttataaaagttatttagaagacaagattttcctttccaatttgagagaccaaacagagaatgctagattctagcctggattcttctgaattaacacattatagaactagagattctcagaataagagtggttgaataataattaaaaatgaaatatttgaaataaaatataatgtttttaggctaagttagaattgtgccgaagatagtctttgggcgcaatattcagccaaaattagctgtttggctttggcttcattctatgcttatggaagtctaagtagccaaatgcctacttctggcatcctggatttactgcttaatcaatgatcttgtgattgcatagattactattgtatgtaattgtagcaacaacactaccaatatgaacaatccatgtcatgcaaaaatattaccatgtcagtattccctttcaaatatcaaagttttccatttgtaacttggggaaaaaatcttatttggtgctttatgtgctaaaatttcatcactgaattgaagttttaatgtacatttattattgcaggtataaaactctgccaagctacctgtgaatgctgttaattttatgtttaaaaaactagtaaatacttacatatatagaaaaatattgagtatgttgagttagtttttgaaaaaatgtaaaaagattgagattaaactgtatcataactctataattcttgtagaattattttgtgaactagaatgtataacattaaaatgaatttaattccatttatctcttaagtctttttttaaagttttatttggcaattctttacatcttgagaggaagactcgaaagcagtgtgtcaactttgccatattaaaatatgatgagaaaaaggaaacatcattggttttgtaatagaatatccatcaagcttttgaaatgcaacctataaaaaatgatagtactaatttttgttaatatcagatgtattcacatatcccagaagttccttctttggagaagataatttatctttgatctcagtttcagctgaatacctacttctgctaaatttggcatttggacaaagcatgtatttaattcacctctcattagacaagcattttaaagataggcattgtatctgtaggtgatagtagacctctagtcaaatataaatcaataatatgttgagttagtcaaattataaatgaacacccagatctctgaaggatactccagaaactgggactctcttctgaagaagagatatgggtatccagaggtcaaggctggtagggaagtttacttttgtaatatatacccttttctgtgattcaattattctggccatgtatgtataccagtttttaagtttcacatcttaaaatacaaaatttaagatgaaaactgaaaagtacatagtaatatctcagtgattcaaattcaaatattaaatatcaatttggcttaacagaatatatataatacaaatgtttctaccaaaactcacttttattactttaaattataagtaaccttagaaaataagtagcgtatcactaaataagttagttaaaaattaacagtatacatactttgcatcataatatatacaattatttgtttaacatttaaaaatcaattactggacacagaaaaagaaatgacattgaaaaaatctttttttttttttttgagtgtaaaaaagctgccaaggagattttttactaataaaaattgctttttacttattttagtgataagtaaaaatcatcttagcagcattttcaaagctgtgttgaaactagcgtagacatttttaaacttcgtgtgccttaaggacatataatctagatatgaattaattttaattaattcttagcacagtgcttgtcatcttgtagatactccacaaactctgaatatgggattaaggaattattattaggccaagtacagtggatcacacctataatcccagcaatttgggaggctgaggctggaggatcacttcagcccaggcgttcaagactagcctgggcaacatagtgagaccctatctttacaaaaaataaaataattagctgagcatgatgacgtatgcctgtggtcggtcacatcccagctacttgggaggctcaggagggaagatcacttcagcccaggtcagggctatagtgatctgtgatcatgccattgcactccagcgagaccatgtcttaagaattattattaagttgtattatctgtggtatgatagacaatttgattaaggaattaccctttcatttactcagctcctgtggtaaaaaccttttaatatttgtggtactggcagtgaataagcacaagcccaagttatcaaccaaaagtagaatagggattcccaattttttgtggccacggaattttaaaattgcataattacaaaactttttggcactactagtatatatatccttgaaataagagaaaccgaataatcttcacttaagataaaagttgtctattaatcataatagtctagtccacagattcccagagtatgtttgccagtacacgagtgttctggccaaattttaaaactctttcaagtaacattctttgagctttctgtgactgcaggcactcttatagacactggggatatagcagtgaagaaaacagacagaaatccctgccatcataaaacatacattttagtgtagaaggcagatgataaataagttaagtaaaatatatagtggtgagcgctaaggagagaaataaaggcaagaataggaaaagcctaagtaacaataggaaaatggggtgaccctcagaaggagtcctgagaaaagggttaatgagtaatgacctgaaaatgagaggaagctctgcgacagagtagctctagagcaaggccttttctttgccttagtgtccccatctctaatatagggataataagagcaataataatgataataatggtttggggtattagattagatatcatatgtgaagcacttagaagaatgtctgccatatcaaaagcactatgtaagtatcagttaaataaaacagagacaaagaaaactctaaagaggactgagttggggttgagggagggggctgtataaacaggagcttagttttgaacatggaaagtttaagaaaacggataaggttacatgggattttcaaatagaaagactgggaggggtatgggatgggatcagttggtgactcctaagtttacacctggtatttaaaacttacgcaaactagtgtaattataaagagtttcttgggtttttcttgctcaaatagaattggaaaacgctgacttcaaaatgagattcaggttttccttgttactacacatctcagaactattttacatgaataataagtccccaagacagagaccacagatctcttagattaacagcatttataagactactgttccatagaacactgttttggaaatactgaacttgttatattactgcctcaagaataacctagattaatcattgcaaaccatcaagccttcaaagctcataaaactttcaaaattgtacaaaatcgattggctagttgtttcacatttcataagtagttttgtaagcatataaaagagctgagcaatttttaggttgaaagctttgtcccaagagcaaggtaatagttttctttttaaaaatgtccccccaaaattatctgtgcattggaatcacctttggagcttcaaaaactactaatgcctgtgtttcccctctaaaattgtggttttattggtgtgggttgtggtaaggcctttagagattttaaaatatgcccaggtgattctaatatgcagacatttgaggatccactgccgctaaaaggaattttttgacttcttttattctctgcctcttatctattatataatttaacactgtgtcttcctgtttttaattaagaaacattatttggattatttgaacaggccatttattcatctaatatttattgagggcctactaactgcctgacatttgttacttcattacaatagcacggttatatcactaattttcagtacaaatgaatagtatcatagaaatctacttttagaaggcaagtggatacttacataactaaattattaataaaatccaaaaactgagttaccagtaacatcgttagttggtataatctctaaaaccatctaagagtcttaaactgagatataaaatgttgttagccactcaatctatgttagaagcaactagtgccatatataattctttagacactggtcttgtattgtagcaatattcatatagagtatgccctttaagagtcacaaaacttccatgtgggcaccaacatgtcttcaagaaatgtatgactctattataatcaatggaatctacaaatatttattgagcacatgccacgcactgttcaaggcagtggagatacaccagtaaaacaagaaattcctactcttataaaacttcagttgtattagaaggagactggtgataacaagaggtagactagatagatagataggttaaatggtttcagaaactgttacatgattttaaaaaaaaagctggatgaggagttaaggaatgctagaagggagggtggggaggggcaggggttgatagtatggttggaaacttagaggaggtgaactttgaaccatctaagtaaagggagggaggaagccaagaacaggttgtgggaacaaccagagcaaaggttggaaagggagcaaatgtttgatacatttgagaagcaacagaaggctggtataacagaatgaaaagtgaagagagagcggtagatgagatcagagagacaggcaagagccagataataagtgcagtgccccataggctatattaaaggttgtttttgtttctgttgttgttgttgttttgtcattttgttttcttctaagtattctcgaaagcctttggaggccttgcctgggaagcgacatgacctaatttacatttatagagagaatacttggatactccatgaagagtaaattcatatattcaaggcaaggtaatagcaggaagacagttggaaggctatagctgtgatccaggcaagagttgacaatacagcagagaggcagctggtaagatggtgaaagttctaatgcaaaagacatgttggtgctatcatgaaaaaggatttgctaatgaagttttcctgttgggacacctaacatacaccaacagataatcaaaatttactgtcagaagagtttcagaattctaggttaaagcaataattatagatctttatataaaaagttgtttcctatgtaacagagaaacctttctttataaagccactttctttttttatggaaaaaaaatgggttgtagtaactttggaaacccgaattatcctgttttttctttaggctaggtccttgtcttaaatctcaaggagccagaatcactgaaatgatagccttcagtaaaactacctgtacgaaattgtgataggcatagctatttaaggttaatgctgtgatgaataatataaccagaagttatgtttggaacagtgagaattcttctgttcctattggctactggccaatacttagatatagtgatatctagatcagggatttttccatttgcagggattggggtgagagagttcattcttctcaccatcatattctgttatcttgccccatctcccgtgactattccaacctgcttcaccccacacatctcttacatcactatagtatctaatgtagatgtatgttcggttgggagaggtagtttgagttgcactttcatctttatattagatgttatagatgcacatagtaattaagaacatagattttggagctacagagcattgagtttgaattctgcccactgatcataaattgggtgatttgggggaacatatttaatgtttctgagccttcttgtaaaatataatgatttctaacctcaggtgaggttaactaagattataaatgtaaagctcttagtaaaaatgccagaatatagtaaatactctcttcattttttcctgtacttatttattcagtagttttaatattttcttaatatagtattttaatagcaaatatactcaagccagactgcctggatttaaatcacaactttaccacgtagaagctgtgtgatcttggacatattttttaacttctctcttagtttgtttccttttccatgaattgcttaatcataccaactctattagtttgtactgaggattaaaaagcacaaggtctataaaaaatatacaaagacacagtactattttgtaagtcaaatactgtaagcaatagtctctcaatactttatttcaaattcatagttaaaaactaaatgattatatctagcttttctatttttattgttgtataattcaaataaatttgcataacctgaatcagggaattgtttaaaatgagtagttgatttactctatcagtcataaatgaaggagaagtttagatataatcttcttaactgatccagttaagtaagcatattggtttgtttaagacaccaaaattaaatttagctatttagttcatccctctgagaaatatcaaaatgaacagataattgtcaaatctcctatttttttgagttgagtgttcactgtttttccctggcatttttgttttcccttttatttatttatttattttgatattattttgaaatctgtttctctttcagttcctcactgatttaaaaacaaaccttaatactagcaataggtaaaaactactcttaaaagttgaatagttcaatttttatttgtatttatttatttatttatttttattttttatttttttgtttttgtttgagagagggtctcactctcacccaggctagagtgcagtggtacaatctcagctcactgcaacctctgcctcccaggttcaggcaattctcccgcctcaacctcctgagtagctgggattacaggtatgtgacatcacacccagctcatttttgtatttttagtagagacagggtttcaccatgttggccaggctggtctcgaactcctggcctcaagtgattcacccacctcagcctcccagagtgctgggattacaggcgtgagccaccatgcctggccctagttcaatttttaaatggaacatgcagggatttttttttaatgcttgatattattctcagtaatttcatatggcattattccccatactattcccaaattggtactctttagaaaataaatgattgtttttcaagacaaaagctttggtgctgatagtgtaaaagtgattttttgttgtatattaatttatgcaggtaacatcactgttgttgttggtattaatctattaataacacaaagctcattatataaactttacctttaccttgttatgctagtgaagctttctttcattttatagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacaggtaaatctcatgagctttattctatatttatctattttcagattttattttcaccattgagacaatgaaaaagaacaatttgtacttggagcaaggacaaaatgtatagaacaacagattagaaatctttagattatttatcttattttatatataatatatatatatatatatatatatatatatatatttttttttttttttaggcagagtcttgctctgtcacccatgctggagtgcagtggcacaatcttggctcactgcaagctccgcctcccgggttcctgccattctcctgccccagcctcccgagtagctgggactacaggcgcccgcccccacgcctggctaattttttgtattttttttagtagagacggggtttcaccatgttagccaggatggtctctatctcctgacctcgtgatcttcctgcttcggcctcccaaagtgctgggattacaggcgtgagccaccgtgcccggcctatattctttatattacagaaaagcagttcaaataccaaaagtttgatggatgtagtttgggagtttgcctgaagaaatttattttttcatgagtgcatgtggaatgattgtatcaatatatttttatgactgatatgattaacctgattgctgagaacattttttaagctaaaaatcagttctttaatttcaaataattaaagatgcatggatatctctcttatatgtttcaaacatatgaattagaaatcagagaacagataaatatataaacacaagcataataagctagatgatggaaaatctggtttgcctttttaattgttgcatgatgaaataagtaaatatataaagataaatatgttggtaattttatgattaaattcaactaaactttcaaagaaaaattttttatttcagttattaatattaaatgtgttatgttagataatttttttgataatttttaactactgtttttagttgtataaaaattgcatttgtgaaaggagcattcatgtacatttgtcctttagctccatttatttaggcatcttgctctctaatcaagtcctttaaagtaatttgcatatagttcataaactaaaatattgttcagtgtgaaaaatctatttttgaaagatgaaatgtaagtgtagaattcttaacataatggcttaggcttatatatagctccaaacttcatatgtaagtgtagtggactgatagtgttctgttatgcaatatgaatatctgccttgccataacaaattctttgtcataaacaaatgttttccaaagaaacagatatttaattatagtacagtttaaaataaaattctataattcagtgatgtttctggccttcatttgagtatcatggaaactttttcttgtttggttttattagtataaagtggtatgctcctgaatgctttcatgctgttgttttaattgagattaatttgatgattgtgtattccaaaacttggttgatttctctgggatctctctaatattcacttatcaaaagtcttcaataattatataaagataaagctaatgaatactgttccaaagtagattttctaaatcaggtgtaactagtagaaatatatgtggtttatacataaatattaaacaaattaaatgatcctagaggtccatatcttctttaaaaagagcatttcaaaatcttatgatggctgtcctattattctcttgaaaataaaggtttttttcacttgaccacatatgaatatacattttttatttagagataaaatgttacattaaggtcataaggcaggattagcatatgatattatacagtcctcttagtttttcatcctttattaacagtcatctttggctctatttctcctctaattggaacatcatctagccttggcagttgaactattcaatagaacgattaaatttttctgactgctctgagctgaattgacctgttttgacctgtttgtcactgatcgtaacctgacaggcgctagcagcctgcaacgattatagctttttgagatgaatctgacgtcgtgttcttttgctacagctcgtcacatgaggagactggggcctctcacactctctatggccatggagtttgcaaatggccaggctgtgaaagcatttgtgaagattttggacagtttttaaagtaggttttttacttttttttggtggggggcgggggctggattatatgggcattttaggcacattgtaaataaacaaaagatgaaggaggaatgtaaaagtccattataagtcatacttcaagatttttgtgggttccaacaagtggattagtaagatcaggcttcatcttttcattaagatattttacaaatcatttagtaaagatttttgaatgaatgaaaaatattagcctacaataatatgtcattaatcactggagacatttataacagaactatttataaaatagaattctcttaagttttaagtttctaattaatattatgtgagtcatcctaatatatatatatatatatatatatatatatatatatatatatatatatatttcagtaaacatattttgaaagttcgttttttaattacaggctttatatattttttaaaatagtagtatttagtatatacatgtaactacaagaattaaaagtgaaagtttatactggctaaaattaaaaaaactacatttcatttacattgttactttttgcccttagaaacatcaagaaacacacatggctatatgtttgtataacagattacatggcttctctagcttaaaaccaaaactttgtgtattcttaatttttaatagaaaattttatttttaaaaaaatcataaggggttctttttcaagtgaaaatcttcatctttcatgaaatgttttccttacacaattctggtgtacaagagatctaaacagagagctacagataggcttatggagatgagtaacttcacaggctgagactcttatgttgtcatggtgcagctaacagggtgagtgaactgtttcatgcttcatcaacaattaagttaattagctcatttgaaattgcactgctttgttgccaggatgttggcagaaacatcaaaaagatgtgtttacaaatggaagactgactacagggtatagagcaaaagcaccttaacaaccaggcacaagtacttggcatcacaataactttataaatgaattacagtaagacttccacaaagatttttttaaccttttatcatttctaactaattaagagaaaatttgcttcatataccactatggctctttgtaaatggccttaaatgaaaatttacacttttcttttatcaaaacagttgtgagctgccatttccttgggtatgaaactgattataagcaaagagaccattaaaaaatgttaggtagctttgctgaagttttcttctctaaaaaaaaaaaaaaaaaaaaaattgttaaagcaaagaaagaacatagggatgttattttaacaaattatttcccatttcctcatgtgaatacattagtccttatcaatgtattattccttgtcattgaaattatgtaaagggctattgttacatttttttaagaatggcaaattatgccataggactaacccactcaccatgcattttattgctccaaattgttctttctgacaaaattcaaaataaatctttaggtgaaaaatacatattttccttcagtatgattttgtttatagcaaattgaaagaagcatttaatttagctcaaatatttatgtttttattactgacaaccagatgaatatgaaccctcattttaccttaaatagtcttctttcatattcatgccagtgttttttaaaaaaccaaagaaattacaattaaaaaagcatttcctttcaattaaatatcaactttggttccaaagactgaccatttttccaacccattaactttatagggatgacagaactaatgacaggaggttttaaagcctttttttagtccatttttattttgatatgctttagattcactaactcaaagatctgtgataaattctgttaacagagctaatgtaagtatactgaccgaagtgaaatatcgattagatatactttctgtagtggcattgtcttcttgaaaatgtgttagaaattggaaaagaaagaaaatatctcattaaatgaattaacttcggttattcaaaaaaagtttatttagcatctgtgtgggaagtgaaaatgaagaaggaaagcataatcacttcgacagattttctcagaggactttgttttcctttgtaataatggagaaaattgaggatttggggagcaaaatgataggtaaattaaaaagctgtgaaagatttttagttctcatttttttgtgtatccattctccctaaattgtatatccagtgtttattataaaagttttttaggaaaatattattgtgcatttttagttctagtatacgctagaaataaattttacagcttcgactttaatttgatatcagataacttatcttttggtttgaaataaaaggaaaccattcatttttactgtttagctgacatatgttacattaaaaagaataatagaaaatatattgagtcccaaattatttttttcacattacatctcaaacaaactggtggaatccctctatttttatcttgcaccaaatgaaaagccattataggacagttgcttatgtataacacatatatatgtacctgtatattatatatatttatgtatcgtatatatttatgtgttatatattcttattcttatatattctttaattcttatatactttttaattgaggcactcttttccattggataaaaaaaattcagactaagttagaagtagttatttgtaacagcaagatgaaagcagaaaatacaaaatgatgaagggagtccaggataaaaaagtgttgaggaaaatttttgagttttagtgtaaagtaggtgttgacagaagctaaccatttttcctgaccactaattgagtatctcactagatcactaaaagcatttaatactagttcttcattactgtcttataaatgtcaaagagtaataacatctgacaaaactttagagtagtttgtctgcaaaattaattatcattccattggaactaagtcagcttgaatatagatgaattgacttttagagcttaagccaaaaggtttacaatataacattccatttagcagacaagtgttcttgaacctgcaagcatagatgccaacacacaaaacatggtctgagatacaaaggtggaaatactctcctttaagaaaggcaggatggctagtgtctataaagatattaatgtattaaagattaattagtagatgggtgtaaatgagttaaatttattacagactaaatttttaaggcttgatactatatgaagagcaatagaaaacagtggaaacagagtagtttatttaatagaaatttgtcttctttttcattgcagctgactactctaaaaattatgctcttacttaatgtgtattgtctatttctttgtctttgactaggcagcatggctacaaaattatgacctcaagaaggcattctttgaacttgtcagaatgttgtttcttttggtgacagtgcccccccatctctgaaaactaaggcatctcacataatcaactaaatgaatagtgacagctaacattttgtatttaaactcctgtcagtgtttaaacataaaaagcttgatgcaaaataaatcaaacaccagtgatatgcaagtgaaacattttgttaaaacagtctttgacatacttggatttaaatctgatccctttttagcttataaaatgctacaactttaaatgaatatgaaaaatccagaggttaaaattgaatcactaatctggattgtatataattgaagttaccacttacattcataatcatctataaaaccttcattaaagagaattatgcattaaataaattggaaggatcctttcctactttttgtcttcctgaagtgcttgcttaattgtttttagaaagaatttgactgcttattatgtatctagttgatactacctacctaacacacagattcaatgcagttttgaaagaacgtcagtcgaccacatcatttattccattttgaaaacaaaaatcaatatacctataaagtgacccataaattagaattggctcacacaatgtttgttcagtgctgcctttctgaaggcaccctttgcattttttcttcacctctctgtttgaactgcagtctcggtaagaggctcattaggggacattagggactggagctaaccagaattccagagcttgctttgctcttattaatgcatagtagagtagaatgcatttaaatatttcataggcattcagtaatttgtgtgtaatcgccttgttagcagaccagtagaagtagaacaggcctgtgaaatggtgttagtgattgcatgggtaatttacagacctttgccatcagaaaaagtattattgttggctagtatgaagctgtgaaatagaactaccaataagtaaaaaaagaaacttaataaaggttttgtaacaagcatcttcagggataacaaccccacttggtccttttgaagcagccgtaatttatgacataggcaatatatcaaatgcataagatagtattatggagcattcatatgaaaagggtatttgcaacattagttccaacttcctgcaagggacatgattaaatgattaatgaaatgtccctttgcatatgcattttgaaacagcaattaggcactgactgagaaaatccaccaatcctctcatttttcagtattatctcattcttgatttataaatcatagagaatttttgaacagtaatatgtagtacctgagatagttataaaaacataaaagagaataatttcggcacaaaatagtcataaattcataaattcataatttaatgttaatacttagcctatttatttagtcttattacattgtatttatatctgacactatttctgtactttgattggcataattaagtagagggaatgaataggcactattcttttacatatcactggtaaacaatagcgaggaaaggagaagggagatgtggcagaggtatgtatgtgtttttcaagttctcagtaatccactggaggctgttctataaatgatcattgaagggctgcaagctagcctataattacaggaaagaaagtggcagctctggcatttcataactatgtgtcctcgaaaagtgctttgtgagtttgtcaccaatgataatttagatagaggctcattactgaacatcacaacactttaaaaacctttcgccttcatacaggagaataaaggactattttaatggcaaggttcttttgtgttccactgaaaaattcaatcaagacaaaacctcattgactattattgtagtctacagtctctattctaataaaagctgcatagataaattgaatagggctctaagacccaagtatataaagcaaacattttgtactttttttagttctttaaatataagatcatgattatcactaagcagtaaacaagtgtagaaaatcattttaaaaagtgatttcacaagaaaatgttatagtgactatgccttttattgcagacctgtatgttctacttatcttgcttcctgcatttacaccctaatctcagtgtataactgaagcatttttgaaaatatgcaacattttttcttatatggaataaatatgtttgtgttccccaagagttcatggtgaaaaaaaaaaaaatcttacggaaacacagcaatatgaagccttaagaatacaacacacaactgtattatgctggtagaaaattgtcttactgttcatttaaattttgggggaaaaaaccctgggaaacaatttttaaatgtagtcttgttatttaggaattggtgacactttctgttaactaacctctggcttactctcacattccatacctccttcccagagagcatgagtgaatgttaatcctcctcacaggttcacagcagaacaaacctgagctgagcttagagagcagacaggtatagtggaagtgcctcccctgcactccagccacactccttcagcttgaccttatccaccttgctattttagtccacagtttctcttcaaccaagactgacattgtgacaagatacatggaggttttgttgttatgatcaataaggaagtattaaaacaaactggtttaatttatcaaggtataggcaaaacaaaggtttctgattgttgaacctaggtatataaacaaataaaccttttgtttataataaaattagtttttatcagcaacacgactttaattactcttcatatgttttacatgtatgtgtcttatattttttctgcctcactgtgatcttcaagaaataaaagtaaataatttgaatggatacaaaaatatatttgttgaattcagtatacccagggctattataaactttttagactattatatcattctaaatgatacttgagcccacttgggtaaatatataaattcttatttaattttgtgaatttcttttcaatatgccatttcacttatatgcaatgatgagaacatctctgaaattgttgccttactttgcccattggtgaaatagatacactaaaggatattgaattacttgaagattactttctttaattttgggaagcatcaaagacattatgtattacaaattacagagtatatattattaaatgttttcatgttcattggagatgctgctttactaaattacttttggtgtattaacataccactcattaaagagatctctttaaacatcgtatagaaataataatggtagactaatgcatattcaaggttagagtgagatcatttggaatgatttatttttcaccattttaaaaataagattaggacaaatgtctcctagttagaacactggacatgaaatgtcaatcttgttattctaatgatcaaaatcttcaatgaagttctttattactaacagcaaatcatcattcttcataaatattgattttccacgggagataatttgccatctttacctaattcataccacatcccaatgaaataaaacattgctcactaaaatacatcatagaaacaacaaactgttctaacaatgaaatagaaaattgcctttgttctcttcagaattgcttttggttcatagatttgtaagctgttctttttaaaaaaatggcataaataatatgacttctactataactctgtaaatggtctggggaaatattttttgtaataacaggtaagcttagagacagtgacattttctgccatttgaagagtctctttacattacataaataattttcacacaacacgtaagcagctttaacttttatctgtatggtttcaaggctttctgttttcccagtgcacagaaacatctgacttcagtgtagtgctttttaagtgtagcctatgccactaagatcgacatcactttacattctgttttgtgtcttctgtttgtttaggcaccttaacaatgaacacgcattggatgaccgaagcactgctcagtgtcgagtgcaaatgcaggtggtgcaacagttagaaatacaggtttgttaaatgctcacttaatggactcttcttagatttctggtgtgttacattgagtactgagcagattctgggtctttcagcctgcattttactgtcttagccattcaatactaatggagatacatgatattttagattgttttaaacagtggcaattattgataacccaggaaggattgctgtagtttaatgtacttatcacttatatttttataacttttagaccacatcatatgaatttgaatatcccaaagtagtcagatctaagtttttcccaatgcaggttagaccaaactcccaattagatatgattttattacagagagagtttttacatgagctaaccttgatttttttccatgagattttatattgtggcaaatgaccagtctttacaatatcatgcctgtaatataactaacaccctgaaagataaccttttcttttctttttctagtgtaattttaattaaagaacttgcaaaaaaatctttatacttttctttagctagattaaaggcagataatttatatcacagatgataatacacacatgttgtatcaaatgcgactgttttaagttaaaaaaaattctattaatgttgaaacagaatttggccagaagtaaatagaatccatagaactaatcagaaagcaattatgtcacccaaagtctgactcttgttatgttttactaagcctttaaggctttctctaacatttcatcttaaagatgatcttttctcaatgaaactaatactttgtttcatttaaagatacgatttttcttcatattttattacatcttgtttcaaattcgtgtcattgcttttttcagagagcttactaaaaatattttgttttgacttgtaaaataatcaagaacacaaaaagcaccatcactgttaaagtttaaaaagctttttataaacctgtttaccatttataaaaggtagactaaaacataagagttcagctgatgatacagtcttcggtgtgaccaattacgcctattttttttttttccttctgctgtgtctgactgatggaaaagtaaggtccgtcagttgtaatgagacccagtgcaagtgtagatgccgactccgtggcagagttcagcgtttcacactgcctggtctctgtcactctattgaattagattgatgatggcagattgcagggggcactaactggacctcagtgggaatgcatgaagtgtgtagacaatcctggcaggcactttgctttgagaacccttcaccccttcacagctgttggcactagtccattgctaacagtgtccacaggactttaaagagacaccatgggccttctaatttatggtttcagtaacttgttactgtcgaggcaactgtgacctcaattctcttattgacaaaaagatgaagtgttatttgttttctctgaccttttaacataagaaaagctataaaagtttttccccctccatagctaaagttgcatttcctcccagataagttcctcctgacgcagacttttacatgcaggaatcattattctgaggcaagctcaatgataagatgtatcactgcaataaaatagctgtatcagtcatttctaaaacgcttctgatctcactctttcttaacagctttctaaagaacgcgaacgtcttcaagcaatgatgacccacttgcacatgcgaccctcagagcccaaaccatctcccaaacctgtaagtgcatattgctttataaacagtaaatagctctaccaatgtaacagactaagaaaatgaacaatttagtgacagttagaaaacaatgagtgtgatgaaaaatacggcaataaaatgaaagtaaaatgtaatcgcttgtcaaattgtatgtttcttttaaagtaatgctatcttttacaaaatctatccaatctacaccatgggtgacactaaacaaagtacacctatcagtgcacaaatcactgccttggggctgaagccagagagaatttctttctgtgataggtttacagatattaaaaggaaaccctctttgagcaacctgaaaagtagtaacgtacaaatcacaaatagaaatgtatgtggtgtgctgaagaaggcagatcaatcataagatacatataaatagatttttatcaaatattttgtcataaaggtcatggtatttttatagccaattttgtcctatttctttggttatttcagttaagatgtatttcacatattgatatgttttattgtacattatacataatgttaatttagagagcttatcacaaaaagaagtgggtgattatagaaaattaagactagtttccctcaaaaatatatgagatttaagctcaacctaataatgtagtcagatacacagactgttgaaaagaatatactttatctcattcagctgatgtttgcacagaaacttagagtaatcaactgggtaactctagtttaatgaaagtttgtgggatactttttaataaggcccctgtatagcctgatgattaatgattgccagtcttgcctctccctctatttctttaggtaaaaaacgaacatttggctttgtagttggggaggagacagaagacgactatattgtctaattttctcgtaatgtctcgatatttcttatcaattaatatcattcaaaatttacttttttggtagagattatatttaaactctgctaagctatttctgcttttcctttgtactgtatttaaaggtagagtactgtaaaaacatttttataggaagaaaaaagacatgtatctttttgaatctgacatactactttttaaactctaaaatgtctgagtttttaattaaaaaataaacttttgaggatatttttgatcatgtatttatgactatcttttcaataagattttttccctcattgcttctatagtataatgtagataatcacaagacagaataattgtctcataaatccatcaggaatctgcagcttgttcttttccaattaattattcaatcagtaaattttttaaaactccagcagcatggttacatctacactaataaagaattttcagcctataatccaaaacagaacctgaatgaataatgtaattgaagtattacattacataaaccacacatgaaagaattgctaattgcgtttgatggtgggcaattaatacagaatcttagatgagcgatttaaaatgaaaccacagtttaaagttccctttggaactagcgatatctgaagtgaggaactgcactattggcctcagatcggtttgaaagaagagtggtcaatactcacaaccctcaaaaaaaccctaaaatttatacactgtacaaaagaagcttcaaaattgatatgatagtagagatttatggtctcatgtgcagtggctcagttgagacggcccacacatttgataaatattaatagcatgaatttattaccaaggagaaatgagctctgttggttcatcactgcacccttaacagctatcagtacatgaacacaaggtgcaaccgcactgtctattatatgaccccatttactctctgaatggtacttcattaaatggtaccttttggccatgctatggatggatgaaaatcaaagttatcaaggctgcgagtcctgaataatgagtttcacccaaccttgactatattcagatgagtcgaggtggctaagtgctcatcctagcttttacatgcttccctttagttaataataaattctattttatcagtttgtgattgtacgcctacaaacatggtatgcttattattacaggatagcagaataagttaccgtaacattgtaaacatgagtggtttgcattgaaaaagatgtatttctcctgttttgataacttttaatgtgttggtaaaatgtacagagtcctgtcacatattcatcaggatagatatttcattgatttgcaatttggtattttctgggttctgaatgtgccattttgttctggtttgcaattctaaaagtgaatatgtaaccatccagctgagttttttttttttatgtatggggaaatacttttcttttcttttcatatttttaacatacaagtatgtaatgctgatattttctaaatataacattgttatgtatatgttctgatgttgatattttagaatatttgcaaagtcacatacagatgcatatggatgtttctgcgtttctcaaactatgcagaggtgctcttgtacatcccagagaagcttacaccagtggctttgtaaatgtattaaatgcagcatttaggagcattatctcagctttcatttatgtacgtaacaaagaattccctacccagtctgctggcttagagtatgaacagaataacagtttgtggtttattgggaacagatgcacttggggatgccttcagggtgccagtcgttattattagactgctaatgtgcttcttctggctgctgcccaagaaagaagaacaattagctggcatatgttaatttttgtttccctaacaaatctctctactgactaaatgtgtaaaacaaatccacaaagaaagatcaatcaatgctgtacacatcattttctccccattaaaaaaaggtttatcttaagaagtgtatttgggatttaaaaagacaggtgttactaaattataaacaaaagctgaatattctctatttagccactcagcagttactttatagagaaacagaacaattagaattagttttcttgcttggttgtatagcctactgtataaagtaaaaaggaccacagattacctcttgtgaataccatttaaatgtatggcagaaatctttttatgtgctgccctgcgtaaaaattttgctaattggatgctatttatgacaaaagatgtgtggtaaatatgacagaggtgatatgagttttttgataatgaagaacagggcctttctgaggggagtaatgaagggcacactgttaaacagcttgcatacattctcaccttttttcttttttctaccctgacacccactttccagggtacatcagcattaagtgacactgtaatcataaattgaaaatgatacttccagaggaattggcaaacttgacatttcattatatttgttaacacatgccacaaatgattgttagtgaggaaattccatttccctctctccatctccaatcagatttaatttgaaaattttcagcctcctccggctaatttgcaattaagacaattttgtttgaatatgtagtaatgtcattaccattccaccaaattagcaaggagactaaaggtcagtgtaaaattttccagctggctggagcctctcagctgagatttgggactacgaaaaaaaaaattctggcatcaacagaatagcaacttactttaaatcctattggttaaaaacattgcatccttcattattgctgttgttgttgcttcactgggggctagacctgtttaacaacaggggcagtcgataatcagcaactggcttttatttaaggaaatagactttcgctgaagtctggtaggacaagaaggagttagtcctaagtaacattgcagtagattattagataacctctaacagaatcctctaattagagttcttatgatacaatttcatcctgtaattagttgagattggctgcttccttttgcagcttattagtggcaacacagctgtagaagtgaatccactctcatttgtcaaacctttttaagtctttttctcttgtctctacctttttcctgcccttctcttgggcctttgcagctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcaggtaggatatgaatgctcagtagagcacttttactttgggagaggaaaactgtagctgaatcaactgtacatgagccattccagagcacatggaaactcatgtcatgatttatggagtgataattgttttattcctggtaaatgcgaataggggcgattccctaagcacatcatgatgaattgttctgaatggttgttcaggaaagaaagctctccccattagagacactgagagatgtcaggatagcccatattatcacctcaattcaatgagatcctttgatctctgagggaagactagcaatccaacatctgtgagtaatgctgtagagaagagccattttaaaggacatatacgaaacaccgttgctttatgcaagacagttcgaggtttacatgaatcagtctttatcaaggtgctacctaaatgaaatcctgtcacagctcctaatgccgatatggcactttgcttaagaagcattgtgagaggcaaactttgcatttgcatggaacacaaatttaccttctttccctctcttacccttctcccttctcccttctcccccacctgccatccctggggcatctggataatcacagtcacacattgtaaccattattagcccagagccccctctttacaaaaagtgaagttattttcaacatgggtgcttggtgagggttttgtgtttcctttgttagatggccacagatgcagaggtgagcctgttgtactttaagacgtattagatgagagaagcaaagattatagcaggggggcaaaagtccatgtaagaatatgcctaaactacccactttaaagaagacctggctattggaaaaaaatcctcaggccagcaggtcgcacagttcctgaagtaccacgaacaactaggggatgcttcatttgcacttcgcgtcagaaatggccttttccatataattcaattccactgtcatgctttgtgctttcactagtcggtggcttcctcactgaatcactttaccaatactagaggaaatatgaaaattcaattatatataatgtgaattattagcagaattaacacctagtttttatttttatagaaattgccccaaactatgaattttataaaaatgcagatgtcagacctccatttacttatgcaactctcataaggcaggtaagtagaaggaaaattaactttgcctgattaaattaaaattcattaatgcttaaagtaaggcttggatgagaaagtgtcatctagtctagttagtaaaccattattttatgtcactatgtatcttgtctcatttcaggctatcatggagtcatctgacaggcagttaacacttaatgaaatttacagctggtttacacggacatttgcttacttcaggcgtaatgcagcaacttggaaggtaactacttttccagcagttttaagatgcctaccacagttccttacagatagcacaaggaacatttatttacatgacataagcagattagtatctgcttcccctctttttaacctgcctcttgaatggaatgaattccctctctcaaaatgacaagtgaaagaataattttgcctctgatatagttttctaatttggtgcaattaatagctgaggcttggcagagaaacgagggcctgacacactaattacagtgtgagcactcaatcatcaacacctttgttagtcgcacgatattactttttctcttgcatattattggctaattagtttgaaaaatgtctgtttggcttgtgcaacaaatggaggctgtaggttttgtcttggtctgcgccttttgtacacatcatacctcatcatacagactgaagctgccacgggagtgagggccatggctactcagctggaactaaaagaaagtaaagtgaatattatcctgtcagaacataaatgcagtttatttacttagacaaaagggttcatctatatccctgtccaaacaacttcattgtctgaatcttacaaggagctaaaaaaaatgttagttctatgcatctatttttggaaaagagcaacgtctggaaagataagggcactcagcacagactaagtgaactgttttattttcatttcaaaaagcagtcagaaacatcaattagccacaagccatttggtacaaaaggggaaaatgtttgtagctgagaagaatagaagcagcaaagcactcatcagcatactgagaattttagactgtgagatatgctaaagtgaattaatttggatttaaaatgcaaattaatctggcaagcgattacagatatatgggtgtgagactcagaatgcttttaatttacaaaatgtttagatgttattagttgctacagtatgtggtactgtaaatgaatgtaattgttttatttatgcacagttacttggtatatattttatatgatgaaaatgtcagcaataatctgtgcaatatatgttttggagggttttcagtaagtaatatttatgttgaagatttacattttcttttgttactcattagataatgtagtgaatgttctgtcatttgatttactgaaagaaattttaaaagaaatgaaagatgatttaatatcttagcttggtctcatctccacagctcggggatccagcaaaactcattctgaacagcttattagttctaactatatcgcatgcataataaaactgctcatttgctcattaatattaaaattatcatattcagagccatgggcattaagatcaatcaaatccttggatttcaggtcagatgctagatctgcttatgctttttttttttttttaagaattcttctcttaaagcaagctagaagcagcttgtctttgtgcaaatagaaagaaatcttcatcaaaattactttgttttatattcgtaatataagaattaggtcatttctgagtacttgatagctatatgccaatgtaccttgacaggaagaaatatttgcttaggtttataatttgaattatggaaaatactcgatgtaaattatgtggtctactgtatatttattatagctaaaattttaggaatgacttaatttgatttaaaaatgttaattttgagtaattactctaatacaaagctgccaacaacatctgtttgcataatcattatgggaagaaaatgtactctcgggttccagcaacgactttcagttgagtttaacatttacaaaaactttattgggaatttaaaaaaaatctaagtgtgtcttattgtgcaggcagaacgttacactgtaacttgcatatgtcatctaataatacagaataaacaaactctgtcatttcattattatagggatgaaaagaatgcctttgagcttatttgcataaagctttgtgcaaaattaacacttgtcactgtagcctttttagcattttaatacctcaagcaggactggttcctttggacatttcaaccatccctgagtgcttgatcagggacacaacagagctgacctaattgttctggcattttcaaagatactgtaatttgtacagatattgcaatttagaccagttcaatgaaaggaaggttttgacacagctattattaaaatagcttggaaggttctcttatcacaaagttcaaactgcagattccagtaatttgtaagtttgaggtttgtaatatcatgccatattttgattttaatacttaaacatgttaagatttttcactctgattaagtaagatcaataatgtagtatgttgggctgccttattagacaatattatttttgccattttttcttctcttctgtctgctttagaatgcagtacgtcataatcttagcctgcacaagtgttttgttcgagtagaaaatgttaaaggagcagtatggactgtggatgaagtagaataccagaagcgaaggtcacaaaagataacagggtatgtttgtgatagttttgtaatcctgtatcctgcatccaccaggaaaagtaaatactttaagttggggctggggtggggagacagttagcttaatggcatgctaacattctcgtggcaatgaactgcattttgaatctaggtgtaggtggcataaatcaacagtagcctatcaattttttccgtaaaaatcatcacaggactgttttggttactattttcatcaattcagcatttctacgtgtatcaaaaaattgtttttcaagatggaacatgctttctagagagagagagacacttcctgtttttatacaatacagtagcttccagtagaagctaaaaggctttttaaaaatgaggctaattttgcatatactggtaatcctgaaagtacctttttatgaagaatttatgagaagactttataaaaaaataaggctatttctttctcaaatactctcattttccaagacaaactttatcatttggacctaccttgcttttactccaactgtgatggttttctgtacagtgctgttgagatgaccaaggtatactagtgtgagaaaattactggctgggaagtttgaattttgtttgttttatagagaacaagactgccaaacttaagtccaaaatatagtaaaacacattttgtgcttaattaactccatgtagaatttacttattcccccaaacttctactgcctaatatgaagagatgactttaatgtattttatctactattggcccttgacttttgatttctatagtcaagtatgtgctaatatctgcattcaagaaatagttaatctattatgttgtcatttaattaactatggtaatttgttatgttaattcatggtgttctagaaattaaccattaattatttcctgattgcatcaaattggtaattgtattgtatatttacattttaaaaattttgtatgaatgttgctgttcttgcaaatgaaaccttcacacaactagaaacaggtttaaggatcctcaaggtcatgtctcactttgcctgcacaccatagtctaaagtttcatttcttatattcctggatcaagttaatatgtattatgtaaagggttatatatttgaactctattaccttatttttgtggccttatatacatccagtatggactattagtgtgagacaagccagaacatacctttatagctgagactattgatatccacgttttatattttgacgtataaatgatctttatatattttttttttcagaagtccaaccttagtaaaaaatatacctaccagtttaggctatggagcagctcttaatgccagtttgcaggtaatgtactttcccagttttgttgtatttgaatgtttagggctttttttttttttttggcatgtctttgtatttaggtgagattgtgattgttcttaatagttggtttatcatccaagttgtagtacctagtactatagacagcactggttctaattcaagctacttttgatgtgaagaagcaagaaggacaccctgtggcaaaatctggaacattcggtttttctgcaaatgctagattgatagaagcttttgcaagtgcatgtggctatgcattaatatttatccaggctgtcataaatgatttcacatcatagtgttggggaacatggtgcttgtggcaaaaactttaaagtttaattataatatataatttttatgcagaggaggcaaatgtcaggacttctttattttgagttaagttctggtcatttttaaaaagcagagcaactgcttatattaaactggagtatttcattacatgtgtggattcaacatttcattatgccaaaggttgaaacatgagtgagaaagtttgctgcaaggacgattgtttatattttcacatcgcacttaatttccttgcatctctgccacaagtagccagttaggaattttttttcaatacattttcttttaatccttctaaaatatctatttccttattggaacccattaaaagaagatacatgttttaaaaattattttaaatgccattttgaaagttgttttacacaatcttcatttcactaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatgtaagtgtggttaacagactctctaaagggaagaatctatattaatatgcttcttaaatttagaatttttccgaagtttttactgttgtataagttgaaaatacaaatttaagtggagttaaattataccacgttcataatatgacaaagtttatcaaaatcggttttaatacattttttagatgatctcaaatgccatatcattattgttccagcagtcctctacagattcttagtactactaatgttctcttgggcataaagcagtgtctaagatgaatctacatcttatgggtcattatcacaggttggtttgtatggtgcaataagaatattcatgatggaataagttgttataatttttactactgttacaggtctctaaaccatttatgaactgtagtcaagatattgcccgtttttataaaccgtaaaatgcaaagtttaactgttacccacagtttacatagaagctagtaaagtgtaactttttgcctgtcttataaatcagtgtaattatatattcaactttaattactggtgcgtctgtattggcaaggtttataagttatattttatgcaagactacgattcctattaatatttagcagctaactatatcattaattactctaaaatctttcccctaccatctttttaatggagctgagagagtagaagtattaaaaattgatgtattgtggatttcagaaatgtgtatgacactttaaaaaatgcctggcttttttcaatagtgtcaagcatattagtgtcttgtttactaaaatgtattgaccgtggtagaactcagaattcagaggaaaaaaatttgaatctgaataatgaagtgtgtacaccatttagctgtcaggatgagttgtaactcatgtctatacttcctagaattaaacgtcacatacttctaattacaagttattgaggtaagattagtcctttctcttatttttatattgtcttcctttatttttccattcaatatctagtaaaaggtctgaaagagaaatatactctttatatgtacccattttcacttgacctttcacagggtattatttttgttgtctttttaaacattttattctgtttgacatattaatgtctttaagagcatatgtctaattgctttatagcacttttactctatcacctttgcaaaaaaacacactgtgaacattaatgtgttttatcaggttccgaaccattacttcttcaaaatggaaaaatttgtcttttggaagtgtctttagatgtattaaataatacaaaagaagtaataaaactcaaagtagaaagaacatgaatatatgctcttgagagtagcagtattctaaaacaaggaaagtgagcaatcagacaaaagtttttatatccccgctttatcctatcagccatgcagtgggtagcttgcggccttttactttcagcagttctcctgagtatgctaaatagtttattaaacattatatttcagaacctgaaataataattgggctaaattgtagcattgttagatgggtattggttttcgtttggcaattgctacttcagatttttcattttattattaaagtttatgaaatatttcagagaaatattagtatgtattagataacacctaaaaattatacatataatgtccagtatattgctgctatgggtttataaagttaaaatgttcatagttaatatataacacattttatgcttatatgaattagtgcatttgaaaaatacttatgtttattttccatagataaaaggaaagcttgttgaaaatagatttcagaaaaaataaaaattaaaaggggtgtaaattgtccctttttctaagttacatcaattataatcaaaattttaattatcttgcagaatattgagtcaatattcattcttaaatctgtttctgctgctgtcattaggtttttgcataatagatgtcaaatatttatgaaatatttcagtattctctatcttcacctttataaagttctaagatgcttgtacttaaaccatgttatgtattttattaggcaaaggaagaaatattgatataattctctcagtcaaaatttggttgtatataattgagtgcatttcaaaatactttggaaattaaacagaagtgaaataatttttataagacatttacacagcatacttatatctcctaaaatgaagtcatgttacttgttgttttagaggggggtagttgtgctgatgcaattgtaaacaagaggaaatagggcctagtggtatcagaacataggttgtaatgccagactgtcagggttccaattccagattcaatagttaatagccaagtgggattgagcacgttacttaatatttctttcctcagctttctcagctgaaaatgaggtaagtaataatataatgcttcttatttcataagattcttgcaaagaaatgagtgcctggaacataataattgctccttgtgtctctcatagtgctgtgtaaataggtactgatttatattacttcctgtaatacctcactgttactaaaaatgagtctataaacaatatgaaaatatttttgtattttgcattttaaggcactctgtaagtttgtggttagtcattgacaatccattttcttctccttctttcttctcttataattcataaagtaagcagatccataaaactgaaacaacatgaggccaggcatggtggcttatgccagtaatcccagcattttgggaggggagggcaggaggattgcttgaggccaggagttcaagaccagcctggacaacatagtgagaccctgtatctacaaaaataattttaaaaattagttgggtgtggtgtgtgcctgtatacagtcctagctactcaggaggctcaggcaggaggaaagcttgagcctagggatttcgaggttacagtaagctatgatcatggcatggaactccagcctgcaccacagagtgagactctatctgtctctaaaaataaaaagtgaaacaatatgacacaaggacttattataggtagcacattacctatatttaggtatctgaagttacctagttatctgaagttttaggcaatgaacaactcaaatagaacaacaccaacccagaatcagggacatatcatatatgtcttgaaaacattgagtattagttttgaaaggaatttagaatacataagggaatacagaatttagaatataggaagtgggaccagactgctgagtgacttaaatatcaagctatggtgtttgtttcagtcagtatacagcttgttactgttatcagcttgactttgacacgatcagagctatgcattagaaacagtaatctaaaagtgtactgaatgaaccatgacttaatgaaactcatgtataggagaatattctgaaattgggagagactggaagcagaaaattaattcttgcagtatgtaatatctaacataaatacaagactaggagtggagagcaaagaatccgtataagaagtgctgtgaaaagaaatactggaacaagacaaccaactgaatgctagggacaaagatgaaaattgggaaaattcatagccactttaaaagtctgcaaagagaaatagaggacacaagaagcaggttcaaaagggaagatgaagaattcagcattttacttggggaatctaagaacctaaatatcattagattcaggagaaaacactgatgatttttttgattagtcttttaattcaactccattatagtaaaattctacgtggtagaatatgaatatatacctgaggttgatagagaagatgttttttgaagcattagaagagtgaaatagatatggtctggtgcttcttataaattgtggagaacaagagatgccaaattatcatttatattccatactccatattttaacccttttcttccactccagttttcgctttattcagtgtcctgaataaaggcctcttccaaattaatgcatttatttcgctcacactatttgaatttctgccctttcccttttctggttcccctctactctcttacctaaattctacctttccttacaggactagtcctatttttttccaagagggtttcccaaggaattttactatcaggaccatgcactctgctccacacttctaaagagcttgcttcttataccactattttggactaatactttgtgtacatgtgtgagtgtgtgtgcacacacacatgcacgtgtgtttatgcatacatctgtgttcattttgcagatatccctaccttaatgacaagtcccttaaagacagattatgttttagacaaattatagtataagaaacccctagcacattctactatgtcaagtaagcattcagtaaaagtatgatactgattatgaagaatcaaattggagattacagttaattagaaatggtttgacttcacaaagtcatacttacaggagtataaaatccagggttctcctagaatcttgaagaaggagaacaaaagagattttaggaagaacagatgagtaaatataatgttcatttttatgtgggagctaggaaaattttctaaatattggtagactatactttttaagttttatttttagttttaattatttaattaatatgtattttataaccattccaatttacgtgagtaaaaagtggctttccactttgagtagaatgaggccccttcacctcattcatgattatcatctcaaccttgttaaattttatttacattgttgaaagattgaaaacaattatcaaaatttaggcaatataaatcagatgaatgtttaaggggatttcagcaaaaagtaggaaaggttaaagccaggcgacattgtatagattctacataataattttttgaaacttctttgactgttttgactcaactacttattgattcaaagcaagtacttcacttttaaaacatttttattttctattgctgttcaaagagtcctagaaacatccatggtatgaataaaggaaaacgatactttaagtgtccgagctgagtatcattacaatttataatattgttcccttatgcacctgaatgccaatattccctttttcagagttatatagaatatgaattaaaaatgactagcttcctattttatatagacatttacctctagaaatgtttaaaaggttaggattgatgctgtctttaagcagaaataatttgaacttactgtcttctctcatgtgttggacttaagtgaatgacaagagaagaaaacatgtagactagacttggaagatgaacgcatactgatacaatcaatccatttcctgtctacttcccaacctaaggcaagaagtggaatttaatgatgtacccaccatgtttagttctactcagttgtttctttttgtattaagtaattgtttctcctcattctcaataagttggttcagatttgctgttcttcctcgtataaaatgttaaatgagattcccatttttatattttcaaatgttgtgtctagagatcaaaataaatattcttccctagctggttttgtatggagtgctttactcacaaagcctcccattgtcatgtcattcccctttctgattattagagtctcctactgttgagttttcagatcctttcttatatgtcattcgtgactcctcttagcctctatgtcatttcaaagttttgcttgctaaatattctatattcgttgtaggtgattattattttctcctgttagtaaagcaattatctgaagcttgaaagttaaaaatcttttatatagaacgttaatatattggatataatataagaaaattaatttattgtgtccttagaaggcaaacccttaggagaagaaaataaaaggctgaggtgtcatataaaatgagtaatgtagtattatttcagaattgcaatagagatcataagatttttttaaaatttgcatgtcaaatacctattcaacatgctccagaactttgtatacatcacataccccagtcatctttacgacatcctcttcttcttcataacctcagttgttaaatcatttcaatattatactttggcctctcctgttgataatattttgccccatagatagctaaaaggtgctcctagacccccactggattaaacagtattgaaaatctaaactttgaattgtcataaaacataatgaaaacataagtatataatattacatttcttccttctattctctttggctgccttaaaatttaaattgctatttgtaggcacatgtagaatttgcaggccctacatctttggttgctgatattttatataaaatccctgcagatcttagatctctctgtggacaaaagcatttgaaattggagtggctctgtatttcagaggaaagcagttggaagtgactgacaacttgagcaaatggaatgctctttctcattgttttcattcctgttaagcctgtactatttttgctgatgttttggatcattatgttgcattgcaaaactctaaacatgaaagctttttttttgcctcacatatgttttcatagattttttaattaacaatttttcagtcaactttatttttttctaacatttctggtcctcttacaatttttcttctgatttaattccatttttatttaatctctaaactttttgattaactttgctagttagctttgagatctctttccaaagtgattgtacggaacttatgattatgccagctctttatcatctcatctttttttttaagtgctacaagtgtggatcagtagctgctgttcttttttacattattccagtccccatatgtggcacttacaactcattccagttcctcttaataaaaagatcctcccagagatacagtgcctttagctaaaaagtgttatatccccaccgtgccaattgtggaaatttgtgggttcctatagggttttgaaggatttttatagaactatacagatgatatctgacatccgtggtgtgaattcccacaatttaggcaaagtgtgtcagaaaacttttagctgacagtattgtagcttactgcttctgttttatatctaaagaaggtactttgttagaacatacaacttcatttgcactaacactagttgagccttaaaaatttcctttctctaacggtcaaaaaatgattactgaattgttaaagaaggaataatgcttcagtgttaactttttcctactttcagtaccagggaaaaaaaaatgaagatcttatgtactgctatggtattttaatttcagctttcaacataggacttgggaagtggatatatgttggtgaccagaggcatttaataaataaagagacttacataaagtcaactgttatttcagaatattcctttatgtgaaggtaatttacatgataagaaattatgtcttaatgtatgttaaaattatgtgcattttgactgctctggcagaatttaacatttaccactcacagaagtaacatgttatacttgaatatttcaacaccaagaaacacagggttactacattccatagccagtttctatcattagaatagcattaaataatttatataagcaacagatgatcaaatcatttcacataactactgaaaactaacattgtttagtttgttcaacattatcttcgttttgaagacaaaatggagtaaaatcagcaagacacttaaaaggtaggaggataagtcaccattgatcacacaatgttatcttactataaaaaaatgtatatttgcaacattaaatctgaggttccttttgttaattagcaagtcaaagaaaggacatggtgcactagatttagaataaaaagtacatattgttggccgggtgcaatggctcatgcctgtaatcccagcactttgggaggccgaggtgggcagatcacctgaggtcaggagttcaagaccagcctggccaatgtggcaaaaccccgtctctactaaaaatacaaaaattagctgggcacggtggtgggggcctgtaatcccagctacttgggaggctgaggcaggagaatcgcttgaacccaggtggcagaagttgcagtgagctgagatcatgccgctgtgctccagccaatgtgacagagagagactccatctcaaaaaaaaaaatacctattacttttactccttattcatctaatgtttagaactaatagagtatataactaagcaaaactcaactcaggagaaagccagagtttgagtcagttctgtggtttaatttgagcaaaggaatagtgtcagcaaagcagtcagtctcagaaattaggcagacctagtgacatgagcaatatgtagtccaggtggaaagtagcctctgtaaaggtattccaagagacagcttcaccaggagccagcagcggatgggtaacaactccaatagggcagtttggtaataggaaatagataacagttgagaaaaatgggtaagccacatccaaaagagcggccacagcagtagggctcaactacaggactttgaggtcaagatcttcaaggagccagaatgcaagttattaaatgtaattacagataaactggaacatacaccccgtatactcatattacctatctgatttttgaccgaaacttggtccaacaaacaaggcaataatacaatttttggattaatgattcaaaggagaattggagcagcaacaagactgacttaaaggcaaagtgtcaagatgtgatttagacttgatttgtctggccaacagattagaactataagggtccttagatccacagctgaatgtttattgatagttttcatttatgagacacaaattggaatggtaccaattgtatctcagtggccaactaatttgtgtctgagattgggcacaatatatttgtacttatcaatgatgaaagtaattacatctgtattttcattttattacagctgttttggtcacaactatcacttttataaagtcaaatagagtgataaatcctgaagaattacattgtgttttttaaaaattgactctacagcaaaactatctgaaatcatttgggagtgagcctaaatcatgtaaaatctagttcctttaaacaatattgcataaatttaaatacatttcaatataatgttgaaggtaagagcaggaaattctagagaaatgttgatatacatatatatgcatgcagtttataaaattatgtttcactgacctttacccacctttttgttgtttctttggactgatactgactttttttttgagatggagttttgctcttgttgcccaggctggagtgcattggcacgatgtcagctcaatgcaacctctgcctcgcaggttcaagcaattctcctgtctcagcctcctgagtagctgggattacaggcgcctgccactacacccgactaatttttttatatttttagtagagacggggtttcaccatgttggccaggatggtctcaaactgctggcctcaggtgatccacccgcctcgacctcccaaagtgttgggattataggcatgagccactgcacctggctggactgatactgactttaaatggaactttataactccaccatggaatcagatctcttgagttgtaagacataattataagctgtcatcattaagaatggaaatacatcttagaagagaaaatttcatcacaattatctgaaaataatattcctttgccatcaatgtaagtggctgtagtttttagaaaatagactaagggaggataacatttttatcctggcttatgatgaaaattaaaatttattttgatatttatcattagccagggagaaaaggtgtgggaaatttagtagggcattttctacttatatcctggataaaggaaactatacagttaatatggaatgcagaatcatttgttcactccaatttttgaatccccttttcataattgcacatttctttaagaaggaatattttcgttttcatcaagggataaggaaggggattatttctgtagaactgaagtgatagtctgagtactgtcctactggcagctactacacgcaaactcattcaaataaaaacaaagatcaattcatttaacactagaatttttatgtcaaacgttattttaatttaccaaagagaaaatcttgaaagataagaaatgggaataaagaactacataagtaataactctgttgtataatttcttagctgataaaagttaagaacatcttttttcatttctagtagttttaattggacatctttcttattcagtcctataaataagagagctaaatacttttctaaaatatatttaaatttagaatgtaaaaatctaaatttctgtggagaaagcctggtttattctttccaaagaagaaaatcttaagtagaatgaaataaacaatttatttatgactgtataattaactttaattactcacatttaattatgttatccttagatcagctatcatttttaaaactattaatatgatttctgttaaaagtgaacttttctacttaaaactttaatgtcacttatattattagcttttttctgtgatatgttaaataaactaaaacattgcccaagtggagaaacaaaatattactctaaaagttacataggcttaagaaatgaattgtgtatcatttcaacctggcttcacacaaatggagataactaaacttgtcgttttcatgaatctaccatcaaattccaaaaataattatgtaatttcctttatatatactagaaagaaatgagtaaaattgaatttccataatttccatttttatataagcttcctaaatgagcgtcagttattttaccagttttcctgtgaaactttgtctttgaaaaaaaaaatactttttttgtatgttctgtttgacaagattatattttccaatatgtcagtcaatacaggttagctactcaaaagtaacaaacgaaaacaaaagttaagctcaggaaaagacatttaaataatcctgtgcttatattttgaattcattttatatcttgaaaacagcaatgtgatagagtaagaatgagggttttgccattggcaaattggaactaatttgaatgaaaatttattcaatcacgtatgaaattgataaagtgctaatcatgtatttattttgaatgcttagatacaaagagttatcatcattaattcaataatcttgtgccaagtactgaagattgtagatggttaaaaggaaatctcatctttttacagcatttttttaatctagaaggttttgatgctattttgagaagtagtcaaaatctatgcatatcaatggggaacatatgactatagaaatagtctattttgatatttttaaatgtcattaattaacacttaggacataacagtatataagaggtaaaaaagaattcataggattatacacattttctttattttattttattattattattattattattttttgagacggagtcccgctgtgtagccgaggctggagtgcggtggtgcagtctcagctcactgcaagctccgcctccccagttcaggcgattctcttgcctcagcctcctgagtagctgggactacagatatacaccaccaggcccggctatttttttttttttttttttttttttgtatttttagtagagacagggtttcttcttgttggccagtctggtctcgaactcctgacctcaggtgatccacccgcctcggcctcccaaagtgctgggattataggcgtgagccactgtacccggcctaaaacgttttctatcggagctagttaatgtattccacgtaagttaaaggtatccaaattaaattaagtagaagtaaggatcaagacactttaaaataaaggatttgaacaagtctatattgaatgggtaatagatgcatagttcaaggcctacacgtatttcagtatatttaatatgtttctcaaagtactgtatgtgtaagttcatttgacaggataaagattgctgtattggatgaaataaaatcagtttccataataaaattgaagcatcagagtcctaacactgtttgtgaggatagctttttgaatgttctctggggagtaacattttttagagtaatgatccatgattactatagaagtatgttataaccttcaggtgtgctaggacaggtgacaaggctaatcatatctatagggttttttaaaagatgtatttcttttgtaaaaagttggttattacttttaaaaggtatgctatttgaatgtattggatattttctcaaataactagaattataataagaaaagatgtaagggaactgcctgcaattagtaaaccacatacaaacaattttaatctagtctaaaatttagtcttgctttatagatgtttgataaaattaacatcacattgctggaaattgcccatattgtagatatgagtcataaattttaaggttttgttaacagcatttgttgacatttgtcataaatgaggtcaggagattgagaccatccaggccaacatgatgaaatcccatctctactaaaaatacaaaaaattagccgggcgtggttgcgcgcgcctgtagtcccagctactcaggaggctgaggcaggagaatcgcttgaaccctggaggcggaggttgcagtgagccaagatcacgccactgcactccagcctgggtgacagagcaagaatccgtctcaaaaaacaaaaaaaaaaaaaacaaaaaaaacaaacttaagacttaaaatctttattgttaacttcatggaattaataaacactacatttctaagacctgattgaatttacattgtgccagtgagtctctcataagtgatgacagtatataaatattctagtcattaacagttgctagtatctaaataagcaaagaagagaagacatacgattctgtttaacatctattgacttaaattttgaacatctcagtttaagagaactttattcatagtacttttttattctaaagaaaaataggaacggtttatagaacctttgacttagtcttgagtaaggagaaaatgaataaaatttacattactctagggaatacactgtgtgaccacaatctaaaaggataaactttccctttataatttgactgtttgctttggggtataggcaagcattaacattacttctgccgttcattagtgccttaggtactggtaaatttcacaacaaaagcaacacatatctttaggcaacaaatttgtttatcagtgtatcagtgtagttagtcctctgtttgtgtttttggaatataccattaatgagggtgcctgtaaaacggtgagattaaactaatcaactgctactttgcaatgttttcaagtagaggcttgcgaggttgaatgacccatagatgagtttcattcatttaaattggataaacatttattgaacacctattgtgtgcatcatactctgctagaaactgtggcagatgatcagtgtctacagaggtaaaactctaatagagggataagtacataggtagtatcagagaccaagtgaaggaatgaagagagttgcaaacaaattgtgaagttaacgactgaagcatgcacatgcacgtggggaatcggaaaagacttcatgaaggatgtaattttgagttaggcttaacgtgaagtcaatttttaggttgaactttatgaaattattcatatttgacttttttttcctaaaaaatagccatcttaaatatttcaacctagtattgagcagagatgaggaaaggcaggatagatgtaaaggacatgaggagatgagcaaagatgactaaaacatgggtttcattcagagaacaatgactcctttgcctcaggacaggcagcctgcctggatggggatacctgtaaaatgagattgcccaggagtttgagagccgtgtcttatagctcattaatttccaagctgctgatatgtagggacatgtcttaaagtttaccaattatttggagatgcttagcaagaccctcaagggtatatgttacctacttttaaaaagccctacatgctttctttttggtacttctctccggaaataagtgactcttttttttttttttttttttttttttgcttgagaaactagcaacactttatgccccttccttcaatattcagttttaaaatacatttttccttaaaatgctataagaacacagatgaaaaattaaaaataaataaaatgtccggagaccacacatcaatgttattgtggccattgctgttactagcaccttaatcacagagaagactgtgggggaatttgttttcagaatcttttcatcttcactttctaatatccaaactttctgagatttgctttcattgttcttgagcaattaaaataacaaatacaaagattcatagaggttgcttgagagtcatgaaaagttaagatgtattttaactattttattgatttttaactttcagtatttttgacagatttaggctcttctatctccttggtagagcataaaaataaatatttcatgtacattgtaaatttatagagttcagtgatgcgatctcagctcactgcaacctccacctcccaggctcaagcaatctgcctgcctcaacctcccacatagttgggatcataggcacccgccaccatgcccggctaatttttatatttttagcagagatggggtttcaccatgttagccaggctggtctcgaattcctgacctcatgtgatctgcccaccacagcatcccaaagtgctgggattacaggcgtgaaccaccatgcccggcctacattgtaaatttaaaagtagtaaatgcaaaacctttttactccacaatctataaattcttcacttctttccatcatttctaatcctaccaccttagtggtctgaccatcattaacttattacattgttttagtaagttcccatttggcaactctacctgcagtgtcctacactaagcctatattttgcaaattcatcttcataaaactcttctaccttttcattatcctgctcaaaaaccatcaatagcctttgagcagagattaaatttaaacatcatgaaatgcaactcaaaggccttctcgtcatctgacctcattctgctattctaacacacacacacaaacacacacattatcaccagccctccttcccccatgcactctcagggttcccataacatgtctacgattctccattttccctatttctacttggaatgtcctctctctcatctctgcataataaaaattttcacctattacaaagcctacgtgaagctacaccactctccttcatagagcattcactgattgaattgtcagaatttatctccccttttataatactttacctactgtggcatcgatcacattctgccttgtgttaaagttacttgtatatatgtcttacctcctctattagactgaaaacttgatgataacaggaaccattgtccatgcagcccctatcccagaagtttacacacaatgtctattgactggatttttttttccacttctaacaagaagtaacttttatcctcctagaggtacttcaatttcagttactgactttgtacctctgtcagggaataataacaaactttatatctctccattactcaatacttcttttcctccagttgtctaaagaagttccaaatatgtttctaaatctcacaaatgtaaattaaattaaattaggagctggctatgcctttttaaagcaagaaacataaattggtgtttttttgtagtaaggaaaatgttgtgtactgaaaggatcgattaaatttgaagtcttctatttcatggatagaacaatttttaaagcatttgcaatgaattcctggattctggtttgcagaaatattcattatttgccaaatgggtaatttgcactaaaaccaaagttgatgttacttcagtgcccccttaaaggaatgtgatgttttatcaggcttagaaaaactgctaccgaaatagacataaatcccttctatctgtgaccttctatggcaaaggtttgtactctttaatctgtaataatacaagtacttggcatattttaggtgcccagtaaataaatgaatcactggaaagactataccccttacactcctgccttaagctatgttaaaaatttactgggaggctgaggcaggagaatcacttgaacccgggaaggcggaggttgcagtgagccgagatcgtgccactgcactccagcctgggcaacaagagtgagactccatctaaaaaaaaaaaaaaaaagttgaagaaagtagtgcataaatttcaaaagaaagtagtgcataaatttcaaaacagttggggaaaaatggcataatgaatgtctgaaccatctagaggagatcttcagatccagaaaggggattttatcactaggatagtcaagaatttctttcttatttccatctgaacttgctcttactttactgtctttaatgaattatattaagaatacatgtgttttaatactgttagaacattggcattcttcttagaggcaggtaagttttccaattgttaactcctatatcacttaattgctgcttttcttaggacacttagggctggcaatcttgtacatttattcttcttatactagactacaagttctttgatagatattaaaagcctcactgtgccttatatatagaagatactcaataaatttttgttaaattagagattggcaataggagataatgttaatatgttattggcttttcctgtcaaagtgagatagatgctctttacatcattatagatacaggggacttgactcaatccacagcataaaggaattcctggaggaaaattttactttctaacaggcttatatatattgctacatctcttaacattttcattatttagtcatattaagttaatctatttaagatttttctagctatatgtatattatcagaacttcataagccccaacgtgcatagcaaatgtgatgcaggcctggatcaatgcatgtgtgttaatgattttaatgacaagctaatgatgaggcctgtgatggatgactgaggagaataaatgtaggagcactatgtacagagcatttcaatgatggctcctctactttgaaatttactttcttctattgatctgaaagtgatctgagacataacctggccatttagcttagtattttagtgacccagaatttgttggtaaataattttttgaaataactatagcaatatgttgtgttgttttatttccttttattaccttaaatatacttttaaggaaatgtgactagtcattaataactctaatgccaggcatctgatatgtgaggcacatatatctttgtttcatgacttttttgatacagtaataaaaatctcactttagaaagaggtactgaaatacttttagagctaacgaaactcatacaatgttaaaaaattaatttctgtatttataccatactttcttccaaaaaaggttataaggttattatatattatgactgtgttcagtgtttattgatcacttatgatccaggcactatttcaagtgttttacatatatgcaaaataatttaacccttataacgagtgtataaggtagtcactgttttccttgctttatgggtaaagaaatggagatacagagaggttaagtagcttttccaaggttgcatagctaggaagtaaaggagccaggcttcaaacccaagtattttatcctcaaagcccactatattcttaaccacttaatcactgctgcgtgactaaatattataaatgaggtgcctaaactattcagtattactttataccacacttagaagttaatcaaaatactaaatacataaaatgcatcacggtaaattatattcttataattaaaaacctgtagataattctacatgggaagaggattcataatgaagaattggtgagcacagaagagagtttctttaaagggattcttttattttatttgaattgtcagggatgtttctatgttggaaagaagcatttttgtagaagaaaacctaattatcctatggtagtgtaacacatggcaccttgaggaatttgcagaaatgaacaccacagaaatatccactttttcttttggaaataaatttaatggctctcatatcctataatggggcaaaaagagaaagaaattgccaatggtcacaagaagagactaattctctattcatcctgcaacccacaaaaggcatgtgacttcattgtgttccatttcttttgagttggcatataacttgacaagtgaaaacatggagcactttgagatccaaaaatgagaaacctaatataaatagagataattataatagaaattttatttttattgaaaagtattccaccaatttataggcataggtactgtaactttcaagcaattttattttctggaatgttgaaacttctcttgctactttttatttcttcctctgatattatattgatggcaaatagatcttttaaaaattgagcattaataattattgactttagtttgctttattttaggaaattagcagcctaacattgtagccattggggaaaattttatgtacagcttttccagctctgatatctttttcactagttctgaatggaaaatattccaagcatacttaatttcagagaggtatcttctctattagaattagtacaatgaaactcttaatgctttcatggcacgtaatttattcactcaacatgtatttacagtgtgctatgtgtaatgtctcctaatctgtgaagttcacagtctacttggcaagataaagtatagctaccagtagaaagagttaagtgacagtgcatgtgattaaggcaatatggggtagcagaaataacaaggttccaagaaactttctagaggccagtgtgtgtgtttgtctttgctgtgcctgtatgcagacacttggacagtcactcctttactcatctgtaaaatgggaagtcaaataacaataacgttacattagacccagttttaggattcaaagattctgtagttttgagcatcagaatgaccaaaaaatgctttgaaggttcagagtagatcaatgaaatgtggacaggtcgaagtctttaggaagtagacacagtgtgagttgatccttaagtggtagacaggatgtgtgtgaataggcagaacagggtagaaggatgttccatgtatacacggagagttgaaaggggaaacacagcctgagtctccatgaaaaatgtcttggagtaggaactgaaaacaatgtttctgggatgatgttttgagtctgaaatggaagttattattcagaaatattgttgtacatagataggtggagatcagatcatagtttattgagaatggctgaagggttttggcttaatcctctttcaaccccgtgtctcagttcagacatatttttatacagggcagtggcataatgaaataaataaagttcttgcatattaaaattttctgagactacaatagaacatacctctttagtcattcttttaaaaaatttttctttatgtgctttttttagagacagggtcttgctttgttgcccaggctggcattcctcccacctcagcctcccaagtagctggaactacaaacatggcttctatgtgcttgtttctaaataatacatcctatttgttattcctctttatcaagaacttgagatttttccacgaggttctttattcaaataaaacccaacccagaaaggttaacccaaatgaaccagatgcatatgatattgcagtggttttgctgaggaaacagttgaaatgcagcagggaattcagaaactgtcagaggagggggacctccgaggtcttctcaagtgtcctgagtatattaaaacacactcctgcacaagagctcacatctgtatctaaaactatgggatgcccaaaataatgcttattgacttgaaggcagtcgcctagaccactgctagatatctatgtatgtggcttgacagtaactcaatatctatttagaaatactcagctattaaagcctgaaagttttgaatattcgcaggtcatttgtgagacctaattagaaaaaggtgctattatattaaccttaggagtttattctaattatttccagcacttaagagtttagcattctctaaaatgttctgaatcccagattaaaccaggaaaatctaaaattataaattcaacttataactattattaaatcaaaatgtcattttttataaaagtcattaaatgaatatgaactttcacaagtaatctcattagaagatttcatttgtatattttagagggtactttgggaatattaacattagatttaataaatgtttaaaattctgtacatgtctacatgaaactgtaatatactagttggttcatctattattttactcaaactattggctagttcaatgaaccatttattcaaagtagttgttgagttcagttggttgatttttttttttttagtcagaatagttgggttagtctttaaaataacactgataaacatctcttatggatttaggcttttactattctgaaatcacctatgggtatacactcatacacaagcacacatgcttatgtgcagatctttccccctctcccagtggtgcatgttttcctgatagcacatttctaacttgcaaattggctatggaacatttcattaggaccaaatagtttatttatcacgttaaatagtacctacaaactagtcatatgcatacagtagagtcctatttttttatttataagtaattctttaatacatagtattagttatttgggggcctatggttttattttataatttgtcatgcaggataacaaatagtaccttggagacttaaaactatgatagttttccttatataaataatatagtgctttggatttagcatgaaacataagcataataacttcagtttcgttaactatcaatgacaaactcttcttggctttttatttgtattggctaaaatataatttcatgatttaccccacagtgtgcaataatggcgttataatggccacttcgagtagtctctaacctatccctagggagtaatctatactgagaattgagagattaaagctgatagtagcttgatacagtagcaacatttgggaaattccagtgttctttgcccatgcatgcaaaatgttagaacagttccagaaattgtcaccccagaattggcttttctgatgggctttaaagtacttttcttatatctgtgatactcctctaataacagttacaatgttctggaatctcatttacaactattcacttttaaaggaaataatacagtgtctagggaaaagatgagctgctaaagcaactccacccttcaaaaaaagttgtcataagatgaactatgaacttaataaggatgatccaggaatatcaagaggaaaaccggtgtttttttttttaaaaaaaaagccaaaatgtgaaccgtcttcaccatataacactagtgacaaaataaactaattagaattgtattaaactttttgccccatctaaatatactttcatatatgtagtgccatttctaatcaaatagtttacaaataataagttttttttatactatagtctatgcaatacctgaaattataatacttctttttttttttttgagatggagtctcactctgtcacccaggctggagtccagtggcactgtttcgccttactgcaacttcctgctcccaggttcaggtgattttcccacctcggcctcccgagtaactgggattacaggtgcatgccaccatgcccagctaatttttgtatttttattagaaacagagtttcaccatgttggccagactggtcttgaacccctgacctcaagtgatccacccaccttggcctcccaaagtgctgggataacaggagtgagccacagcgcctggccaatacttaagtctttatgcttttaatgttcggctttttagaggccactacggaaatatcaaagtaaatgttcttgtagcacattctaatatccagttcaaattttaacttcagtttcaaattttgtcttgtctcattgctggcaaaataatgtatatatcttcattttctcaagattggtaaacattttgtttgtttttctttgtttgcttttgcttaaaagaatatgtctctataaggacatctggaaatagctatgaaaataaaatctaaaaattttgtgtgtatcaaagttattaaaatattctgagcacgtatactttttaatataattatttttaaattatgattattataaaatttttaagacattttcttttaatgagccgaacacttctggaataatgtcaatgagcacgttcaaagggacattaggattccattgcagttgaactttctaggatccaaatgttttttgaatttgcatgcactatagtaatatattgaatgagttttaaagtgccctaagtgctttgttatttaggaacttttataaatgttgtatacttgttgctgaataacgtgaaatgtctgtttctgagctttgaattatcatgtgataagagtttcctaaatattatttgattctacggcatatagatttttatattgattaagcgttatttccatttagtttacgggagagttttgatgaacacccaagcaaagcaacttccctatgttctacaaatccagttggagcatagctaatatggtttgtgtcaccataaagaatgacaatacaccttatttcattacaataccctgctaatgacgttatttgcatccccagcatatctgtgcatgattgctggtaataatcctgtcataatatgcaagtgtctcattccaattgcagcaatgaattcaaagatacatagtacacatttatttctgacttcataatgcttgctactcattaaatactaaatgactcatccagacatcttaatatagcgtaaagtgactgagagaactaataatttttctcgctttttcaaaccacacaagcatgttactgatttataattaatgcaaattaaagctgtaagctatgtttttcacattaggagaaggctttgctatgttttatgactgcagcgaccttgtgtagtctagactcatttgtctctctttaatatttttaagtacatatctctaggattgtttgttaccatatgataaatatatgtgacagtacttttagaagcctttctttgtcataaagctcaagggaccctctccaaccaaaataaatttacatccctaggtaatcattcacattgaattagttatttaaggatattttatttactatgtaatgagttctaattaaattttaaatttttctgtgaatcttacattaagaagtaactcatgaagagaattctcaagaattttctagcctcttatatcaaatatttgtcacgttttaaaactaagaagacacacacacatatatacatacatatacatagaccaatagagatggctacaagtagttcgttagtatggatgaaactgcaaaagccatttgtgaagtaaaaaatgagataaaactttttcataaacttcagggtttttttttttttaatgtgcttgtccttttcctttcttttaatttttccttttattcttctctcctacaaacacacacaaacacatgcatacatgcatacacacacacacacacacacacacacacacacacacacatctttttttttttttggcctagttttgcatttcaacatgcctggttcaaactagctaaaaaacgtataatttactaacaccaaacaacaaaaacaaaggctaaagcaaaacaaaacctagagtttcccaataaggtttgaataataagttattttgcaatgaatattaaagcaatctgtaccttttaaaattcaaagttacttctttcattgaagaatgtagcccaagagatggctggcaggttcagtcactttctaatgcaaaaactacttgataaggaatctttttcacagcgattccctgatgcatctaatgtgtcaaaaataattaggttttggcagatttatcattgcagcacaaccctttccttgagtagggcctggatcatgttaagccttgtttgtgtgtgacagcattattattcttgttagttgcccacttccccatcactctgttgtgtttggaaatttcccacctcaacttgtgttcctcaccagtgtttttcttcatttattgacagttggtaaagatattcattaatttagagttaaaaacatgtatcatcatgtctcactgaactgctgggctgaagttgattaatggcaaagccttgctgcaggttttcctcttgagaagcagctcaccagccctgcatattaatgagagattgcggtttctattaacaagataaaaaagcaggtcttctcttgcttcaggggtaaaaaggatgaaaaaatttgattatttatatataatatttttccaccaggtgctatctgtgaaccttcatagcttttctcagatcacagtgacctttctaatatattttggggttggagggataataaacttcagcatatacgttgtttaagtttctataattatattgctgtggctggtgtattcatgacaaaattttaattacataatgtatgtacacacacaaacatataaatatgtgcttgaaaatagtactggcatcctggtaatgaagacgctttagccccagtttattttctgaaatttgttagtatttgtttcaacaaacacccaatctagcttatactggagagtttaaaaaaaaatagaaataaaattgttaaactcaagtctgaaatatttagaaataacagttgttatcttaattgctagcaaaaaactgaatggtaaaagtgttttcatttgttgattaattatttgtttactattcctttactaccactccttgattatcatgttataacaaatactaacaaaacatttcattcagaaagaatgctgagaaacggatgttttacctaaacagcacagcatgtgggagtccatgctcacattactctgaatccctgcctccacagttctactgtaaagtatcatgtctctaaccagctcacgcaatcaatcagatgcccttttaagaaagacaatgttgtgtcttcttgccttttttcagttgacctcttcactgcaaagttggccaaactctgtttgttgcttacttagtaaaattttggtgtatctacatgtttttcagacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaagggacatatcactgaccttcataaccactccacaaccatgaatatttgacaaatttttactgtgactatttattaagcatggataaaggagacagccctaaaggaacttactaagccagccctttgggattcagtaccaacaggcaaattgcttgttttcttcttcttcttcttctttttttttttttttttagaaaaaaagacaaaaactgattttcttgaaaaaaaaaaatgaactgttctttctataatggctttgcccatttaaaaaatgtggctcttaagggttcatgaaatgactgaatatgaggatacatgtcctgtagaaagcaaatgcgcctcatatactgccaaaaatagtgttagtttcattaatgtgaattttccagcattcagtagttgtaatgttagaaacaattgctggtcaagttcaacttgttgctattgtttttaatttgcacaggagtagtatcagaaattagtgtcactgcttgtatctagctgaattttaaacaacagaacattagttttttatgttggtgccaccaactgtaaatgacataagttagttattacaaaacacagtaattagactgttgcaaccatctaaaaccttaggcttccagtctgtgctgttagtgttaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacaaatatgacaaaaaccacaactaaaaaatgttaattcagtcacagagtaatcttctgaggccaaaagtccatctaaatgcaatgaagatttgctttcattaaagacagaggtgaggacaaaatccgcagtggaagttatgatatgctagaaagcaacaaatgtggatcactgaccaaaacgattatgtacttgatgcaaatgcagattgcatattgttatatat

SEQ ID NO: 2 - protein, 715 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 3 - DNA, 3573 bp, Homo sapiens
gattttgtgtacgattgtccacggacgccaaaacaatcacagagctgcttgatttgttttaattaccagcacaaaatgccatcagtctgggacgtgatcgggcagaggtgtactcacagtagtgtaaatactgctgtaaatagttgtctgatggtggctttgacagtgagctagcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaagggacatatcactgaccttcataaccactccacaaccatgaatatttgacaaatttttactgtgactatttattaagcatggataaaggagacagccctaaaggaacttactaagccagccctttgggattcagtaccaacaggcaaattgcttgttttcttcttcttcttcttctttttttttttttttttagaaaaaaagacaaaaactgattttcttgaaaaaaaaaaatgaactgttctttctataatggctttgcccatttaaaaaatgtggctcttaagggttcatgaaatgactgaatatgaggatacatgtcctgtagaaagcaaatgcgcctcatatactgccaaaaatagtgttagtttcattaatgtgaattttccagcattcagtagttgtaatgttagaaacaattgctggtcaagttcaacttgttgctattgtttttaatttgcacaggagtagtatcagaaattagtgtcactgcttgtatctagctgaattttaaacaacagaacattagttttttatgttggtgccaccaactgtaaatgacataagttagttattacaaaacacagtaattagactgttgcaaccatctaaaaccttaggcttccagtctgtgctgttagtgttaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacaaatatgacaaaaaccacaactaaaaaatgttaattcagtcacagagtaatcttctgaggccaaaagtccatctaaatgcaatgaagatttgctttcattaaagacagaggtgaggacaaaatc

SEQ ID NO: 4 - protein, 740 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQELLPETKLCICGHSSGDGHPHNTFAVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 5 - DNA, 3708 bp, Homo sapiens
cttgaacctttgtcacccctcacgttgcacaccaaagacataccctagtgattaaatgctgattttgtgtacgattgtccacggacgccaaaacaatcacagagctgcttgatttgttttaattaccagcacaaaatgccatcagtctgggacgtgatcgggcagaggtgtactcacagtagtgtaaatactgctgtaaatagttgtctgatggtggctttgacagtgagctagcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggaattgcttccagaaacaaaattatgtatctgtggccactcttctggtgatgggcatcctcacaacacatttgcagtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaagggacatatcactgaccttcataaccactccacaaccatgaatatttgacaaatttttactgtgactatttattaagcatggataaaggagacagccctaaaggaacttactaagccagccctttgggattcagtaccaacaggcaaattgcttgttttcttcttcttcttcttctttttttttttttttttagaaaaaaagacaaaaactgattttcttgaaaaaaaaaaatgaactgttctttctataatggctttgcccatttaaaaaatgtggctcttaagggttcatgaaatgactgaatatgaggatacatgtcctgtagaaagcaaatgcgcctcatatactgccaaaaatagtgttagtttcattaatgtgaattttccagcattcagtagttgtaatgttagaaacaattgctggtcaagttcaacttgttgctattgtttttaatttgcacaggagtagtatcagaaattagtgtcactgcttgtatctagctgaattttaaacaacagaacattagttttttatgttggtgccaccaactgtaaatgacataagttagttattacaaaacacagtaattagactgttgcaaccatctaaaaccttaggcttccagtctgtgctgttagtgttaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacaaatatgacaaaaaccacaactaaaaaatgttaattcagtcacagagtaatcttctgaggccaaaagtccatctaaatgcaatgaagatttgctttcattaaagacagaggtgaggacaaaatc

SEQ ID NO: 6 - protein, 623 aa, Homo sapiens
MMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 7 - DNA, 3691 bp, Homo sapiens
cttgaacctttgtcacccctcacgttgcacaccaaagacataccctagtgattaaatgctgattttgtgtacgattgtccacggacgccaaaacaatcacagagctgcttgatttgttttaattaccagcacaaaatgccatcagtctgggacgtgatcgggcagaggtgtactcacagtagtgtaaatactgctgtaaatagttgtctgatggtggctttgacagtgagctagcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcagccatgagacagacttgtggttcttcaacagctgtgcagagcagagacgtaaatttaaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaagggacatatcactgaccttcataaccactccacaaccatgaatatttgacaaatttttactgtgactatttattaagcatggataaaggagacagccctaaaggaacttactaagccagccctttgggattcagtaccaacaggcaaattgcttgttttcttcttcttcttcttctttttttttttttttttagaaaaaaagacaaaaactgattttcttgaaaaaaaaaaatgaactgttctttctataatggctttgcccatttaaaaaatgtggctcttaagggttcatgaaatgactgaatatgaggatacatgtcctgtagaaagcaaatgcgcctcatatactgccaaaaatagtgttagtttcattaatgtgaattttccagcattcagtagttgtaatgttagaaacaattgctggtcaagttcaacttgttgctattgtttttaatttgcacaggagtagtatcagaaattagtgtcactgcttgtatctagctgaattttaaacaacagaacattagttttttatgttggtgccaccaactgtaaatgacataagttagttattacaaaacacagtaattagactgttgcaaccatctaaaaccttaggcttccagtctgtgctgttagtgttaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacaaatatgacaaaaaccacaactaaaaaatgttaattcagtcacagagtaatcttctgaggccaaaagtccatctaaatgcaatgaagatttgctttcattaaagacagaggtgaggacaaaatc

SEQ ID NO: 8 - protein, 168 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQELLPETKLCICGHSSGDGHPHNTFAVPVSVAMMTPQVITPQQMQQILQQQVLSPQQLQALLQQQQAVMLQQVMWVTCFGVLA

SEQ ID NO: 9 - DNA, 955 bp, Homo sapiens
  misc_feature (26)..(26): n is a, c, g, or t
aagaattcggcacgagcagtgagctngcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggaattgcttccagaaacaaaattatgtatctgtggccactcttctggtgatgggcatcctcacaacacatttgcagtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcaggtaatgtgggttacctgctttggtgttctagcatgacttagaaggtgtgcacttattttgaaagtgcagtgggcatatttgacaattcagtctccatttgaaaggacaacctattagcgtgctagaacataaatgtaacattttaattatagaactcgtaagaaaatctagattgcttatttttttaaaaaaattattaaagtctagaataagagcagagagaggtgtaatttgaaaatggaatgagccaatatttaaatattcaaatgcttaggtacattcaatttgttgacttgccaaaaaaaaaaaaaaaaaaa

SEQ ID NO: 10 - protein, 381 aa, Homo sapiens
MMTPQVIIPQQMQQILQQQVLSPQQLQALLQQQQAVMLQQDFLDSGLENFRAALEKNQQLQEFYKKQQEQLHLQFLQQQQQQQQQQQQQQQQQQQQQQQQQQQQQQQQQQQQQQQHPGKQAKEQQQQQQQQQQLAAQQLAFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPNICKVTYRCIWMFLRFSNYAEVLLYIPEKLTPVAL

SEQ ID NO: 11 - DNA, 8323 bp, Homo sapiens
ggccgggggccgccgcgggtgccacctcccgggacgctgcccacggcgtccccggtcgcgatcaaacaaataatagaagtctgttgaagctttgtcctgacctatcctggaagtagaagaatactctggagaaaggagcattgtaagcacaaagaataagattcctcaatcctgagagagagaattaaagtctaactctccaaattttcaacaggagttttgagtaaagaaaatggagaagtttgtgggagagaaaggtctactctaatgtgatgctgggattgaattttcctaggactccgtttcaaggttaattcgaaagtcttgtggaagatctggaagaaacagccagtactcatatatatagaaagaggagttggatgtttgtcaataaactcatctggctttaaataaatcaaaacaaaaagaataatttgctactagtcttccaacatgtttgaagaacagcctgctgtttggggaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcagccatgagacagacttgtggttcttcaacagctgtgcagagcagagacgtaaatttaaggtgcctgtgtcagtggccatgatgactccccaggtgatcatccctcagcaaatgcagcagatccttcagcaacaagtcctgtctcctcagcagctacaagcccttctccaacaacagcaggctgtcatgctgcagcaggattttttggattctggattggaaaatttcagagctgccttggaaaaaaatcaacaactacaagagttttacaagaaacagcaagagcagttacatcttcagtttttgcagcagcagcagcaacagcagcagcagcaacaacagcagcaacaacagcagcagcaacaacaacaacaacagcagcaacaacagcagcagcagcagcaacagcagcagcagcaacagcatcctggaaagcaagcgaaagagcagcagcagcagcagcagcagcaacagcaattggcagcccagcagcttgccttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctaatatttgcaaagtcacatacagatgcatatggatgtttctgcgtttctccaactatgcagaggtgctcttgtacatcccagagaagcttacaccagtggctttgtaaatgtattaaatgcagcatttaggagcattatctcagctttcatttatctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatgtaagtgtggttaacagactctctaaagggaagaatctatattaatatgcttcttaaatttagaatttttctgaagtttttactgttgtataagttgaaaatacaaatttaagtggagttaaattataccacgttcattatatgacaaagtttatcaaaatcggttttaatacattttttagatgatctcaaatgccatatcattattgttccagcagtcctctacagattcttagtactactaatgttctcttgggcataaagcagtgtctaagatgaatctacatcttatgggtcattatcacaggttggtttgtatggtgcaataagaatattcatggtggaataagttgttataatttttactactgttacaggtctctaaaccatttatgaactgtagtcaagatattgcccgtttttataaaccgtaaaatgcaaagtttaactgttacccacagtttacatagaagctagcaaagtgtaactttttgcctgtcttataaatcagtgtaattatatattcaactttaattactggtgcgtctgtattggcaaggtttataagttatattttatgcaagactacgattcctattaatatttagcagctaactatatcattaattactctaaaatctttcccctaccatctttttaatggagctgagagagtagaagtattaaaaattgatgtattgtggatttcagaaatgtgtatgacactttaaaaaatgcctggcttttttcaatagtgtcaagcatattagtgtcttgtttactaaaatgtattgaccgtggtagaactcagaattcagaggaaaaaatttgaatctgaataatgaagtgtgtacaccatttagctgtcaggatgagttgtaactcatgtctatacttcctagaattaaacgtcacatacttctaattacaagttattgaggtaagattagtcctttctcttatttttatattgtcttcctttatttttccattcaatatctagtaaaaggtctgaaagagaaatatactctttatatgtacccattttcacttgacctttcacagggtattatttttgttgtctttttaaacattttattctgtttgacatattaatgtctttaagagcatatgtctaattgctttatagcacttttactctatcacctttgcaaaaaaacacactgtgaacattaatgtgttttatcaggttccgaaccattacttcttcaaaatggaaaaatttgtcttttggaagtgtctttagatgtattaaataatacaaaagaagtaataaaactcaaagtagaaagaacatgaatatatgctcttgagagtagcagtattctaaaacaaggaaagtgagcaatcagacaaaagtttttatatccccgctttatcctatcagccatgcagtgggtagcttgcggccttttactttcagcagttctcctgagtatgctaaatagtttattaaacattatatttcagaacctgaaataataattgggctaaattgtagcattgttagatgggtattggttttcgtttggcaattgctacttcagatttttcattttattattaaagtttatgaaatatttcagagaaatattagtatgtattagataacacctaaaaattatacatataatgtccagtatattgctgctatgggtttataaagttaaaatgttcatagttaatatataacacattttatgcttatatgaattagtgcatttgaaaaatacttatgtttattttccatagataaaaggaaagcttgttgaaaatagatttcagaaaaaataaaaattaaaaggggtgtaaattgtccctttttctaagttacatcaattataatcaaaattttaattatcttgcagaatattgagtcaatattcattcttaaatctgtttctgctgctgtcattaggtttttgcataatagatgtcaaatatttatgaaatatttcagtattctctatcttcacctttataaagttctaagatgcttgtacttaaaccatgttatgtattttattaggcaaaggaagaaatattgatataattctctcagtcaaaatttggttgtatataattgagtgcatttcaaaatactttggaaattaaacagaagtgaaataatttttataagacatttacacagcatacttatatctcctaaaatgaagtcatgttacttgttgttttagaggggggtagttgtgctgatgcaattgtaaacaagaggaaatagggcctagtggtatcagaacataggttgtaatgccagactgtcagggttccaattccagattcaatagttaatagccaagtgggattgagcacgttacttaatatttctttcctcagctttctcagctgaaaatgaggtaagtaataatataatgcttcttatttcataagattcttgcaaagaaatgagtgcctggaacataataattgctccttgtgtctctcatagtgctgtgtaaataggtactgatttatattacttcctgtaatacctcactgttactaaaaatgagtctataaacaatatgaaaatatttttgtattttgcattttaaggcactctgtaagtttgtggttagtcattgacaatccattttcttctccttctttcttctgttataattcataaagtaagcagatccataaaactgaaacaacatgaggccaggcatggtggcttatgccagtaatcccagcattttgggaggggagggcaggaggattgcttgaggccaggagttcaagaccagcctggacaacatagtgagaccctgtatctacaaaaataattttaaaaattagttgggtgtggtgtgtgcctgtatacagtcctagctactcaggaggctcaggcaggaggaaagcttgagcctagggatttcgaggttacagtaagctatgatcatggcatggaactccagcctgcaccacagagtgagactctatctgtctctaaaaataaaaagtgaaacaatatgacacaaggacttattataggtagcacattacctatatttaggtatctgaagttacctagttatctgaagttttaggcaatgaacaactcaaatagaacaacaccaacccagaatcagggacatatcatatatgtcttgaaaacattgagtattagttttgaaaggaatttagaatacataagggaatacagaatttagaatataggaagtgggaccagactgctgagtgacttaaatatcaagctatggtgtttgtttcagtcagtatacagcttgttactgttatcagcttgactttggcacgatcagagctatgcattagaaacagtaatctaaaagtgtactgaatgaaccatgacttaatgaaactcatgtataggagaatattctgaaattgggagagactggaagcagaaaattaattcttgcagtatgtaatatctaacataaatacaagactaggagtggagagcaaagaatccgtataagaagtgctgtgaaaagaaatactggaacaagacaaccaactgaatgctagggacaaagatgaaaattgggaaaattcatagccactttaaaagtctgcaaagagaaatagaggacacaagaagcaggttcaaaagggaagatgaagaattcagcattttacttggggaatccaagaacctaaatatcattagattcaggagaaaacactgatgatttttttgattagtcttttaattcaactccattatagtaaaattctacgtggtagaatatgaatatatacctgaggttgatagagaagatgttttttgaagcattagaagagtgaaatagatatggtctggtgcttcttataaattgtggagaacaagagatgccaaattatcatttatattccatactccatattttaacccttttcttccactccagctttcgccttattcagtgtcctgaataaaggcctcttccaaattaatgcatttatttcgctcacactatttgaatttctgccctttcccttttctggttcccctctactctcttacctaaattctacctttccttacaggactagtcctatttttttccaagagggtttcccaaggaattttactatcaggaccatgcactctgctccacacttctaaagagcttgcttcttataccactattttggactaatactttgtgtacatgtgtgagtgtgtgtgcacacacacatgcacgtgtgtttatgcatacatctgtgttcattttgcagatatccctaccttaatgacaagtcccttaaagacagattatgttttagacaaattatagtataagaaacccctagcacattctactatgtcaagtaagcattcagtaaaagtatgatactgattatgaagaatcaaattggagattacagttaattagaaatggtttgacttcacaaagtcatacttacaggagtataaaatccagggttctcctagaatcttgaagaaggagaacaaaagagattttaggaagaacagatgagtaaatataatgttcatttttatgtgggagctaggaaaattttctaaatattggtagactatactttttaagttttatttttagttttaattatttaattaatatgtattttataaccattccaatttacgtgagtaaaaagtggctttccactttgagtagaatgaggccccttcacctcattcatgattatcatctcaaccttgttaaattttatttacattgttgaaagattgaaaacaattatcaaaatttaggcaatataaatcagatgaatgtttaaggggatttcagcaaaaagtaggaaaggttaaagccaggcgacattgtatagattctacataataattttttgaaacttctttgactgttttgactcaactacttattgattcaaagcaagtacttcacttttaaaacatttttattttctattgctgttcaaagagtcctagaaacatccatggtatgaataaaggaaaacgatactttaagtgtccgagctgagtatcattacaatttataatattgttcccttatgcacctgaatgccaatattccctttttcagagttatatagaatatgaattaaaaatgactagcttcctattttatatagacatttacctctagaaatgtttaaaaggttaggattgatgctgtctttaagcagaaataatttgaacttactgtcttctctcatgtgttggacttaagtgaatgacaagagaagaaaacatgtagactagacttggaagatgaacgcatactgatacaatcaatccatttcctgtctacttcccaacctaaggcaagaagtggaatttaatgatgtacccaccatgtttagttctactcagttgtttctttttgtattaagtaattgtttctcctcattctcaataagttggttcagatttgctgttcttcctcgtataaaatgttaaatgagattcccatttttatattttcaaatgttgtgtctagagatcaaaataaatattcttccctaaaaaaaaaaaaaaaaaaa

SEQ ID NO: 12 - protein, 381 aa, Homo sapiens; identical to SEQ ID NO: 10

SEQ ID NO: 13 - DNA, 1593 bp, Homo sapiens
atgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctgctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatga

SEQ ID NO: 14 - protein, 623 aa, Homo sapiens; identical to SEQ ID NO: 6

SEQ ID NO: 15 - DNA, 2285 bp, Homo sapiens
ttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcagccatgagacagacttgtggttcttcaacagctgtgcagagcagagacgtaaatttaaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaaggg

SEQ ID NO: 16 - protein, 715 aa, Homo sapiens; identical to SEQ ID NO: 2

SEQ ID NO: 17 - DNA, 2472 bp, Homo sapiens
agtgagctagcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaagggacatatcactgaccttcataaccactccacaaccatgaatatttgacaaatttttactgtgactatttattaagcatggataaaggagacagccctaaaggaacttactaagccagccctttgggattcagtaccaacaggca

SEQ ID NO: 18 - protein, 95 aa, Homo sapiens
ASHTLYGHGVCKWPGCESICEDFGQFLKHLNNEHALDDRSTAQCRVQMQVVQQLEIQLSKERERLQAMMTHLHMRPLEPKPSPKPVSAYCFINSK

SEQ ID NO: 19 - DNA, 391 bp, Homo sapiens
gggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgacccttagagcccaaaccatctcccaaacctgtaagtgcatattgctttataaacagtaaatagctctaccaatgtaacagactaagaaaatgaacaatttagtgacagttagaaaacaatgagtgtgatgaaaaatacggcaataaaatgaaagtaaaatgtaa

SEQ ID NO: 20 - protein, 174 aa, Homo sapiens
AGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPVSAYCFINSK

SEQ ID NO: 21 - DNA, 627 bp, Homo sapiens
agctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctgtaagtgcatattgctttataaacagtaaatagctctaccaatgtaacagactaagaaaatgaacaatttagtgacagttagaaaacaatgagtgtgatgaaaaatacggcaataaaatgaaagtaaaatgtaa

SEQ ID NO: 22 - protein, 95 aa, Homo sapiens
ASHTLYGHGVCKWPGCESICEDFGQFLKHLNNEHALDDRSTAQCRVQMQVVQQLEIQLSKERERLQAMMTHLHMRPSEPKPSPKPVSAYCFINSK

SEQ ID NO: 23 - DNA, 395 bp, Homo sapiens
gggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctgtaagtgcatattgctttataaacagtaaatagctctaccaatgtaacagactaagaaaatgaacaatttagtgacagttagaaaacaatgagtgtgatgaaaaatacggcaataaaatgaaagtaaaatgtaaaaag

SEQ ID NO: 24 - protein, 732 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQDFLDSGLENFRAALEKNQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 25 - DNA, 2807 bp, Homo sapiens
aattaccagcacaaaatgccatcagtctgggacgtgatcgggcagaggtgtactcacagtagtgtaaatactgctgtaaatagttgtctgatggtggctttgacagtgagctagcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcaggattttttggattctggattggaaaatttcagagctgccttggaaaaaaatcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaagggacatatcactgaccttcataaccactccacaaccatgaatatttgacaaatttttactgtgactatttattaagcatggataaaggagacagccctaaaggaacttactaagccagccctttgggattcagtaccaacaggcaaattgcttgttttcttcttcttcttcttctttttttttttttttttaggaaaaaagacaaaaactgattttcttgaaaaaaaaaaatgaactgttctttctataatggctttgcccatttaaaaaatgtggctcttaagggttcatgaaatgactgaatatgaggatacatgtcctgtag

SEQ ID NO: 26 - protein, 456 aa, Homo sapiens
MQESATETISNSSMNQNGMSTLSSQLDAGSRDGRSSGDTSSEVSTVELLHLQQQQALQAARQLLLQQQTSGLKSPKSSDKQRPLQELLPETKLCICGHSSGDGHPHNTFAVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPVSAYCFINSK

SEQ ID NO: 27 - DNA, 1368 bp, Homo sapiens
atgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctacatctgcaacaacagcaggctctccaggcagcaagacaacttcttttacagcagcaaacaagtggattgaaatctcctaagagcagtgataaacagagaccactgcaggaattgcttccagaaacaaaattatgtatctgtggccactcttctggtgatgggcatcctcacaacacatttgcagtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagcctccagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctgtaagtgcatattgctttataaacagtaaa

SEQ ID NO: 28 - protein, 431 aa, Homo sapiens
  misc_feature (327)..(327): Xaa can be any naturally occurring amino acid
MQESATETISYSSMNQNGMSTLSSQLDAGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARXDSSSHEETGASHTLYGHGVCKWPGCESICEDFGQFLKHLNNEHALDDRSTAQCRVQMQVVQQLEIQLSKERERLQAMMTHLHMRPSEPKPSPKPVSAYCFINSK

SEQ ID NO: 29 - DNA, 1293 bp, Homo sapiens
atgcaggaatctgcgacagagacaataagctacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcagcaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacragacagctcgtcacatgaggagactggggcctctcacacyctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctgtaagtgcatattgctttataaacagtaaa

SEQ ID NO: 30 - protein, 415 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSE

SEQ ID NO: 31 - DNA, 1257 bp, Homo sapiens
aggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagag

SEQ ID NO: 32 - protein, 222 aa, Homo sapiens
MMQESATETISNSSMNQNGMSTLSSQLDVGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLL

SEQ ID NO: 33 - DNA, 766 bp, Homo sapiens
gtctactctaatgtgatgctgggattgaattttcctaggactccgtttcaaggttaattcgaaagtcttgtggaagatctggaagaaacaggtattaagtcatgatgcaggaatctgcgacagagacaataagcaacagttcaatgaatcaaaatggaatgagcactctaagcagccaattagatgttggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttct

SEQ ID NO: 34 - protein, 149 aa, Homo sapiens
MMTPQVITPQQMQQILQQQVLSPQQLQALLQQQQAVMLQQDFLDSGLENFRAALEKNSKWFIQQQLQEFYKKQQEQLHLQLLQQQQQQQQQQQQQQQQQQQQQQQQQQQQQQQQQQQQQQQQHPGKQAKEQQQQQQQQQQLAAQQLVFQ

SEQ ID NO: 35 - DNA, 1509 bp, Homo sapiens
cggcggccgcgtccgctggctgcggcttcctcggccccccccctccccgggcgcgccccccactcgcggcagcagctgcccggactcgcgcgtgggtgtgttgtttgggggcttctgcctcgccgccgcgggtgccacctcccgggacgctgcccacggcgtccccggtcgcgatcaaacaaataatagaagtctgttgaagctttgtcctgacctatcctggaagtagaagaatactctggagaaaggagcattgtaagcacaaagaataagattcctcaatcctgagagagagaattaaagtctaactctccaaatgtctactctaatgtgatgctgggattgaattttcctaggactccgtttcaaggttaattcgaaagtcttgtggaagatctggaagaaacaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctggagtgcagtggtgcaatcataactcactgcaacctggaactcctgggctcaagcagtcctccgacctcagcctctgaagtagctaggactacaggaggatgttgtctcttacacccaggttatttgttaaagtctttggatctatacttaactgatgacctagaagagcgattctcaaaagtgtgagtcctggaccagcagcaacagcatcaccgggaacttgttagaaatgcacattctcaggccactccctagacctactgaatcagaaaccctggggttggagctcagcaattaccctgcaggctctccaggcagcaagacaacttcttttacagcagcaaacaagtggattgaaatctcctaagagcagtgataaacagagaccactgcagccatgagacagacttgtggttcttcaacagctgtgcagagcagagacgtaaatttaaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcaggattttttggattctggattggaaaatttcagagctgccttggaaaaaaattcaaaatggtttattcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccag

SEQ ID NO: 36 - DNA, 558 bp, Homo sapiens
ttttcctgcccttctcttgggcctttgcagctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaaaaaa

SEQ ID NO: 37 - protein, 740 aa, Homo sapiens; identical to SEQ ID NO: 4

SEQ ID NO: 38 - DNA, 2594 bp, Homo sapiens
gcttgaaccttgtcacccctcacgtgcacaccaaagacataccctagtgattaaatgctgatttgtgtacgatgtccacggacgccaaaacaatcacagagctgcttgattgttttaattatccagcacaaaatgccatcagtctgggacgtgatcgggcagaggtgtactcacagtagtgtaaatactgctgtaaatagtgtctgatggtggcttgacagtgagctagcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctacatctgcaacaacagcaggctctccaggcagcaagacaacttcttttacagcagcaaacaagtggattgaaatctcctaagagcagtgataaacagagaccactgcaggaattgcttccagaaacaaaattatgtatctgtggccactcttctggtgatgggcatcctcacaacacatttgcagtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgaga

SEQ ID NO: 39 - RNA, 25 nt, Artificial
  siRNA sequence
gacaggcaguuaacacuuaaugaaa

SEQ ID NO: 40 - RNA, 25 nt, Artificial
  siRNA sequence
uuucauuaaguguuaacugccuguc

SEQ ID NO: 41 - RNA, 25 nt, Artificial
  siRNA sequence
caguuuaggcuauggagcagcucuu

SEQ ID NO: 42 - RNA, 25 nt, Artificial
  siRNA sequence
aagagcugcuccauagccuaaacug

SEQ ID NO: 43 - RNA, 25 nt, Artificial
  siRNA sequence
cagagagauugaagaagagccuuua

SEQ ID NO: 44 - RNA, 25 nt, Artificial
  siRNA sequence
uaaaggcucuucuucaaucucucug

SEQ ID NO: 45 - DNA, 21 bp, Artificial
  oligonucleotide
atggaagacaatggcattaaa